(19) United States
(12) Patent Application Publication
(10) Pub. No.: US 2025/0127757 A1
(43) Pub. Date: Apr. 24, 2025

(54) COMPOSITIONS COMPRISING MDMA AND METHODS OF USING SAME

(71) Applicant: LYKOS THERAPEUTICS, INC., San Jose, CA (US)

(72) Inventors: Jay Nair, Mullica Hill, NJ (US); Berra Yazar-Klosinski, Freedom, CA (US); Julian Northen, South Shields, Tyne and Wear (GB); Arul Balasundaram, Maidstone (GB); Linda Hakes, Leichlingen (DE); John Briston, Birchington (GB)

(21) Appl. No.: 19/005,427

(22) Filed: Dec. 30, 2024

Related U.S. Application Data
(63) Continuation of application No. 18/611,564, filed on Mar. 20, 2024.
Application No. 18/611,564 is a continuation of application No. PCT/US2023/082367, filed on Dec. 4, 2023.
(60) Provisional application No. 63/463,170, filed on May 1, 2023.
(60) Provisional application No. 63/463,169, filed on May 1, 2023.
(60) Provisional application No. 63/449,928, filed on Mar. 3, 2023.
(60) Provisional application No. 63/430,287, filed on Dec. 5, 2022.

(51) Int. Cl. A61K 31/36; A61K 9/48; A61P 25/18

(52) U.S. Cl. CPC A61K 31/36; A61K 9/4816; A61K 9/4858; A61P 25/18

(57) ABSTRACT

The present disclosure relates to 3,4-methylenedioxymethamphetamine (MDMA) or a pharmaceutically acceptable salt and/or solvate thereof, pharmaceutical compositions and dosage forms containing these particles, and methods of using pharmaceutical compositions and dosage forms containing these particles to treat subjects suffering from disorders of the central nervous system.

### CLAIM OF PRIORITY

This application is a continuation of U.S. patent application Ser. No. 18/611,564, filed on Mar. 20, 2024, which is a continuation of PCT Application No. PCT/US2023/082367 filed on Dec. 4, 2023, which claims priority to U.S. Provisional Application No. 63/430,287 filed on Dec. 5, 2022; U.S. Provisional Application No. 63/449,928 filed on Mar. 3, 2023; U.S. Provisional Application No. 63/463,169 filed on May 1, 2023; U.S. Provisional Application No. 63/463,170 filed on May 1, 2023; the entire contents of all of which are hereby incorporated by reference.

### FIELD

The present disclosure relates to 3,4-methylenedioxymethamphetamine (MDMA) or a pharmaceutically acceptable salt and/or solvate thereof, pharmaceutical compositions and dosage forms containing these particles, and methods of using pharmaceutical compositions and dosage forms containing these particles to treat subjects suffering from disorders of the central nervous system (CNS)

### BACKGROUND

Central nervous system (CNS) disorders can have a devastating impact on the afflicted individuals, their families, and society at large. These disorders can be challenging to treat, as the therapies often have significant undesired side effects. As such, novel treatments for such disorders are needed.

Eating disorders (EDs) and post-traumatic stress disorder (PTSD) are psychiatric disorders that can have a devastating impact on the afflicted individuals, their families, and society at large. Significantly higher rates of PTSD or PTSD symptoms have been associated with EDs and ED symptoms, and vice versa. EDs and PTSD also share several common risk factors that may contribute their co-occurrence. Both disorders are associated with high degrees of morbidity and mortality, including suicide and self-harm.

EDs are characterized by severe disturbances in eating behavior and body weight (1) and frequently occur in adolescents, often leading to multiple psychiatric and somatic complications as well as having a significant negative impact on quality of life, and even mortality (2, 3). See, e.g., Schmidt, et al., Lancet Psychiatry, 2016; 3:313-15 and Pasold et al., Clin Child Psychol Psychiatry. 2014; 19:299-312. Indeed, individuals with EDs have significantly elevated mortality rates than the same age cohort in the general population. See, e.g., Crow et al., Am J Psychiatry. 2009; 166:1342-6.

These disorders can be challenging to treat, as many existing therapies exhibit low efficacy and often have significant undesired side effects. As such, novel treatments for these disorders are needed.

MDMA has been studied in late stage clinical trials for the treatment of subjects with post-traumatic stress disorder (PTSD). Earlier-stage clinical trials exploring its efficacy in treating a variety of disorders are ongoing.

MDMA has multiple solid-state forms, including nhydrous MDMA hydrochloride (MDMA HCl) and a MDMA hydrochloride hydrate (MDMA·HCl hydrate) that readily forms under ambient conditions, particularly during grinding and in high-humidity environments. In addition, the free base of MDMA is an oil at room temperature. This results in significant difficulty in preparing consistent, stable, safe, and effective dosage forms of MDMA. As such, novel MDMA formulations are needed.

### SUMMARY

The present disclosure is based, in part, on surprising and unexpected discoveries related to particle size in formulations of MDMA and use of such formulations in therapy.

Some embodiments provide a composition comprising MDMA, or a pharmaceutically acceptable salt and/or solvate thereof, and one or more pharmaceutically acceptable excipients, wherein the average particle size of the MDMA is from about 50 μm to about 400 μm.

Some embodiments provide a composition comprising MDMA, or a pharmaceutically acceptable salt and/or solvate thereof, and one or more pharmaceutically acceptable excipients, wherein the average particle size of the MDMA is from about 50 μm to about 600 μm.

Some embodiments provide a composition comprising MDMA, or a pharmaceutically acceptable salt and/or solvate thereof, and one or more pharmaceutically acceptable excipients, wherein the average particle size of the MDMA is about 420 μm.

Some embodiments provide a composition comprising MDMA HCl and one or more pharmaceutically acceptable excipients, wherein the average particle size of the MDMA HCl is from about 50 μm to about 400 μm.

Some embodiments provide a composition comprising MDMA HCl and one or more pharmaceutically acceptable excipients, wherein the average particle size of the MDMA HCl is from about 50 μm to about 600 μm.

Some embodiments provide a composition comprising MDMA HCl and one or more pharmaceutically acceptable excipients, wherein the average particle size of the MDMA HCl is about 420 μm.

Some embodiments provide a dosage form comprising a composition comprising MDMA, or a pharmaceutically acceptable salt and/or solvate thereof, and one or more pharmaceutically acceptable excipients, wherein the average particle size of the MDMA is from about 50 μm to about 400 μm, and optionally one or more additional pharmaceutically acceptable excipients.

Some embodiments provide a dosage form comprising a composition comprising MDMA, or a pharmaceutically acceptable salt and/or solvate thereof, and one or more pharmaceutically acceptable excipients, wherein the average particle size of the MDMA is from about 50 μm to about 600 μm, and optionally one or more additional pharmaceutically acceptable excipients.

Some embodiments provide a dosage form comprising a composition comprising MDMA, or a pharmaceutically acceptable salt and/or solvate thereof, and one or more pharmaceutically acceptable excipients, wherein the average particle size of the MDMA is about 420 μm, and optionally one or more additional pharmaceutically acceptable excipients.

Some embodiments provide a dosage form comprising a composition comprising MDMA HCl and one or more pharmaceutically acceptable excipients, wherein the average particle size of the MDMA HCl is from about 50 μm to about 400 μm, and optionally one or more additional pharmaceutically acceptable excipients.

Some embodiments provide a dosage form comprising a composition comprising MDMA HCl and one or more pharmaceutically acceptable excipients, wherein the average particle size of the MDMA HCl is from about 50 μm to about 600 μm, and optionally one or more additional pharmaceutically acceptable excipients.

Some embodiments provide a dosage form comprising a composition comprising MDMA HCl and one or more pharmaceutically acceptable excipients, wherein the average particle size of the MDMA HCl is about 420 μm, and optionally one or more additional pharmaceutically acceptable excipients.

Some embodiments provide a method of treating PTSD a subject in need thereof, comprising administering to the subject a dosage form as described herein (i.e., comprising MDMA or a pharmaceutically acceptable salt and/or solvate thereof and one or more pharmaceutically acceptable excipients).

Some embodiments provide a method of treating an eating disorder a subject in need thereof, comprising administering to the subject a dosage form as described herein (i.e., comprising MDMA or a pharmaceutically acceptable salt and/or solvate thereof and one or more pharmaceutically acceptable excipients).

In any of the methods described herein, the subject being treated by administration of particles comprising crystalline MDMA, or a pharmaceutically acceptable salt and/or solvate thereof, during one or more psychotherapy sessions may suffer from one or more symptoms of disordered eating.

In any of the methods described herein, the subject being treated by administration of particles comprising crystalline MDMA, or a pharmaceutically acceptable salt and/or solvate thereof, during one or more psychological interventions may suffer from one or more symptoms of disordered eating.

In any of the methods described herein, the subject being treated by administration of particles comprising crystalline MDMA, or a pharmaceutically acceptable salt and/or solvate thereof, during one or more medication sessions may suffer from one or more symptoms of disordered eating.

All publications, patents, patent applications, publications, and information available on the internet and mentioned in this specification are herein incorporated by reference (including all figures, drawings, and supplementary material) to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### BRIEF DESCRIPTION OF THE DRAWINGS

FIG. 1 shows exemplary coarse MDMA hydrochloride particles isolated from the synthetic process.

FIG. 2 shows exemplary particles comprising MDMA after milling.

FIG. 3 shows the particle size distribution (PSD) of the milled particles of FIG. 2.

FIG. 4 shows an HPLC chromatogram for coarse MDMA particles isolated from the synthetic process.

FIG. 5 shows the XRPD spectra of MDMA·HCl monohydrate (5A), MDMA Form III (5B), and MDMA Form II (5C).

FIG. 6 shows the schedule of dosing and medication sessions for MDMA, in which the doses are expressed on a free base basis of MDMA.

FIG. 7 shows an integrated forest plot of treatment effect for the MAPP1 and MAPP2 clinical trials.

FIG. 8 shows the results of forced degradation studies on MDMA·HCl.

FIG. 9 shows the chemical stability of MDMA·HCl upon storage of API for up to 35 months at 25 degrees celsius at 60% relative humidity (RH)

FIG. 10 shows the chemical stability of MDMA·HCl upon storage of API for up to 60 months at 25 degrees celsius at 60% relative humidity (RH).

FIG. 11 shows the chemical stability of MDMA·HCl upon storage of API for up to 12 months at 40 degrees celsius at 75% relative humidity (RH).

FIG. 12 shows the MDMA Drug Product Manufacturing Process Flow Diagram.

FIG. 13 shows dissolution profiles of the drug product from clinical batches M-1 and M-2 at pH 1.2, 4.5 and 6.8.

FIG. 14 shows Correlations Between Continuous Covariates. BALB=baseline albumin; BALP=baseline alkaline phosphatase; BALT=baseline alanine aminotransferase; BAST=baseline aspartate aminotransferase; BBILI=baseline bilirubin; BBMI=baseline body mass index; BBSA=baseline body surface area; BCRCL=baseline creatinine clearance; BEGFR=baseline estimated glomerular filtration rate; BLBW=baseline lean body weight; BSCR=baseline serum creatinine; BWT=baseline body weight.

FIG. 15 shows MDMA Concentration versus Time by Dose Level (Semilog scale). Finer lines are subject-level profiles. For Example 8B, fasted and fed concentration data for each subject are included on the same plot. Thick lines show a LOESS fit of the data.

FIG. 16 shows MDA Concentration versus Time by Dose Level (Semilog scale). Finer lines are subject-level profiles. For Example 8B, fasted and fed concentration data for each subject are included on the same plot. The two thick lines show a LOESS fit of the data.

FIG. 17 shows MDMA Concentration versus Time Stratified by Fed/Fasted State for the Example 8B Study. Thin lines are subject-level profiles colored by subject fasting status. The two thick lines correspond to a LOESS fit of the data

FIG. 18 shows MDMA (FIG. 18A) and MDA (FIG. 18B) Concentration versus Time Stratified by CYP2D6 Metabolizer Status in the Example 8B Study. Thin lines are subject-level profiles. Fasted and fed concentration data for each subject are included on the same plot. The two thick lines correspond to a LOESS fit of the data. All subjects received 120 mg MDMA·HCl.

FIG. 19 shows the model structure for the two stage PPK model. Modeling was performed in two stages. MDMA was modeled in the first stage (top, right). Individual post hoc data from the first stage was used to model MDA in the second stage (bottom two boxes). Abbreviations: ALAG=absorption lag time; CL/F=apparent central clearance of MDMA; CLM=apparent clearance of MDA; D1=duration of zero order input; fmet=fraction of MDMA metabolized to MDA; ka=absorption rate; QM=peripheral clearance of MDA; V2/F=apparent central volume of MDMA; V3=apparent central clearance of MDA; V4=peripheral clearance of MDA.

FIG. 20 shows GOF Plots for the Base Model of MDMA for All Data. Dots are individual data points, and solid lines are smoothed LOESS lines. In the two plots in the upper row, left and center, dashed lines are lines of identity. In the plot on the top right, and the plots on the bottom left and center, dashed lines show the boundaries of the CWRES±5 interval. One outlier data point was observed at 148 hours after dose and excluded from the plot. Abbreviations: CWRES=conditional weighted residuals; GOF=goodness-of-fit; LOESS=locally weighted scatterplot smoothing.

FIG. 21 shows GOF Plots for the Base Mpodel of MDMA for Example 8B Study. Dots are individual data points, and solid lines are smoothed LOESS lines. In the two plots in the upper row, left and center, dashed lines are lines of identity. In the plot on the top right, and the plots on the bottom left and center, dashed lines show the boundaries of the CWRES±5 interval. One outlier data point was observed at 148 hours after dose in the Example 8A dataset and excluded from the plot. Abbreviations: CWRES=conditional weighted residuals; GOF=goodness-of-fit; LOESS=locally weighted scatterplot smoothing.

FIG. 22 shows GOF Plots for the Base Model of MDA for All Data. Dots are individual data points, and solid lines are smoothed LOESS lines. In the two plots in the upper row, left and center, dashed lines are lines of identity. In the plot on the top right, and the plots on the bottom left and center, dashed lines show the boundaries of the CWRES±5 interval. One outlier data point was observed at 148 hours after dose and excluded from the plot. Abbreviations: CWRES=conditional weighted residuals; GOF=goodness-of-fit; LOESS=locally weighted scatterplot smoothing.

FIG. 23 shows GOF Plots for the Base Model of MDA for MPFK Study. Dots are individual data points, and solid lines are smoothed LOESS lines. In the two plots in the upper row, left and center, dashed lines are lines of identity. In the plot on the top right, and the plots on the bottom left and center, dashed lines show the boundaries of the CWRES±5 interval. One outlier data point was observed at 148 hours after dose in the Example 8A dataset and excluded from the plot. Abbreviations: CWRES=conditional weighted residuals; GOF=goodness-of-fit; LOESS=locally weighted scatterplot smoothing.

FIG. 24 shows GOF Plots for the Final Model of MDMA for All Data. Dots are individual data points, and solid lines are smoothed LOESS lines. In the two plots in the upper row, left and center, dashed lines are lines of identity. In the plot on the top right, and the plots on the bottom left and center, dashed lines show the boundaries of the CWRES±5 interval. Abbreviations: CWRES=conditional weighted residuals; GOF=goodness-of-fit; LOESS=locally weighted scatterplot smoothing.

FIG. 25 shows GOF Plots for the Final Model of MDMA for MPKF Study. Dots are individual data points, and solid lines are smoothed LOESS lines. In the two plots in the upper row, left and center, dashed lines are lines of identity. In the plot on the top right, and the plots on the bottom left and center, dashed lines show the boundaries of the CWRES±5 interval. Abbreviations: CWRES=conditional weighted residuals; GOF=goodness-of-fit; LOESS=locally weighted scatterplot smoothing.

FIG. 26 shows GOF Plots for the Final Model of MDA for All Data. Dots are individual data points, and solid lines are smoothed LOESS lines. In the two plots in the upper row, left and center, dashed lines are lines of identity. In the plot on the top right, and the plots on the bottom left and center, dashed lines show the boundaries of the CWRES±5 interval. One outlier data point was observed at 148 hours after dose and excluded from the plot.

FIG. 27 shows GOF Plots for the Final Model of MDA for MPFK Study. Dots are individual data points, and solid lines are smoothed LOESS lines. In the two plots in the upper row, left and center, dashed lines are lines of identity. In the plot on the top right, and the plots on the bottom left and center, dashed lines show the boundaries of the CWRES±5 interval. Abbreviations: CWRES=conditional weighted residuals; GOF=goodness-of-fit; LOESS=locally weighted scatterplot smoothing.

FIG. 28 shows the prediction-corrected VPC for MDMA. Dots are observed data points; indicated solid lines are the observed median; dashed lines are observed p5 and p95. Indicated shaded areas are the 95% PI of the simulated median, and the indicated middle shaded areas is the 95% PI of the simulated p5 and p95. Abbreviations: CI=confidence interval; p5=5th percentile; p95=95th percentile; PI=prediction interval; VPC=visual predictive check.

FIG. 29 shows the prediction-corrected VPC for MDA. Dots are observed data points; indicated solid lines are the observed median; dashed lines are observed p5 and p95. Indicated shaded areas are the 95% PI of the simulated median, and the indicated middle shaded areas is the 95% PI of the simulated p5 and p95. Abbreviations: CI=confidence interval; p5=5th percentile; p95=95th percentile; PI=prediction interval; VPC=visual predictive check.

FIG. 30 shows the prediction-corrected VPC for MDMA Stratified by Study. Dots are observed data points; indicated solid lines are the observed median; dashed lines are observed p5 and p95. Indicated shaded areas are the 95% PI of the simulated median, and the indicated middle shaded areas is the 95% PI of the simulated p5 and p95. Abbreviations: CI=confidence interval; p5=5th percentile; p95=95th percentile; PI=prediction interval; VPC=visual predictive check.

FIG. 31 shows the prediction-corrected VPC for MDA Stratified by Study. Dots are observed data points; indicated solid lines are the observed median; dashed lines are observed p5 and p95. Indicated shaded areas are the 95% PI of the simulated median, and the indicated middle shaded areas is the 95% PI of the simulated p5 and p95. Abbreviations: CI=confidence interval; p5=5th percentile; p95=95th percentile; PI=prediction interval; VPC=visual predictive check.

FIG. 32 shows the Forest plot of Covariate Effects on AUC₀₋₄₄ and Cₘₐₓ for MDA. For all covariate scenarios, all other covariates were maintained at values for a reference individual. The reference individual was defined as a white male weighing 70 kg, aged 25 years, with baseline creatine clearance of 113 mL/min, and fed a light meal. Left side of the plot: Number to the right of body weight correspond to the 5th and 95th percentile of the population in kg. Numbers to the right of categorical covariates correspond to numbers of subjects in each category (Non-reference: reference). Right side of the plot. Values in boxes represent data shown in the graph, summarized the median and 95% confidence interval for AUC (left box) and Cₘₐₓ (right box). AUC refers to AUC₀₋₄₄. Abbreviations: CI=confidence interval.

FIG. 33 shows Simulated Profiles of MDMA and MDA Up To 44 Hours Post Dose. Single dose and split dose profiles are shown on the same set of axes for all analytes and dose levels. Dashed vertical lines indicate dose times (time=0 and 104 minutes after the first dose).

FIG. 34 shows Simulated Profiles of MDMA and MDA Up To 4 Hours Post Dose. Single dose and split dose profiles are shown on the same set of axes for all analytes and dose levels. Dashed vertical lines indicate dose times (time=0 and 104 minutes after the first dose).

FIG. 35 shows a comparison of EAT-26 scores obtained by subjects, before and after completing MDMA- or placebo-assisted therapy.

FIG. 36 and FIG. 37 show the time course of improvement in Clinician Administered PTSD Scale for DSM-5 (CAPS-5) total severity score versus placebo. Each show a treatment difference between MDMA and placebo after 3 medication sessions with psychological intervention. The onset of treatment efficacy was observed after the second session in both studies. Between the second and third treatment sessions, the difference between the groups appeared to continue to increase in both studies. The primary efficacy measure was change from baseline in the CAPS-5 total severity score at 18 weeks, which was evaluated 2 months after the final dosing session. The CAPS-5 is a 30-item, clinician-rated scale used to assess severity of PTSD symptoms and diagnostic status according to DSM-5 criteria, as described herein. Scores on the CAPS 5 range from 0 to 80, with higher scores indicating more severe PTSD symptoms. In both studies, a secondary efficacy measure was change from baseline in Sheehan Disability Scale (SDS) mean item scores at 18 weeks. The modified SDS is a 3-item, clinician-rated scale measuring the degree of functional impairment in work, family, and social life. Scores on the SDS mean items range from 0 to 10, with higher scores indicating more functional impairment. In these studies, the efficacy assessments were administered by blinded independent raters 18 to 30 days after medication sessions 1 and 2.

### DETAILED DESCRIPTION

Reference will now be made in detail to certain embodiments of the present disclosure, examples of which are illustrated in the accompanying structures and formulas. While the present disclosure will be described in conjunction with the enumerated embodiments, it will be understood that the present disclosure is not limited to these embodiments. On the contrary, the present disclosure is intended to cover all alternatives, modifications, and equivalents that can be included within the scope of the present disclosure as defined by the claims. One skilled in the art will recognize many methods and materials similar or equivalent to those described herein, which could be used in the practice of the present disclosure.

Any of the embodiments described herein, including those described under different embodiments of the present disclosure and different parts of the specification (including embodiments described only in the Examples) can be combined with one or more other embodiments of the present disclosure, unless explicitly disclaimed or improper. Combinations of embodiments are not limited to the specific combinations claimed via the multiple dependent claims.

In some embodiments, MDMA is isolated as a pharmaceutically acceptable salt of the freebase form of MDMA. This salt is a substantially crystalline solid with a significant number of particles with volume diameters in excess of 600 microns (m), as determined by laser diffraction. Formulation testing revealed that these larger particles are inadequate for batch consistency and desirable dissolution parameters, creating the need for MDMA solids with reduced particle size and improved particle size uniformity.

MDMA has multiple solid-state forms, including hydrates of MDMA·HCl that readily forms under ambient conditions, particularly during grinding and in high-humidity environments. In addition, the free base of MDMA is an oil at room temperature. To maintain batch consistency in MDMA pharmaceutical formulations, a method of particle size reduction and formulation that does not result in hydrate formation is needed.

Provided herein are particles comprising crystalline MDMA, or a pharmaceutically acceptable salt and/or solvate thereof, that have a smaller average diameter. Some embodiments provide a method to reduce particle size as measured by Dv90 that avoids hydrate formation.

In some embodiments, the present disclosure provides particles comprising crystalline MDMA, or a pharmaceutically acceptable salt and/or solvate thereof, substantially less than about 610 μm, with a Dv90 below about 420 μm and a particle size range of less than about 400 μm. The improved bulk solid properties of crystalline MDMA, or a pharmaceutically acceptable salt and/or solvate thereof, with reduced particle size and increased particle size uniformity provide acceptable batch consistency during the formulation process, enabling the production of pharmaceutically acceptable compositions or pharmaceutically acceptable dosage forms, or salts thereof, as well as a dissolution rate suitable for a high solubility/permeability drug product.

In some embodiments, the present disclosure provides a method of producing crystalline MDMA, or a pharmaceutically acceptable salt and/or solvate thereof, comprising particles substantially less than about 610 μm, with a Dv90 below 420 μm and a particle size range of less than 400 μm, that avoids hydrate formation and maintains suitable flowability in the milled product.

In some embodiments, the present disclosure provides pharmaceutical compositions and dosage forms manufactured from crystalline MDMA, or a pharmaceutically acceptable salt and/or solvate thereof, comprising particles substantially less than about 610 μm, with a Dv90 below 420 μm and a particle size range of less than 400 μm.

In some embodiments, the present disclosure provides a method of treating an eating disorder in a subject, comprising administering to the subject a therapeutically effective amount of particles comprising 3,4-methylenedioxymethamphetamine (MDMA), or a pharmaceutically acceptable salt and/or solvate thereof, wherein the particles comprise particles that are substantially less than about 610 μm.

In some embodiments, the present disclosure provides a method of treating an eating disorder in a subject, comprising administering to the subject a therapeutically effective amount of particles comprising crystalline 3,4-methylenedioxymethamphetamine (MDMA), or a pharmaceutically acceptable salt and/or solvate thereof, wherein the particles comprise particles that are substantially less than about 610 μm.

In some embodiments, the present disclosure provides a method of treating subjects suffering from an eating disorder by providing a pharmaceutical composition containing crystalline MDMA, or a pharmaceutically acceptable salt and/or solvate thereof, comprising particles substantially less than about 610 μm, with a Dv90 below 420 μm and a particle size range of less than 400 μm in a therapeutic setting.

In any of the compositions described herein, substantially all of the crystalline MDMA, or a pharmaceutically acceptable salt and/or solvate thereof, particles are less than about 610 μm, and the Dv90 is below about 400 μm. In some cases, the Dv90 is from about 0.01 μm to about 400 μm. In some cases, less than 10% of the particles have a particle size (Dv10) below about 10 μm. In some cases, from about 0% to about 10% of the particles have a particle size (Dv10) from about 0.01 μm to about 10 μm. In some cases, the median particle size (Dv50) is from about 100 μm to about 200 μm.

In any of the compositions described herein, the chemical purity is greater than 98% and no single impurity is present in an amount greater than 0.5% as determined by HPLC. In some cases, wherein the chemical purity is greater than 99% and no single impurity is present in an amount greater than 0.5% as determined by HPLC.

In any of the compositions described herein, the MDMA, or a pharmaceutically acceptable salt and/or solvate thereof, particles are substantially free of MDMA·HCl monohydrate.

In any of the compositions described herein, the dissolution rate in water is greater than or equal to 80% of the mass of the particles in 30 minutes.

In some cases, the compositions described herein comprises about 1 mg to about 150 mg of particles comprising crystalline MDMA, or a pharmaceutically acceptable salt and/or solvate thereof on a free base basis of MDMA. In some cases, the compositions described herein include about 35 mg to about 45 mg of particles comprising crystalline MDMA, or a pharmaceutically acceptable salt and/or solvate thereof on a free base basis of MDMA. In some cases, the compositions described herein include about 55 mg to about 65 of particles comprising crystalline MDMA, or a pharmaceutically acceptable salt and/or solvate thereof on a free base basis of MDMA. In some cases, the compositions described herein include about 75 mg to about 85 mg of particles comprising crystalline MDMA, or a pharmaceutically acceptable salt and/or solvate thereof on a free base basis of MDMA. In some cases, the compositions described herein include about 95 mg to about 105 mg of particles comprising crystalline MDMA, or a pharmaceutically acceptable salt and/or solvate thereof on a free base basis of MDMA. In some cases, the compositions described herein include comprising about 115 mg to about 125 mg of particles comprising crystalline MDMA, or a pharmaceutically acceptable salt and/or solvate thereof on a free base basis of MDMA.

In any of the compositions described herein, the particles comprising crystalline MDMA, or a pharmaceutically acceptable salt and/or solvate thereof, are prepared by a process comprising the step of reducing MDMA particle size and increasing MDMA particle size uniformity by screen-milling under an inert atmosphere. In some cases, the coarse MDMA particles do not undergo an additional size-reducing process.

In some cases, the median particle size (Dv50) of coarse MDMA particles is greater than 400 μm. In some cases, the coarse MDMA particles are substantially free of MDMA HCl monohydrate. In some cases, the coarse MDMA particles comprise crystalline MDMA·HCl.

In some cases, the coarse MDMA particles are heated to a temperature of 50-70° C. in an environment with an ambient pressure below 1 atmosphere, prior to entering the screen mill. In some cases, the coarse MDMA particles are fed into the screen mill in the absence of applied pressure. In some cases, the coarse MDMA particles are fed into the screen mill at ambient temperature (e.g., RT). In some cases, inert atmosphere in the method is substantially free of moisture. In some cases, the inert atmosphere comprises substantially dry nitrogen gas.

In some cases, the compositions described herein can additionally include a diluent. In some cases, the diluent is a sugar alcohol. In some cases, the diluent has a moisture content of less than 0.25% by mass, prior to blending (e.g., prior to blending with the other components of the composition).

In some cases, the compositions described herein can additionally include a lubricant. In some cases, the lubricant includes a pharmaceutically acceptable salt of a saturated fatty acid.

In some cases, the dosage form (e.g., oral dosage form) can be a capsule. In some cases, the dosage form (e.g., oral dosage form) can be a tablet. In some cases, the dosage form is a capsule. In some cases, the dosage form is a tablet.

In some cases, the dosage form (e.g., oral dosage form), includes one or more individual dosage units. In some cases, the dosage form includes one individual dosage unit. In some cases, the dosage form includes at least two individual dosage units. In some cases, the dosage form includes at least three individual dosage units. In some cases, each of the one or more individual dosage units comprises a capsule.

In some cases, the one or more individual dosage units are administered during a single psychotherapy session. In some cases, the one or more individual dosage units are administered at different times during the single psychotherapy session.

In some cases, the one or more individual dosage units are administered during a single medication session. In some cases, the one or more individual dosage units are administered at different times during the single medication session.

In some cases, the one or more individual dosage units are administered during a single psychological intervention. In some cases, the one or more individual dosage units are administered at different times during the single psychological intervention.

In some embodiments, the total dose of MDMA, or a pharmaceutically acceptable salt thereof, is given as a divided dose. For example, a first dose of about 68 mg and a second dose of about 34 mg for about a 102 mg total dose; or a first dose of about 100 mg and a second dose of about 50 mg for about a 150 mg total dose; or three equal doses of, for example, about 34 mg or about 50 mg, for a total dose of about 102 mg or about 150 mg, respectively.

In some cases, about 34 mg of the particles comprising crystalline MDMA, or a pharmaceutically acceptable salt and/or solvate thereof on a free base basis of MDMA, are administered. In some cases, wherein the about 34 mg of particles comprising crystalline MDMA, or a pharmaceutically acceptable salt and/or solvate thereof on a free base basis of MDMA, are administered in one dose. In some cases, the about 34 mg of particles comprising crystalline MDMA, or a pharmaceutically acceptable salt and/or solvate thereof on a free base basis of MDMA, are administered in two doses.

In some cases, about 50 mg of the particles comprising crystalline MDMA, or a pharmaceutically acceptable salt and/or solvate thereof on a free base basis of MDMA, are administered. In some cases, wherein the about 50 mg of particles comprising crystalline MDMA, or a pharmaceutically acceptable salt and/or solvate thereof on a free base basis of MDMA, are administered in one dose. In some cases, the about 50 mg of particles comprising crystalline MDMA, or a pharmaceutically acceptable salt and/or solvate thereof on a free base basis of MDMA, are administered in two doses.

In some cases, about 100 mg of the particles comprising crystalline MDMA, or a pharmaceutically acceptable salt and/or solvate thereof on a free base basis of MDMA, are administered. In some cases, wherein the about 100 mg of particles comprising crystalline MDMA, or a pharmaceutically acceptable salt and/or solvate thereof on a free base basis of MDMA, are administered in one dose. In some cases, the about 100 mg of particles comprising crystalline MDMA, or a pharmaceutically acceptable salt and/or solvate thereof on a free base basis of MDMA, are administered in two doses.

In some cases, about 120 mg of the particles comprising crystalline MDMA, or a pharmaceutically acceptable salt and/or solvate thereof on a free base basis of MDMA, are administered. In some cases, the about 120 mg of particles comprising crystalline MDMA, or a pharmaceutically acceptable salt and/or solvate thereof on a free base basis of MDMA, are administered in one dose. In some cases, the about 120 mg of particles comprising crystalline MDMA, or a pharmaceutically acceptable salt and/or solvate thereof on a free base basis of MDMA, are administered in two doses.

In some cases, about 140 mg of the particles comprising crystalline MDMA, or a pharmaceutically acceptable salt and/or solvate thereof on a free base basis of MDMA, are administered. In some cases, about 140 mg of particles comprising crystalline MDMA, or a pharmaceutically acceptable salt and/or solvate thereof on a free base basis of MDMA, are administered in one dose. In some cases, the about 140 mg of particles comprising crystalline MDMA, or a pharmaceutically acceptable salt and/or solvate thereof on a free base basis of MDMA, are administered in two doses.

In some cases, about 160 mg of the particles comprising crystalline MDMA, or a pharmaceutically acceptable salt and/or solvate thereof on a free base basis of MDMA, are administered. In some cases, the about 160 mg of particles comprising crystalline MDMA, or a pharmaceutically acceptable salt and/or solvate thereof on a free base basis of MDMA, are administered in one dose. In some cases, the about 160 mg of particles comprising crystalline MDMA, or a pharmaceutically acceptable salt and/or solvate thereof on a free base basis of MDMA, are administered in two doses.

In some cases, the therapeutically effective amount of the MDMA particles is orally administered. In some cases, the therapeutically effective amount of the MDMA particles is administered in a capsule. In some cases, the therapeutically effective amount of the MDMA particles is administered in a tablet.

In some cases, the therapeutically effective amount of the MDMA particles is administered as one or more individual dosage units during a single psychological intervention. In some cases, the therapeutically effective amount of the MDMA particles is administered at different times during a single psychological intervention.

In any of the methods described herein, the individual dosage units can be administered during one or more psychological interventions. In some cases, the dosage units can be administered during one psychological intervention. In some cases, the dosage units can be administered during two psychological interventions. In some cases, the dosage units can be administered during three psychological interventions. In some cases, the dosage units can be administered during more than three psychological interventions.

In some cases, the therapeutically effective amount of the MDMA particles is administered as one or more individual dosage units during a single psychotherapy session. In some cases, the therapeutically effective amount of the MDMA particles is administered at different times during a single psychotherapy session.

In any of the methods described herein, the individual dosage units can be administered during one or more psychotherapy sessions. In some cases, the dosage units can be administered during one psychotherapy session. In some cases, the dosage units can be administered during two psychotherapy sessions. In some cases, the dosage units can be administered during three psychotherapy sessions. In some cases, the dosage units can be administered during more than three psychotherapy sessions.

In some cases, the therapeutically effective amount of the MDMA particles is administered as one or more individual dosage units during a single medication session. In some cases, the therapeutically effective amount of the MDMA particles is administered at different times during a single medication session.

In any of the methods described herein, the individual dosage units can be administered during one or more medication sessions. In some cases, the dosage units can be administered during one medication session. In some cases, the dosage units can be administered during two medication sessions. In some cases, the dosage units can be administered during three medication sessions. In some cases, the dosage units can be administered during more than three medication sessions.

In any of the methods described herein, the administration of particles comprising crystalline MDMA, or a pharmaceutically acceptable salt and/or solvate thereof, during one or more psychotherapy sessions (or medication sessions or psychological interventions) can be used to treat a subject suffering from one or more symptoms of disordered eating. In some cases, the subject may have one or more symptoms of disordered eating associated with an eating disorder. In some cases, the subject may have an eating disorder. In some cases, the subject may have a clinically diagnosed eating disorder. In some cases, the subject can be receiving one or more treatments or therapies for a clinically diagnosed eating disorder. In some cases, the subject may have a history of receiving one or more treatments or therapies for a clinically diagnosed eating disorder. In some cases, the subject may not have received any treatments or therapies for an eating disorder.

In any of the methods described herein, the administration of particles comprising crystalline MDMA, or a pharmaceutically acceptable salt and/or solvate thereof, during one or more psychotherapy sessions (or medication sessions or psychological interventions) can be used to treat a subject suffering from an eating disorder. In some cases, the eating disorder is co-associated with a post-traumatic stress disorder. In some cases, the eating disorder is anorexia nervosa. In some cases, the eating disorder is bulimia nervosa. In some cases, the eating disorder is binge eating disorder. In some cases, the eating disorder is orthorexia. In some cases, the eating disorder is eating disorder not otherwise specified (EDNOS). In some cases, the eating disorder is purging disorder. In some cases, the eating disorder is rumination disorder. In some cases, the eating disorder is atypical anorexia nervosa. In some cases, the eating disorder is avoidant/restrictive food disorder. In some cases, the eating disorder is other specified feeding or eating disorder (OSFED).

In any of the methods described herein, the subject being treated for an eating disorder by administration of particles comprising crystalline MDMA, or a pharmaceutically acceptable salt and/or solvate thereof, during one or more psychotherapy sessions (or medication sessions or psychological interventions) may have a body mass index (BMI) below, above, or within the “Healthy Weight” range (18.5 to 24.9). In some cases, the subject may have a BMI in the “underweight” range (i.e., below 18.5). In some cases, the subject may have a BMI in the “overweight” range (i.e., 25.0 to 29.9). In some cases, the subject may have a BMI in the “obese” range (i.e., above 30.0). BMI is calculated by dividing a subject's weight in kilograms by square of the same subject's height in meters.

### Definitions

As used herein, the term “composition” is intended to encompass a product comprising the specified ingredients in the specified amounts, as well as any product which results, directly or indirectly, from combinations of the specified ingredients in the specified amounts. A composition can refer to a product suitable for administration to a subject, but for clarity, compositions for pharmaceutical use are generally referred to as “dosage forms” herein.

As particles are often non-spherical, it is difficult and complex to provide dimensional descriptions of these non-spherical particles. As used herein, “volume diameter” refers to the diameter of a sphere with a volume equivalent to that of the non-spherical particle. In some embodiments, the particle sizes described herein are measured using a laser diffraction technique that correlates light scattering to particle volume, from which effective length or effective diameter is calculated.

The distribution is based on a measurement of thousands of particles. Particle samples can be in dry form, in slurry form, or in the form of suspension. In one embodiment, the particle sample is suspended in a solution of cyclohexane. In another embodiment, the instrument used to determine particle size and distribution is a Malvern Mastersizer 3000 with a red light source max 4 mW He—Ne, 632.8 nm, blue light source (nominal) 10 mW LED, 470 nm, with a Reverse Fourier (convergent beam) lens arrangement and effective focal length of 300 mm. See, e.g., USP <429> and EP 2.9.31.

MDMA particle size distribution, in particular d90, d10, and d50 values, can be determined by also well-known methods in the art including, but not limitated to sieve analysis, laser diffraction methods (in addition to those specifically described herein), photoanalysis, and/or optical counting methods. One may also be able to use Raman spectroscopy in conjunction with scanning electron microscopty to analyze the tablet itself to analyze particles of MDMA.

As used herein, particle size is expressed in terms of volume diameter and the particle size distribution is expressed in terms of Dv50, Dv10, and Dv90. A Dv90 value, for example, represents that 90% of particles formed are below a certain threshold. For instance, a Dv90 below 420 μm means that 90% of particles formed have a lower diameter than 420 μm. As used herein, “Dv50”, also known as the median particle diameter, corresponds to the value for which 50% of the particles have a lower volume diameter, and 50% of the particles have a higher volume diameter. “Dv90” corresponds to the value for which 90% of the particles have a lower volume diameter, and 10% of the particles have a higher volume diameter. “Dv10” corresponds to the value for which 10% of the particles have a lower volume diameter, and 90% of the particles have a higher volume diameter.

As used herein, “particle size range” corresponds to a value obtained by subtracting the Dv10 from the Dv90. The “Dv10-Dv90 range” can be calculated from the Dv10 and Dv90 obtained from a single sample, or it can be calculated by averaging the Dv10 and Dv90 values obtained, individually, from a plurality of samples taken from the same batch.

Unless specifically stated or obvious from context, as used herein, the term “about” is understood as within a range of normal tolerance in the art, for example, within 10% of the stated value. As used herein, “about” a specific value also includes the specific value, for example, about 10% includes 10%. When used to describe a range, for example, from about 5 to about 8, the range can include 10% lower than the lower bound and 10% higher than the upper bound.

As used herein, the term “MDMA” refers to the compound 3,4-methylenedioxymethamphetamine, having the structure:

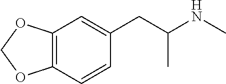

In some embodiments, the MDMA is racemic. In some embodiments, the MDMA is (S)-MDMA, in some embodiments, the MDMA is (R)-MDMA. In some embodiments, the MDMA is a non-racemic (i.e., scalemic) mixture of (S)-MDMA and (R)-MDMA.

The term “treating” refers to administering a therapy in an amount, manner, or mode effective to improve a condition, symptom, or parameter associated with a disease or disorder. The term “treating” or “treatment” covers the treatment of a disease or disorder described herein, in a subject, such as a human, and includes: (i) inhibiting a disease or disorder, i.e., arresting its development; (ii) relieving a disease or disorder, i.e., causing regression of the disease or disorder; (iii) slowing progression of the disease or disorder; and/or (iv) inhibiting, relieving, or slowing progression of one or more symptoms of the disease or disorder.

The term “therapeutic” as used herein means a treatment. A therapeutic effect is obtained by suppression, remission, or eradication of a disease state.

The term “prevent” or “preventative” as used herein means a prophylactic treatment. A preventative effect is obtained by delaying the onset of a disease state or decreasing the severity of a disease state when it occurs.

The term “therapeutically effective amount”, “prophylactically effective amount”, or “effective amount” refers to an amount of the agent that, when administered, is sufficient to cause the desired effect. For example, an effective amount of MDMA can be an amount sufficient to have a beneficial effect on the subject (e.g., to lessen symptoms of disease or disorder). The therapeutically effective amount of the agent may vary depending on the disease being treated and its severity as well as the age, weight, etc., of the subject to be treated. In the methods described herein, the therapeutic compounds can be administered to a subject having one or more signs or symptoms of a disease or disorder.

The term “pharmaceutically acceptable” indicates that the compound, or salt or composition thereof is compatible chemically and/or toxicologically with the other ingredients comprising a formulation and/or the subject being treated therewith.

As used herein, the term “pharmaceutically acceptable salts” refers to salts that retain the desired biological activity of the subject compound and exhibit minimal undesired toxicological effects. These pharmaceutically acceptable salts can be prepared in situ during the final isolation and purification of the compound, or by separately reacting the purified compound in its free base form with a suitable acid. Suitable acids include pharmaceutically acceptable inorganic acids and pharmaceutically acceptable organic acids. Representative pharmaceutically acceptable acid addition salts include hydrochloride, hydrobromide, nitrate, methylnitrate, sulfate, bisulfate, sulfamate, phosphate, acetate, hydroxyacetate, phenyl acetate, propionate, butyrate, isobutyrate, valerate, maleate, hydroxymaleate, acrylate, fumarate, malate, tartrate, citrate, salicylate, p-aminosalicyclate, glycollate, lactate, heptanoate, phthalate, oxalate, succinate, benzoate, o-acetoxybenzoate, chlorobenzoate, methylbenzoate, dinitrobenzoate, hydroxybenzoate, methoxybenzoate, mandelate, tannate, formate, stearate, ascorbate, palmitate, oleate, pyruvate, pamoate, malonate, laurate, glutarate, glutamate, estolate, methanesulfonate (mesylate), ethanesulfonate (esylate), 2-hydroxyethanesulfonate, benzenesulfonate (besylate), p-aminobenzenesulfonate, p-toluenesulfonate (tosylate),napthalene-2-sulfonate, ethanedisulfonate, and 2,5-dihydroxybenzoate.

As used herein, “MDMA HCl” refers to a hydrochloride salt of MDMA with the molar ratio of MDMA to HCl being about 1:1. MDMA·HCl, which is known to form one major crystal form (Form I) and at least four hydrate forms that incorporate 0.25-1 water of hydration (Shulgin, A. T. J. Psychoact. Drugs 1986, 18, 291-304) as well as two additional anhydrous forms (Form II and Form III). A polymorphic screening established Form I is the most stable of Forms I-III (see Nair et. al., ACS Omega 2022, 7, 1, 900-907, which is incorporated by reference herein in its entirety). Form I and Form II reversibly convert into the known monohydrate; upon dehydration, the monohydrate formed from Form I will revert back to Form I, and the monohydrate formed from Form II will revert back to Form II. Form III, spontaneously converts to Form I after 2.5 weeks storage at ambient conditions. In some embodiments, MDMA·HCl is Form I. In some embodiments, MDMA·HCl is Form II. In some embodiments, MDMA·HCl is Form III. In some embodiments, the MDMA·HCl is anhydrous MDMA·HCl.

The term “psychological intervention” refers to a relationship aimed at promoting a better adaptation of a subject to a given situation and thereby optimizing the subject's personal resources in relation to autonomy, self-knowledge and self-help. In other words, psychological intervention aims to produce a personal change leading to higher (e.g., more improved) functional results.

The term “psychotherapy session” refers to a period of time during which communication (e.g., oral communication) between a subject and a therapist to improve psychological functioning, well-being, and coping mechanisms in the subject occurs. In some embodiments, the therapist is a person who has received training to administer therapy (e.g., a psychiatrist, psychologist, or licensed social worker).

In some instances, “sessions” is used to collectively denote a medication session, psychological intervention, and/or a psychotherapy session.

The term “administering” or “administration” of a therapy (e.g., MDMA) to a subject includes any route of introducing or delivering a compound to a subject to perform its intended function. Administration can be carried out by any suitable route, including orally, intranasally, parenterally (intravenously, intramuscularly, intraperitoneally, or subcutaneously), or topically. Administration includes self-administration and administration by another person.

The term “subject” and “patient” are used interchangeably herein and refer to any animal amenable to the methods described herein. In some embodiments, the subject is a mammal. In some embodiments, the mammal is a mouse, a rat, a guinea pig, a non-human primate, a dog, a cat, or a domesticated animal (e.g., horse, cow, pig, goat, sheep). In some preferred embodiments, the subject is a human.

“Optional” or “optionally” means that the subsequently described event or circumstance can or cannot occur, and that the description includes instances where the event or circumstance occur and instances where it does not.

The term “substantially” is used herein to refer to greater than 90% (e.g., greater than 92%, greater than 94%, greater than 96%, greater than 98%, or greater than 99%). For example, the composition is substantially free of MDMA·HCl hydrate, i.e., of the MDMA present in the composition, less than 10% is MDMA·HCl hydrate (e.g., less than 8%, less than 6%, less than 4%, less than 2%, or less than 1% is MDMA·HCl hydrate). For example, the MDMA is “substantially pure”, meaning that the MDMA contains less than 10% of compounds or substances that are not MDMA (e.g., less than 8%, less than 6%, less than 4%, less than 2%, or less than 1% of compounds or substances that are not MDMA). In some embodiments, substantially pure MDMA contains greater than 90% (e.g., greater than 92%, greater than 94%, greater than 96%, greater than 98%, or greater than 99%) of a single solid form (e.g., polymorph), a single salt, or a single solvate of MDMA.

To provide a more concise description, some of the quantitative expressions herein are recited as a range from about amount X to about amount Y, wherein “X” is one numerical limit of the range and “Y” is the other numerical limit of the range. It is understood that wherein a range is recited, the range includes but is not limited to the recited upper and lower bounds, but rather includes the full range from about amount X through about amount Y, or any sub-range therein.

### Particles of MDMA

Consistent dosing, e.g., achieving consistent dose uniformity, to provide safe and effective MDMA levels for treatment has been challenging. Current manufacturing and formulation protocols for therapeutic MDMA provides formulations containing particles with an average particle size greater than 600 micrometers (m or micron) (e.g., as determined by laser diffraction). Formulation testing revealed that these larger particles are inadequate for batch consistency and do not have desirable dissolution parameters, creating the need for MDMA solids with reduced particle size and improved particle size uniformity.

MDMA has multiple solid-state forms, including MDMA·HCl hydrate that readily forms under ambient conditions, particularly during grinding and in high-humidity environments. The hydrate is significantly more hygroscopic than the MDMA, and can adsorb up to two additional molar equivalents of surface moisture when fine particles are exposed to a high-humidity environment for an extended period of time. It is therefore necessary to reduce MDMA particle size in an environment that is unfavorable for hydrate formation.

The present disclosure provides a solution to these problems.

Some embodiments provide particles of 3,4-methylenedioxymethamphetamine (MDMA), or a pharmaceutically acceptable salt and/or solvate thereof having an average particle size from about 50 μm to about 600 μm.

Some embodiments provide particles of 3,4-methylenedioxymethamphetamine (MDMA), or a pharmaceutically acceptable salt and/or solvate thereof having an average particle size from about 50 μm to about 400 μm.

Some embodiments provide particles of 3,4-methylenedioxymethamphetamine (MDMA), or a pharmaceutically acceptable salt and/or solvate thereof having an average particle size from about 50 μm to about 450 μm.

Some embodiments provide particles of 3,4-methylenedioxymethamphetamine (MDMA), or a pharmaceutically acceptable salt and/or solvate thereof having an average particle size of about 420 μm.

In some embodiments, the particle comprises MDMA. In some embodiments, the particle comprises MDMA·HCl. In some embodiments, the particle comprises anhydrous MDMA·HCl. In some embodiments, the particle is MDMA. In some embodiments, the particle is MDMA·HCl. In some embodiments, the particle is anhydrous MDMA·HCl. In some embodiments, the particles have desirable bulk properties and processability for preparing dosage forms suitable for administration to a subject.

MDMA isolated from chemical synthesis is a highly pure, coarse solid with varying particle size. The coarse MDMA, with a typical Dv90 from 800 to 1600 μm and a typical particle size range from 500 μm to 1100 μm, does not yield a uniform blend. The particle size distribution of the MDMA, or a pharmaceutically acceptable salt and/or solvate thereof, in the resulting dosage form is uneven, leading to an unacceptably high rate of batch failure.

In some embodiments, the particles are substantially less than about 610 μm. In some embodiments, substantially all of the particles have particle sizes and/or volume diameters below about 610 μm. In some embodiments, substantially all of the particles have at least one dimension less than about 610 μm. In some embodiments, the particles are substantially less than about 600 μm. In some embodiments, substantially all of the particles have particle sizes and/or volume diameters below about 600 μm. In some embodiments, substantially all of the particles have at least one dimension less than about 600 μm.

In some embodiments, the average particle size of the MDMA is from about 50 μm to about 450 μm. In some embodiments, the average particle size of the composition is from about 50 μm to about 100 μm, about 100 μm to about 150 μm, about 150 μm to about 200 μm, about 200 μm to about 250 μm, about 250 μm to about 300 μm, about 350 μm to about 400 μm, about 400 μm to about 450 μm, about 50 μm to about 150 μm, about 150 μm to about 250 μm, about 250 μm to about 450 μm, about 200 μm to about 450 μm, about 100 μm to about 300 μm, about 300 μm to about 450 μm, or about 400 μm to about 450 μm.

In some embodiments, the average particle size of the MDMA is from about 50 μm to about 400 μm. In some embodiments, the average particle size of the composition is from about 50 μm to about 100 μm, about 100 μm to about 150 μm, about 150 μm to about 200 μm, about 200 μm to about 250 μm, about 250 μm to about 300 μm, about 350 μm to about 400 μm, about 50 μm to about 150 μm, about 150 μm to about 250 μm, about 250 μm to about 400 μm, about 200 μm to about 400 μm, about 100 μm to about 300 μm, or about 200 μm to about 400 μm.

In some embodiments, the average particle size of the MDMA is from about 400 μm to about 450 μm. In some embodiments, the average particle size of the MDMA is about 400 μm, about 405 μm, about 410 μm, about 415 μm, about 420 μm, about 425 μm, about 430 μm, about 435 μm, about 440 μm, about 445 μm, or about 450 μm.

In some embodiments, the average particle size of the MDMA is from about 415 μm to about 425 μm. In some embodiments, the average particle size of the MDMA is about 415 μm, about 416 μm, about 417 μm, about 418 μm, about 419 μm, about 420 μm, about 421 μm, about 422 μm, about 423 μm, about 424 μm, or about 425 μm. In some embodiments, the average particle size of the MDMA is about 420 μm.

In some embodiments, the average particle size of the MDMA is from 50 μm to 450 μm. In some embodiments, the average particle size of the composition is from 50 μm to 100 μm, 100 μm to 150 μm, 150 μm to 200 μm, 200 μm to 250 μm, 250 μm to 300 μm, 350 μm to 400 μm, 400 μm to 450 μm, 50 μm to 150 μm, 150 μm to 250 μm, 250 μm to 450 μm, 200 m to 450 μm, 100 μm to 300 μm, 300 μm to 450 μm, or 400 μm to 450 μm.

In some embodiments, the average particle size of the MDMA is from 50 μm to 400 μm. In some embodiments, the average particle size of the composition is from 50 μm to 100 μm, 100 μm to 150 μm, 150 μm to 200 μm, 200 μm to 250 μm, 250 μm to 300 μm, 350 μm to 400 μm, 50 μm to 150 μm, 150 μm to 250 μm, 250 μm to 400 μm, 200 μm to 400 μm, 100 m to 300 μm, or 200 μm to 400 μm.

In some embodiments, the average particle size of the MDMA is from 400 μm to 450 μm. In some embodiments, the average particle size of the MDMA is 400 μm, 405 μm, 410 μm, 415 μm, 420 μm, 425 μm, 430 μm, 435 μm, 440 μm, 445 μm, or 450 μm.

In some embodiments, the average particle size of the MDMA is from 415 μm to 425 μm. In some embodiments, the average particle size of the MDMA is 415 μm, 416 μm, 417 μm, 418 μm, 419 μm, 420 μm, 421 μm, 422 μm, 423 μm, 424 μm, or 425 μm. In some embodiments, the average particle size of the MDMA is 420 μm.

In some embodiments, the particles have a Dv10 from about 5 μm to about 40 μm, a Dv50 from about 100 μm to about 200 μm, a Dv90 from about 250 μm to about 420 μm, to a particle size range from about 250 μm to about 350 μm.

In some embodiments, the Dv10 value of the particles is from about 5 μm to about 40 μm, about 5 μm to about 30 μm, about 5 μm to about 20 μm, about 5 μm to about 15 μm, about 10 μm to about 40 μm, about 15 μm to about 40 μm, about 20 μm to about 40 μm, about 25 m to about 40 μm, about 10 μm to about 35 μm, about 15 μm to about 35 μm, about 18 μm to about 32 μm, about 20 μm to about 30 μm, about 20 μm to about 25 μm, about 25 μm to about 30 μm, about 5 μm, about 10 μm, about 15 μm, about 20 μm, about 21 μm, about 22 μm, about 23 μm, about 24 μm, about 25 μm, about 26 μm, about 27 μm, about 28 μm, or about 29 μm.

In some embodiments, the Dv50 value of the particles is from about 100 μm to about 200 μm, about 110 μm to about 190 μm, about 120 μm to about 180 μm, about 100 μm to about 200 μm, about 100 μm to about 200 μm, about 100 μm to about 200 μm, about 100 μm to about 200 μm, about 100 μm to about 200 μm, about 100 μm to about 200 μm, about 100 μm to about 200 μm, about 100 μm to about 200 μm, or about 100 μm to about 200 μm.

In some embodiments, the Dv90 value of the particles is from about 0.01 μm to about 400 μm, from about 250 μm to about 420 μm, from about 250 μm to about 400 μm, from about 250 μm to about 380 μm, from about 270 μm to about 380 μm, from about 270 μm to about 360 μm, from about 270 μm to about 350 μm, from about 270 μm to about 420 μm, from about 290 μm to about 420 μm, from about 290 μm to about 400 μm, from about 290 μm to about 380 μm, from about 310 μm to about 420 μm, from about 310 μm to about 400 μm, from about 310 μm to about 380 μm, from about 330 μm to about 420 μm, from about 330 μm to about 400 μm, from about 330 μm to about 380 μm, from about 350 μm to about 420 μm, from about 350 μm to about 400 μm, or from about 370 μm to about 420 μm. In some embodiments, the Dv90 value of the particles is about 420 μm. In some embodiments, the Dv90 value of the particles is 420 μm.

In some embodiments, the particles are more uniformly distributed (e.g., have a smaller particle size range) than are the crude MDMA particles isolated from the synthetic process. In some embodiments, the particles have a particle size range that is less than about 600 μm (e.g., less than about 500 μm, less than about 420 μm, or less than about 400 μm). In some embodiments, the particle size range is about 5 μm to about 500 μm (e.g., about 5 μm to about 420 μm, about 20 μm to about 353 μm, about 20 μm to about 326 μm, about 21 μm to about 353 μm, about 21 μm to about 342 μm, about 21 μm to about 326 μm, about 24 μm to about 353 μm, about 29 μm to about 342 μm, about 34 μm to about 341 μm, about 200 μm to about 400 μm, about 230 μm to 380 μm, or about 250 μm to 350 μm.

In some embodiments, the particles have a Dv10 from 5 μm to 40 μm, a Dv50 from 100 m to 200 μm, a Dv90 from about 250 μm to 420 μm, to a particle size range from 250 μm to 350 μm.

In some embodiments, the Dv10 value of the particles is from 5 μm to 40 μm, 5 μm to 30 μm, 5 μm to 20 μm, 5 μm to 15 μm, 10 μm to 40 μm, 15 μm to 40 μm, 20 μm to 40 μm, 25 μm to 40 μm, 10 μm to 35 μm, 15 μm to 35 μm, 18 μm to 32 μm, 20 μm to 30 μm, 20 μm to 25 μm, 25 μm to 30 μm, 5 μm, 10 μm, 15 μm, 20 μm, 21 μm, 22 μm, 23 μm, 24 μm, 25 μm, 26 μm, 27 μm, 28 μm, or 29 μm.

In some embodiments, the Dv50 value of the particles is from 100 μm to 200 μm, 110 μm to 190 μm, 120 μm to 180 μm, 100 μm to 200 μm, 100 μm to 200 μm, 100 μm to 200 μm, 100 μm to 200 μm, 100 μm to 200 μm, 100 μm to 200 μm, 100 μm to 200 μm, 100 μm to 200 μm, or 100 μm to 200 μm.

In some embodiments, the Dv90 value of the particles is from 0.01 μm to 400 μm, from 250 μm to 420 μm, from 250 μm to 400 μm, from 250 μm to 380 μm, from 270 μm to 380 μm, from 270 μm to 360 μm, from 270 μm to 350 μm, from 270 μm to 420 μm, from 290 μm to 420 μm, from 290 μm to 400 μm, from 290 μm to 380 μm, from 310 μm to 420 μm, from 310 μm to 400 μm, from 310 μm to 380 μm, from 330 μm to 420 μm, from 330 μm to 400 μm, from 330 μm to 380 μm, from 350 μm to 420 μm, from 350 μm to 400 μm, or from 370 μm to 420 μm.

In some embodiments, the particles are more uniformly distributed (e.g., have a smaller particle size range) than are the crude MDMA particles isolated from the synthetic process. In some embodiments, the particles have a particle size range that is less than 600 μm (e.g., less than 500 μm, less than 420 μm, or less than 400 m). In some embodiments, the particle size range is 5 μm to 500 μm (e.g., 5 μm to 420 μm, 20 μm to 353 μm, 20 μm to 326 μm, 21 μm to 353 μm, 21 μm to 342 μm, 21 μm to 326 μm, 24 μm to 353 μm, 29 μm to 342 μm, 34 μm to 341 μm, 200 μm to 400 μm, 230 μm to 380 μm, or 250 μm to 350 μm.

In some embodiments, the MDMA, or a pharmaceutically acceptable salt and/or solvate thereof are synthesized using the cGMP process disclosed in, for example, Scheme 1 of Nair, et al., ACS Omega 2022.

In some embodiments, the present disclosure provides, in part, particles less than about 610 μm in the composition as well as in the dosage form. In some embodiments, the present disclosure provides, in part, particles less than about 600 μm in the composition as well as in the dosage form. More specifically, MDMA particles with reduced particle size and increased particle size uniformity with a Dv90 below about 420 μm provide acceptable batch consistency enabling the production of pharmaceutically acceptable dosage forms.

Some embodiments provide a method of producing particles of MDMA, or a pharmaceutically acceptable salt and/or solvate thereof, wherein substantially all of the particles are less than about 610 μm, with a Dv90 less than 420 μm and a particle size range of less than 400 μm. In some embodiments, the method produces substantially no MDMA HCl hydrate. In some embodiments, the particles produced have a higher flowability than coarse MDMA particles.

Some embodiments provide a dosage form manufactured from the composition described herein, i.e., comprising MDMA particles substantially less than about 610 μm, with a Dv90 below 420 μm and a particle size range of less than 400 μm.

Some embodiments provide dosage forms manufactured from MDMA particles larger than 610 μm, but are reduced to the desired particle size of less than 610 μm with a Dv90 below 420 μm during the manufacturing of the finished product by milling or other means with one or more pharmaceutically acceptable excipients.

In some embodiments, substantially all of the particles are (i) less than about 610 μm, and (ii) have a Dv90 below about 400 μm. In some embodiments, the Dv90 is from about 0.01 μm to about 400 μm. In some embodiments, less than 10% of the particles have a particle size below about 10 μm (i.e., the particles have a Dv10 of about 10 μm). In some embodiments, from about 0% to about 10% of the particles have a particle size (Dv10) from about 0.01 μm to about 10 μm. In some embodiments, the median particle size (Dv50) is from about 100 μm to about 200 μm.

In some embodiments, the particles described herein are substantially free of MDMA·HCl monohydrate.

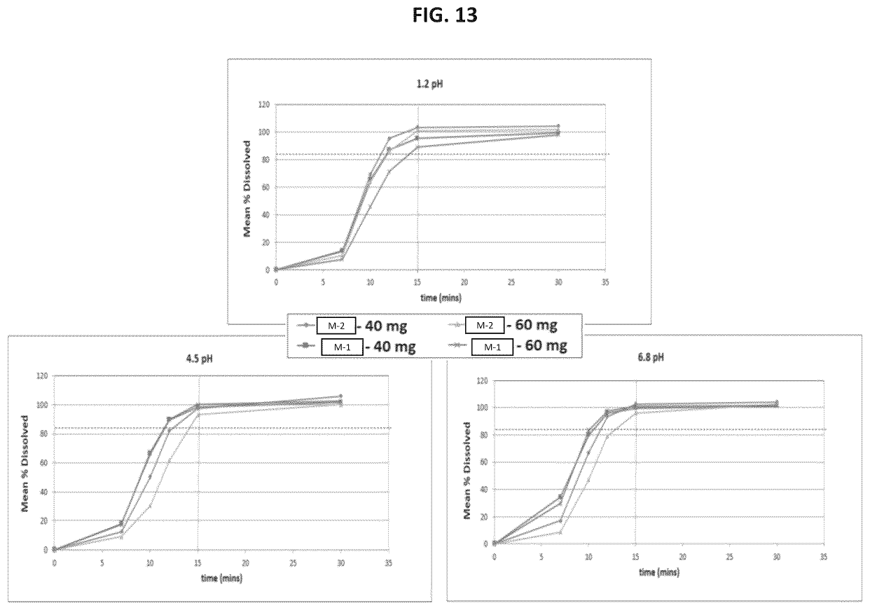

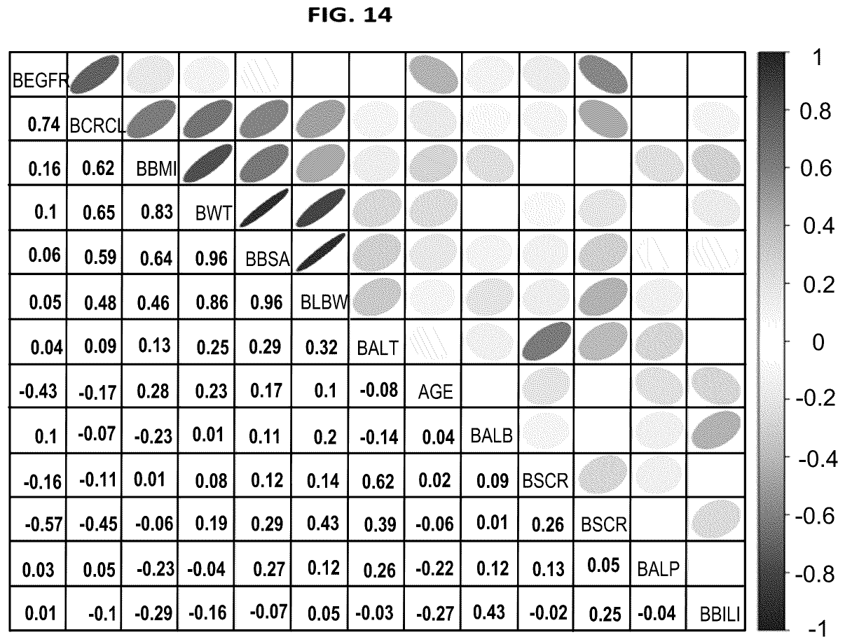

In some embodiments, the dissolution rate in water is greater than or equal to 50% (e.g., 60%, 70%, 80%, 85%, 90%, 95%, 98%, or 99%) of the mass of the MDMA in 30 minutes. In some embodiments, the dissolution rate in water is greater than or equal to 80% of the mass of the MDMA in 30 minutes. In some embodiments, the dissolution rate in water is greater than or equal to 95% of the mass of the MDMA in 30 minutes. In some embodiments, the dissolution rate in water is greater than or equal to 99% of the mass of the MDMA in 30 minutes. In some embodiments, the dissolution rate in water is greater than or equal to 90% of the mass of the MDMA in 15 minutes. In some embodiments, the dissolution rate in water is greater than or equal to 90% of the mass of the MDMA in 15 minutes. In some embodiments, the dissolution rate in water is greater than or equal to 95% of the mass of the MDMA in 15 minutes. In some embodiments, the dissolution rate in water is greater than or equal to 95% of the mass of the MDMA in 15 minutes. In some embodiments, the water has a pH of 1.2. In some embodiments, the water has a pH of 4.5. In some embodiments, the water has a pH of 6.8. In some embodiments, the dissolution profile of the compositions and/or unit dosage forms described herein is substantially similar to that described in FIG. 13.

In some embodiments, the MDMA, or a pharmaceutically acceptable salt and/or hydrate thereof, is amorphous. In some embodiments, the MDMA, or a pharmaceutically acceptable salt and/or hydrate thereof, is substantially amorphous. In some embodiments, the MDMA, or a pharmaceutically acceptable salt and/or hydrate thereof, contains less than 10% (e.g., less than 9%, less than 8%, less than 7%, less than 6%, less than 5%, less than 4%, less than 3%, less than 2%, or less than 1% crystalline MDMA, or a pharmaceutically acceptable salt and/or hydrate thereof.

In some embodiments, the MDMA, or a pharmaceutically acceptable salt and/or hydrate thereof, is crystalline. In some embodiments, the MDMA, or a pharmaceutically acceptable salt and/or hydrate thereof, is substantially crystalline. In some embodiments, the MDMA, or a pharmaceutically acceptable salt and/or hydrate thereof, is greater than 90% (e.g., greater than 92%, greater than 94%, greater than 96%, greater than 98%, or greater than 99%) crystalline. In some embodiments, the MDMA, or a pharmaceutically acceptable salt and/or hydrate thereof, contains less than 10% (e.g., less than 9%, less than 8%, less than 7%, less than 6%, less than 5%, less than 4%, less than 3%, less than 2%, or less than 1%) amorphous MDMA, or a pharmaceutically acceptable salt and/or hydrate thereof.

In some embodiments, the MDMA, or a pharmaceutically acceptable salt and/or hydrate thereof, is free of MDMA monohydrate. In some embodiments, the MDMA, or a pharmaceutically acceptable salt and/or hydrate thereof, is substantially free of MDMA monohydrate. In some embodiments, the MDMA, or a pharmaceutically acceptable salt and/or hydrate thereof, is present in one or more forms as described in Nair, et al., supra. In some embodiments, the particles described herein comprise MDMA in the form of the free base. In some embodiments, the particles described herein comprise MDMA in the form of anhydrous MDMA·HCl. In some embodiments, the particles described herein comprise MDMA·HCl Form I, anhydrous. In some embodiments, the particles described herein comprise MDMA·HCl Form II, anhydrous. In some embodiments, the particles described herein comprise MDMA·HCl Form III, anhydrous. In some embodiments, the particles described herein comprise MDMA·HCl in a mixture of Forms I, II, and III, each substantially free of MDMA monohydrate.

In some embodiments, the particles are prepared by a process comprising screen milling coarse particles under an inert atmosphere to reduce the particle size and increase the particle size uniformity of the coarse particles. In some embodiments, the coarse particles are particles isolated from the chemical synthesis of the MDMA. In some embodiments, the coarse particles do not undergo an additional size-reducing process.

In some embodiments, the median particle size (Dv50) of the coarse particles is greater than 400 μm. In some embodiments, the coarse particles are substantially free of a hydrate (e.g., monohydrate) of a pharmaceutically acceptable salt of MDMA. In some embodiments, the coarse particles are substantially free of MDMA·HCl monohydrate.

In some embodiments, the coarse particles are heated to a temperature of 50-70° C. in an environment having an ambient pressure below 1 atmosphere, before entering the screen mill. In some embodiments, the coarse particles are fed into the screen mill in the absence of applied pressure. In some embodiments, the inert atmosphere is substantially free of moisture. In some embodiments, the inert atmosphere comprises substantially dry nitrogen gas. In some embodiments, the inert atmosphere comprises substantially dry argon gas.

In some embodiments, the particles consist essentially of MDMA, or a pharmaceutically acceptable salt and/or solvate thereof.

In certain embodiments, the coarse MDMA crystals used to form the MDMA particles of the present disclosure are synthesized using the cGMP process disclosed in, for example, Scheme 1 of Nair et. al., ACS Omega 2022. The chemical purity of these coarse MDMA crystals as determined by a validated HPLC methodology may exceed 98% of total peak area. In some embodiments, the chemical purity of the coarse MDMA crystals exceeds 99% of total peak area. In some embodiments, the chemical purity of the coarse MDMA crystals exceeds 99.5% of total peak area. In some embodiments, the chemical purity of the coarse MDMA crystals exceeds 99.9% of total peak area.

### Methods of Manufacturing MDMA

The MDMA particles of the present disclosure can be prepared by any suitable processes known in the art. In certain embodiments, the MDMA particles of the present disclosure are prepared by a process described herein.

In some embodiments, the present disclosure provides new processes for preparing the MDMA particles of the present disclosure.

The form of crystalline MDMA isolated from the multi-kilogram cGMP synthesis described by Nair et. al., ACS Omega 2022, 7, 1, 900-907 is a highly pure, coarse solid with varying particle size. The coarse MDMA, having a typical Dv90 from 800 μm to 1600 μm and a typical particle size range from 500 μm to 1100 μm, does not yield a uniform blend. The distribution of the MDMA, or a pharmaceutically acceptable salt and/or solvate thereof, and one or more pharmaceutically acceptable excipients, in the resulting dosage form is uneven, leading to an unacceptably high rate of batch failure. In some embodiments, the median particle size (Dv50) of the coarse particles is greater than 400 μm. In some embodiments, the coarse particles are substantially free of MDMA·HCl monohydrate.

In one embodiment, the process comprises the step of reducing MDMA particle size from the typical Dv90 of from 800 μm to 1600 μm and/or typical particle size range from 500 m to 1100 μm by screen milling under an inert atmosphere. Screen milling processes known in the art can be used in the processes of the present disclosure. In one embodiment, screen milling in the processes of the present disclosure is performed using a conical screen miller, e.g., a Ytron-Quadro Comill.

One process of the present disclosure comprises the step of screen milling a batch of coarse particles comprising crystalline MDMA, or a pharmaceutically acceptable salt and/or solvate thereof, until the desired particle size reduction and increased particle uniformity are achieved.

In a first process, the coarse particles comprising crystalline MDMA, or a pharmaceutically acceptable salt and/or solvate thereof, are substantially dried under a vacuum at 50-70° C., then fed into a screen mill under an inert atmosphere that may comprise substantially dry nitrogen or any other substantially dry gas. The solids are fed into the mill in the absence of applied pressure, and captured in a collection bag upon exit.

In certain embodiments, the coarse particles comprising crystalline MDMA, or a pharmaceutically acceptable salt and/or solvate thereof, are fed into the screen mill in batches of approximately 250 grams, 500 grams, 1000 grams, or 2000 grams. In certain embodiments, the milling process is conducted at a rate of approximately 10 grams per minute, 15 grams per minute, 20 grams per minute, 25 grams per minute, or 50 grams per minute.

In certain embodiments, the screen milling in the processes described above is carried out by hand-feeding the coarse crystalline MDMA, or a pharmaceutically acceptable salt and/or solvate thereof, into the screen mill to avoid compacting and buildup within the mill.

In certain embodiments, a mill speed of 3000, 4000, 5000, 6000, 7000, or 8000 rpm is used.

In certain embodiments, the screen used in the processes described above is a stainless-steel conical screen.

In some embodiments, any one of the processes described above further comprises recovering and storing the MDMA particles after the screen milling step.

### Particles of MDMA and Excipient(s)

In some embodiments, the present disclosure provides 3,4-methylenedioxymethamphetamine particles that have desirable bulk properties and processability for drug product manufacturing.

MDMA isolated from the current chemical synthesis is a highly-pure, crystalline solid that is dimensionally unsuitable for drug product manufacturing. The coarse MDMA, with a typical Dv90 from about 800 μm to about 1600 μm and a typical particle size range from about 500 μm to about 1100 μm, does not blend satisfactorily with excipients during the formulation process. The distribution of API and excipient compound(s) in the resultant pharmaceutical composition is uneven, leading to an unacceptably high rate of batch failure and variability in dissolution profile and pharmacokinetics. Furthermore, pharmaceutical compositions formulated from coarse MDMA particles do not reliably formulate into dosage strengths to deliver MDMA at a rate sufficient to ensure a reproducible subject experience.

MDMA has multiple solid-state forms, including MDMA·HCl monohydrate that readily forms under ambient conditions, particularly during grinding and in high-humidity environments. The hydrate is significantly more hygroscopic than the API, and can adsorb up to two additional molar equivalents of surface moisture when fine particles are exposed to a high-humidity environment for an extended period of time. It is therefore necessary to reduce MDMA particle size in an environment that is unfavorable for hydrate formation.

It was initially proposed that acceptable MDMA particle size could be achieved by ball-milling the coarse MDMA crystals in the presence of a non-aqueous liquid dispersant. This was undesirable due to the high purity of the MDMA isolate, which was suitable for formulation without an additional purification step. It was unexpectedly discovered that MDMA particles with the reduced particle size and more uniform particle size range necessary for drug product manufacturing can be produced under dry conditions using a screen mill, under an inert atmosphere.

Some embodiments provide a composition comprising 3,4-methylenedioxymethamphetamine (MDMA), or a pharmaceutically acceptable salt and/or solvate thereof, and one or more pharmaceutically acceptable excipients, wherein the average particle size of the composition is from about 50 μm to about 610 μm. Some embodiments provide a composition comprising 3,4-methylenedioxymethamphetamine (MDMA), or a pharmaceutically acceptable salt and/or solvate thereof, and one or more pharmaceutically acceptable excipients, wherein the average particle size of the composition is from about 50 μm to about 450 μm. Some embodiments provide a composition comprising 3,4-methylenedioxymethamphetamine (MDMA), or a pharmaceutically acceptable salt and/or solvate thereof, and one or more pharmaceutically acceptable excipients, wherein the average particle size of the composition is from about 50 μm to about 400 μm.

In some embodiments, the average particle size of the composition is from about 50 m to about 610 μm. In some embodiments, the average particle size of the composition is from about 350 μm to about 420 μm. The excipients can be any excipients described herein, for example, magnesium stearate and/or mannitol.

In some embodiments, the average particle size of the composition is from about 50 m to about 100 μm, 100 μm to about 150 μm, 150 μm to about 200 μm, 200 μm to about 250 μm, 250 μm to about 300 μm, 350 μm to about 400 μm, 50 μm to about 150 μm, 150 μm to about 250 μm, 250 μm to about 400 μm, 200 μm to about 400 μm, 100 μm to about 300 μm, 200 m to about 400 μm, 100 μm to about 300 μm, 200 μm to about 400 μm, 400 μm to about 600 μm, 300 μm to about 400 μm, or 350 μm to about 550 μm. In some embodiments, the average particle size of the composition is from about 350 μm to about 450 μm. In some embodiments, the average particle size of the composition is from 350 μm to 450 μm. In some embodiments, the average particle size of the composition is about 420 μm. In some embodiments, the average particle size of the composition is 420 μm.

In some embodiments, the composition has desirable bulk properties and processability for preparing dosage forms suitable for administration to a subject.

MDMA isolated from the current chemical synthesis is a highly pure, coarse solid with varying particle size. The coarse MDMA, having a typical Dv90 from 800 μm to 1600 μm and a typical particle size range from 500 μm to 1100 μm, does not yield a uniform blend. The distribution of the MDMA, or a pharmaceutically acceptable salt and/or solvate thereof, and one or more pharmaceutically acceptable excipients, in the resulting dosage form is uneven, leading to an unacceptably high rate of batch failure.

It was initially proposed that acceptable MDMA particle size could be achieved by ball-milling the coarse MDMA particles in the presence of a non-aqueous liquid dispersant. This was undesirable due to the high purity of the MDMA isolate, which was suitable for formulation without an additional purification step. It was unexpectedly discovered that MDMA particles with the reduced particle size and more uniform particle size range necessary for drug product manufacturing can be produced under dry conditions using a screen mill, under an inert atmosphere. Alternatively, particles of the desired particle size can be produced by milling of the blends of the MDMA, or a pharmaceutically acceptable salt and/or solvate thereof, and other pharmaceutically acceptable excipient(s) or by other processes such as wet granulation, forming particles of a composition comprising MDMA, or a pharmaceutically acceptable salt and/or solvate thereof, and one or more pharmaceutically acceptable excipients.

In some embodiments, the composition particles are substantially less than about 610 μm. In some embodiments, substantially all of the particles may have volume diameters below about 610 μm. In some embodiments, substantially all of the particles may have at least one dimension less than about 610 μm.

In some embodiments, the Dv10 value of the particles is from about 5 μm to about 40 μm, about 5 μm to about 30 μm, about 5 μm to about 20 μm, about 5 μm to about 15 μm, about 10 μm to about 40 μm, about 15 μm to about 40 μm, about 20 μm to about 40 μm, about 25 m to about 40 μm, about 10 μm to about 35 μm, about 15 μm to about 35 μm, about 18 μm to about 32 μm, about 20 μm to about 30 μm, about 20 μm to about 25 μm, about 25 μm to about 30 μm, about 5 μm, about 10 μm, about 15 μm, about 20 μm, about 21 μm, about 22 μm, about 23 μm, about 24 μm, about 25 μm, about 26 μm, about 27 μm, about 28 μm, or about 29 μm.

In some embodiments, the Dv50 value of the particles is from about 100 μm to about 200 μm, about 110 μm to about 190 μm, about 120 μm to about 180 μm, about 100 μm to about 200 μm, about 100 μm to about 200 μm, about 100 μm to about 200 μm, about 100 μm to about 200 μm, about 100 μm to about 200 μm, about 100 μm to about 200 μm, about 100 μm to about 200 μm, about 100 μm to about 200 μm, or about 100 μm to about 200 μm.

In some embodiments, the Dv90 value of the particles is from about 0.01 μm to about 400 μm, from about 250 μm to about 420 μm, from about 250 μm to about 400 μm, from about 250 μm to about 380 μm, from about 270 μm to about 380 μm, from about 270 μm to about 360 μm, from about 270 μm to about 350 μm, from about 270 μm to about 420 μm, from about 290 μm to about 420 μm, from about 290 μm to about 400 μm, from about 290 μm to about 380 μm, from about 310 μm to about 420 μm, from about 310 μm to about 400 μm, from about 310 μm to about 380 μm, from about 330 μm to about 420 μm, from about 330 μm to about 400 μm, from about 330 μm to about 380 μm, from about 350 μm to about 420 μm, from about 350 μm to about 400 μm, or from about 370 μm to about 420 μm.

In certain embodiments, and the particle size range (Dv90-Dv10) of the particles is less than about 600 μm. In certain embodiments, and the particle size range (Dv90-Dv10) of the particles is less than about 550 μm. In certain embodiments, and the particle size range (Dv90-Dv10) of the particles is less than about 500 μm. In certain embodiments, and the particle size range (Dv90-Dv10) of the particles is less than about 450 μm. In certain embodiments, and the particle size range (Dv90-Dv10) of the particles is less than about 400 μm. In certain embodiments, and the particle size range (Dv90-Dv10) of the particles is less than about 350 μm. In certain embodiments, and the particle size range (Dv90-Dv10) of the particles is less than about 300 μm. In certain embodiments, and the particle size range (Dv90-Dv10) of the particles is less than about 275 μm.

In some embodiments, the composition particles are more uniformly distributed (e.g., have a smaller particle size range) than are the crude MDMA particles isolated from the synthetic process (i.e., the coarse particles). In some embodiments, the composition particles have a particle size range that is less than about 600 μm (e.g., less than about 500 μm, less than about 420 μm, or less than about 400 m). In some embodiments, the particle size range is about 5 μm to about 500 μm (e.g., about 5 μm to about 420 μm, about 20 μm to about 353 μm, about 20 μm to about 326 μm, about 21 μm to about 353 μm, about 21 μm to about 342 μm, about 21 μm to about 326 μm, about 24 μm to about 353 μm, about 29 μm to about 342 μm, about 34 μm to about 341 μm, about 200 μm to about 400 μm, about 230 μm to 380 μm, or about 250 μm to 350 μm.

In some embodiments, the MDMA, or a pharmaceutically acceptable salt and/or solvate thereof, and one or more pharmaceutically acceptable excipients, used to form the particles are synthesized using the cGMP process disclosed in, for example, Scheme 1 of Nair, et al., supra.

In some embodiments, the present disclosure provides, in part, particles less than about 610 μm in the composition as well as the dosage form. More specifically, composition particles with reduced particle size and increased particle size uniformity with a Dv90 below about 420 μm provide acceptable batch consistency enabling the production of pharmaceutically acceptable dosage forms.

Some embodiments provide a method of producing a composition comprising particles substantially less than about 610 μm, with a Dv90 less than 420 μm and a particle size range of less than 400 μm, wherein the particles comprise MDMA, or a pharmaceutically acceptable salt and/or solvate thereof, and one or more pharmaceutically acceptable excipients. In some embodiments, the method produces substantially no hydrate (e.g., monohydrate) of a pharmaceutically acceptable salt of MDMA (e.g., MDMA·HCl). In some embodiments, the particles produced have a higher flowability than coarse MDMA particles.

Some embodiments provide a dosage form prepared from a composition described herein. In some embodiments, the composition comprises particles substantially less than about 610 μm, with a Dv90 below 420 μm and a particle size range of less than 400 μm.

Some embodiments provide a dosage form comprising a composition comprising particles substantially less than about 610 μm, with a Dv90 less than 420 μm and a particle size range of less than 400 μm, wherein the particles comprise MDMA, or a pharmaceutically acceptable salt and/or solvate thereof, and one or more pharmaceutically acceptable excipients.

Some embodiments provide a dosage form prepared by a method comprising:
blending particles having an average particle size greater than 610 μm with one or more pharmaceutically acceptable excipients;
changing the average particle size of the particles to less than 610 μm and a Dv90 below 420 μm;
wherein the particles comprise MDMA, or a pharmaceutically acceptable salt and/or solvate thereof, and one or more pharmaceutically acceptable excipients.

In some embodiments, the changing comprises milling the particles with the one or more pharmaceutically acceptable excipients.

In some embodiments, substantially all of the particles are (i) less than about 610 μm, and (ii) have a Dv90 lesser than about 400 μm. In some embodiments, the Dv90 is from about 0.01 μm to about 400 μm. In some embodiments, less than 10% of the particles have a particle size (Dv10) below about 10 μm. In some embodiments, from about 0% to about 10% of the particles have a particle size (Dv10) from about 0.01 μm to about 10 μm. In some embodiments, the median particle size (Dv50) is from about 100 μm and about 200 μm. In some embodiments, the median particle size (Dv50) is from about 70 μm and about 250 μm.

In some embodiments, the particles are substantially free of MDMA·HCl monohydrate.

In some embodiments, at least 80% of the mass of the MDMA particles dissolves in water in 30 minutes or less.

In some embodiments, the MDMA, or a pharmaceutically acceptable salt and/or hydrate thereof, is amorphous. In some embodiments, the MDMA, or a pharmaceutically acceptable salt and/or hydrate thereof, is substantially amorphous. In some embodiments, the MDMA, or a pharmaceutically acceptable salt and/or hydrate thereof, is crystalline. In some embodiments, the MDMA, or a pharmaceutically acceptable salt and/or hydrate thereof, is substantially crystalline. In some embodiments, the MDMA, or a pharmaceutically acceptable salt and/or hydrate thereof, is free of MDMA·HCl monohydrate. In some embodiments, the MDMA, or a pharmaceutically acceptable salt and/or hydrate thereof, is substantially free of MDMA·HCl monohydrate. In some embodiments, the MDMA, or a pharmaceutically acceptable salt and/or hydrate thereof, comprises one or more forms as described in Nair, et al., supra.

In certain embodiments, the coarse MDMA crystals used to form the MDMA particles of the present disclosure are synthesized using the cGMP process disclosed in, for example, Scheme 1 of Nair et. al., ACS Omega 2022. In some embodiments, the particles described herein comprise MDMA in the form of the free base. In some embodiments, the particles described herein comprise MDMA in the form of anhydrous MDMA·HCl. In some embodiments, the particles described herein comprise MDMA·HCl Form I, anhydrous. In some embodiments, the particles described herein comprise MDMA·HCl Form II, anhydrous. In some embodiments, the particles described herein comprise MDMA·HCl Form III, anhydrous. In some embodiments, the particles described herein comprise MDMA·HCl in a mixture of Forms I, II, and III, each substantially free of MDMA monohydrate.

The chemical purity of these coarse MDMA crystals as determined by a validated HPLC methodology may exceed 98% of total peak area. In some embodiments, the chemical purity of the coarse MDMA crystals exceeds 99% of total peak area. In some embodiments, the chemical purity of the coarse MDMA crystals exceeds 99.5% of total peak area. In some embodiments, the chemical purity of the coarse MDMA crystals exceeds 99.9% of total peak area.

In any of the compositions described herein, the particles of the composition are prepared by a process comprising a step of reducing average particle size and increasing particle size uniformity by screen-milling under an inert atmosphere.

In some embodiments, the composition includes a diluent. In some embodiments, the diluent is a sugar alcohol. In some embodiments, the diluent has a moisture content of less than 0.25% by mass, prior to blending with the MDMA, or a pharmaceutically acceptable salt and/or hydrate thereof.

In some embodiments, in the compositions described herein the desired particle size and particles size uniformity is achieved in the process of making the finished dosage form by milling or other means.

In some embodiments, the compositions described herein can additionally include a lubricant. In some embodiments, the lubricant includes a pharmaceutically acceptable salt of a saturated fatty acid.

In some embodiments, the particles are prepared by a process comprising the step of reducing particle size and increasing particle size uniformity by screen milling. In some embodiments, the coarse particles do not undergo an additional size-reducing process.

In some embodiments, the median particle size (Dv50) of the coarse particles is greater than 400 μm. In some embodiments, the coarse particles are substantially free of MDMA·HCl monohydrate.

In some embodiments, the coarse particles are heated to a temperature of 50-70° C. in an environment having an ambient pressure below 1 atmosphere, before entering the screen mill. In some embodiments, the coarse particles are fed into the screen mill in the absence of applied pressure. In some embodiments, the inert atmosphere is substantially free of moisture. In some embodiments, the inert atmosphere comprises substantially dry nitrogen gas. In some embodiments, the inert atmosphere comprises substantially dry argon gas.

In some embodiments, the particles consist essentially of MDMA, or a pharmaceutically acceptable salt and/or solvate thereof, and one or more pharmaceutically acceptable excipients.

### Dosage Forms

Some embodiments provide a dosage form comprising a composition comprising 3,4-methylenedioxymethamphetamine (MDMA), or a pharmaceutically acceptable salt and/or solvate thereof, and one or more pharmaceutically acceptable excipients, wherein the average particle size of the composition in the dosage form is from about 50 μm to about 400 μm. Some embodiments provide a dosage form comprising a composition comprising 3,4-methylenedioxymethamphetamine (MDMA), or a pharmaceutically acceptable salt and/or solvate thereof, and one or more pharmaceutically acceptable excipients, wherein the average particle size of the composition in the dosage form is from about 50 μm to about 600 μm.

Some embodiments provide a dosage form comprising a composition comprising 3,4-methylenedioxymethamphetamine (MDMA), or a pharmaceutically acceptable salt and/or solvate thereof, and one or more pharmaceutically acceptable excipients, wherein the average particle size of the composition in the dosage form is from about 350 μm to about 450 μm.

Some embodiments provide a dosage form comprising a composition comprising 3,4-methylenedioxymethamphetamine (MDMA), or a pharmaceutically acceptable salt and/or solvate thereof, and one or more pharmaceutically acceptable excipients, wherein the average particle size of the MDMA, or a pharmaceutically acceptable salt and/or solvate thereof, is from about 50 μm to about 400 μm.

Exemplary, non-limiting pharmaceutically acceptable excipients are described below. Additional excipients and general methods for preparing the types of dosage forms described herein can be found in, for example, Remington: The Science and Practice of Pharmacy, 23^{rd} Edition (Elsevier Science, Amsterdam, N L. 2020). Such pharmaceutically acceptable excipients include, but are not limited to, binders, glidants, disintegrants, lubricants, carriers, diluents, buffers, tonicity modifying agents, polymers, thickening agents, penetration enhancers, surfactants, and solubility enhancers. See, e.g., Remington's, supra. Some pharmaceutically acceptable excipients can be in more than one of the foregoing sub-categories. Pharmaceutically acceptable excipients also include dosage form coatings, for example, an extended release coating, abuse-deterrent coating, or a film-coating.

Pharmaceutical carriers or excipients in accordance with the present disclosure can be selected for their compatibility with a given dosage form. Exemplary excipients for oral formulations include, but are not limited to: diluents, such as microcrystalline cellulose, starch, mannitol, calcium hydrogen phosphate anhydrous or co mixtures of silicon dioxide, calcium carbonate, microcrystalline cellulose and talc; disintegrants, such as sodium starch glycolate or croscarmellose sodium; binders, such as povidone, co povidone or hydroxyl propyl cellulose; lubricants, such as magnesium stearate or sodium stearyl fumarate; glidants, such as colloidal silicon dioxide; and film coats, such as Opadry II white or PVA based brown Opadry II. Exemplary excipients for topical formulations include, but are not limited to: polymers, such as xanthan gum or hydroxypropyl methylcellulose; preservatives, such as methyl- and propylparaben; surface-acting agents such as sodium lauryl sulfate, phosphatidylcholine, betaines, or polyoxyethylene sorbitan fatty acid esters; and penetration enhancers such as ethanol, dimethyl sulfoxide, dimethyl isosorbide, isopropyl myristate or propylene glycol. Exemplary excipients for respiratory dosage forms include, but are not limited to: propellants such as heptafluoropropane and other hydrofluorocarbons; surface-active agents such as sorbitan trioleate, oleic acid, or sorbitan sesquioleate; solubility enhancers such as ethanol, propylene glycol, or glycerol; flow improvers such as lactose; buffering agents such as sodium citrate or sodium phosphate; osmolality-modifying agents such as sodium chloride or mannitol; antioxidants; and preservatives. Exemplary excipients for parenteral dosage forms include, but are not limited to: bulking agents such as sucrose, mannitol, or sorbitol; buffering agents such as sodium citrate, tris base-65, tris acetate, or sodium phosphate; antioxidants such as acetone sodium bisulfite or ascrobyl palmitate; solubilizing agents such as polyvinyl pyrollidone or lecithin; preservatives such as benzalkonium chloride, paraben propyl, phenol, or thimerosal; lyoprotectants such as sucrose, trehalose, or mannitol; chelating agents such as calcium disodium EDTA or calteridol; and solvents and cosolvents such as castor oil, PEG 300, N-methyl-2-pyrrolidone, or propylene glycol.

In a specific embodiment, a pharmaceutical composition of the present disclosure comprises particles comprising crystalline MDMA, or a pharmaceutically acceptable salt and/or solvate thereof, substantially less than about 610 μm, with a Dv90 below 420 μm and a particle size range of less than 400 μm, a binder comprising a polyalcohol, and a lubricant comprising a pharmaceutically acceptable salt of a saturated fatty acid.

In some embodiments, pharmaceutically acceptable excipients used herein have reduced hygroscopicity and/or low residual moisture content.

In some embodiments, the pharmaceutically acceptable excipients used herein are independently selected from the group consisting of: microcellulose, lactose (e.g., α-lactose monohydrate), starch, mannitol, calcium hydrogen phosphate anhydrous, silicon dioxide, calcium carbonate, microcellulose, talc, sodium starch glycolate, croscarmellose sodium, povidone, copovidone or hydroxyl propyl cellulose, magnesium stearate, sodium stearyl fumarate, and colloidal silicon dioxide. In some embodiments, the pharmaceutically acceptable excipient comprises α-lactose monohydrate. In some embodiments, the pharmaceutically acceptable excipient comprises magnesium stearate and mannitol.

In some embodiments, the dosage form comprises particles substantially less than about 610 μm, with a Dv90 below 420 μm and a particle size range of less than 400 μm. The dosage forms comprise MDMA, or a pharmaceutically acceptable salt and/or solvate thereof, in accordance with any embodiment as described herein. The dosage forms can be intended for topical, oral, nasal, mucosal, respiratory, transdermal, or parenteral administration.

The dosage forms provided herein include, but are not limited to, solid formulations such as tablets, capsules, pills, wafers, films, and lozenges, or liquid formulations such as aqueous solutions, elixirs, and syrups. Solid and liquid formulations in accordance with the present disclosure may also be incorporated into liquid or solid comestibles.

In some embodiments, the dosage form comprises encapsulated pharmaceutical formulations provided by any other embodiment as described herein. Capsules used for the dosage form can be hard-shelled or soft-shelled. The capsules may comprise collagenous gelatin, fish gelatin, hydroxypropyl methylcellulose, starch, pullulan, polyvinyl acetate, or any other material known to a person skilled in the art to be useful for encapsulating dosage forms.

Oral dosage forms provided by the present disclosure can be solid formulations such as tablets, capsules, pills, wafers, films, and lozenges, or liquid formulations such as aqueous solutions, elixirs, and syrups. Solid and liquid formulations in accordance with the present disclosure may also be incorporated into liquid or solid comestibles.

In certain embodiments, the oral dosage forms provided by the present disclosure may comprise encapsulated pharmaceutical formulations provided by any other embodiment of the present disclosure. Capsules used for the oral dosage form of the present disclosure can be hard-shelled or soft-shelled. The capsules may comprise collagenous gelatin, fish gelatin, hydroxypropyl methylcellulose, starch, pullulan, polyvinyl acetate, or any other material known to a person skilled in the art to be useful for encapsulating oral dosage forms.

Topical dosage forms provided by the present disclosure can be liquid formulations such as aqueous solutions and emulsions, which can be applied directly to the skin and/or mucous membranes, or aerosolized for respiratory administration. Alternatively, topical dosage forms provided by the present disclosure can be formulated as creams, foams, gels, lotions, and ointments.

Respiratory dosage forms provided by the present disclosure may comprise solid compositions formulated for use in dry-powder inhalers, or liquid compositions formulated for use in metered-dose inhalers or nebulizers.

Parenteral dosage forms provided by the present disclosure may comprise liquid solutions, suspensions, emulsions, or reconstituted lyophilized powders, suitable for administration by injection.

In some embodiments, the dosage form comprises liquid formulations formulated for topical administration, such as aqueous solutions and emulsions, which can be applied directly to the skin and/or mucous membranes, or aerosolized for respiratory administration. Alternatively, topical dosage forms provided by the present disclosure can be formulated as creams, foams, gels, lotions, and ointments.

In some embodiments, the dosage form comprises solid compositions formulated for respiratory or inhalation administration, for example, for use in dry-powder inhalers, or liquid compositions formulated for use in metered-dose inhalers or nebulizers.

In some embodiments, the dosage form comprises a liquid solutions formulated for parenteral administration, such as suspensions, emulsions, or reconstituted lyophilized powders, suitable for administration by injection.

In some embodiments, the dosage form is in the form of a capsule, for example a cellulose-based capsule containing the composition described herein. In some embodiments, the dosage form is a hydroxypropylmethylcellulose (HPMC) capsule. In some embodiments, the capsule is a gelatin capsule.

In some embodiments, the dosage form comprises about 1 mg to about 150 mg of MDMA, or a pharmaceutically acceptable salt and/or solvate thereof, on a free base basis of MDMA. In some embodiments, the dosage form comprises about 17 mg to about 126 mg of MDMA, or a pharmaceutically acceptable salt and/or solvate thereof, on a free base basis of MDMA. In some embodiments, the dosage form comprises about 1 mg to about 50 mg of MDMA, or a pharmaceutically acceptable salt and/or solvate thereof, on a free base basis of MDMA. In some embodiments, the dosage form comprises about 68 mg to about 100 mg of MDMA, or a pharmaceutically acceptable salt and/or solvate thereof, on a free base basis of MDMA. In some embodiments, the dosage form comprises about 50 mg to about 130 mg of MDMA, or a pharmaceutically acceptable salt and/or solvate thereof, on a free base basis of MDMA. In some embodiments, the dosage form comprises about 25 mg to about 75 mg of MDMA, or a pharmaceutically acceptable salt and/or solvate thereof, on a free base basis of MDMA. In some embodiments, the dosage form comprises about 50 mg to about 100 mg of MDMA, or a pharmaceutically acceptable salt and/or solvate thereof, on a free base basis of MDMA. In some embodiments, the dosage form comprises about 75 mg to about 125 mg of MDMA, or a pharmaceutically acceptable salt and/or solvate thereof, on a free base basis of MDMA. In some embodiments, the dosage form comprises about 100 mg to about 150 mg of MDMA, or a pharmaceutically acceptable salt and/or solvate thereof, on a free base basis of MDMA.

In some embodiments, the amount of MDMA, or a pharmaceutically acceptable salt and/or solvate thereof, is expressed on a free base basis of MDMA.

In some embodiments, the dosage form comprises about 12.5 mg, about 15 mg, about 20 mg, about 25 mg, about 30 mg, about 34 mg, about 35 mg, about 37.5 mg, about 40 mg, about 45 mg, about 50 mg, about 55 mg, about 60 mg, about 62.5 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg of MDMA, or a pharmaceutically acceptable salt and/or solvate thereof, on a free base basis of MDMA.

In some embodiments, the dosage form comprises 12.5 mg, 15 mg, 20 mg, 25 mg, 30 mg, about 34 mg, 35 mg, 37.5 mg, 40 mg, 45 mg, 50 mg, 55 mg, 60 mg, 62.5 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, or 150 mg of MDMA, or a pharmaceutically acceptable salt and/or solvate thereof, on a free base basis of MDMA.

In some embodiments, the dosage form comprises about 12.5 mg, about 15 mg, about 20 mg, about 25 mg, about 30 mg, about 34 mg, about 37.5 mg, about 40 mg, about 50 mg, about 60 mg, about 62.5 mg, about 75 mg, about 80 mg, about 100 mg, or about 125 mg of MDMA, or a pharmaceutically acceptable salt and/or solvate thereof, on a free base basis of MDMA.

In some embodiments, the dosage form comprises 12.5 mg, 15 mg, 20 mg, 25 mg, 30 mg, about 34 mg, 37.5 mg, 40 mg, 50 mg, 60 mg, 62.5 mg, 75 mg, 80 mg, 100 mg, or 125 mg of MDMA, or a pharmaceutically acceptable salt and/or solvate thereof, on a free base basis of MDMA.

In some embodiments, the MDMA, or a pharmaceutically acceptable salt and/or solvate thereof is MDMA hydrochloride (MDMA·HCl). In some embodiments, the amount of MDMA is on the basis of MDMA·HCl. In some embodiments, the dosage form comprises about 1 mg to about 180 mg (e.g., about 20 mg to about 150 mg, about 30 mg to about 140 mg, about 40 mg to about 130 mg, about 30 mg to about 70 mg, about 40 mg to about 60 mg, about 80 mg to about 120 mg, about 120 mg to about 180 mg, about 30 mg to about 50 mg, about 35 mg to about 45 mg, about 55 mg to about 65 mg, about 20 mg, about 40 mg, about 60 mg, about 80 mg, about 120 mg, about 150 mg, or about 180 mg) MDMA·HCl. In some embodiments, the dosage form comprises about 120 mg to about 180 mg MDMA·HCl. In some embodiments, the dosage form comprises about 20 mg to about 150 mg MDMA·HCl. In some embodiments, the dosage form comprises about 80 mg to about 120 mg MDMA·HCl. In some embodiments, the dosage form comprises about 40 mg to about 60 mg MDMA·HCl. In some embodiments, the dosage form comprises about 20 mg MDMA·HCl. In some embodiments, the dosage form comprises about 40 mg MDMA·HCl. In some embodiments, the dosage form comprises about 60 mg MDMA·HCl. In some embodiments, the dosage form comprises about 80 mg MDMA·HCl. In some embodiments, the dosage form comprises about 100 mg MDMA·HCl. In some embodiments, the dosage form comprises about 120 mg MDMA·HCl. In some embodiments, the dosage form comprises about 150 mg MDMA·HCl. In some embodiments, the dosage form comprises about 180 mg MDMA·HCl.

In some embodiments, the dosage form comprises particles substantially less than about 610 μm, with a Dv90 below 420 μm and a particle size range of less than 400 μm, and one or more pharmaceutically acceptable excipients.

In some embodiments, the dosage form comprises particles substantially less than about 610 μm that are prepared by a product comprising milling a mixture of MDMA with one or more pharmaceutically excipients as described herein.

In some embodiments, the dosage form comprises particles substantially less than about 610 μm, with a Dv90 below 420 μm and a particle size range of less than 400 μm, a binder comprising a polyalcohol, and a lubricant comprising a pharmaceutically acceptable salt of a saturated fatty acid.

In some embodiments, the dosage form is substantially free of a hydrate of a pharmaceutically acceptable salt of MDMA. In some embodiments, the dosage form is substantially free of MDMA·HCl monohydrate. In some embodiments, the dosage form comprises no detectable MDMA·HCl monohydrate.

In some embodiments, each of the dosage form is a tablet. In some embodiments, the dosage form is a capsule. In some embodiments, the dosage form includes one or more individual dosage units, for example, in some embodiments, the dosage form is a blister pack. In some embodiments, the dosage form includes one individual dosage unit. In some embodiments, the dosage form includes at least two individual dosage units. In some embodiments, the dosage form includes three individual dosage units. In some embodiments, the dosage form includes at least three individual dosage units. In some embodiments, each of the one or more individual dosage units comprises a capsule. In some embodiments, each of the one or more individual dosage units comprises a tablet. In some embodiments, each of the one or more individual dosage units comprises a capsule. In some embodiments, each of the one or more individual dosage units is a tablet. In some embodiments, each of the one or more individual dosage units is a capsule.

In some embodiments, the one or more individual dosage units are administered during a single psychotherapy session. In some embodiments, the one or more individual dosage units are administered at different times during the single psychotherapy session.

In some embodiments, the one or more individual dosage units are administered during the course of a psychological intervention. In some embodiments, the one or more individual dosage units are administered at different times during the psychological intervention.

In some embodiments, the one or more individual dosage units are administered during a single medication session. In some embodiments, the one or more individual dosage units are administered at different times during the single medication session. In a medication session, there is another individual present during the administration (in person or virtually). Any of the methods described herein that reference a psychological intervention and/or psychotherapy session can be conducted as a medication session.

As used herein, an individual dosage unit (i.e., a tablet or capsule provided in a blister pack) has the same characteristics of the dosage forms described herein that are not comprised of individual dosage units (i.e., the dosage forms such as tablets, capsules, and the like). As such, each individual dosage unit is a dosage form comprising MDMA, or a pharmaceutically acceptable salt and/or solvate thereof (for example, about 34 mg MDMA, or a pharmaceutically acceptable salt and/or solvate thereof; or about 50 mg MDMA, or a pharmaceutically acceptable salt and/or solvate thereof).

In some embodiments, the dosage form is a blister pack comprising three individual dosage units comprising the MDMA compositions described herein. In some embodiments, the dosage form is a blister pack comprising three capsules comprising the MDMA compositions described herein. In some embodiments, the dosage form comprises 102 mg of MDMA. In some embodiments, the dosage form comprises 150 mg of MDMA. In some embodiments, each individual dosage unit comprises 34 mg of MDMA. In some embodiments, each individual dosage unit comprises 50 mg of MDMA.

Some embodiments provide a dosage form comprising a capsule comprising gelatin, wherein the capsule contains a composition comprising:
MDMA, wherein substantially all of the MDMA particles have a particle size less than about 610 μm, a Dv90 below 420 μm, and a particle size range of less than 400 km; and
α-lactose monohydrate.

Some embodiments provide a dosage form comprising a capsule comprising gelatin, wherein the capsule contains a composition comprising:
(a) MDMA, or a pharmaceutically acceptable salt thereof; and
(b) α-lactose monohydrate;
wherein substantially all of the particles of the composition have a particle size less than about 610 μm, a Dv90 below 420 μm, and a particle size range of less than 400 μm.

Some embodiments provide a dosage form comprising a capsule comprising gelatin, wherein the capsule contains a composition comprising:
(a) MDMA·HCl, wherein substantially all of the MDMA·HCl particles have a particle size less than about 610 km, a Dv90 below 420 km, and a particle size range of less than 400 μm; and
(b) α-lactose monohydrate.

Some embodiments provide a dosage form comprising a capsule comprising gelatin, wherein the capsule contains a composition comprising:
(a) MDMA·HCl; and
(b) α-lactose monohydrate;
wherein substantially all of the particles of the composition have a particle size less than about 610 μm, a Dv90 below 420 μm, and a particle size range of less than 400 μm.

Some embodiments provide a dosage form comprising a capsule comprising gelatin, wherein the capsule contains a composition comprising:
(a) about 34 mg or about 50 mg MDMA, or a pharmaceutically acceptable salt thereof, wherein substantially all of the MDMA·HCl particles have a particle size less than about 610 μm, a Dv90 below 420 μm, and a particle size range of less than 400 μm; and
(b) α-lactose monohydrate.

Some embodiments provide a dosage form comprising a capsule comprising gelatin, wherein the capsule contains a composition comprising:
(a) about 34 mg or about 50 mg MDMA, or a pharmaceutically acceptable salt thereof; and
(b) α-lactose monohydrate;
wherein substantially all of the particles of the composition have a particle size less than about 610 μm, a Dv90 below 420 μm, and a particle size range of less than 400 μm.

Some embodiments provide a dosage form comprising:
(a) about 50% particles comprising MDMA, or a pharmaceutically acceptable salt thereof, wherein substantially all of the MDMA particles have a particle size less than about 610 μm, a Dv90 below 420 μm, and a particle size range of less than 400 μm;
(b) about 0.1% to about 2% by weight of magnesium stearate; and
(c) about 48% to about 49.9% by weight of mannitol.

Some embodiments provide a dosage form comprising:
(a) about 50% particles consisting essentially of MDMA, or a pharmaceutically acceptable salt thereof, wherein substantially all of the MDMA particles have a particle size less than about 610 μm, a Dv90 below 420 μm, and a particle size range of less than 400 μm;
(b) about 0.1% to about 2% by weight of magnesium stearate; and
(c) about 48% to about 49.9% by weight of mannitol.

Some embodiments provide a dosage form consisting essentially of:
(a) about 50% particles consisting essentially of MDMA, or a pharmaceutically acceptable salt thereof, wherein substantially all of the MDMA particles have a particle size less than about 610 μm, a Dv90 below 420 μm, and a particle size range of less than 400 μm;
(b) about 0.1% to about 2% by weight of magnesium stearate; and
(c) about 48% to about 49.9% by weight of mannitol.

Some embodiments provide a dosage form comprising a capsule comprising hydroxypropylmethylcellulose (HPMC), wherein the capsule contains a composition comprising:
(a) about 34 mg or about 50 mg MDMA- or a pharmaceutically acceptable salt thereof, wherein substantially all of the MDMA particles have a particle size less than about 610 μm, a Dv90 below 420 μm, and a particle size range of less than 400 μm;
(b) about 0.1% to about 10% by weight of magnesium stearate; and
(c) about 25% to about 75% by weight of mannitol.

Some embodiments provide a dosage form comprising a capsule comprising hydroxypropylmethylcellulose (HPMC), wherein the capsule contains a composition comprising:
(a) about 34 mg or about 50 mg MDMA- or a pharmaceutically acceptable salt thereof;
(b) about 0.1% to about 10% by weight of magnesium stearate; and
(c) about 25% to about 75% by weight of mannitol;
wherein substantially all of the particles of the composition have a particle size less than about 610 μm, a Dv90 below 420 μm, and a particle size range of less than 400 μm.

Some embodiments provide a dosage form comprising a capsule comprising hydroxypropylmethylcellulose (HPMC), wherein the capsule contains a composition comprising:
(a) about 34 mg or about 50 mg MDMA- or a pharmaceutically acceptable salt thereof, wherein substantially all of the MDMA·HCl particles have a particle size less than about 610 μm, a Dv90 below 420 μm, and a particle size range of less than 400 μm;
(b) about 0.1% to about 10% by weight of magnesium stearate; and
(c) about 25% to about 75% by weight of mannitol.

Some embodiments provide a dosage form comprising a capsule comprising hydroxypropylmethylcellulose (HPMC), wherein the capsule contains a composition comprising:
(a) about 34 mg MDMA, or a pharmaceutically acceptable salt thereof,
(b) about 0.1% to about 10% by weight of magnesium stearate; and
(c) about 25% to about 75% by weight of mannitol;
wherein substantially all of the particles of the composition have a particle size less than about 610 μm, a Dv90 below 420 μm, and a particle size range of less than 400 μm.

Some embodiments provide a dosage form comprising a capsule comprising hydroxypropylmethylcellulose (HPMC), wherein the capsule contains a composition comprising:
(a) about 50 mg MDMA, or a pharmaceutically acceptable salt thereof, wherein substantially all of the MDMA·HCl particles have a particle size less than about 610 μm, a Dv90 below 420 μm, and a particle size range of less than 400 μm;
(b) about 0.1% to about 10% by weight of magnesium stearate; and
(c) about 25% to about 75% by weight of mannitol.

Some embodiments provide a dosage form comprising:
(a) about 34 mg of particles comprising MDMA, or a pharmaceutically acceptable salt thereof;
(b) about 0.1% to about 2% by weight of magnesium stearate; and
(c) about 45% to about 55% by weight of mannitol;
wherein substantially all of the particles of the MDMA, or a pharmaceutically acceptable salt thereof, have a particle size less than about 610 μm, a Dv90 below 420 μm, and a particle size range of less than 400 μm.

Some embodiments provide a dosage form comprising:
(a) about 50 mg of particles comprising MDMA, or a pharmaceutically acceptable salt thereof;
(b) about 0.1% to about 2% by weight of magnesium stearate; and
(c) about 45% to about 55% by weight of mannitol;
wherein substantially all of the particles of the MDMA, or a pharmaceutically acceptable salt thereof, have a particle size less than about 610 μm, a Dv90 below 420 μm, and a particle size range of less than 400 μm.

Some embodiments provide a dosage form consisting of:
(a) about 34 mg of particles of MDMA, or a pharmaceutically acceptable salt thereof;
(b) about 0.1% to about 2% by weight of magnesium stearate; and
(c) about 45% to about 55% by weight of mannitol;
wherein substantially all of the particles of the MDMA, or a pharmaceutically acceptable salt thereof; have a particle size less than about 610 μm, a Dv90 below 420 μm, and a particle size range of less than 400 μm.

Some embodiments provide a dosage form consisting of:
(a) about 50 mg of particles of MDMA, or a pharmaceutically acceptable salt thereof;
(b) about 0.1% to about 2% by weight of magnesium stearate; and
(c) about 45% to about 55% by weight of mannitol;
wherein substantially all of the particles of the MDMA, or a pharmaceutically acceptable salt thereof; have a particle size less than about 610 μm, a Dv90 below 420 μm, and a particle size range of less than 400 μm.

Some embodiments provide a dosage form comprising a capsule comprising hydroxypropylmethylcellulose (HPMC), wherein the capsule contains a composition comprising:
(a) about 50 mg MDMA, or a pharmaceutically acceptable salt thereof,
(b) about 0.1% to about 10% by weight of magnesium stearate; and
(c) about 25% to about 75% by weight of mannitol;
wherein substantially all of the particles of the composition have a particle size less than about 610 μm, a Dv90 below 420 μm, and a particle size range of less than 400 μm.

Some embodiments provide a dosage form comprising a capsule comprising hydroxypropylmethylcellulose (HPMC), wherein the capsule contains a composition comprising:
(a) about 34 mg MDMA on a free base basis of MDMA, wherein substantially all of the MDMA particles have a particle size less than about 610 μm, a Dv90 below 420 μm, and a particle size range of less than 400 μm;
(b) about 0.1% to about 10% by weight of magnesium stearate; and
(c) about 25% to about 75% by weight of mannitol.

Some embodiments provide a dosage form comprising a capsule comprising hydroxypropylmethylcellulose (HPMC), wherein the capsule contains a composition comprising:
(a) about 34 mg MDMA on a free base basis of MDMA;
(b) about 0.1% to about 10% by weight of magnesium stearate; and
(c) about 25% to about 75% by weight of mannitol;
wherein substantially all of the particles of the composition have a particle size less than about 610 μm, a Dv90 below 420 μm, and a particle size range of less than 400 μm.

Some embodiments provide a dosage form comprising a capsule comprising hydroxypropylmethylcellulose (HPMC), wherein the capsule contains a composition comprising:
(a) about 50 mg MDMA on a free base basis of MDMA, wherein substantially all of the MDMA particles have a particle size less than about 610 μm, a Dv90 below 420 μm, and a particle size range of less than 400 μm;
(b) about 0.1% to about 10% by weight of magnesium stearate; and
(c) about 25% to about 75% by weight of mannitol.

Some embodiments provide a dosage form comprising a capsule comprising hydroxypropylmethylcellulose (HPMC), wherein the capsule contains a composition comprising:
(a) about 50 mg MDMA on a free base basis of MDMA;
(b) about 0.1% to about 10% by weight of magnesium stearate; and
(c) about 25% to about 75% by weight of mannitol;
wherein substantially all of the particles of the composition have a particle size less than about 610 μm, a Dv90 below 420 μm, and a particle size range of less than 400 μm.

In some embodiments, the dosage form comprises about 0.1% to about 10% (e.g., about 0.1% to about 8%, about 0.1% to about 5%, about 0.1% to about 4%, about 0.1% to about 2%, about 0.5% to about 1.5%, or about 1%) by weight of magnesium stearate. In some embodiments, the dosage form comprises about 1% by weight of magnesium stearate.

In some embodiments, the dosage form comprises about 25% to about 75% (e.g., about 25% to about 65%, about 25% to about 55%, about 25% to about 50%, about 25% to about 35%, about 35% to about 75%, about 50% to about 75%, about 35% to about 65%, about 40% to about 60%, about 45% to about 55%, or about 49%) by weight of mannitol. In some embodiments, the dosage form comprises about 49% by weight of mannitol.

In some embodiments, the dosage form comprises about 25% to about 75% (e.g., about 25% to about 65%, about 25% to about 55%, about 25% to about 50%, about 25% to about 35%, about 35% to about 75%, about 50% to about 75%, about 35% to about 65%, about 40% to about 60%, about 45% to about 55%, or about 50%) by weight of MDMA·HCl. In some embodiments, the dosage form comprises about 50% by weight of MDMA·HCl.

Some embodiments provide a dosage form comprising a capsule comprising hydroxypropylmethylcellulose (HPMC), wherein the capsule contains a composition comprising:
(a) about 60 mg MDMA·HCl, wherein substantially all of the MDMA·HCl particles have a particle size less than about 610 μm, a Dv90 below 420 μm, and a particle size range of less than 400 μm;
(b) about 1% by weight of magnesium stearate; and
(c) about 49% by weight of mannitol.

Some embodiments provide a dosage form comprising a capsule comprising hydroxypropylmethylcellulose (HPMC), wherein the capsule contains a composition comprising:
(a) about 60 mg MDMA·HCl;
(b) about 1% by weight of magnesium stearate; and
(c) about 49% by weight of mannitol;
wherein substantially all of the particles of the composition have a particle size less than about 610 μm, a Dv90 below 420 μm, and a particle size range of less than 400 μm.

### MDMA-Assisted Therapy

Some embodiments provide a method for treating a subject suffering from an eating disorder (ED) comprising administering a pharmaceutical composition comprising crystalline MDMA, or a pharmaceutically acceptable salt and/or solvate thereof, in a therapeutic environment.

MDMA-assisted therapy has emerged a highly efficacious integrated intervention for subjects with treatment-resistant PTSD (Jerome et al., 2020 Jerome et al., 2020 Jerome et al., 2020; Mitchell, J. M. et al., 2021 Mitchell, J. M. et al., 2021 Mitchell, J. M. et al., 2021; Mithoefer et al., 2019 Mithoefer et al., 2019 Mithoefer et al., 2019; Mithoefer et al., 2018 Mithoefer et al., 2018 Mithoefer et al., 2018; Sessa et al., 2019 Sessa et al., 2019 Sessa et al., 2019; Wang et al., 2021 Wang et al., 2021 Wang et al., 2021). Significantly higher rates of PTSD or PTSD symptoms have been associated with EDs and ED symptoms and vice versa (Dansky et al., 1997 Dansky et al., 1997 Dansky et al., 1997; Ferrell et al., 2020 Ferrell et al., 2020 Ferrell et al., 2020; Hudson et al., 2007 Hudson et al., 2007 Hudson et al., 2007; Mitchell et al., 2012 Mitchell et al., 2012 Mitchell et al., 2012). EDs and PTSD share several common risk factors that may contribute to their co-occurrence, including female gender, history of personal and/or family psychiatric disorder, history of child maltreatment or other prior traumas, higher trauma dose and severity, personality factors, and lack of social supports (Brewerton, 2018 Brewerton, 2018 Brewerton, 2018). Both EDs and PTSD have high degrees of morbidity and mortality, including suicide and self-harm (Arcelus et al., 2011 Arcelus et al., 2011 Arcelus et al., 2011; Fichter and Quadflieg, 2016 Fichter and Quadflieg, 2016 Fichter and Quadflieg, 2016; Gradus et al., 2010 Gradus et al., 2010 Gradus et al., 2010, 201520152015; Himmerich et al., 2019 Himmerich et al., 2019 Himmerich et al., 2019; Lee et al., 2014 Lee et al., 2014 Lee et al., 2014; Mandelli et al., 2018 Mandelli et al., 2018 Mandelli et al., 2018; Papadopoulos et al., 2009 Papadopoulos et al., 2009 Papadopoulos et al., 2009; Preti et al., 2011 Preti et al., 2011 Preti et al., 2011; Roberts et al., 2020 Roberts et al., 2020 Roberts et al., 2020; Smink et al., 2012 Smink et al., 2012 Smink et al., 2012; Stein et al., 2010 Stein et al., 2010 Stein et al., 2010). Individuals with both disorders (ED-PTSD) have significantly greater psychiatric and medical comorbidity, higher symptom severity, higher treatment dropout rates, worse prognosis, and poorer quality of life (Brewerton, 2018 Brewerton, 2018 Brewerton, 2018; Brewerton et al., 2020 Brewerton et al., 2020 Brewerton et al., 2020; Trottier, 2020 Trottier, 2020 Trottier, 2020).

Many have identified eating disorder treatment as a speculative target for empathogen-assisted therapy, typically using MDMA as a secondary therapeutic agent in conjunction with other substances. However, whether any of one these medicaments is actually, rather than speculatively, useful in treating subjects suffering from eating disorders had not yet been explored until as described in the present application.

Indeed, the pharmaceutical compositions comprising particles comprising crystalline MDMA, or a pharmaceutically acceptable salt and/or solvate thereof, substantially less than about 610 μm described herein have been demonstrated to reduce symptoms of disordered eating in subjects suffering from comorbid PTSD, when administered in a structured, therapeutic program. Subjects with eating disorder symptoms meeting the “at-risk” (score≥11) and “clinical” (score≥20) thresholds established by the EAT-26 questionnaire saw meaningful and clinically-significant score reductions following treatment with the MDMA-containing pharmaceutical compositions described herein.

In some embodiments, the composition and unit dosage forms described herein comprise 120 mg of MDMA and are formulated to provide a Cₘₐₓ of MDMA of about 100 ng/mL to about 500 ng/mL in the subject after administering the composition or unit dosage form. In some embodiments, the composition and unit dosage forms described herein comprise 100 mg of MDMA and are formulated to provide a Cₘₐₓ of MDMA of about 100 ng/mL to about 500 ng/mL in the subject after administering the composition or unit dosage form. In some embodiments, the composition and unit dosage forms described herein are formulated to provide a Cₘₐₓ of MDMA of about 150 ng/mL to about 450 ng/mL, about 175 ng/mL to about 400 ng/mL, about 200 ng/mL to about 320 ng/mL, about 220 ng/mL to about 300 ng/mL, about 218 ng/mL to about 258 ng/mL, about 228 ng/mL to about 248 ng/mL, about 240 ng/mL to about 280 ng/mL, about 250 ng/mL to about 275 ng/mL, about 255 ng/mL to about 270 ng/mL, about 158 ng/mL to about 164 ng/mL, about 238 ng/mL, or about 261 ng/mL in the subject after administering the composition or unit dosage form. In some embodiments, the Cₘₐₓ is about 261 ng/mL. In some embodiments, the Cₘₐₓ is about 238 ng/mL.

In some embodiments, the composition and unit dosage forms described herein are formulated to provide an AUC₀₋ₜ in the subject after administering the composition or unit dosage form of about 2500 h*ng/mL to about 5000 h*ng/mL, about 3000 h*ng/mL to about 4500 h*ng/mL, about 3000 h*ng/mL to about 4200 h*ng/mL, about 3000 h*ng/mL to about 3800 h*ng/mL, about 3000 h*ng/mL to about 3600 h*ng/mL, about 3000 h*ng/mL to about 3400 h*ng/mL, about 3000 h*ng/mL to about 3300 h*ng/mL, about 3000 h*ng/mL to about 3250 h*ng/mL, about 3050 h*ng/mL to about 3200 h*ng/mL, about 3100 h*ng/mL to about 3150 h*ng/mL, about 3300 h*ng/mL to about 4000 h*ng/mL, about 3570 h*ng/mL to about 3770 h*ng/mL, about 3500 h*ng/mL to about 3600 h*ng/mL, about 3520 h*ng/mL to about 3580 h*ng/mL, about 3620 h*ng/mL to about 3730 h*ng/mL, about 3123 h*ng/mL, or about 3670 h*ng/mL. In some embodiments, the AUC₀₋ₜ is about 3550 h*ng/mL. In some embodiments, the AUC₀₋ₜ is about 3670 h*ng/mL. In some embodiments, the AUC₀₋ₜ is about 3123 h*ng/mL.

In some embodiments, the composition and unit dosage forms described herein are formulated to provide an AUC₀₋₇₂ in the subject after administering the composition or unit dosage form of about 2500 h*ng/mL to about 5000 h*ng/mL, about 3000 h*ng/mL to about 4500 h*ng/mL, about 3000 h*ng/mL to about 3700 h*ng/mL, about 3200 h*ng/mL to about 3500 h*ng/mL, about 3300 h*ng/mL to about 3440 h*ng/mL, about 3500 h*ng/mL to about 4200 h*ng/mL, about 3700 h*ng/mL to about 3900 h*ng/mL, about 3750 h*ng/mL to about 3850 h*ng/mL, about 3800 h*ng/mL to about 4000 h*ng/mL, about 3870 h*ng/mL to about 3900 h*ng/mL, or about 3880 h*ng/mL. In some embodiments, the AUC₀₋₇₂ is about 3800 h*ng/mL. In some embodiments, the AUC_{0-inf} is about 3880 h*ng/mL.

In some embodiments, the composition and unit dosage forms described herein are formulated to provide an AUC_{0-inf} in the subject after administering the composition or unit dosage form of about 2500 h*ng/mL to about 5000 h*ng/mL, about 3000 h*ng/mL to about 4500 h*ng/mL, about 3500 h*ng/mL to about 4200 h*ng/mL, about 3700 h*ng/mL to about 3900 h*ng/mL, about 3750 h*ng/mL to about 3850 h*ng/mL, about 3800 h*ng/mL to about 4000 h*ng/mL, about 3870 h*ng/mL to about 3900 h*ng/mL, about 3368 h*ng/mL, or about 3890 h*ng/mL. In some embodiments, the AUC_{0-inf} is about 3800 h*ng/mL. In some embodiments, the AUC_{0-inf} is about 3890 h*ng/mL. In some embodiments, the AUC_{0-inf} is about 3368 h*ng/mL.

In some embodiments, the composition and unit dosage forms described herein are formulated to provide a Tₘₐₓ in the subject after administering the composition or unit dosage form of about 30 minutes to about 10 hours, about 30 minutes to about 4 hours, about 45 minutes to about 3 hours, about 2 hours to about 8 hours, about 4 hours to about 10 hours, about 6 hours to about 10 hours, about 2 hours to about 6 hours, about 3 hours to about 5 hours, about 1 hour to about 3 hours, about 1.5 hours to about 2.5 hours, about 1.7 hours to about 2.3 hours, or about 2 hours. In some embodiments, the Tₘₐₓ is about 4 hours. In some embodiments, the Tₘₐₓ is about 2 hours.

In some embodiments, the composition and unit dosage forms described herein are formulated to provide a t_{1/2} in the subject after administering the composition or unit dosage form of about 2 hours to about 20 hours, about 3 hours to about 20 hours, about 4 hours to about 20 hours, about 4 hours to about 15 hours, about 4 hours to about 12 hours, about 4 hours to about 8 hours, about 4 hours to about 6 hours, about 6 hours to about 20 hours, about 8 hours to about 20 hours, about 10 hours to about 20 hours, about 13 hours to about 20 hours, about 16 hours to about 20 hours, about 18 hours to about 20 hours, about 6 hours to about 12 hours, about 7 hours to about 11 hours, about 8 hours to about 10 hours, about 8 hours to about 8 hours, about 8.36 hours, about 8.7 hours, or about 9 hours. In some embodiments, the t_{1/2} is about 8.36 hours. In some embodiments, the t_{1/2} is about 8.7 hours. In some embodiments, the t_{1/2} is about 9 hours.

In some embodiments, the composition and unit dosage forms described herein are formulated to provide a T_{lag} in the subject after administering the composition or unit dosage form of about 0 hours to about 2 hours, about 0.25 hours to about 1.5 hours, about 0.25 hours to about 0.75 hours, about 0.75 hours to about 1.25 hours, about 0.5 hours, about 0.6 hours, about 0.75 hours, or about 1 hour. In some embodiments, the T_{lag} is about 0.5 hours. In some embodiments, the T_{lag} is about 0.5 hour. In some embodiments, the measured T_{lag} is about 0.6 hour.

In some embodiments, the composition and unit dosage forms described herein are formulated to provide a CL/F in the subject after administering the composition or unit dosage form of about 1 L/h to about 100 L/h, about 1 L/h to about 70 L/h, about 10 L/h to about 60, about 20 L/h to about 50 L/h, about 20 L/h to about 40 L/h, about 25 L/h to about 35 L/h, about 30 L/h to about 40 L/h, about 32 L/h to about 36 L/h, about 35 L/h to about 40 L/h, about 34.5 L/h, about 29.5 L/h, or about 37.5 L/h. In some embodiments, the CL/F is about 368 L/h. In some embodiments, the CL/F is about 34.5 L/h. In some embodiments, the CL/F is about 368 L/h. In some embodiments, the CL/F is about 37.5 L/h. In some embodiments, the CL/F is about 29.5 L/h.

In some embodiments, the composition and unit dosage forms described herein are formulated to provide a Vd/F in the subject after administering the composition or unit dosage form of about 100 L to about 800 L, about 200 L to about 700 L, about 300 L to about 600 L, about 300 L to about 450 L, about 320 L to about 420 L, about 400 L to about 500 L, about 400 L to about 420 L, about 410 L to about 450 L, about 420 L to about 440 L, about 425 L to about 435 L, about 371.7 Vd/F, about 412 L, or about 430 L. In some embodiments, the Vd/F is about 430 L. In some embodiments, the Vd/F is about 412 Vd/F. In some embodiments, the Vd/F is about 371.7 Vd/F.

### Methods of Use

Some embodiments provide a method of treating a subject in need thereof, comprising administering to the subject a dosage form as described herein (i.e., comprising MDMA or a pharmaceutically acceptable salt and/or solvate thereof and one or more pharmaceutically acceptable excipients).

In some embodiments, the dosage form is administered in a therapeutic setting, for example, in an in-patient and/or out-patient setting. In some embodiments, the dosage form is administered during the course of a psychological intervention. In some embodiments, the dosage form is administered in a psychotherapy session (e.g., a single psychotherapy session, or medication session or psychological intervention). In some embodiments, the dosage form is administered over multiple psychotherapy sessions (or medication session or psychological intervention, e.g., administration during a single session, administration at every other session, and the like).

Some embodiments provide a method of treating a subject having one or more central nervous system (CNS) disorders, comprising administering to the subject a dosage form as described herein (i.e., comprising MDMA or a pharmaceutically acceptable salt and/or solvate thereof and one or more pharmaceutically acceptable excipients).

In some embodiments, the one or more CNS disorders are independently mood, anxiety, or trauma-linked disorders.

In some embodiments, the one or more CNS disorders are independently autism spectrum disorders, neuropsychiatric diseases or disorders; or neurodegenerative diseases.

In some embodiments, the one or more CNS disorders are independently post-traumatic stress disorder (PTSD), anxiety disorder, major depressive disorder, obsessive compulsive disorder, bipolar disorder, dysthymic disorder; Parkinson's disease, epilepsy, recurrent migraines, stroke, or post-concussion syndrome; alcohol use disorder; attention deficit hyperactivity disorder (ADHD), anorexia nervosa, bulimia, an eating disorder, binge eating disorder, or autism.

In some embodiments, the one or more CNS disorders is PTSD. In some embodiments, the one or more CNS disorders is treatment-resistant PTSD.

In some embodiments, the one or more CNS disorders is an eating disorder.

Diagnosis of an eating disorder can use a variety of assessments, tests, or questionnaires. Non-limiting examples of eating disorder assessments include the eating attitudes test, Ben-Tovim Walker body attitudes questionnaire, the body attitudes test, the eating disorder inventory, and the SCOFF questionnaire.

The Eating Attitudes Test (EAT or EAT-26) is a standardized self-reported questionnaire of symptoms and concerns characteristic of eating disorders. The EAT-26 uses a six-point scale based on how often the individual engages in specific behaviors. The questions can be answered: Always, Usually, Often, Sometimes, Rarely, and Never. Completing the EAT-26 yields a “referral index” based on three criteria: 1) the total score based on the answers to the EAT-26 questions; 2) answers to the behavioral questions related to eating symptoms and weight loss, and 3) the individual's body mass index (BMI) calculated from their height and weight. In some cases, an individual can be recommended for additional care if the individual meets a “cut off” score or threshold on one or more criteria. For example, if an individual score higher than a 20, the individual should be referred to a qualified professional for potential diagnosis of an eating disorder. (See, for example, Garner, D. M., & Garfinkel, P. E. (1979). Psychological Medicine, 9, 273-279.)

The Ben-Tovim Walker Body Attitudes Questionnaire (body attitude questionnaire, BAQ) is a 44 item self-report questionnaire divided into six subscales that measures an individual's attitude towards their own body. The six subscales measured by the BAQ are: overall fatness, self-disparagement, strength, salience of weight, feelings of attractiveness, and consciousness of lower body fat. (See, for example, Ben-Tovim and Walker (1991). Psychological Medicine. 21 (3): 775-84.)

The body attitudes test (BAT) measures an individual's subjective body experience and attitudes towards one's own body. It is a questionnaire composed of twenty items which yields four different factors that evaluate the internal view of the individual's own body. Subjects are asked to score each statement 0-5, 0 meaning they do not relate to the statement at all, and 5 meaning the statement frequently describes their sentiment. The following are examples of questions asked in the assessment:
1. I feel displeased when comparing my body to others.
2. I do not recognize my body as my own.
3. My body is too wide.
4. I am pleased with my body shape.
5. I feel the need to lose weight.
6. I see my breasts as too big.
7. I feel the need to conceal my body in looser clothing.
8. I avoid my reflection because it upsets me.
9. I do not struggle with relaxing.
10. I feel like every aspect of my body is broad.
11. My body negatively weighs on me.
12. There is a dissonance between my body and I.
13. At times, I feel like my body is swollen.
14. I feel threatened by my physical appearance.
15. I take great pride in my body size.
16. I feel like I look pregnant.
17. I always feel very tense.
18. I tend to be jealous of other people's looks.
19. Aspects of my physical appearance scare me.
20. I often scrutinize my own reflection.

The answers to these questions are analyzed to provide information regarding four factors that evaluate the individual's subjective view on their body: negative appreciation of body size, lack of familiarity with one's own body, general body dissatisfaction, and rest factor. (See, for example, Probst et al (1995) Eating Disorders, 3 (2): 133-144.).

The eating disorder inventory is a self-report questionnaire used to assess the presence of eating disorders, (a) anorexia nervosa both restricting and binge-eating/purging type; (b) bulimia nervosa; and (c) eating disorder not otherwise specified including binge eating disorder. There are three versions of the inventory. (See, e.g., Garner et al. 1983. Int. J. Eating Dis. 2 (2): 15-34.).

The original eating disorder inventory (called the eating disorder inventory, EDI) is a 64 questions test, divided into eight subscales. Each question is on a 6-point scale (ranging from “always” to “never”), rated 0-3. The score for each sub-scale is then summed. The 8 subscale scores on the EDI are drive for thinness (an excessive concern with dieting, preoccupation with weight, and fear of weight gain), bulimia, body dissatisfaction, ineffectiveness (assessment of feelings of inadequacy, insecurity, worthlessness and having no control over their lives), perfectionism, interpersonal distrust (reluctance to form close relationships), interoceptive awareness (measures the ability of an individual to discriminate between sensations and feelings, and between the sensations of hunger and satiety), and maturity fears (the fear of facing the demands of adult life).

The eating disorder inventory-2 (EDI-2) retains the original format of the EDI with the inclusion of 27 new items divided into three additional subscales: asceticism (the avoidance of sexual relationships), impulse regulation, and social insecurity (social fears and insecurity). EDI-2 is used for both males and females over age 12

The eating disorder inventory-3 (EDI-3) contains 91 items divided into twelve subscales rated on a 0-4 point scoring system. Three items on the EDI-3 are specific to eating disorders, and 9 are general psychological scales that are relevant to eating disorders. The inventory yields six composite scores: eating disorder risk, ineffectiveness, interpersonal problems, affective problems, over control, and general psychological maladjustment.

The SCOFF questionnaire is a series of five questions used to indicate the presence of an eating disorder. (See, for example, Morgan et al. (2000) West J Med. 172 (3): 164-5.).

Some embodiments provide a method for treating an eating disorder in a subject in need thereof comprising administering a pharmaceutical composition comprising particles comprising crystalline MDMA, or a pharmaceutically acceptable salt and/or solvate thereof, or a pharmaceutically acceptable salt and/or solvate thereof, substantially less than about 610 μm, with a Dv90 below 420 μm and a particle size range of less than 400 μm.

Some embodiments provide a method for treating an eating disorder in a subject in need thereof comprising (a) determining that the subject has an eating disorder; and (b) administering a pharmaceutical composition comprising particles comprising crystalline MDMA, or a pharmaceutically acceptable salt and/or solvate thereof, substantially less than about 610 μm, with a Dv90 below 420 μm and a particle size range of less than 400 μm.

Some embodiments provide a method for treating an eating disorder, comprising administering to a subject previously identified or diagnosed as having an eating disorder, a pharmaceutical composition comprising particles comprising crystalline MDMA, or a pharmaceutically acceptable salt and/or solvate thereof, substantially less than about 610 μm, with a Dv90 below 420 μm and a particle size range of less than 400 μm.

In some embodiments, the method further comprises providing psychotherapy to the subject, for example, in an in-patient and/or out-patient setting.

In certain embodiments, the pharmaceutical composition can be administered in a therapeutic setting. The therapeutic setting can be a medical facility (e.g., a hospital), a practitioner's office, a private home, an outdoor space, or any other building or physical environment designated for MDMA-assisted therapy in accordance with the present disclosure

In certain embodiments, the subject can be suffering from one or more central nervous system disorders, including mood, anxiety, or trauma-linked disorders, such as post-traumatic stress disorder, anxiety disorder, major depressive disorder, obsessive compulsive disorder, bipolar disorder, dysthymic disorder; neurological disorders such as Parkinson's disease, epilepsy, recurrent migraines, stroke, or post-concussion syndrome; alcohol use disorder; attention deficit hyperactivity disorder (ADHD); eating disorders such as anorexia nervosa, bulimia, or binge eating disorder; autism and autism spectrum disorders; neuropsychiatric diseases or disorders; or neurodegenerative diseases. In some embodiments, the subject is suffering from an eating disorder.

The pharmaceutical composition can be administered in any pharmaceutically acceptable dosage form, including dosage forms provided in accordance with any embodiment of the present disclosure. The pharmaceutical composition can be administered on one occasion, or on multiple individual occasions.

In certain embodiments, the pharmaceutical composition is administered during the course of a psychological intervention. Such administration can be a single administration at a single time period and/or multiple administrations over a period of time, for example, throughout the duration of a psychological intervention.

In certain embodiments, the pharmaceutical composition is administered during two individual psychological interventions, three individual psychological interventions, four individual psychological interventions, five individual psychological interventions, six individual psychological interventions, seven individual psychological interventions, eight individual psychological interventions, nine individual psychological interventions, or ten individual psychological interventions. The individual psychological interventions may occur at regular intervals, e.g., every two weeks, or at non-regular intervals that may vary in accordance with a subject's individual needs or protocols established for treating the subject's indicated disease or disorder.

In certain embodiments, the pharmaceutical composition is administered during two individual psychotherapy sessions, three individual psychotherapy sessions, four individual psychotherapy sessions, five individual psychotherapy sessions, six individual psychotherapy sessions, seven individual psychotherapy sessions, eight individual psychotherapy sessions, nine individual psychotherapy sessions, or ten individual psychotherapy sessions. The individual psychotherapy sessions may occur at regular intervals, e.g., every two weeks, or at non-regular intervals that may vary in accordance with a subject's individual needs or protocols established for treating the subject's indicated disease or disorder.

In certain embodiments, the pharmaceutical composition is administered during two individual medication sessions, three individual medication sessions, four individual medication sessions, five individual medication sessions, six individual medication sessions, seven individual medication sessions, eight individual medication sessions, nine individual medication sessions, or ten individual medication sessions. The individual medication sessions may occur at regular intervals, e.g., every two weeks, or at non-regular intervals that may vary in accordance with a subject's individual needs or protocols established for treating the subject's indicated disease or disorder.

In some embodiments, an oral dosage form comprising the pharmaceutical composition in accordance with any embodiment of the present disclosure is administered to a subject suffering from a central nervous system disease or disorder. The oral dosage form is administered in a therapeutic setting (i.e., in the context of therapy) during multiple individual psychological interventions, wherein at least one therapist is present. The oral dosage form is administered in a therapeutic setting during multiple individual psychotherapy sessions, wherein at least one therapist is present. The oral dosage form is administered in a therapeutic setting during multiple individual medication sessions, wherein at least one other individual is present.

The dosage form can be administered in any pharmaceutically acceptable dosage form, including dosage forms provided in accordance with any embodiment as described herein. The dosage form can be administered on one occasion, or on multiple individual occasions.

In some embodiments, the dosage form is administered during an individual psychological intervention. In some embodiments, the dosage form is administered during individual psychotherapy. In some embodiments, the dosage form is administered during individual medication sessions. The individual sessions may occur at regular intervals, e.g., every two weeks, or at non-regular intervals that may vary in accordance with a subject's individual needs or protocols established for treating the subject's indicated disease or disorder.

In some embodiments, the dosage form in accordance with any embodiment is orally administered to a subject during multiple individual sessions, wherein at least one therapist is present.

In some embodiments, the dosage form is orally administered in two separate dosage components (e.g., a split or divided dose), an initial dose and a supplementary dose, during the same psychological intervention (or medication session). In some embodiments, the dosage form is orally administered in two separate dosage components, an initial dose and a supplementary dose, during the same psychotherapy session. The initial dose may comprise about 25 to about 150 mg of MDMA, or a pharmaceutically acceptable salt and/or solvate thereof, and the supplementary dose may comprise about 10 mg to about 70 mg of MDMA, or a pharmaceutically acceptable salt and/or solvate thereof. In some embodiments, the initial dose is about twice the amount of MDMA, or a pharmaceutically acceptable salt and/or solvate thereof, as the supplemental dose. In some embodiments, the initial dose is about 68 mg of MDMA, or a pharmaceutically acceptable salt and/or solvate thereof, and the supplemental dose is about 34 mg of MDMA, or a pharmaceutically acceptable salt and/or solvate thereof. In some embodiments, the initial dose is about 100 mg of MDMA, or a pharmaceutically acceptable salt and/or solvate thereof, and the supplemental dose is about 50 mg of MDMA, or a pharmaceutically acceptable salt and/or solvate thereof.

In some embodiments, the initial and supplementary dosage components are physically separated from each other (e.g., as two capsules, two tablets, or one capsule and one tablet) and are provided in a kit (e.g., a blister pack). In some embodiments, initial and supplementary dosage components are both part of one dosage form (e.g., a pill, a tablet, or a capsule).

In some embodiments, the central nervous system disorder is a trauma-linked disorder or a stressor-linked disorder. In some embodiments, the central nervous system disorder is a mood disorder. In some embodiments, wherein the central nervous system disorder is an anxiety disorder. In some embodiments, the central nervous system disorder is post-traumatic stress disorder.

In some embodiments, the administering is performed during a psychological intervention. In some embodiments, the administering is performed during a psychotherapy session. In some embodiments, the administering is performed during a medication session. In some embodiments, a dosage form comprising about 100 mg of MDMA is administered. In some embodiments, about 100 mg of MDMA is administered in one dose. In some embodiments, about 100 mg of MDMA is administered in two doses.

In some embodiments, a dosage form comprising about 120 mg of MDMA is administered. In some embodiments, about 120 mg of MDMA is administered in one dose. In some embodiments, the about 120 mg of MDMA is administered in two doses.

In some embodiments, the dosage form comprising about 140 mg of MDMA is administered. In some embodiments, about 140 mg of MDMA is administered in one dose. In some embodiments, the about 140 mg of MDMA is administered in two doses.

In some embodiments, the dosage form comprising about 160 mg of MDMA is administered. In some embodiments, the about 160 mg of MDMA is administered in one dose. In some embodiments, the about 160 mg of MDMA is administered in two doses.

In some embodiments, the dose of MDMA, or a pharmaceutically acceptable salt and/or solvate thereof, administered in one day is about 80 mg to about 170 mg on a free base basis of MDMA. In some embodiments, the dose of MDMA, or a pharmaceutically acceptable salt and/or solvate thereof, administered in one day is about 102 mg to about 150 mg on a free base basis of MDMA. In some embodiments, the dose of MDMA, or a pharmaceutically acceptable salt and/or solvate thereof, administered in one day is about 102 mg on a free base basis of MDMA. In some embodiments, the dose of MDMA, or a pharmaceutically acceptable salt and/or solvate thereof, administered in one day is about 150 mg on a free base basis of MDMA. In some embodiments, the dose of MDMA, or a pharmaceutically acceptable salt and/or solvate thereof, administered in one day should not exceed about 150 mg on a free base basis of MDMA.

In some embodiments, the dose of MDMA·HCl, or a pharmaceutically acceptable salt and/or solvate thereof, administered in one day is about 90 mg to about 210 mg, on a free base basis of MDMA. In some embodiments, the dose of MDMA, or a pharmaceutically acceptable salt and/or solvate thereof, administered in one day is about 120 mg to about 180 mg, on a free base basis of MDMA. In some embodiments, the dose of MDMA, or a pharmaceutically acceptable salt and/or solvate thereof, administered in one day is about 120 mg, on a free base basis of MDMA. In some embodiments, the dose of MDMA, or a pharmaceutically acceptable salt and/or solvate thereof, administered in one day is about 180 mg, on a free base basis of MDMA. In some embodiments, the dose of MDMA, or a pharmaceutically acceptable salt and/or solvate thereof, administered in one day should not exceed about 180 mg, on a free base basis of MDMA.

In some embodiments, the dosage form comprising the therapeutically effective amount of MDMA is orally administered. In some embodiments, the dosage form comprising the therapeutically effective amount of MDMA is administered in a capsule. In some embodiments, the dosage form comprising the therapeutically effective amount of MDMA is administered in a tablet.

In some embodiments, the dosage form comprising the therapeutically effective amount of MDMA is administered as one or more individual dosage units during a single psychotherapy session (or medication session or psychological intervention). In some embodiments, the dosage form comprising the therapeutically effective amount of MDMA is administered at different times during a single psychotherapy session (or mediciation session or psychological intervention).

Some embodiments provide a method of treating PTSD in a subject in need thereof, comprising:
administering one or more individual dosage units during a session;
wherein each individual dosage unit comprises a composition comprising about 20 mg to about 150 mg MDMA and/or a pharmaceutically acceptable salt or solvate thereof on a free base basis of MDMA;
wherein substantially all of the MDMA particles have a particle size less than about 610 μm, a Dv90 below 420 μm, and a particle size range of less than 400 μm.

Some embodiments provide a method of treating PTSD in a subject in need thereof, comprising:
administering one or more individual dosage units during a session;
wherein each individual dosage unit comprises a composition comprising about 20 mg to about 150 mg MDMA and/or a pharmaceutically acceptable salt or solvate thereof on a free base basis of MDMA;
wherein substantially all of the particles of the composition have a particle size less than about 610 μm, a Dv90 below 420 μm, and a particle size range of less than 400 μm.

Some embodiments provide a method of treating PTSD in a subject in need thereof, comprising:
administering one or more individual dosage units during a first session;
administering one or more individual dosage units during a second session at least 21 days after the first session;
administering one or more individual dosage units during a third session at least 21 days after the second session;
wherein each individual dosage unit comprises a composition comprising about 20 mg to about 150 mg MDMA and/or a pharmaceutically acceptable salt or solvate thereof on a free base basis of MDMA;
wherein substantially all of the MDMA particles have a particle size less than about 610 μm, a Dv90 below 420 μm, and a particle size range of less than 400 μm.

Some embodiments provide a method of treating PTSD in a subject in need thereof, comprising:
administering one or more individual dosage units during a first session;
administering one or more individual dosage units during a second session at least 21 days after the first session;
administering one or more individual dosage units during a third session at least 21 days after the second session;
wherein each individual dosage unit comprises a composition comprising about 20 mg to about 150 mg MDMA and/or a pharmaceutically acceptable salt or solvate thereof on a free base basis of MDMA;
wherein substantially all of the particles of the composition have a particle size less than about 610 μm, a Dv90 below 420 μm, and a particle size range of less than 400 μm.

Some embodiments provide a method of treating PTSD in a subject in need thereof, comprising:
administering one or more individual dosage units during a session;
wherein each individual dosage unit comprises a composition comprising about 20 mg to about 150 mg MDMA and/or a pharmaceutically acceptable salt or solvate thereof on a free base basis of MDMA;
wherein substantially all of the MDMA particles have an average particle size of about 420 μm.

Some embodiments provide a method of treating PTSD in a subject in need thereof, comprising:
administering one or more individual dosage units during a session;
wherein each individual dosage unit comprises a composition comprising about 20 mg to about 150 mg MDMA and/or a pharmaceutically acceptable salt or solvate thereof on a free base basis of MDMA;
wherein substantially all of the MDMA particles have an average particle size of about 420 μm.

Some embodiments provide a method of treating PTSD in a subject in need thereof, comprising:
administering one or more individual dosage units during a first session;
administering one or more individual dosage units during a second session at least 21 days after the first session;
administering one or more individual dosage units during a third session at least 21 days after the second session;
wherein each individual dosage unit comprises a composition comprising about 20 mg to about 150 mg MDMA and/or a pharmaceutically acceptable salt or solvate thereof on a free base basis of MDMA;
wherein substantially all of the MDMA particles have an average particle size of about 420 μm.

Some embodiments provide a method of treating PTSD in a subject in need thereof, comprising:
administering one or more individual dosage units during a first session;
administering one or more individual dosage units during a second session at least 21 days after the first session;
administering one or more individual dosage units during a third session at least 21 days after the second session;
wherein each individual dosage unit comprises a composition comprising about 20 mg to about 150 mg MDMA and/or a pharmaceutically acceptable salt or solvate thereof on a free base basis of MDMA;
wherein substantially all of the MDMA particles have an average particle size of about 420 μm.

In some embodiments, the one or more individual dosage units administered during a session is one individual dosage unit. In some embodiments, the one or more individual dosage units administered during a session is two individual dosage units. In some embodiments, the one or more individual dosage units administered during a session is three individual dosage units.

In some embodiments, when the one or more individual dosage units is two or more individual dosage units, the method comprises administering the individual dosage units at different times during the session. In some embodiments, when the one or more individual dosage units is two or more individual dosage units, the method comprises administering the individual dosage units at the same time during the session.

In some embodiments, the one or more individual dosage units is three individual dosage units; two of the individual dosage units are administered at the same time; and the third individual dosage unit is administered at a different time during the session (e.g., after a period of time). In some embodiments, the one or more individual dosage units is three individual dosage units; the first and second of the individual dosage units are administered at the same time; and the third individual dosage unit is administered after the first and second individual dosage units during the session. In some embodiments, the third individual dosage unit is administered (i.e., after a period of time) about 5 minutes to about 5 hours (e.g., about 15 minutes to about 5 hours, about 30 minutes to about 5 hours, about 1 hour to about 5 hours, about 1.5 hours to about 5 hours, about 2 hours to about 5 hours, about 3 hours to about 5 hours, about 5 minutes to about 4 hours, about 5 minutes to about 3 hours, about 5 minutes to about 2 hours, about 5 minutes to about 1 hour, about 30 minutes to about 4 hours, about 45 minutes to about 3 hours, about 1 hour to about 2.5 hours, about 1 hour to about 2 hours, about 1 hour and 15 minutes to about 1 hour and 45 minutes, about 1 hour and 15 minutes to about 2 hours and 15 minutes, about 1.5 hours to about 2 hours, about 1.5 hours, about 1 hour and 45 minutes, or about 2 hours) after the first and second individual dosage units. In some embodiments, the third individual dosage unit is administered about 1.5 hours to about 2 hours after the first and second individual dosage units.

Some embodiments provide a method of treating PTSD in a subject in need thereof, comprising:
administering to the subject a first dosage unit comprising about 68 mg of MDMA, or a pharmaceutically acceptable salt thereof on a free base basis of MDMA; and
after a period of time, administering to the subject a second dosage unit comprising about 34 mg of MDMA, or a pharmaceutically acceptable salt thereof on a free base basis of MDMA.

Some embodiments provide a method of treating PTSD in a subject in need thereof, comprising:
administering to the subject a first dosage unit comprising about 100 mg of MDMA, or a pharmaceutically acceptable salt thereof on a free base basis of MDMA; and
after a period of time, administering to the subject a second dosage unit comprising about 50 mg of MDMA, or a pharmaceutically acceptable salt thereof on a free base basis of MDMA.

Some embodiments provide a method of treating PTSD in a subject in need thereof, comprising:
administering to the subject a first dosage unit comprising about 68 mg of MDMA, or a pharmaceutically acceptable salt thereof on a free base basis of MDMA; and
after a period of time, administering to the subject a second dosage unit comprising about 34 mg of MDMA, or a pharmaceutically acceptable salt thereof on a free base basis of MDMA;
wherein substantially all of the MDMA particles have a particle size less than about 610 μm, a Dv90 below 420 μm, and a particle size range of less than 400 μm.

Some embodiments provide a method of treating PTSD in a subject in need thereof, comprising:
administering to the subject a first dosage unit comprising about 100 mg of MDMA, or a pharmaceutically acceptable salt thereof on a free base basis of MDMA; and
after a period of time, administering to the subject a second dosage unit comprising about 50 mg of MDMA, or a pharmaceutically acceptable salt thereof on a free base basis of MDMA;
wherein substantially all of the MDMA particles have a particle size less than about 610 μm, a Dv90 below 420 μm, and a particle size range of less than 400 μm.

Some embodiments provide a method of treating an eating disorder in a subject in need thereof, comprising:
administering to the subject a first dosage unit comprising about 68 mg of MDMA, or a pharmaceutically acceptable salt thereof on a free base basis of MDMA; and
after a period of time, administering to the subject a second dosage unit comprising about 34 mg of MDMA, or a pharmaceutically acceptable salt thereof on a free base basis of MDMA.

Some embodiments provide a method of treating an eating disorder in a subject in need thereof, comprising:
administering to the subject a first dosage unit comprising about 100 mg of MDMA, or a pharmaceutically acceptable salt thereof on a free base basis of MDMA; and
after a period of time, administering to the subject a second dosage unit comprising about 50 mg of MDMA, or a pharmaceutically acceptable salt thereof on a free base basis of MDMA.

Some embodiments provide a method of treating an eating disorder in a subject in need thereof, comprising:
administering to the subject a first dosage unit comprising about 68 mg of MDMA, or a pharmaceutically acceptable salt thereof on a free base basis of MDMA; and
after a period of time, administering to the subject a second dosage unit comprising about 34 mg of MDMA, or a pharmaceutically acceptable salt thereof on a free base basis of MDMA;
wherein substantially all of the MDMA particles have a particle size less than about 610 μm, a Dv90 below 420 μm, and a particle size range of less than 400 μm.

Some embodiments provide a method of treating an eating disorder in a subject in need thereof, comprising:
administering to the subject a first dosage unit comprising about 100 mg of MDMA, or a pharmaceutically acceptable salt thereof on a free base basis of MDMA; and
after a period of time, administering to the subject a second dosage unit comprising about 50 mg of MDMA, or a pharmaceutically acceptable salt thereof on a free base basis of MDMA;
wherein substantially all of the MDMA particles have a particle size less than about 610 μm, a Dv90 below 420 μm, and a particle size range of less than 400 μm.

In some embodiments, the dosage form comprising the therapeutically effective amount of MDMA is administered as one or more individual dosage units during a single psychological intervention. In some embodiments, the dosage form comprising the therapeutically effective amount of MDMA is administered at different times during a single psychological intervention.

Some embodiments provide a method of treating PTSD in a subject in need thereof, comprising:
administering one or more individual dosage units during a psychological intervention;
wherein each individual dosage unit comprises a composition comprising about 20 mg to about 150 mg MDMA and/or a pharmaceutically acceptable salt or solvate thereof on a free base basis of MDMA;
wherein substantially all of the MDMA particles have a particle size less than about 610 μm, a Dv90 below 420 μm, and a particle size range of less than 400 μm.

Some embodiments provide a method of treating PTSD in a subject in need thereof, comprising:
administering one or more individual dosage units during a psychological intervention;
wherein each individual dosage unit comprises a composition comprising about 20 mg to about 150 mg MDMA and/or a pharmaceutically acceptable salt or solvate thereof on a free base basis of MDMA;
wherein substantially all of the particles of the composition have a particle size less than about 610 μm, a Dv90 below 420 μm, and a particle size range of less than 400 μm.

In some embodiments, the one or more individual dosage units administered during a psychological intervention is one individual dosage unit. In some embodiments, the one or more individual dosage units administered during a psychological intervention is two individual dosage units. In some embodiments, the one or more individual dosage units administered during a psychological intervention is three individual dosage units.

In some embodiments, when the one or more individual dosage units is two or more individual dosage units, the method comprises administering the individual dosage units at different times during the psychological intervention. In some embodiments, when the one or more individual dosage units is two or more individual dosage units, the method comprises administering the individual dosage units at the same time during the psychological intervention.

In some embodiments, the one or more individual dosage units is three individual dosage units; two of the individual dosage units are administered at the same time; and the third individual dosage unit is administered at a different time during the psychological intervention. In some embodiments, the one or more individual dosage units is three individual dosage units; the first and second of the individual dosage units are administered at the same time; and the third individual dosage unit is administered after the first and second individual dosage units during the psychological intervention. In some embodiments, the third individual dosage unit is administered about 5 minutes to about 5 hours (e.g., about 15 minutes to about 5 hours, about 30 minutes to about 5 hours, about 1 hour to about 5 hours, about 1.5 hours to about 5 hours, about 2 hours to about 5 hours, about 3 hours to about 5 hours, about 5 minutes to about 4 hours, about 5 minutes to about 3 hours, about 5 minutes to about 2 hours, about 5 minutes to about 1 hour, about 30 minutes to about 4 hours, about 45 minutes to about 3 hours, about 1 hour to about 2.5 hours, about 1 hour to about 2 hours, about 1 hour and 15 minutes to about 1 hour and 45 minutes, about 1 hour and 15 minutes to about 2 hours and 15 minutes, about 1.5 hours to about 2 hours, about 1.5 hours, about 1 hour and 45 minutes, or about 2 hours) after the first and second individual dosage units. In some embodiments, the third individual dosage unit is administered about 1.5 hours to about 2 hours after the first and second individual dosage units.

In some embodiments, each individual dosage unit comprises a capsule. In some embodiments, the capsule comprises hydroxypropylmethylcellulose (HPMC). In some embodiments, the capsule contains a composition comprising about 20 mg to about 150 mg MDMA and/or a pharmaceutically acceptable salt or solvate thereof on a free base basis of MDMA. In some embodiments, the capsule contains a composition comprising about 20 mg to about 100 mg MDMA and/or a pharmaceutically acceptable salt or solvate thereof on a free base basis of MDMA. In some embodiments, the capsule contains a composition comprising about 20 mg to about 50 mg MDMA and/or a pharmaceutically acceptable salt or solvate thereof on a free base basis of MDMA. In some embodiments, the capsule contains a composition comprising about 50 mg to about 100 mg MDMA and/or a pharmaceutically acceptable salt or solvate thereof on a free base basis of MDMA. In some embodiments, the capsule contains a composition comprising about 100 mg to about 150 mg MDMA and/or a pharmaceutically acceptable salt or solvate thereof on a free base basis of MDMA. In some embodiments, the capsule contains a composition comprising about 20 mg to about 25 mg MDMA and/or a pharmaceutically acceptable salt or solvate thereof on a free base basis of MDMA. In some embodiments, the capsule contains a composition comprising about 25 mg to about 30 mg MDMA and/or a pharmaceutically acceptable salt or solvate thereof on a free base basis of MDMA. In some embodiments, the capsule contains a composition comprising about 30 mg to about 35 mg MDMA and/or a pharmaceutically acceptable salt or solvate thereof on a free base basis of MDMA. In some embodiments, the capsule contains a composition comprising about 35 mg to about 40 mg MDMA and/or a pharmaceutically acceptable salt or solvate thereof on a free base basis of MDMA. In some embodiments, the capsule contains a composition comprising about 45 mg to about 50 mg MDMA and/or a pharmaceutically acceptable salt or solvate thereof on a free base basis of MDMA. In some embodiments, the capsule contains a composition comprising about 50 mg to about 55 mg MDMA and/or a pharmaceutically acceptable salt or solvate thereof on a free base basis of MDMA. In some embodiments, the capsule contains a composition comprising about 55 mg to about 60 mg MDMA and/or a pharmaceutically acceptable salt or solvate thereof on a free base basis of MDMA. In some embodiments, the capsule contains a composition comprising about 60 mg to about 65 mg MDMA and/or a pharmaceutically acceptable salt or solvate thereof on a free base basis of MDMA. In some embodiments, the capsule contains a composition comprising about 65 mg to about 70 mg MDMA and/or a pharmaceutically acceptable salt or solvate thereof on a free base basis of MDMA. In some embodiments, the capsule contains a composition comprising about 70 mg to about 75 mg MDMA and/or a pharmaceutically acceptable salt or solvate thereof on a free base basis of MDMA. In some embodiments, the capsule contains a composition comprising about 75 mg to about 80 mg MDMA and/or a pharmaceutically acceptable salt or solvate thereof on a free base basis of MDMA. In some embodiments, the capsule contains a composition comprising about 80 mg to about 85 mg MDMA and/or a pharmaceutically acceptable salt or solvate thereof on a free base basis of MDMA. In some embodiments, the capsule contains a composition comprising about 85 mg to about 90 mg MDMA and/or a pharmaceutically acceptable salt or solvate thereof on a free base basis of MDMA. In some embodiments, the capsule contains a composition comprising about 90 mg to about 95 mg MDMA and/or a pharmaceutically acceptable salt or solvate thereof on a free base basis of MDMA. In some embodiments, the capsule contains a composition comprising about 95 mg to about 100 mg MDMA and/or a pharmaceutically acceptable salt or solvate thereof on a free base basis of MDMA. In some embodiments, the capsule contains a composition comprising 34 mg MDMA and/or a pharmaceutically acceptable salt or solvate thereof on a free base basis of MDMA. In some embodiments, the capsule contains a composition comprising 50 mg MDMA and/or a pharmaceutically acceptable salt or solvate thereof on a free base basis of MDMA.

In some embodiments, a complete treatment course consists of 3 split doses in combination with psychological intervention. In some embodiments, the first, second, and third split doses are different. In some embodiments, the first split doses is different than the second, and third split doses. In some embodiments, the first, second, and third split doses are the same.

In some embodiments, the first split dose comprises about 100 mg to about 200 mg MDMA and/or a pharmaceutically acceptable salt or solvate thereof on a free base basis of MDMA. In some embodiments, the first split dose comprises about 100 mg to about 105 mg, about 105 mg to about 110 mg, 110 mg to about 115 mg, about 115 mg to about 120 mg, 120 mg to about 125 mg, about 125 mg to about 130 mg, 130 mg to about 135 mg, about 135 mg to about 140 mg, 140 mg to about 145 mg, about 145 mg to about 150 mg, 150 mg to about 155 mg, about 155 mg to about 160 mg, 160 mg to about 165 mg, about 165 mg to about 170 mg, 170 mg to about 175 mg, about 175 mg to about 180 mg, 180 mg to about 185 mg, about 185 mg to about 190 mg, 190 mg to about 195 mg, about 195 mg to about 200 mg, MDMA and/or a pharmaceutically acceptable salt or solvate thereof on a free base basis of MDMA. In some embodiments, the first split dose comprises 102 mg MDMA and/or a pharmaceutically acceptable salt or solvate thereof on a free base basis of MDMA.

In some embodiments, the second split dose comprises about 100 mg to about 200 mg MDMA and/or a pharmaceutically acceptable salt or solvate thereof on a free base basis of MDMA. In some embodiments, the second split dose comprises about 100 mg to about 105 mg, about 105 mg to about 110 mg, 110 mg to about 115 mg, about 115 mg to about 120 mg, 120 mg to about 125 mg, about 125 mg to about 130 mg, 130 mg to about 135 mg, about 135 mg to about 140 mg, 140 mg to about 145 mg, about 145 mg to about 150 mg, 150 mg to about 155 mg, about 155 mg to about 160 mg, 160 mg to about 165 mg, about 165 mg to about 170 mg, 170 mg to about 175 mg, about 175 mg to about 180 mg, 180 mg to about 185 mg, about 185 mg to about 190 mg, 190 mg to about 195 mg, about 195 mg to about 200 mg, MDMA and/or a pharmaceutically acceptable salt or solvate thereof on a free base basis of MDMA. In some embodiments, the second split dose comprises 150 mg MDMA and/or a pharmaceutically acceptable salt or solvate thereof on a free base basis of MDMA.

In some embodiments, the third split dose comprises about 100 mg to about 200 mg MDMA and/or a pharmaceutically acceptable salt or solvate thereof on a free base basis of MDMA. In some embodiments, the second split dose comprises about 100 mg to about 105 mg, about 105 mg to about 110 mg, 110 mg to about 115 mg, about 115 mg to about 120 mg, 120 mg to about 125 mg, about 125 mg to about 130 mg, 130 mg to about 135 mg, about 135 mg to about 140 mg, 140 mg to about 145 mg, about 145 mg to about 150 mg, 150 mg to about 155 mg, about 155 mg to about 160 mg, 160 mg to about 165 mg, about 165 mg to about 170 mg, 170 mg to about 175 mg, about 175 mg to about 180 mg, 180 mg to about 185 mg, about 185 mg to about 190 mg, 190 mg to about 195 mg, about 195 mg to about 200 mg, MDMA and/or a pharmaceutically acceptable salt or solvate thereof on a free base basis of MDMA. In some embodiments, the third split dose comprises 150 mg MDMA and/or a pharmaceutically acceptable salt or solvate thereof on a free base basis of MDMA.

In some embodiments, a split dose comprises 1 to 3 capsules. In some embodiments, a split dose comprises 1 capsule. In some embodiments, a split dose comprises 2 capsules. In some embodiments, a split dose comprises 3 capsules. In some embodiments, a split dose comprises 3 capsules of 34 mg MDMA and/or a pharmaceutically acceptable salt or solvate thereof on a free base basis of MDMA. In some embodiments, a split dose comprises 3 capsules of 50 mg MDMA and/or a pharmaceutically acceptable salt or solvate thereof on a free base basis of MDMA.

In some embodiments, the composition comprises about 0.1% to about 10% by weight of magnesium stearate and about 25% to about 75% by weight of mannitol.

In some embodiments, the composition comprises about 0.1% to about 10% (e.g., about 0.1% to about 8%, about 0.1% to about 5%, about 0.1% to about 4%, about 0.1% to about 2%, about 0.5% to about 1.5%, or about 1%) by weight of magnesium stearate. In some embodiments, the composition comprises about 1% by weight of magnesium stearate.

In some embodiments, the composition comprises about 25% to about 75% (e.g., about 25% to about 65%, about 25% to about 55%, about 25% to about 50%, about 25% to about 35%, about 35% to about 75%, about 50% to about 75%, about 35% to about 65%, about 40% to about 60%, about 45% to about 55%, or about 49%) by weight of mannitol. In some embodiments, the composition comprises about 49% by weight of mannitol.

In some embodiments, the composition comprises about 34 mg to about 50 mg MDMA or a pharmaceutically acceptable salt and/or solvate thereof on a free base basis of MDMA. In some embodiments, the composition comprises about 34 mg MDMA or a pharmaceutically acceptable salt and/or solvate thereof on a free base basis of MDMA. In some embodiments, the composition of the one or more individual dosage units administered during the first session comprises about 34 mg MDMA or a pharmaceutically acceptable salt and/or solvate thereof on a free base basis of MDMA. In some embodiments, the composition comprises about 50 mg MDMA or a pharmaceutically acceptable salt and/or solvate thereof on a free base basis of MDMA. In some embodiments, the composition of the one or more individual dosage units administered during the second and third sessions comprises about 50 mg MDMA or a pharmaceutically acceptable salt and/or solvate thereof on a free base basis of MDMA.

In some embodiments, the MDMA or a pharmaceutically acceptable salt and/or solvate thereof is MDMA·HCl. In some embodiments, the MDMA·HCl is in the form of anhydrous MDMA·HCl. In some embodiments, the MDMA·HCl is in the form of MDMA·HCl Form I, anhydrous. In some embodiments, the MDMA·HCl is in the form of MDMA·HCl Form II, anhydrous. In some embodiments, the MDMA·HCl is in the form of MDMA·HCl Form III, anhydrous. In some embodiments, the MDMA·HCl is in the form of MDMA·HCl in a mixture of Forms I, II, and III, each substantially free of MDMA monohydrate.

In some embodiments, the second session (e.g., psychological intervention, psychotherapy session, or medication session) is at least 21 days (e.g., at least 3 weeks, at least 4 weeks, at least 5 weeks, at least 6 weeks, at least 7 weeks, at least 8 weeks, about 3 weeks to about 4 weeks, about 3 weeks to about 5 weeks, or about 3 weeks to about 6 weeks) after the first session. In some embodiments, the second session is about 3 weeks to about 5 weeks after the first session.

In some embodiments, the third session (e.g., psychological intervention, psychotherapy session, or medication session) is at least 21 days (e.g., at least 3 weeks, at least 4 weeks, at least 5 weeks, at least 6 weeks, at least 7 weeks, at least 8 weeks, about 3 weeks to about 4 weeks, about 3 weeks to about 5 weeks, or about 3 weeks to about 6 weeks) after the second session. In some embodiments, the third session is about 3 weeks to about 5 weeks after the second session.

In some embodiments, the subject consumed no food for at least 5 hours before administering the MDMA, or a pharmaceutically acceptable salt and/or solvate thereof. For example, in some embodiments, the subject consumed no food for at least 6 hours before administering the MDMA, or a pharmaceutically acceptable salt and/or solvate thereof. In some embodiments, the subject consumed no food for at least 8 hours before administering the MDMA, or a pharmaceutically acceptable salt and/or solvate thereof. For example, in some embodiments, the subject consumed no food for at least 10 hours before administering the MDMA, or a pharmaceutically acceptable salt and/or solvate thereof. In some embodiments, the subject consumed no food for at least 10 hours before administering the MDMA, or a pharmaceutically acceptable salt and/or solvate thereof. For example, in some embodiments, the subject consumed no food for at least 12 hours before administering the MDMA, or a pharmaceutically acceptable salt and/or solvate thereof. In some embodiments, the subject consumed no food for at least 14 hours before administering the MDMA, or a pharmaceutically acceptable salt and/or solvate thereof. For example, in some embodiments, the subject consumed no food for at least 16 hours before administering the MDMA, or a pharmaceutically acceptable salt and/or solvate thereof. In some embodiments, the subject consumed no food for at least 18 hours before administering the MDMA, or a pharmaceutically acceptable salt and/or solvate thereof. For example, in some embodiments, the subject consumed no food for at least 20 hours before administering the MDMA, or a pharmaceutically acceptable salt and/or solvate thereof. In some embodiments, the subject consumed no food for at least 22 hours before administering the MDMA, or a pharmaceutically acceptable salt and/or solvate thereof. For example, in some embodiments, the subject consumed no food for at least 24 hours before administering the MDMA, or a pharmaceutically acceptable salt and/or solvate thereof.

In some embodiments, the subject consumed food up to about 6 hours before administering the MDMA. For example, the subject consumed food up to about 5 hours, about 4 hours, about 3 hours, about 2 hours, about 1 hour, about 30 minutes, about 20 minutes, about 15 minutes, about 10 minutes, about 5 minutes, about 1 minute, about 30 seconds, or about 5 seconds before administering the MDMA. For example, the subject consumed food concurrently with administering the MDMA.

In some embodiments, the dose of the MDMA, or a pharmaceutically acceptable salt and/or solvate thereof is 50 mg on a free base basis of MDMA. In some embodiments, the dose of the MDMA, or a pharmaceutically acceptable salt and/or solvate thereof is 100 mg on a free base basis of MDMA. In some embodiments, the MDMA, or a pharmaceutically acceptable salt and/or solvate thereof is MDMA hydrochloride.

In some embodiments, the dose of the MDMA hydrochloride is 120 mg. In some embodiments, the method comprises measuring a Cₘₐₓ of about 100 ng/mL to about 500 ng/mL in the subject after administering the MDMA·HCl. In some embodiments, the dose of the MDMA hydrochloride can be about 112 mg, about 114 mg, about 116 mg, about 118 mg, about 120 mg, about 122 mg, or about 124 mg. In some embodiments, the method comprises measuring a Cₘₐₓ of about 150 ng/mL to about 450 ng/mL, about 175 ng/mL to about 400 ng/mL, about 200 ng/mL to about 320 ng/mL, about 220 ng/mL to about 300 ng/mL, about 218 ng/mL to about 258 ng/mL, about 228 ng/mL to about 248 ng/mL, about 240 ng/mL to about 280 ng/mL, about 250 ng/mL to about 275 ng/mL, about 255 ng/mL to about 270 ng/mL, about 158 ng/mL to about 164 ng/mL, about 238 ng/mL, or about 261 ng/mL. In some embodiments, the method comprises measuring a Cₘₐₓ of about 261 ng/mL. In some embodiments, the method comprises measuring a Cₘₐₓ of about 238 ng/mL. The subranges and specific values can be selected based on factors such as the participant's weight, age, and overall health, as well as the desired therapeutic effect and potential side effects of the MDMA.

In some embodiments, the dose of the MDMA hydrochloride is 100 mg. In some embodiments, the method comprises measuring a Cₘₐₓ of about 100 ng/mL to about 500 ng/mL in the subject after administering the MDMA·HCl. In some embodiments, the dose of the MDMA hydrochloride can be about 112 mg, about 114 mg, about 116 mg, about 118 mg, about 120 mg, about 122 mg, or about 124 mg. In some embodiments, the method comprises measuring a Cₘₐₓ of about 150 ng/mL to about 450 ng/mL, about 175 ng/mL to about 400 ng/mL, about 200 ng/mL to about 320 ng/mL, about 220 ng/mL to about 300 ng/mL, about 218 ng/mL to about 258 ng/mL, about 228 ng/mL to about 248 ng/mL, about 240 ng/mL to about 280 ng/mL, about 250 ng/mL to about 275 ng/mL, about 255 ng/mL to about 270 ng/mL, about 158 ng/mL to about 164 ng/mL, about 238 ng/mL, or about 261 ng/mL. In some embodiments, the method comprises measuring a Cₘₐₓ of about 238 ng/mL. The subranges and specific values can be selected based on factors such as the participant's weight, age, and overall health, as well as the desired therapeutic effect and potential side effects of the MDMA.

In some embodiments, the method comprises measuring an AUC₀₋ₜ in the subject after administering MDMA·HCl. In some embodiments, the measured AUC₀₋ₜ is about 2500 h*ng/mL to about 5000 h*ng/mL, about 3000 h*ng/mL to about 4500 h*ng/mL, about 3000 h*ng/mL to about 4200 h*ng/mL, about 3000 h*ng/mL to about 3800 h*ng/mL, about 3000 h*ng/mL to about 3600 h*ng/mL, about 3000 h*ng/mL to about 3400 h*ng/mL, about 3000 h*ng/mL to about 3300 h*ng/mL, about 3000 h*ng/mL to about 3250 h*ng/mL, about 3050 h*ng/mL to about 3200 h*ng/mL, about 3100 h*ng/mL to about 3150 h*ng/mL, about 3300 h*ng/mL to about 4000 h*ng/mL, about 3570 h*ng/mL to about 3770 h*ng/mL, about 3500 h*ng/mL to about 3600 h*ng/mL, about 3520 h*ng/mL to about 3580 h*ng/mL, about 3620 h*ng/mL to about 3730 h*ng/mL, about 3123 h*ng/mL, or about 3670 h*ng/mL. In some embodiments, the measured AUC₀₋ₜ is about 3550 h*ng/mL. In some embodiments, the measured AUC₀₋ₜ is about 3670 h*ng/mL. In some embodiments, the measured AUC₀₋ₜ is about 3123 h*ng/mL.

In some embodiments, the method comprises measuring an AUC₀₋₇₂ in the subject after administering MDMA·HCl. In some embodiments, the measured AUC₀₋₇₂ is about 2500 h*ng/mL to about 5000 h*ng/mL, about 3000 h*ng/mL to about 4500 h*ng/mL, about 3000 h*ng/mL to about 3700 h*ng/mL, about 3200 h*ng/mL to about 3500 h*ng/mL, about 3300 h*ng/mL to about 3440 h*ng/mL, about 3500 h*ng/mL to about 4200 h*ng/mL, about 3700 h*ng/mL to about 3900 h*ng/mL, about 3750 h*ng/mL to about 3850 h*ng/mL, about 3800 h*ng/mL to about 4000 h*ng/mL, about 3870 h*ng/mL to about 3900 h*ng/mL, or about 3880 h*ng/mL. In some embodiments, the measured AUC₀₋₇₂ is about 3800 h*ng/mL. In some embodiments, the measured AUC_{0-inf} is about 3880 h*ng/mL. In some embodiments, the method comprises measuring an AUC_{0-inf} in the subject after administering MDMA·HCl. In some embodiments, the measured AUC_{0-inf} is about 2500 h*ng/mL to about 5000 h*ng/mL, about 3000 h*ng/mL to about 4500 h*ng/mL, about 3500 h*ng/mL to about 4200 h*ng/mL, about 3700 h*ng/mL to about 3900 h*ng/mL, about 3750 h*ng/mL to about 3850 h*ng/mL, about 3800 h*ng/mL to about 4000 h*ng/mL, about 3870 h*ng/mL to about 3900 h*ng/mL, about 3368 h*ng/mL, or about 3890 h*ng/mL. In some embodiments, the measured AUC_{0-inf} is about 3800 h*ng/mL. In some embodiments, the measured AUC_{0-inf} is about 3890 h*ng/mL. In some embodiments, the measured AUC_{0-inf} is about 3368 h*ng/mL.

In some embodiments, the method comprises measuring a Tₘₐₓ in the subject after administering MDMA·HCl. In some embodiments, the measured Tₘₐₓ is about 30 minutes to about 10 hours, about 30 minutes to about 4 hours, about 45 minutes to about 3 hours, about 2 hours to about 8 hours, about 4 hours to about 10 hours, about 6 hours to about 10 hours, about 2 hours to about 6 hours, about 3 hours to about 5 hours, about 1 hour to about 3 hours, about 1.5 hours to about 2.5 hours, about 1.7 hours to about 2.3 hours, or about 2 hours. In some embodiments, the measured Tₘₐₓ is about 4 hours. In some embodiments, the measured Tₘₐₓ is about 2 hours.

In some embodiments, the method comprises measuring a t_{1/2} in the subject after administering MDMA·HCl. In some embodiments, the measured t_{1/2} is about 2 hours to about 20 hours, about 3 hours to about 20 hours, about 4 hours to about 20 hours, about 4 hours to about 15 hours, about 4 hours to about 12 hours, about 4 hours to about 8 hours, about 4 hours to about 6 hours, about 6 hours to about 20 hours, about 8 hours to about 20 hours, about 10 hours to about 20 hours, about 13 hours to about 20 hours, about 16 hours to about 20 hours, about 18 hours to about 20 hours, about 6 hours to about 12 hours, about 7 hours to about 11 hours, about 8 hours to about 10 hours, about 8 hours to about 8 hours, about 8.36 hours, about 8.7 hours, or about 9 hours. In some embodiments, the measured t_{1/2} is about 8.36 hours. In some embodiments, the measured t_{1/2} is about 8.7 hours. In some embodiments, the measured t_{1/2} is about 9 hours.

In some embodiments, the method comprises measuring a T_{lag} in the subject after administering MDMA·HCl. In some embodiments, the measured T_{lag} is about 0 hours to about 2 hours, about 0.25 hours to about 1.5 hours, about 0.25 hours to about 0.75 hours, about 0.75 hours to about 1.25 hours, about 0.5 hours, about 0.6 hours, about 0.75 hours, or about 1 hour. In some embodiments, the measured T_{lag} is about 0.5 hours. In some embodiments, the measured T_{lag} is about 0.5 hour. In some embodiments, the measured T_{lag} is about 0.6 hour.

In some embodiments, the method comprises measuring a CL/F in the subject after administering MDMA·HCl. In some embodiments, the measured CL/F is about 1 L/h to about 100 L/h, about 1 L/h to about 70 L/h, about 10 L/h to about 60, about 20 L/h to about 50 L/h, about 20 L/h to about 40 L/h, about 25 L/h to about 35 L/h, about 30 L/h to about 40 L/h, about 32 L/h to about 36 L/h, about 35 L/h to about 40 L/h, about 34.5 L/h, about 29.5 L/h, or about 37.5 L/h. In some embodiments, the measured CL/F is about 368 L/h. In some embodiments, the measured CL/F is about 34.5 L/h. In some embodiments, the measured CL/F is about 368 L/h. In some embodiments, the measured CL/F is about 37.5 L/h. In some embodiments, the measured CL/F is about 29.5 L/h.

In some embodiments, the method comprises measuring a Vd/F in the subject after administering MDMA-HCL. In some embodiments, the measured Vd/F is about 100 L to about 800 L, about 200 L to about 700 L, about 300 L to about 600 L, about 300 L to about 450 L, about 320 L to about 420 L, about 400 L to about 500 L, about 400 L to about 420 L, about 410 L to about 450 L, about 420 L to about 440 L, about 425 L to about 435 L, about 371.7 Vd/F, about 412 L, or about 430 L. In some embodiments, the measured Vd/F is about 430 L. In some embodiments, the measured Vd/F is about 412 Vd/F. In some embodiments, the measured Vd/F is about 371.7 Vd/F.

Non-limiting examples of diagnostic tests to assess improvement in the symptoms of PTSD in the subject include the Clinician-Administered PTSD scale for DSM-5 (CAPS-5) and Sheehan Disability Scale (SDS). Further information on the CAPS-5 can be found at Psychol Assess. 2018 March; 30(3):383-395. Further information on the SDS can be found at Int. Clin. Psychopharmacol. 2008; 23(2):70-83.

In some embodiments, a reduction (i.e., improvement) in the CAPS-5 is measured in the subject after administering the MDMA in comparison to before administering the MDMA. In some embodiments, a reduction of at least 1 point (e.g., at least 2 points, at least 4 points, at least 6 points, at least 8 points, at least 10 points, at least 12 points, at least 14 points, at least 15 points, at least 16 points, at least 18 points, at least 20 points, at least 23 points, at least 25 points, at least 30 points, at least 40 points) in the CAPS-5 is measured in the subject after administering the MDMA in comparison to before administering the MDMA. In some embodiments, a reduction of at least 6 points in the CAPS-5 is measured in the subject after administering the MDMA in comparison to before administering the MDMA. In some embodiments, a reduction of at least 10 points in the CAPS-5 is measured in the subject after administering the MDMA in comparison to before administering the MDMA. In some embodiments, a reduction of at least 15 points in the CAPS-5 is measured in the subject after administering the MDMA in comparison to before administering the MDMA. In some embodiments, a reduction of at least 20 points in the CAPS-5 is measured in the subject after administering the MDMA in comparison to before administering the MDMA. In some embodiments, a reduction of at least 23 points in the CAPS-5 is measured in the subject after administering the MDMA in comparison to before administering the MDMA. In some embodiments, the CAPS-5 score of the subject after administering the MDMA is less than or equal to 11 (meeting the definition of being in remission).

In some embodiments, the subject has a CAPS-5 score of least 35 points prior to the first administration of the composition or unit dosage form described herein and a reduction of from 1 to 40 points after the third administration of the composition or unit dosage form described herein.

In some embodiments, a reduction (i.e., improvement) in the SDS is measured in the subject after administering the MDMA in comparison to before administering the MDMA. In some embodiments, a reduction of at least 1 point in the SDS is measured in the subject after administering the MDMA in comparison to before administering the MDMA. In some embodiments, a reduction of at least 2 points in the SDS is measured in the subject after administering the MDMA in comparison to before administering the MDMA. In some embodiments, a reduction of at least 2 points in the SDS is measured in the subject after administering the MDMA in comparison to before administering the MDMA. In some embodiments, a reduction of at least 3 points in the SDS is measured in the subject after administering the MDMA in comparison to before administering the MDMA. In some embodiments, a reduction of at least 4 points in the SDS is measured in the subject after administering the MDMA in comparison to before administering the MDMA. In some embodiments, a reduction of at least 5 points in the SDS is measured in the subject after administering the MDMA in comparison to before administering the MDMA.

### Processes of Preparing MDMA Particles

Some embodiments provide a process for obtaining particles comprising crystalline 3,4-methylenedioxymethamphetamine (MDMA), or a pharmaceutically acceptable salt and/or solvate thereof, wherein the particles comprise particles that are substantially less than about 610 μm; wherein the process comprises:
(a) contacting a salt of MDMA with an organic solvent to obtain a first solution,
(b) heating and stirring the first solution to obtain a second solution,
(c) filtering the second solution to obtain a third solution,
(d) cooling the third solution over a first time period to a first set temperature,
(e) adding crystalline MDMA, or a pharmaceutically acceptable salt and/or solvate thereof seeds to the cooled solution of step (d) to obtain a fourth solution,
(f) stirring the fourth solution of step (e) for a second time period at the first set temperature,
(g) cooling the fourth solution of step (f) over a third time period to a second set temperature,
(h) stirring the fourth solution of step (g) at the second set temperature for a fourth time period to obtain crystalline MDMA or a pharmaceutically acceptable salt and/or solvate thereof,
(i) filtering the solution of step (h) to obtain particles of crystalline MDMA or a pharmaceutically acceptable salt and/or solvate thereof,
(j) drying the particles of MDMA of step (i) at a set drying temperature under a set drying pressure for a set drying time period, and
(k) milling the particles of step (j) under an inert atmosphere at a set milling speed and passing the milled particles through a mesh screen of a set size to obtain particles comprising crystalline 3,4-methylenedioxymethamphetamine (MDMA), or a pharmaceutically acceptable salt and/or solvate thereof.

Some embodiments provide particles comprising crystalline 3,4-methylenedioxymethamphetamine (MDMA), or a pharmaceutically acceptable salt and/or solvate thereof, wherein the particles comprise particles that are substantially less than about 610 μm; obtained by a process comprising:
(a) contacting a salt of MDMA with an organic solvent to obtain a first solution,
(b) heating and stirring the first solution to obtain a second solution,
(c) filtering the second solution to obtain a third solution,
(d) cooling the third solution over a first time period to a first set temperature,
(e) adding crystalline MDMA, or a pharmaceutically acceptable salt and/or solvate thereof seeds to the cooled solution of step (d) to obtain a fourth solution,
(f) stirring the fourth solution of step (e) for a second time period at the first set temperature,
(g) cooling the fourth solution of step (f) over a third time period to a second set temperature,
(h) stirring the fourth solution of step (g) at the second set temperature for a fourth time period to obtain crystalline MDMA or a pharmaceutically acceptable salt and/or solvate thereof,
(i) filtering the solution of step (h) to obtain particles of crystalline MDMA or a pharmaceutically acceptable salt and/or solvate thereof,
(j) drying the particles of MDMA of step (i) at a set drying temperature under a set drying pressure for a set drying time period, and
(k) milling the particles of step (j) under an inert atmosphere at a set milling speed and passing the milled particles through a mesh screen of a set size to obtain particles comprising crystalline 3,4-methylenedioxymethamphetamine (MDMA), or a pharmaceutically acceptable salt and/or solvate thereof.

In some embodiments, the salt of MDMA of step (a) is MDMA·HCl.

In some embodiments, the organic solvent of step (a) is 2-propanol.

In some embodiments, the heating of step (b) is heating to a temperature of about 67° C.

In some embodiments, first time period of step (d) is about 90 minutes.

In some embodiments, the first set temperature of step (d) is about 55° C.

In some embodiments, the second time period of step (f) is about 30 min.

In some embodiments, the second set temperature of step (g) is about 15° C.

In some embodiments, the cooling of step (g) is done at a rate of about 3° C. per hour.

In some embodiments, the fourth time period of step (h) is about 10 hours.

In some embodiments, the set drying temperature of step (j) is about 56° C.

In some embodiments, the set drying pressure of step (j) is about 140 mbar.

In some embodiments, the set drying time of step (j) is about 19 hours.

In some embodiments, the set milling speed of step (k) is about 6000 rotations per minute (RPM).

In some embodiments, the set size of the mesh screen of step (k) is about 610 μM.

### Numbered Embodiments

1. Particles comprising 3,4-methylenedioxymethamphetamine (MDMA), or a pharmaceutically acceptable salt and/or solvate thereof, and one or more pharmaceutically acceptable excipients, wherein the particle is substantially less than about 610 μm.

2. The particles of embodiment 1, wherein the particles have a Dv90 less than about 420 μm, and a particle size range (Dv90-Dv10) less than about 400 μm.

3. The particles of embodiment 2, wherein the particles have a Dv90 less than about 400 μm.

4. The particles of any one of embodiments 1-3, wherein 0-10% of the particles have a particle size (Dv10) from about 0.01 μm to about 10 μm.

5. The particles of any one of the preceding embodiments, wherein the particles have a median particle size (Dv50) from about 100 μm to about 200 μm.

6. The particles of any one of the preceding embodiments, wherein the chemical purity of the particles is from about 98-100% and no single impurity is present in an amount of from about 0.5% to about 100% as determined by HPLC.

7. The particles of any one of the preceding embodiments, wherein the chemical purity of the particles is from 99-100% and no single impurity is present in an amount of from about 0.5% to about 1% as determined by HPLC.

8. The particles of any one of embodiments 1-7, wherein the particles are prepared by a process comprising a step of reducing particle size and increasing particle size uniformity of coarse particles by screen-milling using a screen mill.

9. The particles of embodiment 8, wherein the coarse particles do not undergo an additional size-reducing process.

10. The particles of embodiment 8 or 9, wherein substantially all of the particles are less than about 610 μm.

11. The particles of any one of embodiments 8-10, wherein the median particle size (Dv50) of the coarse particles is from about 300 μm to about 900 μm.

12. The particles of embodiment 11, wherein the coarse particles are substantially free of MDMA·HCl monohydrate.

13. The particles of any of embodiments 8-12, wherein the coarse particles are heated to a temperature of 50-70° C. in an environment with an ambient pressure of from about 0.1-1 atmosphere, before entering the screen mill.

14. The particles of any one of embodiments 8-13, wherein the coarse particles are fed into the screen mill in the absence of applied pressure.

15. The particles of any one of embodiments 8-14, wherein the milling method is conducted in an inert atmosphere that is substantially free of moisture.

16. The particles of embodiment 15, wherein the inert atmosphere comprises substantially dry nitrogen gas.

17. The particles of any one of the preceding embodiments, wherein the particles are substantially free of MDMA·HCl monohydrate.

18. A dosage form comprising the particles of any one of the preceding embodiments and one or more pharmaceutically acceptable excipients.

19. The dosage form of embodiment 18, comprising about 1 mg to about 150 mg of MDMA, or a pharmaceutically acceptable salt and/or solvate thereof, on a free base basis of MDMA.

20. The dosage form of embodiment 18 or 19, comprising about 35 mg to about 45 mg of MDMA, or a pharmaceutically acceptable salt and/or solvate thereof, on a free base basis of MDMA.

21. The dosage form of any one of embodiments 18-20, comprising about 55 mg to about 65 mg of MDMA, or a pharmaceutically acceptable salt and/or solvate thereof, on a free base basis of MDMA.

22. The dosage form of any one of embodiments 18-21, comprising about 75 mg to about 85 mg of MDMA, or a pharmaceutically acceptable salt and/or solvate thereof, on a free base basis of MDMA.

23. The dosage form of any one of embodiments 18-22, comprising about 95 mg to about 105 mg of MDMA, or a pharmaceutically acceptable salt and/or solvate thereof, on a free base basis of MDMA.

24. The dosage form of any one of embodiments 18-23, comprising about 115 mg to about 125 mg of MDMA, or a pharmaceutically acceptable salt and/or solvate thereof, on a free base basis of MDMA.

25. The dosage form of any one of embodiments 18-24, wherein the dissolution rate in water is greater than or equal to 80% of the mass of the MDMA in 30 minutes.

26. The dosage form of any one of embodiments 18-25, wherein the particles are prepared by a process comprising a step of reducing the particle size and increasing particle size uniformity by screen-milling under an inert atmosphere.

27. The dosage form of any one of embodiments 18-26, wherein the dosage form comprises a diluent.

28. The dosage form of embodiment 27, wherein the diluent is a sugar alcohol.

29. The dosage form of embodiment 27 or 28, wherein the diluent has a moisture content from 0% to about 0.25% by mass, prior to blending.

30. The dosage form of embodiment 18, wherein the composition additionally comprises a lubricant.

31. The dosage form of embodiment 30, wherein the lubricant comprises a pharmaceutically acceptable salt of a saturated fatty acid.

32. The dosage form of any one of embodiments 18-31, wherein substantially all of the particles are less than about 610 μm.

33. The dosage form of any one of embodiments 18-32, wherein the Dv90 of the particles is from about 0.01 μm to about 400 μm.

34. The dosage form of any one of embodiments 18-33, wherein from about 0-10% of the particles have a particle size (Dv10) from about 0.01 μm to about 10 μm.

35. The dosage form of any one of embodiments 18-34, wherein the median particle size (Dv50) of the particles is from 100 μm to 200 μm.

36. The dosage form of any one of embodiments 18-35, wherein the chemical purity of the particles is from about 98-100% and no single impurity is present in an amount of from about 0.5% to about 100% as determined by HPLC.

37. The dosage form of any one of embodiments 18-36, wherein the chemical purity of the particles is from about 99-100% and no single impurity is present in an amount of from about 0.5% to about 100% as determined by HPLC.

38. The dosage form of any one of embodiments 18-36, wherein the dosage form is a capsule or a tablet.

39. The dosage form of any one of embodiments 18-38, wherein the dosage form is a capsule.

40. The dosage form of any one of embodiments 18-38, wherein the dosage form is a tablet.

41. The dosage form of any one of embodiments 18-40, comprising one or more individual dosage units.

42. The dosage form of embodiment 41, comprising one individual dosage unit.

43. The dosage form of embodiment 42, comprising at least two individual dosage units.

44. The dosage form of embodiment 43, comprising at least three individual dosage units.

45. The dosage form of any one of embodiments 41-44, wherein each of the one or more individual dosage units comprises a capsule.

46. The dosage form of any one of embodiments 41-45, wherein the one or more individual dosage units are administered during a single psychological intervention.

47. The dosage form of any one of embodiments 41-46, wherein the one or more individual dosage units are administered at different times during the single psychological intervention.

48. The dosage form of any one of embodiments 41-45, wherein the one or more individual dosage units are administered during a single psychotherapy session.

49. The dosage form of any one of embodiments 41-45 or 48, wherein the one or more individual dosage units are administered at different times during the single psychotherapy session.

50. The dosage form of any one of embodiments 41-45, wherein the one or more individual dosage units are administered during a single medication session.

51. The dosage form of any one of embodiments 41-45 or 50, wherein the one or more individual dosage units are administered at different times during the single medication session.

52. A method of treating a central nervous system disorder in a subject, the method comprising: administering to the subject a therapeutically effective amount of the particles of any one of embodiments 1-17 or the dosage form of any one of embodiments 18-51.

53. The method of embodiment 52, wherein the central nervous system disorder is a trauma-linked disorder or a stressor-linked disorder.

54. The method of embodiment 52, wherein the central nervous system disorder is a mood disorder.

55. The method of embodiment 52, wherein the central nervous system disorder is an anxiety disorder.

56. The method of embodiment 52, wherein the central nervous system disorder is post-traumatic stress disorder.

57. The method of any one of embodiments 52-56, wherein the administering is performed during a psychotherapy session.

58. The method of any one of embodiments 52-57, wherein about 34 mg of MDMA, or a pharmaceutically acceptable salt and/or solvate thereof, on a free base basis of MDMA is administered.

59. The method of embodiment 58, wherein the about 34 mg of MDMA, or a pharmaceutically acceptable salt and/or solvate thereof, on a free base basis of MDMA is administered in one dose.

60. The method of any one of embodiments 52-57, wherein about 50 mg of MDMA, or a pharmaceutically acceptable salt and/or solvate thereof, on a free base basis of MDMA is administered.

61. The method of embodiment 60, wherein the about 50 mg of MDMA, or a pharmaceutically acceptable salt and/or solvate thereof, on a free base basis of MDMA is administered in one dose.

62. The method of any one of embodiments 52-57, wherein about 100 mg of MDMA, or a pharmaceutically acceptable salt and/or solvate thereof, on a free base basis of MDMA is administered.

63. The method of embodiment 62, wherein the about 100 mg of MDMA, or a pharmaceutically acceptable salt and/or solvate thereof, on a free base basis of MDMA is administered in one dose.

64. The method of embodiment 62, wherein the about 100 mg of MDMA, or a pharmaceutically acceptable salt and/or solvate thereof, on a free base basis of MDMA is administered in two doses.

65. The method of any one of embodiments 48-53, wherein about 150 mg of MDMA, or a pharmaceutically acceptable salt and/or solvate thereof, on a free base basis of MDMA is administered.

66. The method of embodiment 60, wherein the about 150 mg of MDMA, or a pharmaceutically acceptable salt and/or solvate thereof, on a free base basis of MDMA is administered in one dose.

67. The method of embodiment 60, wherein the about 150 mg of MDMA, or a pharmaceutically acceptable salt and/or solvate thereof, on a free base basis of MDMA is administered in two doses.

68. The method of any one of embodiments 52-67, wherein the dosage form comprising the therapeutically effective amount of MDMA, on a free base basis of MDMA or a pharmaceutically acceptable salt and/or solvate thereof, is orally administered.

69. The method of embodiment 68, wherein the therapeutically effective amount of the MDMA, or a pharmaceutically acceptable salt and/or solvate thereof, is administered in a capsule.

70. The method of embodiment 68, wherein the dosage form comprising the therapeutically effective amount of MDMA, or a pharmaceutically acceptable salt and/or solvate thereof, is administered in a tablet.

71. The method form of any one of embodiments 52-70, wherein the dosage form comprising the therapeutically effective amount of MDMA, or a pharmaceutically acceptable salt and/or solvate thereof, is administered as one or more individual dosage units during a single dosing session.

72. The method form of any one of embodiments 52-70, wherein the dosage form comprising the therapeutically effective amount of MDMA, or a pharmaceutically acceptable salt and/or solvate thereof, is administered at different times during a single psychotherapy session.

73. The particle, dosage form, or method of any one of embodiments 1-72, wherein the MDMA, or a pharmaceutically acceptable salt and/or hydrate is a pharmaceutically acceptable salt.

74. The particle, dosage form, or method of any one of embodiments 1-73, wherein the MDMA, or a pharmaceutically acceptable salt and/or hydrate is present in the form of the hydrochloride salt.

75. A method of treating an eating disorder in a subject, comprising administering to the subject a therapeutically effective amount of particles comprising crystalline 3,4-methylenedioxymethamphetamine (MDMA), or a pharmaceutically acceptable salt and/or solvate thereof, wherein the particles comprise particles that are substantially less than about 610 μm.

76. The method of embodiment 75, wherein the particles comprise crystalline MDMA, or a pharmaceutically acceptable salt thereof.

77. The method of embodiment 75 or 76, wherein the particles comprise crystalline MDMA hydrochloride.

78. The method of any one of embodiments 75-77, wherein the Dv90 of the particles is below about 420 μm, and the particle size range (Dv90-Dv10) of the particles is less than about 400 km.

79. The method of any one of embodiments 75-78, wherein the Dv90 of the particles is below about 400 km.

80. The method of any one of embodiments 75-79, wherein 0-10% of the particles have a particle size from about 0.01 μm to about 10 μm.

81. The method of any one of embodiments 75-80, wherein the median particle size (Dv50) of the particles is from about 100 μm to about 200 μm.

82. The method of any one of embodiments 75-82, wherein the chemical purity of the particles is from about 98-100% and no single impurity is present in an amount from 0.5-100% as determined by HPLC.

83. The method of any one of embodiments 75-82, wherein the chemical purity of the particles is from about 99-100% and no single impurity is present in an amount from 0.5-100% as determined by HPLC.

84. The method of any one of embodiments 75-83, wherein the particles are substantially free of MDMA·HCl monohydrate.

85. The method of any one of embodiments 75-84, wherein the dissolution rate of the particles in water exceeds 80% by mass, in 30 minutes.

86. The method of any one of embodiments 75-85, wherein the eating disorder is anorexia nervosa, atypical anorexia nervosa, bulimia nervosa, binge-eating disorder, rumination disorder, avoidant/restrictive food disorder, orthorexia, purging disorder, or other specified feeding or eating disorder (OSFED).

87. The method of any one of embodiments 75-86, wherein the eating disorder is anorexia nervosa.

88. The method of any one of embodiments 75-86, wherein the eating disorder is atypical anorexia nervosa.

89. The method of any one of embodiments 75-86, wherein the eating disorder is bulimia nervosa.

90. The method of any one of embodiments 75-86, wherein the eating disorder is binge-eating disorder.

91. The method of any one of embodiments 75-86, wherein the eating disorder is rumination disorder.

92. The method of any one of embodiments 75-86, wherein the eating disorder is avoidant/restrictive food disorder.

93. The method of any one of embodiments 75-86, wherein the eating disorder is orthorexia.

94. The method of any one of embodiments 75-86, wherein the eating disorder is purging disorder.

95. The method of any one of embodiments 75-86, wherein the eating disorder is other specified feeding or eating disorder (OSFED).

96. The method of any one of embodiments 75-95, wherein the particles are administered to the subject in a pharmaceutically-acceptable dosage form.

97. The method of any one of embodiments 75-96, wherein the dosage form comprises about 1 mg to about 150 mg of the particles.

98. The method of any one of embodiments 75-97, wherein the dosage form comprises about 35 mg to about 45 mg of the particles.

99. The method of any one of embodiments 75-97, wherein the dosage form comprises about 55 mg to about 65 mg of the particles.

100. The method of any one of embodiments 75-97, wherein the dosage form comprises about 75 mg to about 85 mg of the particles.

101. The method of any one of embodiments 75-97, wherein the dosage form comprises about 95 mg to about 105 mg of the particles.

102. The method of any one of embodiments 75-97, wherein the dosage form comprises about 115 mg to about 125 mg of the particles.

103. The method of any one of embodiments 75-102, wherein the dosage form is an oral dosage form.

104. The method of embodiment 103, wherein the dosage form additionally comprises a diluent.

105. The method of embodiment 104, wherein the diluent is a sugar alcohol.

106. The method of embodiment 103 or 104, wherein the diluent has a moisture content from about 0-0.25% by mass, prior to blending.

107. The method of any one of embodiments 75-106, wherein the composition additionally comprises a lubricant.

108. The method of embodiment 107, wherein the lubricant comprises a pharmaceutically acceptable salt of a saturated fatty acid.

109. The method of any one of embodiments 97-108, wherein the dosage form comprises one or more individual dosage units.

110. The method of embodiment 109, wherein the dosage form comprises one individual dosage unit.

111. The method of embodiment 109, wherein the dosage form comprises at least two individual dosage units.

112. The method of embodiment 109, wherein the dosage form comprises at least three individual dosage units.

113. The method of any one of embodiments 109-112, wherein each of the one or more individual dosage units comprises a capsule.

114. The method of any one of embodiments 109-113, wherein the one or more individual dosage units are administered during a single therapy session.

115. The method of any one of embodiments 109-114, wherein the one or more individual dosage units are administered at different times during the single therapy session.

All publications, patents, patent applications, and information available on the internet and cited in the present disclosure are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

### EXAMPLES

These examples are provided for illustrative purposes only and not to limit the scope of the claims provided herein.

### Example 1. Preparation of MDMA

This example provides methods of preparing high-purity MDMA. To a 50 L reaction vessel were added 4107.3 g crude MDMA·HCl and 41000 mL 2-propanol. The batch temperature was raised to 67.2° C., while stirring, and the mixture was then stirred for 30 minutes at 67.2° C. until all of the solids dissolved. Stress-tests had demonstrated stability for 72 hours at 70-80° C., proving the thermal stability of MDMA·HCl.

The batch was then transferred through a 1.2 μm in-line filter capsule, using positive pressure, to a clean, 50 L reaction vessel, fitted with a jacket that had been pre-heated to 66.1° C. In this new reaction vessel, the batch was cooled to 55.3° C., over the course of 90 minutes. 41.1 g of MDMA·HCl Form I seed crystal (0.18 mol, 0.008 equivalents) were then added, and the batch was stirred at the same temperature for 30 minutes. The batch was cooled to 15.2° C. at a rate of 3° C./hour, then stirred at this temperature for an additional 10 hours.

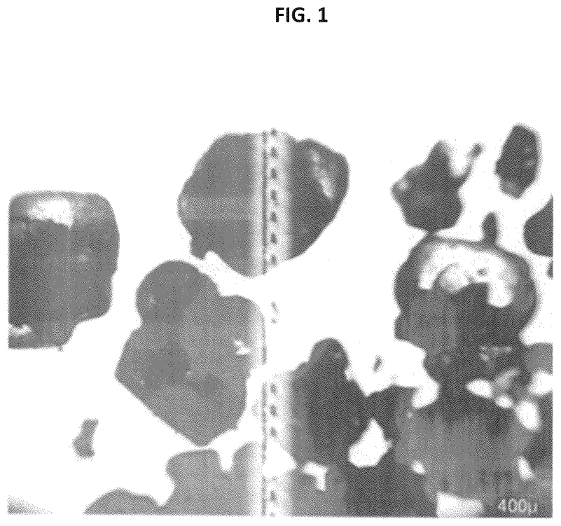

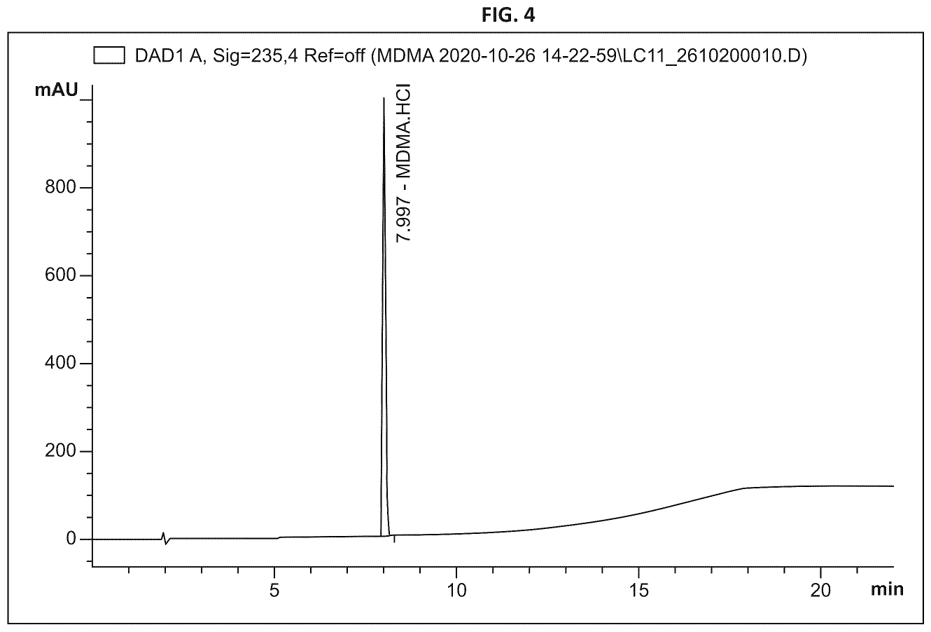

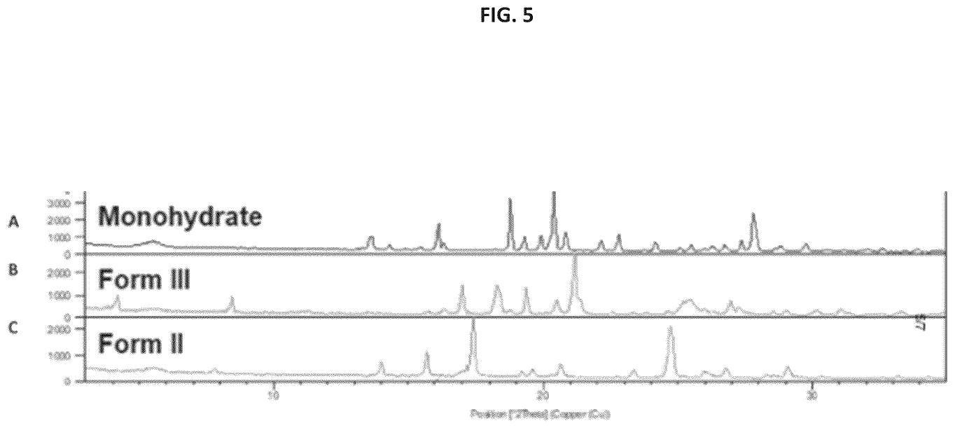

The white suspension was removed from the mother liquor via vacuum filtration over a filter plate fitted with a filter cloth then washed with 8220 mL 2-propanol. The filter cake was transferred to a drying oven, and dried under vacuum (140 mbar) for 19 hours at 56.6° C. The collected MDMA·HCl was a white solid weighing 3548.3 g (85.5% yield; 99.95% peak area, 99.64% w/w by HPLC). No single impurity exceeded 0.02% of peak area by HPLC, and residual solvents (methanol, <6 ppm; 2-propanol, 490 ppm) were found to be within the target range. FIG. 1 shows the coarse MDMA·HCl particles isolated from the synthetic process, and FIG. 4 shows an HPLC chromatogram for coarse MDMA·HCl particles isolated from the synthetic process. Other polymorphic forms were also prepared. FIG. 5 shows the XRPD spectra of MDMA·HCl monohydrate (5A), MDMA·HCl Form III (5B), and MDMA·HCl Form II (5C).

### Example 2. General Description of Screen Milling Process

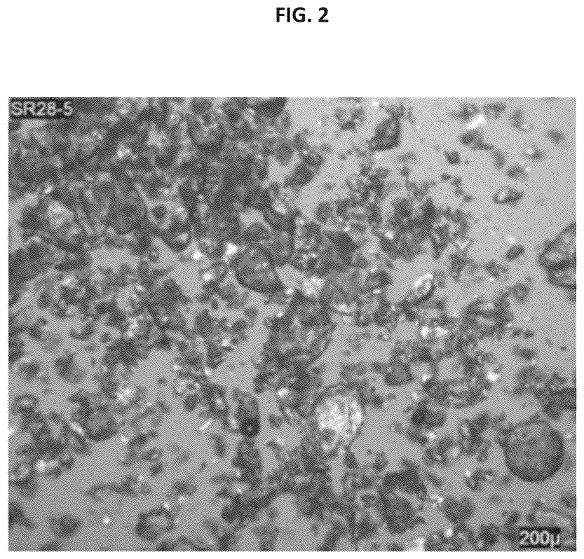

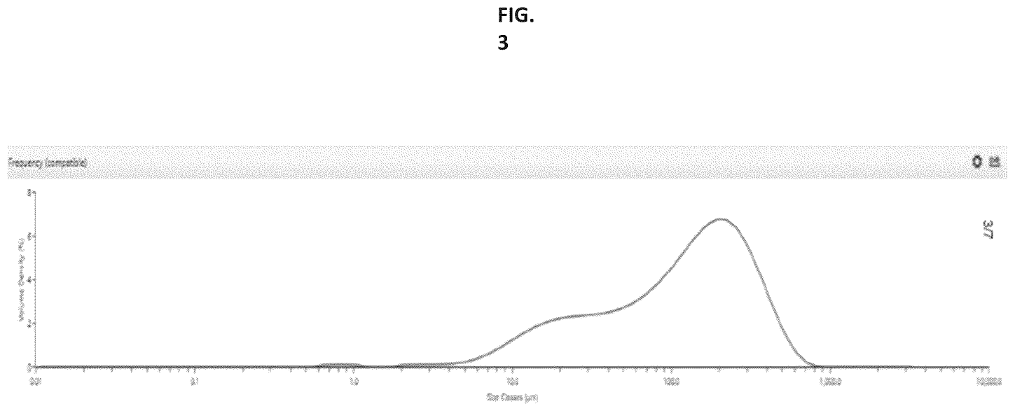

1911 g MDMA·HCl, split into four sub-lots, was fed into an Ytron-Quadro Comill with a stainless steel 610 screen and a rounded mixing drive. The solids were fed into the mill under an inert atmosphere, without pressure applied, and passed directly into a polyethylene collection bag with an earthing cable protecting the equipment from static discharge. A mill speed of 6000 rpm was utilized. The feeding of all four batches was conducted by hand and took place over 30 minutes to avoid a significant build up within the mill. The mass of MDMA recovered from the mill was 1880 g, as measured after analytical sampling. Milling was in general rapid and facile. XRPD data indicated no evidence of MDMA·HCl monohydrate formation in any of the four sub-lots. The milled product was found to be 99.9% MDMA by HPLC (100.0% on a dry basis). Particle size of MDMA recovered from this experiment for each sample/lot is shown in Table 1. The results show that each of the four sub-lots consistently showed a Dv90 of less than 400 μm. FIG. 2 shows exemplary particles comprising MDMA after milling, and FIG. 3 shows the particle size distribution (PSD) of the milled particles of FIG. 2.

TABLE 1
Particle Size of MDMA Measured by Laser Diffraction.

| Sample | Dv90 | Dv50 | Dv10 | |
|---|---|---|---|---|
| CJS194-1 | 342 μm | 170 μm | 29.0 | μm |
| CJS194-2 | 326 μm | 135 μm | 20.0 | μm |
| CJS194-3 | 326 μm | 134 μm | 20.9 | μm |
| CJS194-4 | 353 μm | 161 μm | 23.8 | μm |
| CJS194-5 (blend of lots 1-4) | 341 μm | 151 μm | 23.4 | μm |
| Input 201101 | 844 μm | 512 μm | 376 | μm |

### Example 3. Dosage Form Specifications

### Description of Drug Product

Two dosage strengths of the drug product are available including 34 mg MDMA (equivalent to 40.5 mg MDMA hydrochloride (MDMA·HCl)) and 50 mg MDMA (equivalent to 59.5 mg MDMA·HCl) in hydroxypropyl methylcellulose (HPMC) capsules. The capsules are imprinted and are filled with a composition comprising MDMA·HCl. The appearance of the 34 mg dosage strength capsule is a Size 2, HPMC Swedish Orange/White Capsule imprinted with “MDMA 34” and the appearance of the 50 mg dosage strengths is a Size 2, HPMC Swedish Orange Capsule imprinted with “MDMA 50.”

### Composition of Drug Product

HPMC capsules filled with a powder blend including MDMA·HCl, mannitol, and magnesium stearate. The formulation of the powder blend is a proportional formulation. See Table 2 below for the composition of each of the dosage forms.

TABLE 2
Composition of the MDMA 34 mg and 50 mg Drug Product.

| | | | Strength (Label Claim) | | | |
|---|---|---|---|---|---|---|
| Component and | | | MDMA | | MDMA | |
| Quality Standard | | | 34 mg Capsule | | 50 mg Capsule | |
| (and Grade, if | Reference | | Quantity | | Quantity | |
| Applicable) | to Standard | Function | per Unit | [% w/w] | per Unit | [% w/w] |

Active Substances(s)

| | | | | | | | | |
|---|---|---|---|---|---|---|---|---|
| MDMA•HCl, milled | In-house | Active | 40.50 | mg¹ | 50.0 | 59.50 | mg² | 50.0 |

API

Excipients

| | | | | | | | | |
|---|---|---|---|---|---|---|---|---|
| Mannitol | USP/EP | Diluent | 39.69 | mg | 49.0 | 58.31 | mg | 49.0 |
| (Mannogem Powder) | | | | | | | | |
| Magnesium Stearate | USP- | Lubricant | 0.81 | mg | 1.0 | 1.19 | mg | 1.0 |
| | NF/EP | | | | | | | |
| Total | — | — | 81.00 | mg | 100.0 | 119.00 | mg | 100.0 |

| | | | | | | |
|---|---|---|---|---|---|---|
| Size 2, HPMC | In-house | Capsule | Average | N/A | — | — |
| Swedish Orange/ | | | capsule | | | |
| White Capsules | | | weight 57 | | | |
| | | | mg-65 mg | | | |
| Size 2, HPMC | In-house | Capsule | — | — | Average | N/A |
| Swedish Orange | | | | | capsule | |
| Capsules | | | | | weight 57 | |
| | | | | | mg-65 mg | |

¹40.50 mg of MDMA•HCl is equivalent to 34 mg MDMA free base.
²59.50 mg of MDMA•HCl is equivalent to 50 mg MDMA free base.

The composition and components of the HPMC capsule shells is provided in Table 3 and Table 4 below.

TABLE 3
Composition of the Size 2, HPMC
Swedish Orange/White Capsules

| | Body: | Cap: |
|---|---|---|
| | V44.900, | V22.905, |
| | WHITE | SWEDISH ORANGE |
| Component | OP. V900 | OP. V905 |
| Ferric Oxide, Red, E172, USP-NF | — | 1.1817% |
| Titanium Dioxide, E171, USP/EP | 2.0000% | 0.4916% |
| Hypromellose, USP/EP | QSP 100% | QSP 100% |

TABLE 4
Composition of the Size 2, HPMC Swedish Orange Capsules.

| | Body and Cap: |
|---|---|
| | V22.905, SWEDISH |
| Component | ORANGE OP. V905 |
| Ferric Oxide, Red, E172, USP-NF | 1.1817% |
| Titanium Dioxide, E171, USP/EP | 0.4916% |
| Hypromellose, USP/EP | QSP 100% |

### Printing Ink 10A2 Black

The printing ink consists of shellac, E904 US Pharmacopoeia-National Formulary/European Pharmacopoeia (USP-NF/EP), ferric oxide black, E172 (USP-NF), propylene glycol (USP/EP), strong ammonia solution (USP-NF/EP), and potassium hydroxide (USP-NF/EP).

### Container Closure System

The container closure system is an aluminum blister pack consisting of cold-formable aluminum laminate and push-through blister lidding foil.

### Drug Product Manufacturing Process

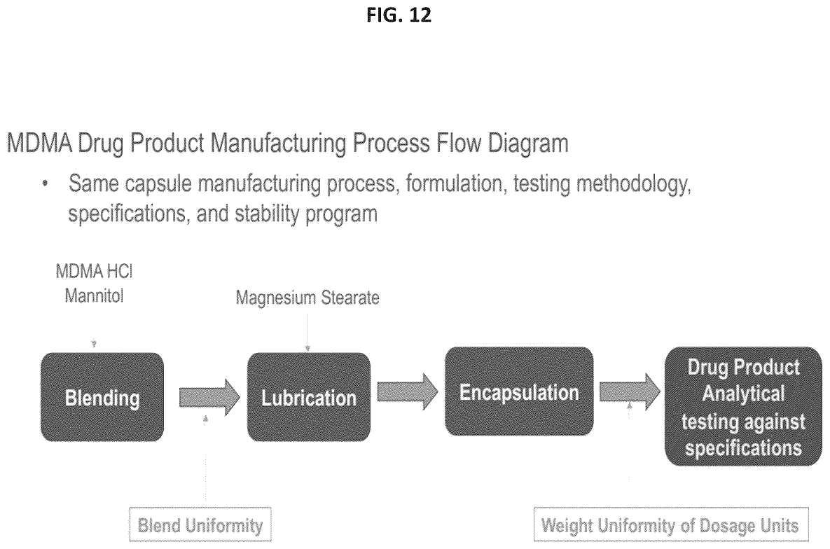

The MDMA Drug Product Manufacturing Process Flow Diagram (FIG. 12) comprises capsule manufacturing process, formulation, testing methodology, specifications, and stability evaluation.

### Example 4. Comparative Dissolution Studies

The dissolution profiles were obtained and compared for 40 mg MDMA·HCl capsules and 60 mg MDMA·HCl capsules used in Phase III clinical studies versus 40 mg MDMA·HCl (34 mg MDMA on a free base basis) and 60 mg MDMA·HCl (50 mg MDMA on a free base basis) capsules. N=12 capsules were compared in pH 1.2, 4.5, and 6.8 dissolution media, with all other dissolution and analytical conditions as described in the Dissolution Studies Procedure.

### Investigation Strategy

MDMA·HCl capsules (40 mg and 60 mg) and the details of these are listed in Table 5 along with the details of capsules that were used.

TABLE 5
MDMA Capsule Details.

| Dose Strength | Equivalent Dose Strength | |
|---|---|---|
| MDMA•HCl | MDMA freebase | Batch Details |
| 40 mg | 34 mg | 99441B1 |
| 40 mg | 34 mg | 200601-010A |
| 60 mg | 50 mg | 99441B2 |
| 60 mg | 50 mg | 200601-010B |

### Dissolution Profile Timepoint Determination

A single dissolution (n=6 capsules) test was performed as described in the Dissolution Studies Procedure. Both strengths of the capsules (see Table 5) were tested and sampled at the following timepoints: 5, 10, 15, 20, 30, 45, and 60 minutes. Samples were analyzed as described in the analytical test method.

### Dissolution Testing

n=12 units of each capsule batches in Table 5 were tested for dissolution as per the Dissolution Studies Procedure, using each of the media described below and the sampling timepoints established in the Dissolution Studies Procedure. Standards were prepared on the day of use and in the same dissolution media as the samples.

### pH 1.2 Dissolution Media Preparation

Prepare 15 L of media as follows: Dissolve 26.3 g NaCl and 111 mL (131.3 g) aqueous hydrogen chloride in 15 L of water. Mix well.

### pH 4.5 Dissolution Media Preparation

Prepare 15 L of media as follows: Dissolve 204.2 g potassium dihydrogen phosphate in 11.2 L water. Adjust the pH with 0.1 M sodium hydroxide or 0.1 M hydrochloric acid as required. Dilute to 15 L with water.

### pH 6.8 Dissolution Media Preparation

Prepare 15 L of media as follows: Mix 3750 mL of 0.2 M potassium dihydrogen phosphate with 1680 mL 0.2 M sodium hydroxide and dilute to 15 L with water.

### Calculation of Results

The mean dissolution profile was plotted and compared for the capsules at each dissolution condition, accounting for the samples removed at the previous timepoints if any. For the comparison of dissolution profiles, where applicable, the similarity factor f2 was estimated by using the following formula:

$$f 2 = 50 \cdot log \{ {[ 1 + ( 1 / n ) {\sum }_{t = 1}^{n} {( {R}_{t} - {T}_{t} )}^{2} ]}^{- 0.5} \cdot 100 \}$$

In the above equation,
f2 is the similarity factor;
n is the number of time points;
R(t) is the mean percent reference drug dissolved at time t after initiation of the study; and
T(t) is the mean percent test drug dissolved at time t after initiation of the study.

### Example 5. Dissolution Studies Procedure

### Introduction

This method describes the procedure for Dissolution Test of MDMA HCl by High Performance Liquid Chromatography-Ultraviolet (HPLC-UV).

### Equipment

Agilent HPLC with Binary pump & UV detector
Dissolution Bath, Apparatus 2 (Paddle)
XBridge Phenyl, 3.5 μm, 4.6×150 mm (Cat. No. 186003335, Waters)
0.45 μm GHP membrane filter (Cat. No. WAT200802, Waters)

### General Statements

All glassware used in sampling and testing was Grade A glassware and was thoroughly cleaned prior to use. Agilent HPLC system was used for the analysis. Mobile Phase A was either Water:TFA—100:0.1 or Acetonitrile:TFA—100:0.1. The Dissolution Media/Diluent was 0.1N HCl and the needle wash was Water:Acetonitrile 50:50. The standard solution of MDMA was 0.1 mg/mL MDMA·HCl in water.

### Dissolution Procedure for Samples

Six capsules were weighed separately dissolution media was prepared to test the sample with the dissolution parameters shown in Table 6.

TABLE 6
Dissolution Parameters.

| Apparatus Type | Apparatus 2, Paddle | |
|---|---|---|
| Dissolution Media | 0.1N HCI | |
| Media volume | 500 | mL |
| Bath Temperature | 37 ± 0.5° | C. |
| Stir Speed | 50 | RPM |
| Filter type | 0.45 μm GHP Membrane Filter | |
| Sinker Type | Wire Sinker | |
| Volume pulled per time point | 5 | mL |

| | |
|---|---|
| Sampling time | 15 min, 30 min |

### Chromatographic Conditions

Table 7 shows the HPLC parameters used.

TABLE 7
HPLC parameters.

| Column | XBridge Phenyl 150 × 4.6 mm, 3 μm | |
|---|---|---|
| Column Oven | 30° | C. |
| Injection Volume | 10 | μL |
| Autosampler Temperature | Ambient | |
| Flow Rate | 1.0 | mL/min |
| Detection | UV 235 nm | |
| Mobile Phase program | MPA:MPB 80:20 Isocratic | |
| Needle Wash | Water:MeCN (50:50) | |
| Run Time | 10 | mins |

### Guideline Injection Sequence

Injected the blank solution until a stable baseline is achieved.
Number of standard solution injectioned prior to sample analysis.
Standard solution injected in duplicate following a maximum of 6 sample injections (not inclusive of diluent blank injections) and at the end of the sequence.

Table 8 shows the injection sequence.

TABLE 8
Injection sequence.

| Solution | Number of Injections |
|---|---|
| Diluent blank | 3 |
| Standard 1 | 5 |
| Standard 2 | 1 |
| Standard 1 (Bracketing STD) | 2 |
| Sample Solution (Up to 6 | 1 per Sample |
| samples) | Solution |
| Standard 1 (Bracketing STD) | 2 |

After each use, the column was purged and cleaned according to standard procedures.

### Calculations

The dissolution calculation for MDMA·HCl is

$$\% dissolved = ( {PA}_{SMP} \times {C}_{STD} \times {VOL}_{VES} \times 100 ) / ( {PA}_{STD} \times LC )$$

where:
PA_{SMP} is sample peak area (MDMA·HCl peak)
VOL_{VES} is volume of dissolution media in the vessel (mL)
C_{STD} is concentration of MDMA·HCl reference standard in the working standard 1 preparation considering the purity of the reference standard (in mg/mL)
PA_{STD} is mean MDMA·HCl peak area of the bracketing assay standard solution 1 injections
LC is labelled claim per capsule (40 mg or 60 mg)

### Results

The results for the dissolution study (FIG. 13) indicate nearly complete dissolution of drug product occurs in 15 minutes at pH 1.2, 4.5, and 6.8°.

### Example 6. Dosage, Administration, and Prescription Information

### Dosage and Administration

### Recommended Dosage

The total dosage of MDMA·HCl included 3 doses in combination with treatment sessions (dose 1: 102 mg; doses 2 and 3: 150 mg each) with interim periods of at least 21 days between doses. The total dose of MDMA·HCl at each of these treatment sessions was provided in an individual package containing 3 capsules. Patients took 2 capsules at the start of the session and take the third capsule 1½ to 2 hours after the first dose. Patients may need to set an alarm to take the second dose.

The MDMA·HCl is for oral use only. The capsules should be swallowed whole and not crushed or chewed. MDMA·HCl can be taken without regard to timing of meals. It is recommended to not exceed 150-mg MDMA·HCl per day.

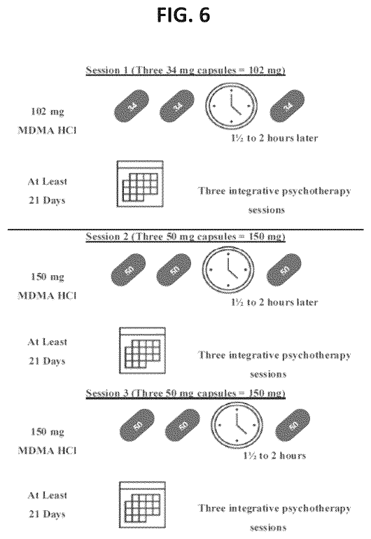

FIG. 6 shows the schedule of dosing and therapy sessions for MDMA·HCl.

### Administration Instructions

Patients were instructed to follow these administration instructions and read the instructions for use before self-administration. Patients were further instructed to take the unopened package with them to the treatment session. At the start of the medication session, the instructions to patients were as follows:
Push 2 capsules through the foil and take with a sip of water.
Sit or lay down in a comfortable position.
Protect eyes from bright light.
Rest and proceed with the treatment session for 1½ to 2 hours
After 1½ to 2 hours have elapsed, sit up and push remaining capsule through foil. Take 1 capsule with a sip of water.
Sit or lay down in a comfortable position.
Protect eyes from bright light.
Rest and proceed with the treatment session.
Remain in the facility until effects have worn off.
Do not engage in potentially hazardous activities such as driving until the next day.

### Important Considerations Prior to Initiating and Between MDMA·HCl Treatments

Before initiating treatment, the patients were instructed that MDMA·HCl must be self-administered under the direct observation of a health care provider during a treatment session. The patients were further instructed not to engage in potentially hazardous activities, such as driving or operating machinery, until the next day after each treatment.

### Blood Pressure Assessment Before Initiating Treatment

Blood pressure was assessed prior to prescribing MDMA·HCl.
If baseline blood pressure was elevated (e.g., >140 mm Hg systolic, >90 mm Hg diastolic), consider the risks of short-term increases in blood pressure and benefit of MDMA·HCl treatment in patients with PTSD.

### Important Considerations Prior to Prescribing Each MDMA·HCl Dose

Cardiovascual status of patients being considered for treatment with MDMA·HCl was assessed. Before initiating treatment, a careful history (including assessment for a family history of sudden death or ventricular arrhythmia) was conducted along with a physical exam to assess the presence of cardiac disease with further cardiac evaluation when warranted.
Before prescribing the second and third doses of MDMA·HCl, any additional cardiac history was collected to assess for a change in cardiovascular status. Concomitant medications were reviewed to ensure that patients are not taking any contraindicated medications (e.g., monoamine oxidaze inhibitors (MAOIs)) before prescribing each dose of MDMA·HCl.

### Therapeutic Program

The safety and efficacy of MDMA·HCl were examined in combination with a specific therapeutic program. Physicians should advise patients that each dose of MDMA·HCl must be self-administered under the direct observation of an appropriately-trained health care provider during a treatment session. The prescriber should discuss the following elements of the therapeutic program.

### Preparatory Sessions

Preparatory session(s) (talk therapy or psychotherapy) address the patient questions and concerns, as well as to prepare them for upcoming treatment sessions with MDMA·HCl were conducted.

Sessions with MDMA·HCl

At the beginning of each of the 3 treatment sessions with MDMA·HCl, the planned approach and the range of experiences that may occur during the session was reviewed with the trained health care provider.

Integration Sessions Following Sessions with MDMA·HCl

Follow-up contact with the trained health care provider was conducted to support successful integration. In clinical trials, integration included 3 sessions (talk therapy or psychotherapy) after each session with MDMA·HCl.

### Post-Administration Observation

During and after MDMA·HCl self-administration at each session, a health care provider observed each patient for approximately 6 hours from first dose of the split dose. Patients should understand that they should not leave the physical setting while still experiencing effects of MDMA·HCl at treatment sessions. Patients should also understand that additional time may be required beyond the planned length of the sessions, if the patient needs additional support. The patients also agreed to accept transport home from treatment sessions with MDMA·HCl.

### Missed Treatment Session(s)

If a patient missed treatment session(s), provided there is no evidence of diversion or abuse, the patient was counseled to re-schedule the missed session and to continue the current psychotherapy schedule. Healthcare providers reiterated the importance of psychological intervention in combination with MDMA·HCl treatment.

Use of MDMA·HCl with Reversible MAOIs Such as Linezolid or Methylene Blue

MDMA·HCl administration was not initiated in patients being treated with a reversible MAOI such as linezolid or intravenous methylene blue. Drug interactions can increase the risk of hypertensive reactions. In some cases, a patient already receiving therapy with MDMA·HCl may require urgent treatment with MAOIs. MDMA·HCl should not be administered again until 5 to 10 times the half life after the last dose of MAOIs, whichever comes first.

### Dosage Forms and Strengths

MDMA·HCl is supplied as single-dose, foil-wrapped capsules in 2 total dosage strengths:
102 mg total dose: MDMA HCl 34 mg, 3 Swedish Orange/White, Size 2 capsules imprinted with “MDMA 34”.
150 mg total dose: MDMAHCl 50 mg, 3 Swedish Orange, Size 2 capsules imprinted with “MDMA 50”.

### Contraindications

TABLE 9
MDMA•Contraindications

| Contraindication | Notes |
|---|---|
| Use of MAOIs | Concomitantly or within 14 days of discontinuing |
| | treatment with MDMA, including reversible MAOIs |
| | such as linezolid or intravenous methylene blue |
| Known | To MDMA or any of the other components of the |
| hypersensitivity | formulation |

### Warnings and Precautions

### Perceptual Changes

Known effects of MDMA·HCl include perceptual changes such as difficulty concentrating and impaired judgment, and physiological effects such as dizziness, impaired gait/balance, and blurred vision. Other known effects of MDMA·HCl include an altered state that may include a range of emotions, thoughts, and physical sensations. Healthcare providers should caution patients about operating hazardous machinery, including automobiles or airplanes, until they are reasonably certain that effects of MDMA·HCl have dissipated (e.g., impaired judgment, thinking, or motor skills). Patients should not engage in hazardous occupations or activities requiring complete mental alertness or motor coordination, such as operating machinery or a motor vehicle or flying an airplane, until the day after taking MDMA·HCl.

### Abuse and Misuse

MDMA·HCl contains the hydrochloride salt of 3,4-methylenedioxymethamphetamine (MDMA) and a schedule II controlled substance (CII), and may be subject to abuse and misuse. Assess each patient's risk for abuse or misuse prior to prescribing MDMA HCl and monitor all patients receiving MDMA·HCl for the development of these behaviors or conditions, including drug-seeking behavior, while taking MDMA·HCl. Prescribe and dispense MDMA·HCl with appropriate precautions to minimize risk of misuse or abuse. Individuals with a history of drug abuse or dependence are at greater risk; therefore, use careful consideration prior to treatment of individuals with a history of substance use disorder and monitor for signs of abuse or dependence. Patients should not be prescribed more than 3 doses of MDMA. Contact local state professional licensing board or state-controlled substances authority for information on how to prevent and detect abuse or diversion of MDMA·HCl.

MDMA·HCl is available only through a restricted program under a Risk Evaluation and Mitigation Strategy (REMS). Contact local state professional licensing board or state controlled substances authority for information on how to prevent and detect abuse or diversion of MDMA·HCl.

### MDMA·HCl Risk Evaluation and Mitigation Strategy (REMS)

MDMA·HCl is available only through a restricted program under a REMS called the MDMA·HCl REMS because of the risks of serious adverse outcomes from abuse and overdose. Important requirements of the MDMA·HCl REMS include the following:

Healthcare settings must be certified in the program and ensure that MDMA·HCl is:
Only dispensed by certified pharmacies.
Self-administered by patients enrolled in the program.
Administered by patients under the direct observation of a health care professional.
Patients are to be monitored by a health care professional for at least 6 hours after administration of MDMA·HCl.
Pharmacies must be certified in the REMS and must only dispense MDMA·HCl to patients enrolled in the program.

### Increase in Blood Pressure and Heart Rate

MDMA·HCl causes transient dose-dependent increases in systolic and/or diastolic blood pressure (BP) and heart rate at all recommended doses. A substantial increase in blood pressure and/or heart rate could occur after any dose even if smaller blood pressure or heart rate effects were observed with previous administrations. Assess blood pressure and control hypertension before initiating treatment with MDMA·HCl. Monitor blood pressure regularly during treatment and treat new-onset hypertension and exacerbations of pre-existing hypertension. In patients whose BP is elevated (as a general guide: >140/90 mm Hg) a decision to delay MDMA·HCl should take into account the balance of benefit and risk in individual patients. Exercise caution when treating patients at higher risk of major adverse cardiovascular events (including stroke, myocardial infarction, and cardiovascular death), particularly patients with known cardiovascular and cerebrovascular disease, preexisting hypertension, and patients with advanced age. Use caution with other drugs that increase blood pressure and heart rate.

Treatment with MDMA·HCl should be discontinued in patients who develop hypertensive crisis or hypersensitive encephalopathy. Refer patients experiencing symptoms of a hypertensive crisis (e.g., chest pain or shortness of breath) or hypertensive encephalopathy (e.g., sudden severe headache, visual disturbances, seizures, diminished consciousness, or focal neurological deficits) immediately for emergency care.

Suicidal Thoughts and Behaviors in Patients with PTSD

Patients with psychiatric disorders, including PTSD, are at increased risk of suicide. All PTSD patients were monitored for clinical worsening and emergence of suicidal thoughts and behaviors, especially at times of any dosing changes preceding MDMA·HCl treatment and after medication sessions where MDMA·HCl was self-administered. Council family members or caregivers of patients to monitor for changes in behavior and to alert the healthcare provider. Consider changing the therapeutic regimen, including possibly discontinuing MDMA·HCl, in patients who are experiencing emergent suicidal thoughts or behaviors that are not resolved with psychological intervention. The effectiveness of MDMA·HCl in preventing suicide or in reducing suicidal ideation or behavior has not been demonstrated. Use of MDMA·HCl does not preclude the need for hospitalization if clinically warranted, even if patients experience improvement after an initial dose of MDMA·HCl.

### Psychiatric Symptoms

Psychiatric adverse reactions have been observed in clinical trials with MDMA·HCl, including anxiety, insomnia, and irritability. MDMA·HCl has not been evaluated in patients with psychosis or bipolar affective disorder Type 1. Exercise caution when treating patients with MDMA·HCl who have a history of psychosis or bipolar disorders. Patients treated with MDMA·HCl should be observed for the possible emergence or exacerbation of psychiatric symptoms. If serious or severe psychiatric symptoms develop in association with the administration of MDMA·HCl, consider discontinuation of MDMA·HCl.

### Serotonin Syndrome

Monoamine reuptake inhibitors, including MDMA·HCl, can precipitate serotonin syndrome, a potentially life-threatening condition. The risk is increased with concomitant use of other serotonergic drugs (including triptans, tricyclic antidepressants, fentanyl, lithium, tramadol, tryptophan, buspirone, amphetamines, and St. John's Wort) and with drugs that impair metabolism of serotonin, i.e., MAOIs. Serotonin syndrome can also occur when these drugs are used alone.

Serotonin syndrome signs and symptoms may include mental status changes (e.g., agitation, hallucinations, delirium, and coma), autonomic instability (e.g., tachycardia, labile blood pressure, dizziness, diaphoresis, flushing, hyperthermia), neuromuscular symptoms (e.g., tremor, rigidity, myoclonus, hyperreflexia, incoordination), seizures, and gastrointestinal symptoms (e.g., nausea, vomiting, diarrhea).

The concomitant use of MDMA·HCl with MAOIs is contraindicated. In addition, do not initiate MDMA·HCl in a patient being treated with MAOIs such as linezolid or intravenous methylene blue. If it is necessary to initiate treatment with an MAOI such as linezolid or intravenous methylene blue in a patient taking MDMA·HCl, discontinue MDMA·HCl before initiating treatment with the MAOI.

Monitor all patients taking MDMA·HCl for the emergence of serotonin syndrome. Discontinue treatment with MDMA·HCl and any concomitant serotonergic agents immediately if the above symptoms occur, and initiate supportive symptomatic treatment. If concomitant use of MDMA·HCl with other serotonergic drugs is clinically warranted, inform patients of the increased risk for serotonin syndrome and monitor for symptoms.

### Drug Interactions

### Drugs Metabolized by CYP2D6

MDMA is a strong CYP2D6 inhibitor. Therefore, coadministration of MDMA with drugs that are primarily metabolized by CYP2D6 may increase the exposures of those drugs. Such drugs include certain antidepressants (e.g., venlafaxine, nortriptyline, imipramine, desipramine, paroxetine, fluoxetine, and sertraline), antipsychotics (e.g., haloperidol, risperidone, thioridazine), beta-blockers (e.g., metoprolol), and Type 1C antiarrhythmics (e.g., propafenone and flecainide). When used concomitantly with MDMA, it may be necessary to decrease the dose of these CYP2D6 substrates or temporarily halt administration, particularly for drugs with a narrow therapeutic index. Drugs that require metabolic activation by CYP2D6 to be effective (e.g., tamoxifen and codeine) theoretically could have reduced efficacy when administered concomitantly with inhibitors of CYP2D6 such as MDMA·HCl. Patients treated concomitantly with MDMA·HCl and such drugs may require temporarily increased doses of the drug.

### Psychostimulants

Concomitant use with psychostimulants (e.g., amphetamines, methylphenidate, modafinil, armodafinil) may increase blood pressure. Closely monitor blood pressure with concomitant use of MDMA·HCl with psychostimulants.

### Monoamine Oxidase Inhibitors (MAOIs)

Concomitant use of MDMA·HCl and other monoamine oxidase inhibitors (MAOIs) within 14 days is contraindicated because of an increased risk of causing hypertensive reactions. At least 14 days should elapse between discontinuation of an MAOI and treatment with MDMA·HCl. Conversely, at least 14 days should be allowed after taking MDMA·HCl before starting an MAOI.

### Serotonergic Drugs

Co-administration with serotonergic drugs such as selective serotonin reuptake inhibitors (SSRIs) and serotonin-norepinephrine reuptake inhibitors (SNRIs) may eliminate or greatly attenuate the effects of MDMA·HCl, and these medications should be tapered in line with the prescriber's clinical judgment.

once obtained at a lower dose).

### Use in Specific Populations

### Geriatric Use

Clinical studies of MDMA·HCl did not include sufficient numbers of patients 65 years of age and older to determine whether they respond differently from younger adult patients.

### Hepatic Impairment

MDMA·HCl has not been studied in patients with hepatic impairment. Use in this population is not recommended.

### Drug Abuse and Dependence

### Controlled Substance

MDMA·HCl contains the hydrochloride salt of 3,4 methylenedioxymethamphetamine (MDMA), a Schedule II Substance under the Controlled Substances Act.

### Drug Abuse

MDMA·HCl produces dose-dependent central nervous system effects, including positive subjective reinforcing effects. The onset of effect is rapid, enhancing its potential for abuse or misuse. Abuse is the intentional, non-therapeutic use of a drug, even once, for its psychological or physiological effects. Misuse is the intentional use, for therapeutic purposes, of a drug by an individual in a way other than prescribed by a healthcare provider or for whom it was not prescribed.

Illicit MDMA has been extensively abused and/or misused. Because illicit use of MDMA has been reported, physicians should carefully evaluate patients for a history of drug abuse and follow such patients closely, observing them for signs of misuse or abuse of illicit MDMA Individuals with a history of drug abuse or dependence may be at greater risk for abuse and misuse of MDMA·HCl. Careful consideration is advised prior to use of individuals with a history of substance use disorder.

All patients treated with MDMA·HCl require careful monitoring for signs of abuse. Proper assessment of the patient, proper prescribing practices and proper handling and storage of the medication are appropriate measures that help to limit abuse or misuse of MDMA·HCl. MDMA·HCl may produce a variety of symptoms broadly characterized as positive reinforcing effects. Monitoring for signs of abuse and misuse is recommended.

### Dependence

Physical dependence is a state that develops as a result of physiological adaptation in response to repeated drug use, manifested by withdrawal signs and symptoms after abrupt discontinuation or significant dosage reduction of a drug.

Tolerance is a physiological state characterized by a reduced response to a drug after repeated administration (i.e., a higher dose of a drug is required to produce the same effect that was once obtained at a lower dose).

As MDMA·HCl administration is limited to 3 individual doses administered at least 21 days apart, dependence and tolerance is not expected to develop when MDMA·HCl is used as prescribed.

### Overdosage

### Clinical Presentation

The prescription unit is restrictive to prevent overdose and therefore is unlikely to occur. The manifestations of acute overdose with illicit MDMA in excess of the recommended dose range may include hyperthermia, hyponatremia, rhabdomyolysis, acute renal failure, seizure, cardiovascular adverse events, disseminated intravascular coagulation, hemorrhage, and death.

### Management of Overdosage

There is no specific antidote for MDMA overdose. In the case of overdose, the possibility of multiple drug involvement should be considered, and supportive care should be provided. Contact a Certified Poison Control Center for the most up to date information on the management of overdosage.

### Clinical Pharmacology

### Mechanism of Action

MDMA·HCl is an Entactogen. The mechanism of action of MDMA·HCl in the treatment of PTSD is not known. MDMA·HCl is a serotonin and other monoamine (norepinephrine and dopamine) reuptake inhibitor as well as a modulator of monoamine transport. MDMA·HCl, in combination with psychological intervention, leads to the modulation of DNA methylation in genes controlling the hypothalamic pituitary adrenal access (resulting in epigenetic malleability). MDMA·HCl increases the release of monoamines such as serotonin, dopamine and norepinephrine into the extraneuronal space.

### Pharmacodynamics

### Cardiac Electrophysiology

The effect of MDMA·HCl on the QTc interval was evaluated in a randomized, crossover study in N healthy subjects. A large increase in heart rate (i.e., >10 bpm) was observed with MDMA·HCl. The totality of evidence from the nonclinical and clinical data indicates a lack of clinically relevant QTc prolongation at the therapeutic dose of MDMA·HCl.

### Pharmacokinetics

MDMA·HCl is a racemic mixture and contains the racemic anhydrous hydrochloride salt of 3,4-methylenedioxymethamphetamine (MDMA), a triple monoamine reuptake releaser and inhibitor. Both enantiomers are pharmacologically active. 3,4-methylendioxyampetamine (MDA) is a metabolite of MDMA and is also pharmacologically active. Peak plasma levels and AUC_{0-inf} of MDA are less than 10% of the corresponding parameters for MDMA. MDA may contribute to the pharmacological effects of MDMA·HCl. The pharmacokinetics of MDMA are non-linear with higher than dose proportional increases in plasma concentration due to auto-inhibition of CYP2D6. The elimination half life of a single 120 mg dose of MDMA·HCl was 9 hours.

### Absorption

The absolute bioavailability of MDMA·HCl in humans is not known because pharmacokinetic studies have not been conducted following intravenous (iv) administration of MDMA·HCl. MDMA·HCl exhibits high solubility and permeability and appears to be well absorbed. In humans, following a single 120 mg dose of MDMA·HCl, peak plasma concentration of MDMA was generally achieved within 2 hours and Cₘₐₓ was 261 ng/mL. The AUC_{0-inf} of MDMA was 3890 h*ng/mL following a single 120 mg dose of MDMA·HCl. Peak plasma levels (Cₘₐₓ) of MDA were 13.3 ng/mL and occurred at 6 hours post dose. The AUC_{0-inf} of MDA was 374 h*ng/mL following a single 120 mg dose of MDMA·HCl.

TABLE 10
Pharmacokinetics of MDMA and MDA following
a single 120 mg dose of MDMA•HCl administered
to fasting healthy males and females

| | | |
|---|---|---|
| PK Parameter | MDMA | MDA |

| | | | | |
|---|---|---|---|---|
| Cₘₐₓ | 261 | (27.0) | 13.3 | (27.6) |
| ng/mL (CV %) | | | | |
| Tₘₐₓ | 2.00 | (2.00, 8.00) | 6.00 | (2.00, 8.00) |
| h (min, max) | | | | |
| AUC_{0-inf} | 3890 | (39.1) | 374 | (38.1) |
| h*ng/mL (CV %) | | | | |
| t_{1/2} | 9.10 | (19.6) | 12.8 | (19.2) |
| h (CV %) | | | | |

### Effect of Food

The Cₘₐₓ and AUC data from a food-effect study involving administration of MDMA·HCl to healthy volunteers under fasting conditions and with a high-fat meal indicated that exposure to the drug is not affected by food. A high fat meal had no effect on the pharmacokinetics of a single 120 mg dose of MDMA·HCl in healthy males and females.

### Distribution

In vitro studies have demonstrated that MDMA is about 16.5% bound to human plasma proteins. MDMA is not a substrate of BCRP, MDR1, OATP1B1, or OATP1B3. A single 120 mg dose of MDMA·HCl resulted in a volume of distribution of 430 L. A single 100 mg dose of MDMA·HCl resulted in a volume of distribution of 372 L.

### Elimination

### Metabolism

MDMA is extensively metabolized in humans. Several parallel metabolic pathways contribute to the metabolism of MDMA including CYP2D6, CYP1A2, CYP3A4, CYP2C19, and CYP2B6. MDMA is a strong inhibitor of CYP2D6 and thus auto-inhibits its own metabolism, leading to higher than dose proportional pharmacokinetics of MDMA.

N-demethylation of MDMA forms an active metabolite, 3,4-methylenedioxyamphetamine (MDA). The parent compound and MDA are further O-demethylated to 3,4-dihydroxymethamphetamine (HHMA) and 3,4-dihydroxyamphetamine (HHA), respectively. Both HHMA and HHA are subsequently O-methylated mainly to 4-hydroxy-3-methoxymethamphetamin (HMMA) and 4-hydroxy-3-methoxyamphetamine (HMA). MDMA is a strong inhibitor of CYP2D6 and thus auto-inhibits its own metabolism, leading to higher than dose proportional pharmacokinetics of MDMA.

### MDMA:

is highly permeable in human Caco-2 cells;
is metabolized primarily by CYP2B6 and CYP2D6 with less contribution by CYP2C19, CYP1A2, CYP3A4, CYP2C9, and CYP2C8;
is a potent reversible and time-dependent CYP2D6 inhibitor;
inhibits the renal transporters MATE1, organic cation transporter (OCT1), and OCT2 at supratherapeutic concentrations;
is not a substrate for human organic anion transporting polypeptide (OATP1B1), OATP1B3, breast cancer resistance protein (BCRP), or multidrug resistance (MDR1);
is not an inhibitor of BCRP, bile salt export pump (BSEP), MDR1, MATE2-K, organic anion transporter (OAT1), OAT3, OATP1B1, and OATP1B3;
has minimal risk of QTc prolongation or Torsade de Pointes (TdP), as the half maximal inhibitory concentration (IC50) for the inhibitory effect of MDMA on human ether-à-go-go-related gene (hERG) potassium current is 206 μM, (Hill coefficient=1.1) (191028.NBQ), a 111-x margin over the clinical plasma concentration of MDMA, following a supratherapeutic single dose of 180 mg MDMA HCl; and
is 16.5% protein bound in human plasma.

### Excretion

Formal ADME studies evaluating the recovery of labeled MDMA have not been conducted. The percentage of unchanged MDMA excreted in urine following orally administered doses of 1.0 and 1.6 mg/kg MDMA was 8% and 11%, respectively. The majority of the dose recovered in the urine was conjugated metabolites.

### Specific Populations

Patients with Renal Impairment

The pharmacokinetics of MDMA·HCl in subjects with renal impairment have not been studied. The percentage of unchanged MDMA excreted in urine following orally administered doses of 1.0 and 1.6 mg/kg MDMA was 8% and 11%, respectively (Schwaninger 2011). Therefore, renal impairment is unlikely to alter the pharmacokinetics of MDMA·HCl in a clinically meaningful way.

Patients with Hepatic Impairment

The pharmacokinetics of MDMA·HCl in subjects with hepatic impairment have not been studied. MDMA·HCl is extensively metabolized by hepatic enzymes.

### Potential for Other Drugs to Affect MDMA·HCl

MDMA·HCl is metabolized via several parallel Cytochrome P450 (CYP) pathways. Therefore, the potential that inhibition of any one pathway will impact the pharmacokinetics of MDMA·HCl in a clinically meaningful way is minimized. Paroxetine administered 20 mg a day for three days to 7 healthy males increased the AUC_{0-inf} of a single 100 mg dose of MDMA by 27% and Cₘₐₓ by 17%. Bupropion 150 mg per day for three days followed by 300 mg a day for four days administered to 16 healthy male and female Caucasian subjects increased the AUC_{0-24 hr} of a single 125 mg dose of MDMA by 33% and Cₘₐₓ by 14%.

### Potential for MDMA·HCl to Affect Other Drugs

MDMA·HCl is a strong CYP2D6 inhibitor. Therefore, when administered in combination with sensitive CYP2D6 substrates, MDMA·HCl may cause significant increase in the plasma levels of those drugs. A single 1.5 mg/kg dose of MDMA administered to 15 healthy males 4 hours before 30 mg dextromethorphan (Days 1, 2, 3, 4, 5 and 8) increased the AUC_{0-8 hr} of dextromethorphan 9.5-fold and Cₘₐₓ 8.5-fold. A single 1.5 mg/kg dose of MDMA HCl administered 4 hours before 30 mg dextromethorphan (Days 1, 2, 5, 8 and 11) to 12 healthy Caucasian females increased the AUC_{0-inf} of dextromethorphan 13.6-fold and Cₘₐₓ 8.3-fold. A single 100 mg dose of MDMA administered to 7 healthy males increased the AUC_{0-8 hr} of paroxetine (20 mg) 3-fold and increased Cₘₐₓ 2.5-fold (Segura 2005). MDMA·HCl is not an inducer of CYPs and did not inhibit CYPs other than CYP2D6 in a clinically meaningful manner.

### Nonclinical Toxicology

### Carcinogenesis, Mutagenesis, Impairment of Fertility

### Carcinogenesis

Based on negative in vivo and in vitro genotoxicity studies, the carcinogenic risk of MDMA·HCl is low. No tumors were reported after daily 28-day repeated dose toxicology studies of MDMA in rats (0, 10, 50, or 100 mg/kg/day) or dogs (0, 3, 9, or 15 mg/kg/day).

### Mutagenesis

MDMA was not mutagenic with or without metabolic activation in the in vitro bacterial reverse mutation assay (Ames test). Additionally, MDMA showed no signs of chromosomal aberrations in CHO-WBL cells with and without metabolic activation.

Oral MDMA was shown to have no signs of genotoxicity in male rats in doses up to 100 mg/kg day over two days. No statistically significant differences in micronucleated polychromatic erythrocytes or bone marrow cytotoxicity were observed across all dose levels.

### Impairment of Fertility

MDMA (0, 2.5, 5, and 10 mg/kg/day) was orally administered to male and female rats before mating. Males were dosed once daily for 28 days prior to pairing and dosing continued until euthanasia. Females were dosed once daily for 21 days prior to pairing and dosing continued though gestational day seven. Mean prostate gland weights decreased in all MDMA-treated males relative to controls, however this did not affect fertility or overall health of the animals. No effect on fertility and reproductive performance was observed up to the highest dose evaluated (10 mg/kg/day).

Animal Toxicology and/or Pharmacology

In 28-day repeat-dose toxicity studies, MDMA was well tolerated with no mortality or significant toxicologic findings after weekly, oral administration to rats (≤20 mg/kg) and dogs (≤4 mg/kg). No morphologic changes, based on neurohistopathology examination, were evident in the brains of rats or dogs treated with MDMA at any dose. The relevance of these findings to humans is unknown.

### How Supplied/Storage and Handling

MDMA·HCl is available as 34-mg or 50-mg capsules.

### Storage

Store at ambient temperature, 150 to 25° C. (590 to 77° F.); excursions permitted from 5° to 30° C. (41° to 86° F.).

### Disposal

MDMA·HCl oral capsules must be handled with adequate security, accountability, and proper disposal, per facility procedure for a Schedule II drug product, and per applicable federal, state, and local regulations.

### Example 7. Clinical Pharmacokinetics of MDMA from Study MPKF

### Introduction

The MPKF was a Phase 1, open-label, randomized sequence, multi-dose, 2-period crossover food effect study in 16 healthy individuals. The MPKF study evaluated plasma concentrations of both MDMA and the active metabolite 3,4-methylenedioxyamphetamine (MDA). Summary PK Parameters of the preliminary data was based on interim analysis are presented in Table 12Table 12Table 12, and Table 13Table 13Table 13 for MDMA and MDA, respectively.

Participants were randomized to receive 1 of 2 conditions before the other:
Fasted Treatment: at least 10 hours of fasting followed by administration of MDMA HCl with 240 mL water.
Fed Treatment: at least 10 hours of fasting followed by a high-fat (approximately 50% of total caloric content of the meal) and high-calorie (approximately 800 to 1000 calories) meal was consumed within 30 minutes prior to IMP administration.

Participants remained at the clinical research unit (CRU) for each Dosing Session from the time of check-in on the night before dosing until discharge 48 hours after dosing for collection of serial blood samples for PK analysis and safety monitoring. IMP was administered on Day 1 following the treatment sequence to which the participant was randomized. An additional outpatient visit occurred 72 hours after dosing to collect a final PK sample, commensurate with the expected PK profiles of MDMA and MDA. Safety assessments were also performed at this visit.

The washout period between Dosing Sessions was at least 14 days. If a participant experienced emesis within 4 hours of administration, they were to be withdrawn from the study and replaced with a new participant. An End of Study Visit was performed 2 to 10 days after the completion of the final Dosing Session 72-hour follow-up. An informal interim analysis was planned to be conducted after at least 5 participants completed at least 1 Dosing Session to examine PK variability.

### Materials and Methods

### The MPKF Study:

Formulation: MDMA·HCl (50/50 racemic mixture) encapsulated with excipients listed in Table 11. The API was a white crystalline powder of pharmaceutical quality, made according to current Good Manufacturing Practices (cGMP) for human use.

Participants: Sixteen healthy individuals were recruited for the study.

Intervention: The study was conducted as a Phase 1, open-label, randomized sequence, multi-dose, 2-period crossover food effect study. Each participant received multiple doses of MDMA, and plasma concentrations of both MDMA and the active metabolite MDA were measured.

Sample Collection: Blood samples were collected at various time points up to 72 hours after dosing. The samples were analyzed using validated liquid chromatography-tandem mass spectrometry (LC-MS/MS) methods.

Pharmacokinetic Analysis: Plasma pharmacokinetic parameters, such as area under the concentration-time curve from time 0 to the last quantifiable concentration (AUC0-t), area under the concentration-time curve from time 0 to 72 hours (AUC0-72), area under the concentration-time curve from time 0 to infinity (AUC0-inf), maximum concentration (Cₘₐₓ), time to maximum concentration (Tmax), elimination half-life (t½), lag time (Tlag), clearance (CL/F), and volume of distribution (Vd/F) were calculated using non-compartmental methods.

Data Analysis: The summary PK parameters were presented in Table 12 for MDMA and Table 13 for MDA. The data were analyzed using descriptive statistics, and the mean and standard deviation were calculated. The results were compared between the two periods and analyzed for food effects. Statistical analyses were performed using appropriate methods, such as analysis of variance (ANOVA) and paired t-tests. A p-value of less than 0.05 was considered statistically significant. Results are shown in Table 12 and 13.

TABLE 11
60 mg MDMA•HCl Formulation Used in Study MPKF.

| | | Proportion relative |
|---|---|---|
| | Amount (mg) | to core weight (% w/w) |
| Component | MPKF Capsules | MPKF Capsules |
| MDMA•HCl | 59.5¹ | 50.0% |
| Mannitol (filler) | 58.3¹ | 49.0% |
| Mg Stearate (lubricant) | 1.19 | 1.0% |
| Total | 119.00 | 100.0% |

¹equivalent to 50 mg MDMA free base

TABLE 12
Preliminary Plasma PK Parameters of MDMA Following
a Single 120 mg Oral Dose of MDMA•HCl Under
Fasting or Fed Conditions (Study MPKF).

| MDMA PK | | 120 mg MDMA Treatment | | | |
|---|---|---|---|---|---|
| Parameters^{a} | Units | Fasted N = 15 | | Fed N = 11 | |
| AUC₀₋ₜ | h*ng/mL | 3123 | (42.5) | 3060 | (46.2) |
| AUC₀₋₇₂ | h*ng/mL | 3368 | (37.9) | 3301 | (42.7) |
| AUC_{0-inf} | h*ng/mL | 3388 | (38.4) | 3318 | (43.3) |
| Cₘₐₓ | ng/mL | 238 | (24.7) | 227 | (21.7) |
| Tₘₐₓ^{b} | h | 2.00 | (2.00, 8.00) | 4.00 | (4.00, 6.00) |
| t_{1/2} | h | 8.7 | (21.8) | 8.0 | (27.2) |
| Tlag^{b} | h | 0.6 | (31.3) | 0.7 | (38.4) |
| CL/F | L/h | 29.5 | (38.4) | 30.1 | (43.3) |
| Vd/F | L | 371.7 | (23.5) | 349.4 | (22.6) |

^{a}Arithmetic Mean (Arithmetic CV %); N
^{b}Median (Min; Max); N

TABLE 13
Plasma PK Parameters of MDA Following a Single 120 mg Oral Dose
of MDMA•HCl Under Fasting or Fed Conditions (Study MPKF).

| | | |
|---|---|---|
| Plasma PK Parameters | Fed (N = 14) | Fasted (N = 15) |

| | | | | |
|---|---|---|---|---|
| AUC₀₋ₜ (h*ng/mL) | 255 | (50.3) | 282 | (36.7) |
| AUC₀₋₇₂ (h*ng/mL) | 322 | (33.5) | 313 | (31.2) |
| AUC_{0-inf} (h*ng/mL) | 369 | (33.3) | 330 | (37.6) |
| Cₘₐₓ (ng/mL) | 12 | (22.6) | 12 | (25.4) |
| Tₘₐₓ (h) | 7 | (4, 12) | 6 | (2, 8) |
| T_{1/2} (h) | 12.8 | (28.1) | 12.6 | (20.0) |
| T_{lag} (h) | 0.7 | (36.9) | 0.6 | (27.5) |
| CL/F (L/h) | 270.7 | (33.3) | 302.8 | (37.6) |
| V_{d}/F (L) | 5000.2 | (23.2) | 5522.7 | (25.9) |

Abbreviation(s): MDA = 3,4-Methylenedioxyamphetamine; AUC₀₋ₜ = Area Under the Plasma Concentration-Time Curve from Time 0 to the Time of the Last Measurable Concentration; AUC₀₋₇₂ = Area Under the Plasma Concentration-Time Curve from Time 0 to 72; AUC_{0-inf} = Area Under the Plasma Concentration-Time Curve from Time 0 to Infinity; Cₘₐₓ = Maximum Observed; Plasma Concentration; Tₘₐₓ = Time of Observed Maximum Plasma Concentration; T_{1/2} = Apparent Terminal Elimination Half-Life; T_{lag} = Delay in Achieving Tₘₐₓ; CL/F = Apparent Total Plasma; Clearance; V_{d}/F = Apparent Volume of Distribution; h = Hours; N = Number of Participants in the Analysis Population.
Note:
Tₘₐₓ is presented as Median (Minimum, Maximum); other parameters are presented as Geometric Mean (Geometric CV %).

TABLE 14
Statistical comparison of AUC₀₋ₜ, AUC₀₋₇₂, AUC_{0-inf}, and Cₘₐₓ for MDMA
following administration in the fasted state compared to the fed state

| | | Treatment | | | | | | |
|---|---|---|---|---|---|---|---|---|
| | | Geometric LSMs | | | | Geometric | Geometric | |
| Parameter | (unit) | Fed | (n) | Fasted | (n) | Mean Ratio | CV % | 90% CI |
| AUC₀₋ₜ | (h*ng/mL) | 3110 | (14) | 3118 | (15) | 1.00 | 17.3 | 0.95-1.05 |
| AUC₀₋₇₂ | (h*ng/mL) | 3340 | (14) | 3363 | (15) | 0.99 | 16.9 | 0.94-1.04 |
| AUC_{0-inf} | (h*ng/mL) | 3357 | (14) | 3382 | (11) | 0.99 | 17.0 | 0.94-1.04 |
| Cₘₐₓ | (ng/mL) | 229 | (14) | 238 | (15) | 0.97 | 16.4 | 0.92-1.01 |

Abbreviation(s): MDMA = 3,4-Methylenedioxymethamphetamine; LSM = Least-Squares Mean; CI = Confidence Interval; AUC₀₋ₜ = Area Under the Plasma Concentration-Time Curve from Time 0 to the Time of the Last Measurable Concentration; AUC₀₋₇₂ = Area Under the Plasma Concentration-Time Curve from Time 0 to 72; AUC_{0-inf} = Area Under the Plasma Concentration-Time Curve from Time 0 to Infinity; Cₘₐₓ = Maximum Observed Plasma Concentration; CV % = Percent Coefficient of Variation; h = Hours; n = Number of Participants with Non-Missing Data.

TABLE 15
Statistical comparison of AUC₀₋ₜ, AUC₀₋₇₂, AUC_{0-inf}, and Cₘₐₓ for MDA
following administration of MDMA in the fasted state compared to the fed state

| | | Treatment | | | | Geometric | | |
|---|---|---|---|---|---|---|---|---|
| | | Geometric LSMs | | | | Mean | Geometric | |
| Parameter | (unit) | Fed | (n) | Fasted | (n) | Ratio | CV % | 90% CI |
| AUC₀₋ₜ | (h*ng/mL) | 254.48 | (14) | 281.65 | (15) | 0.90 | 20.0 | 0.84-0.97 |
| AUC₀₋₇₂ | (h*ng/mL) | 305.71 | (11) | 312.30 | (15) | 0.98 | 15.7 | 0.94-1.02 |
| AUC_{0-inf} | (h*ng/mL) | 328.19 | (8) | 330.63 | (11) | 0.99 | 17.6 | 0.93-1.06 |
| Cₘₐₓ | (ng/mL) | 11.92 | (14) | 12.39 | (15) | 0.96 | 20.0 | 0.90-1.03 |

Abbreviation(s): MDMA-3,4-Methylenedioxymethamphetamine; LSM = Least-Squares Mean; CI = Confidence Interval; AUC₀₋ₜ = Area Under the Plasma Concentration-Time Curve from Time 0 to the Time of the Last Measurable Concentration; AUC₀₋₇₂ = Area Under the Plasma Concentration-Time Curve from Time 0 to 72; AUC_{0-inf} = Area Under the Plasma Concentration-Time Curve from Time 0 to Infinity; Cₘₐₓ = Maximum Observed Plasma Concentration; CV % = Percent Coefficient of Variation; h = Hours; n = Number of Participants with Non-Missing Data.

TABLE 16A
Summary of MDMA PK Plasma Concentrations for fed treatment group

| | Concentration (ng/mL) | | | | | | | | | | |
|---|---|---|---|---|---|---|---|---|---|---|---|
| | Nominal Time (h) | | | | | | | | | | |
| Statistics | 0 | .5 | 1 | 2 | 4 | 6 | 8 | 12 | 24 | 48 | 72 |
| n | 14 | 14 | 14 | 14 | 14 | 14 | 14 | 14 | 14 | 14 | 14 |
| Mean | 0 | 10.22 | 59.83 | 129.08 | 228.43 | 214.86 | 181.29 | 128.90 | 50.47 | 7.41 | 0.81 |
| SD | 0 | 12.11 | 59.30 | 51.51 | 59.02 | 61.34 | 61.35 | 56.82 | 34.94 | 12.21 | 3.02 |
| IQR | 0 | 22.20 | 116.39 | 78.70 | 62.00 | 60.00 | 56.00 | 52.00 | 40.20 | 13.30 | 0.00 |
| Min | 0 | 0.00 | 0.00 | 48.10 | 151.00 | 127.00 | 100.00 | 63.50 | 16.90 | 0.00 | 0.00 |
| Median | 0 | 4.12 | 47.40 | 132.00 | 224.50 | 208.50 | 179.50 | 131.50 | 41.35 | 0.00 | 0.00 |
| Max | 0 | 30.60 | 157.00 | 207.00 | 395.00 | 386.00 | 357.00 | 295.00 | 151.00 | 43.90 | 11.30 |
| CV % | NC | 118.5 | 99.1 | 39.9 | 25.8 | 28.5 | 33.8 | 44.1 | 69.2 | 164.8 | 374.2 |
| Geom. Mean | NC | 18.49 | 46.16 | 117.79 | 222.34 | 207.69 | 173.15 | 119.51 | 41.99 | 14.24 | 11.30 |
| CV % Geom. | NC | 55.8 | 199.7 | 49.9 | 23.8 | 27.1 | 31.5 | 41.1 | 68.2 | 71.7 | NC |
| Mean | | | | | | | | | | | |

Abbreviation(s): MDMA-3,4-Methylenedioxymethamphetamine; NC = Not Calculated; h = Hours; SD = Standard Deviation; IQR = Inter-Quartile Range; Min = Minimum; Max = Maximum; CV % = Percent Coefficient of Variation; n = Number of Participants with Non-Missing Data.

TABLE 16B
Summary of MDMA PK Plasma Concentrations for fasted treatment group

| | Concentration (ng/mL) | | | | | | | | | | |
|---|---|---|---|---|---|---|---|---|---|---|---|
| | Nominal Time (h) | | | | | | | | | | |
| Statistics | 0 | .5 | 1 | 2 | 4 | 6 | 8 | 12 | 24 | 48 | 72 |
| n | 15 | 15 | 15 | 15 | 15 | 15 | 15 | 15 | 15 | 15 | 15 |
| Mean | 0.000 | 23.107 | 110.520 | 210.007 | 225.467 | 193.467 | 166.020 | 117.693 | 50.073 | 7.611 | 0.417 |
| SD | 0.000 | 40.611 | 78.431 | 93.698 | 59.482 | 50.815 | 48.320 | 42.617 | 26.197 | 9.928 | 1.614 |
| IQR | 0.000 | 21.000 | 83.900 | 80.000 | 88.000 | 70.000 | 61.000 | 52.700 | 43.300 | 13.800 | 0.000 |
| Min | 0.000 | 0.000 | 0.000 | 38.300 | 160.000 | 122.000 | 92.300 | 62.700 | 20.500 | 0.000 | 0.000 |
| Median | 0.000 | 0.000 | 124.000 | 211.000 | 218.000 | 199.000 | 165.000 | 120.000 | 53.600 | 6.500 | 0.000 |
| Max | 0.000 | 119.000 | 308.000 | 440.000 | 388.000 | 324.000 | 274.000 | 236.000 | 120.000 | 35.400 | 6.250 |
| CV % | NC | 175.8 | 71.0 | 44.6 | 26.4 | 26.3 | 29.1 | 36.2 | 52.3 | 130.5 | 387.3 |
| Geom. Mean | NC | 39.298 | 111.004 | 186.023 | 219.164 | 187.692 | 159.657 | 111.266 | 44.151 | 12.252 | 6.250 |
| CV % Geom. | NC | 136.5 | 62.0 | 62.3 | 24.3 | 25.7 | 29.7 | 35.5 | 56.7 | 61.5 | NC |
| Mean | | | | | | | | | | | |

Abbreviation(s): MDMA = 3,4-Methylenedioxymethamphetamine; NC = Not Calculated; h = Hours; SD = Standard Deviation; IQR = Inter-Quartile Range; Min = Minimum; Max = Maximum; CV % = Percent Coefficient of Variation; n = Number of Participants with Non-Missing Data.

TABLE 17
Summary of MDA PK Plasma Concentrations

| | Concentration (ng/mL) | | | | | | | | | | |
|---|---|---|---|---|---|---|---|---|---|---|---|
| | Nominal Time (h) | | | | | | | | | | |
| Statistics | 0.0 | .5 | 1 | 2 | 4 | 6 | 8 | 12 | 24 | 48 | 72 |
| n | 14 | 14 | 14 | 14 | 14 | 14 | 14 | 14 | 14 | 9 | 3 |
| Mean | 0.000 | 0.121 | 1.052 | 3.509 | 9.218 | 11.312 | 11.458 | 10.788 | 6.420 | 2.230 | 1.249 |
| SD | 0.000 | 0.319 | 1.122 | 1.711 | 2.052 | 2.891 | 3.108 | 3.447 | 3.019 | 1.492 | 0.838 |
| IQR | 0.000 | 0.000 | 2.220 | 3.340 | 2.250 | 4.040 | 3.610 | 3.650 | 3.010 | 1.380 | 1.578 |
| Min | 0.000 | 0.000 | 0.000 | 1.440 | 5.790 | 6.380 | 6.070 | 5.470 | 2.970 | 0.548 | 0.622 |
| Median | 0.000 | 0.000 | 0.800 | 3.180 | 8.975 | 11.650 | 10.650 | 10.800 | 6.195 | 1.870 | 0.924 |
| Max | 0.000 | 1.060 | 2.580 | 6.430 | 13.300 | 15.900 | 17.700 | 19.300 | 14.500 | 5.440 | 2.200 |
| CV % | NC | 263.2 | 106.6 | 48.8 | 22.3 | 25.6 | 27.1 | 32.0 | 47.0 | 66.9 | 67.1 |
| Geometric | NC | 0.822 | 2.074 | 3.125 | 9.010 | 10.953 | 11.061 | 10.283 | 5.866 | 1.832 | 1.081 |
| Mean | | | | | | | | | | | |
| CV % | NC | 37.1 | 18.7 | 54.1 | 22.5 | 27.3 | 28.5 | 33.5 | 45.5 | 76.9 | 72.0 |
| Geometric | | | | | | | | | | | |
| Mean | | | | | | | | | | | |

### Discussion

MDMA exhibits high PK variability. The unbound maximum concentration (Cₘₐₓ) of MDMA following a single 120 mg dose is estimated to be 0.66 μM, based on the PK data from the MPKF study demonstrating a Cₘₐₓ of 261 ng/ml following a single 120 mg dose of MDMA, and literature reporting that in humans MDMA is 51% bound to plasma proteins (Wan Aasim W R, Tan S C, Gan S H. Interspecies In Vitro Evaluation of Stereoselective Protein Binding for 3, 4-Methylenedioxymethamphetamine. Journal of Chemistry. 2017; 2017).

Sixteen participants were enrolled, randomized to Fed/Fasted or Fasted/Fed states sequence of Dosing Session, and received a single dose of 120 mg MDMA HCl (equivalent to 100 mg MDMA free base) per Dosing Session. Procedures were identical, except for the high-calorie meal during the Fed state. Fourteen (87.5%) participants completed the study: 2 (12.5%) participants discontinued from the study and 15 (93.8%) participants were evaluated for PK.

Following a single administration of 120 mg of MDMA HCl, MDMA plasma concentrations and PK parameters (Cₘₐₓ, AUC₀₋ₜ, AUC_{0-inf}, AUC₀₋₇₂, and AUC_{% ext}) were not significantly different when comparing the fed and the fasted conditions. A delay in Tmax in the fed state has been observed (Cₘₐₓ reached at a median of 4 versus 2 hours in the fed and fasted states, respectively), while the first measurable concentrations (T_{lag}) were similar in both groups. Mean elimination half-life was 8 to 9 hours in both groups; similar CL/F and V_{d}/F were also observed. The rate and extent of exposure for MDA after MDMA administration were similarly not affected by food.

MDMA is metabolized in part by CYP2D6 and the active metabolite MDA, while not formed largely via the CYP2D6 pathway, is metabolized to an inactive metabolite by CYP2D6. While this study was not powered to detect statistical differences in CYP2D6 metabolizer status (8 [50%] intermediate and 8 [50%] normal metabolizers were enrolled), it appears that participants with intermediate CYP2D6 metabolizer status exhibit slightly higher plasma PK values of MDMA (approximately 26%) and MDA (approximately 13%) relative to normal metabolizers. These differences were not clinically meaningful.

Interestingly, 1 participant exhibited relatively high MDMA plasma PK parameter values (AUC_{0-inf} and Cₘₐₓ) during the Fed and Fasted dosing sessions; this participant was a female with low BMI and had an intermediate CYP2D6 metabolizer status and no other explanation was found. MDMA was well tolerated in this participant and no AEs were reported. This supports tolerability of MDMA at Cₘₐₓ as high as 440 ng/mL and AUC0-inf of 8923 h*ng/mL. A single dose of 120 mg MDMA HCl was, overall, well tolerated: 6 participants (37.5%) reported TEAEs; all AEs were mild in severity, and all resolved. Five participants (31.3%) experienced headache; 2 participants (12.5%) experienced nausea and vomiting during the fasted session—1 participant vomited within 4 hours of dosing and was discontinued from treatment. No SAEs nor AESI were reported.

Longitudinally from screening to the end of the study, MDMA had no significant impact on biologic laboratory parameters; post-baseline clinical laboratory abnormalities reported were defined as non-clinically significant.

MDMA impact on vital signs was anticipated and aligned with previous observations. The time course of changes in SBP, DBP, and temperature were different in the fed and fasted groups, with a delay in largest mean changes in the fed group, occurring at 4 hours versus 2 hours during the fasted condition. This observation is congruent with the lag in Tmax observed when comparing the 2 conditions. The time course of changes in HR was, however, identical in both groups. The magnitude of change was different for SBP and temperature, with a non-clinically significant difference of about +5 mmHg and +0.05° C. in largest mean changes when comparing the fasted condition to the fed one. For DBP and HR, the magnitude of change was identical across groups. The frequencies of potentially clinically significant blood pressures (SBP and DBP) and temperatures were similar in both groups. No participant experienced SBP≥160 mmHg or DBP≥100 mmHg, nor temperature>38.3° C. at any timepoint. For HR, the frequency of potentially clinically significant events was slightly higher in the fasted group. This data suggests that food might be responsible for a delay in reaching Cmax and has some impact on clinical parameters but not all (such as HR); however, other PK parameters are identical and clinical influence is absent.

On ECG parameters, MDMA had no significant impact. Specifically, there was no change in mean QTcF over time above 10 ms at any timepoint. A linear mixed-effect model describing the CFB in QTcF interval relative to MDMA concentration was used to simulate the CFB QTcF at the upper 95% CI for the observed Cmax. The simulated CFB QTcF at the upper 95% CI for Cmax predicts a decrease in QTcF of at least 0.43 ms in both Fasted and Fed states. Further, no participant experienced QTcF intervals above 450 ms in any group; only 1 participant (6.3%) in the fasted group had a mean QTcF increase from baseline above 30 ms at the 12-hour timepoint post-dose.

MDMA produced some degree of change in the SE scores in this study, with a prominent elevation trend in “body perception changes,” “difficulty concentrating,” “compassion for others,” “compassion for self,” and “meaningful experience” scores. Those increases from baseline were observed between 0.5 to 4 hours after dosing. “Body perception changes” and “difficulty concentrating” scores showed a trend toward return to baseline at the end of the session; “compassion for others,” “compassion for self,” and “meaningful experience” scores persisted throughout the dosing session. The other scores remained relatively stable in comparison to baseline across the sessions. No abnormal C-SSRS scores were recorded during the study.

Importantly, this study underlines the safety and tolerability of MDMA on clinical and biological parameters, as well as constitutes another important element of evidence (in conjunction with preclinical—human ether-a-go-go related gene (hERG) and dog cardiovascular (CV) studies, and clinical data gathered across the Phase 2 and 3 studies) that MDMA has low to no potential to impact the QT/QTc interval, as demonstrated by the linear regression model and the ECG data.

Overall, the results from this study suggest that the administration of MDMA in fasted or fed states resulted in similar PK, clinical, biological, electrophysiological and pharmacodynamic parameters, indicating an equivalent relative bioavailability; therefore, MDMA can be administered with or without food. In some embodiments, the compositions and dosage forms described herein are to be administered to a subject in a fed state. In some embodiments, the compositions and dosage forms described herein are to be administered to a subject in a fasted state.

### Example 8. Population PK (PopPK) Analysis

This example describes the methodology that was used to characterize the PPK of MDMA and its active minor metabolite MDA and the related model-based simulations.

### Methods

### Evaluable Subjects

For the PPK analysis, an individual was defined as evaluable if the following criteria were satisfied:
1. Received at least one dose of MDMA
2. Had at least one measurable MDMA observation with associated sampling time and dosing information for the MDMA modeling
3. Had at least one measurable paired MDMA/MDA observation with associated sampling time and dosing information for the MDMA modeling

### Covariate Variables

The evaluation of the impact of covariates on the MDMA and MDA PPK models focused on the most clinically relevant covariates. Prior to covariate selection, a correlation matrix was generated to determine the correlation between covariates. Only the most significant covariate among the highly correlated covariates was included in the model. Detailed descriptions of covariates tested are provided in Table 18.

TABLE 18
Description of Covariates and Associated Derivation Methods

| Covariate | MDMA | MDA | |
|---|---|---|---|
| (Abbreviation) | Parameters | Parameters | Rationale |
| Body weight | CL and CVd | CL and CVd | Disposition may be |
| | | | body size dependent |
| Age | CL and CVd | CL and CVd | Parameters may |
| | | | change with aging |
| Race | CL and CVd | CL and CVd | Parameters may |
| | | | change with race |
| Sex | CL and CVd | CL and CVd | Parameters may differ |
| | | | between the sexes |
| Albumin | CL and CVd | CL and CVd | Clinical interest |
| Hepatic impairment/ | CL | CL | Clinical interest |
| liver function (ALT) | | | |
| Renal | CL | CL | Clinical interest |
| function (CrCl) | | | |
| Fasting status | Absorption | | Food may impact |
| | constant rate | | absorption processes |

CL = Clearance,
CVd = central volume of distribution,
ALT = alanine aminotransferase;
CrCl = creatinine clearance;
PK = pharmacokinetic

CrCl was calculated according to the Cockcroft-Gault equation:

$$CrCl = \frac{( 140 - age ) \times bodyweight ( kg )}{serum creatinine ( mg / dL ) \times 72} \times 0.85 ( if female )$$

### Modeling and Simulation Analyses

The analyses was carried out according to the United States (US) Guidance for Industry: Population Pharmacokinetics, US Guidance for Industry: Exposure Response, and the European Union (EU) Guidance on Reporting the Results of Population Pharmacokinetic Analyses and followed best practices as outlined by Dykstra et al and Wade et al.

### Software

Software Nonlinear mixed-effects modeling software (NONMEM®; version 7.4; ICON, Hanover, MD, US), a software package for nonlinear mixed-effects analysis was used for the PPK modeling. R (version 4.0.2) was used for simulations (e.g., to derive exposure measures for subsequent model application analysis).

R was used for data preparation, graphical analysis, model diagnostics, and statistical summaries. Xpose® (version 0.4.11) and Perl-speaks-NONMEM® (PsN version 4.8.1; Department of Pharmacy, Uppsala University, Uppsala, Sweden) were also used for model diagnostics and facilitation of tasks such as model running and covariate testing.

### Population PK Analysis

Algorithms for the Development of the Population PK Model Plasma concentration-time data was analyzed using a nonlinear mixed effects modeling approach. The first-order conditional estimation method of NONMEM with interaction (FOCE INTER) was used for PPK model development.

### Model Diagnostics

Several standard diagnostic plots were used throughout model development to assess the ability of each model to describe the observed data. These diagnostic plots included the following:
Observed (DV) versus individual (IPRED) and population (PRED) predictions
Conditional weighted residuals (CWRES)/individual weighted residuals (IWRES) and their absolute values versus PRED/IPRED
CWRES versus time/time since last dose (TSLD)
Above plots stratified by factors, such as dose and cohort
Plots of individual observations versus time with overlaid individual and population fits
Pairwise plots of individual f estimates
Box plots and scatter plots of f's stratified by covariates of interest The utility of the diagnostic plots involving individual post hoc parameters was dependent on a reasonable level of η- and ε-shrinkage. As a general rule, if estimates of shrinkage exceed 30%, plots based upon random effects or residuals need to be interpreted with caution.

### Structural Population PK Model Development

One-, and two-compartment models were evaluated for MDMA based on the shape of the observed concentration-time profile. MDMA absorption was evaluated in two ways: as a first-order process and as a sequential zero-order process followed by first-order absorption. MDA was developed as a sequential model, with one-, two, and three-compartment models tested.

### Random Effects in the Population PK Model

The inter-individual random effects on the parameters were introduced and retained if their inclusion did not cause model instability and if their estimates were not close to zero. They were modeled assuming a log-normal distribution as given by the following expression:

θₖᵢ=θₖ×e^{ηki}

where θₖᵢ denotes the k^{th} parameter value for the i^{th} patient, θₖ denotes the typical parameter value, and ηki denotes the inter-individual random effect for the i^{th} patient—assumed to have mean of 0 (zero) and variance ωk². Collectively, the vector of random effects (across the parameters indexed by k) has the covariance matrix Ω. Covariance matrix structures including diagonal and blocked diagonal structures were evaluated after the completion of covariate model building. The residual error structure was assumed to follow an additive, proportional, or combined additive and proportional error model described by the following:

Yᵢⱼ=Cᵢⱼ×(1+ε1ᵢⱼ)+ε2ᵢⱼ

where Yᵢⱼ is the j^{th} observed concentration for the i^{th} subject, Cᵢⱼ is the corresponding predicted concentration, and ε1ᵢⱼ (proportional) and εε2ᵢⱼ (additive) are the residual errors under the assumption that ε˜N(0, σ²).

The residual error model was optimized until no trends were visible in residual plots (in particular, absolute values of individual weighted residuals [IWRES] versus individual predictions [IPRED]).

### Covariate Selection for the Population PK Model

Covariates of interest for the PK model are listed in Table 42. The subset of covariate-parameter relationships to be included was selected based on exploratory graphical analysis, mechanistic plausibility, and scientific and clinical interest. For a covariate to be included in the formal covariate analysis, the following conditions applied:
The covariate was available in at least 80% of subjects.
For categorical covariates, a minimum number of 20% of subjects should be in each category.
If covariates showed a correlation of >0.5, only one of the correlated covariates was included in the formal analysis. This was either the covariate with the strongest influence as determined by exploratory graphical analysis or the variable that is most meaningful from a clinical, biological, or practical perspective. Continuous covariates were preferred over categorized covariates with the same meaning.

Covariates that did not fulfil these criteria were evaluated graphically in an exploratory manner if deemed meaningful from a clinical, biological, or practical perspective. However, graphical covariate analysis can be relied on only if η-shrinkage in the respective population model parameters is low. Covariates that fulfilled the above criteria were tested in a stepwise process. The covariate selection was performed using a forward addition process followed by backward elimination. The likelihood ratio test was used to evaluate the significance of incorporating or removing fixed effects into the population model based on significance levels that were set a priori. For forward addition and backward elimination, significance levels of 0.01 and 0.001 were employed, respectively. The improvement of the model relative to the base model was compared after each of the covariates was added univariately, and the model with the largest improvement was kept for the next evaluation step, given that there was an overall statistical significance supporting the inclusion of the respective covariate. During the backward elimination process, covariates were removed from the model one at a time if their deletion led to insignificant model deterioration. The most insignificant covariate was removed first, and the procedure was repeated until no further insignificant covariate relationship was detected. All continuous covariates were incorporated into the population model using a scaled structure based on either the median value of the covariate in the population or a standard value of the covariate (e.g., 70 kg for body weight). This approach ensured that covariate effects are relative to an individual in the middle of the population distribution for that covariate. All categorical covariates were incorporated into the population model using a proportional structure with the most common level of the covariate being the reference. This approach ensured that categorical covariate effects were relative to a reference group or category. The mathematical structures of the covariate models are:

$$\begin{matrix}Continuous \\ {P}_{ki} = {θ}_{k} \times ( \frac{{X}_{ij}}{M ( {X}_{j} )} \text{?}\end{matrix}$$

$$\begin{matrix}Categorical \\ {P}_{ki} = {θ}_{k} \times ( 1 + \text{?}\end{matrix}$$

$$\text{?} \text{indicates text missing or illegible when filed}$$

where Pₖᵢ is the population estimate of the parameter Pₖ for subject i, Xᵢⱼ is the value of continuous covariate Xⱼ for subject i, or an indicator variable for subject i for categorical covariate Xⱼ with value 1 for the nonreference category and 0 for the reference category, M(Xⱼ) is the median of covariate Xⱼ in the analysis dataset, θₖ is the typical value of the parameter Pₖ, and θⱼ is a coefficient that reflects the effect of covariate Xⱼ on the parameter.

To ensure that the model that emerged from the covariate testing process did not neglect any important covariate effects, random effects from the tentative final model were plotted versus potential covariates and evaluated for residual trends in parameter covariate relationships. The effect of covariates retained following backward elimination were assessed and covariates lacking in clinical relevance (i.e. <10% change) were excluded from the final model.

### Final Population PK Model

At the end of covariate testing, alternative variance-covariance structures for Q were evaluated including partial and full block structures. Such a structure was deemed suitable if it provided a statistically significant (p<0.001) improvement in the model objective function value and if it improved model stability as measured by the condition number and/or a successful covariance step. The model that results from this step of the model building process was considered the final model.

Further criteria for accepting a model as the final run included the following:
Successful minimization (for gradient methods)
No estimates close to a boundary
Relative standard errors of the estimates should preferably be <30% for fixed effect parameters and <50% for random effect parameters.
No unacceptable trends in goodness-of-fit (GOF) plots
The model allows for the goal of the analysis to be met
There is plausibility of the parameter estimates

### Evaluation of the Final Population PK Model

A nonparametric bootstrap analysis was conducted to evaluate the stability of the final model and to estimate confidence intervals (CIs) for the model parameters. The bootstrap analysis was performed with 1000 replicates of the dataset, generated by random resampling of subjects from the original dataset with replacement. The final model was repeatedly fitted to bootstrap replicates of the dataset. CIs were calculated based on the distribution of the parameter estimates from the bootstrap runs. Visual predictive checks (VPCs) with prediction correction were used to evaluate the predictive ability of the final model. Plots of observed data distributions were compared to simulated distributions to demonstrate the model's ability to adequately predict the data on which the model is based. VPCs were based on 1000 simulations and stratified by covariates of interest

### Model Application

### Estimates of Individual Exposure

The final PPK models were used to predict rich concentration-time profiles for MDMA and MDA based on subject-level posterior Bayes estimates of the PK parameters. Exposure metrics, including AUC₀₋₄₄ (44 hours after dose, corresponding to approximately 5 half-lives of MDMA) and Cmax at steady state were derived based on these concentration-time profiles. Descriptive statistics were derived, and results summarized. Results were also summarized within subgroups based on clinically important covariates that were identified during the covariate analysis.

### Simulations to Optimize MDMA Dosing

The final PPK model was used to perform.

### QC Procedures

QC was performed in compliance with applicable Certara standard operating procedures (SOPs). Data management, modeling analyses, and the final report were reviewed by consultants who were not involved in the respective tasks. All QC findings were documented.

### Results

### PK Measurements

The PK dataset for MDMA consisted of 2968 MDMA PK observations and 2968 MDA PK observations from 67 subjects. 451 MDMA PK observations and 451 MDA PK observations were from placebo dosing and not included in the PPK analysis. One subject was excluded entirely from analysis because only the placebo arm was available. Observation records were excluded if they were below the limit of quantitation (BLQ), had missing time, or had pre-dose concentrations. The PPK analysis for MDMA was conducted on 2012 measurable PK observations from 65 subjects. The PPK analysis for MDA was conducted on 1871 measurable PK observations from 65 subjects. Number of evaluable subjects, measurable observations, excluded observations, and BLQ observations across studies are summarized by study and treatment group in Table 22. Rich concentration-time profiles were available for all evaluated subjects. BLQ observations after administration of the first dose (“post-treatment BLQ”) accounted for 13.6% of all observations for MDMA and 19.7% of all observations for MDA. However, nearly all recorded BLQs were measured during the washout period. A sensitivity test was later performed to evaluate the impact of this record on the developed model and did not result in any significant changes to model stability or parameter estimates.

### Exploratory PK Analysis

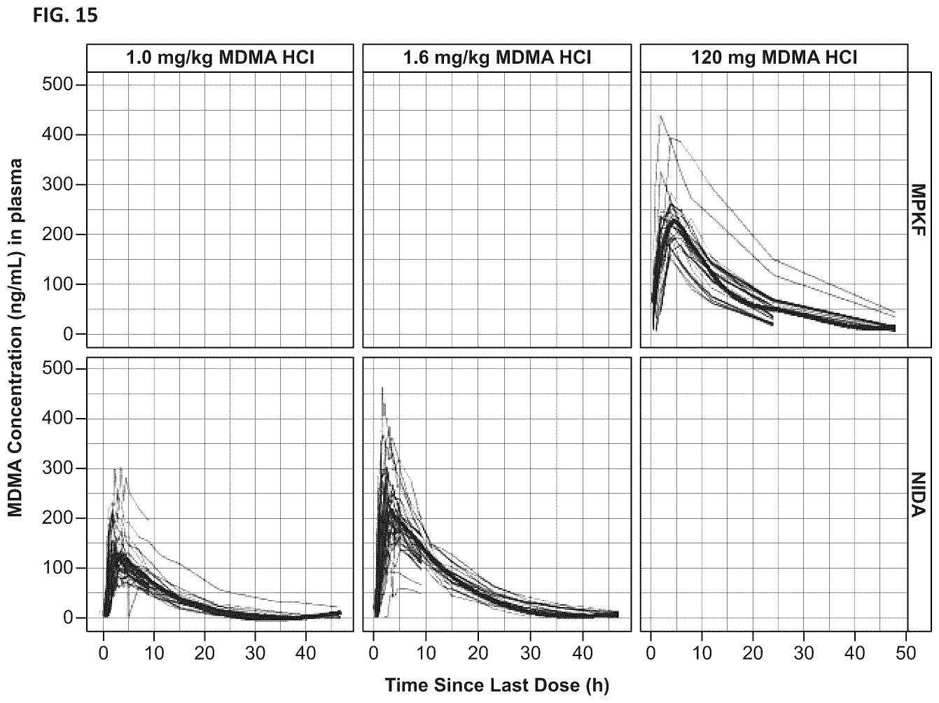

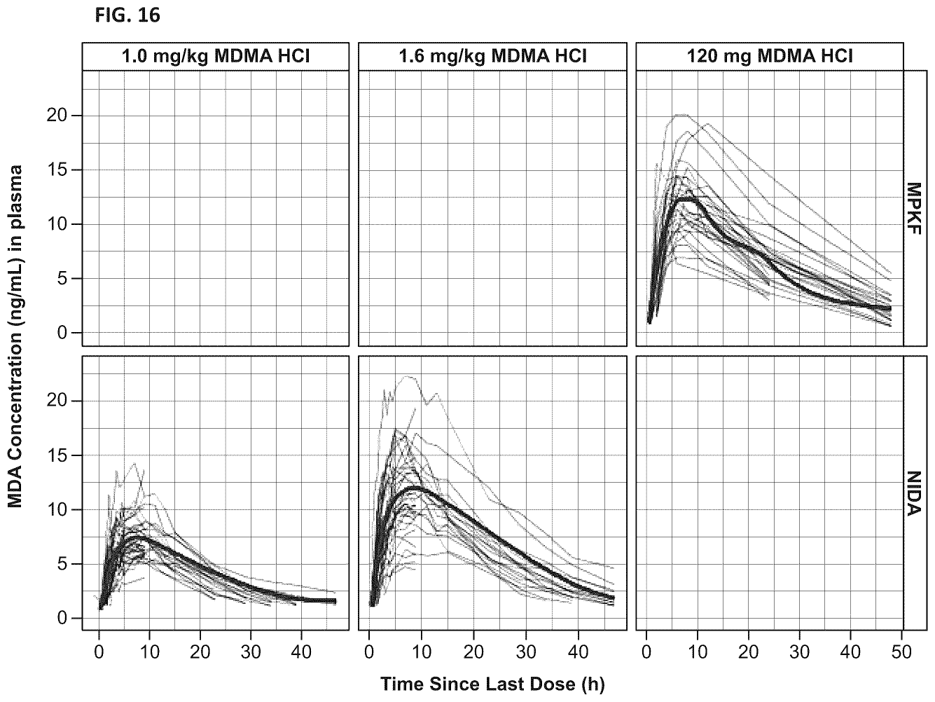

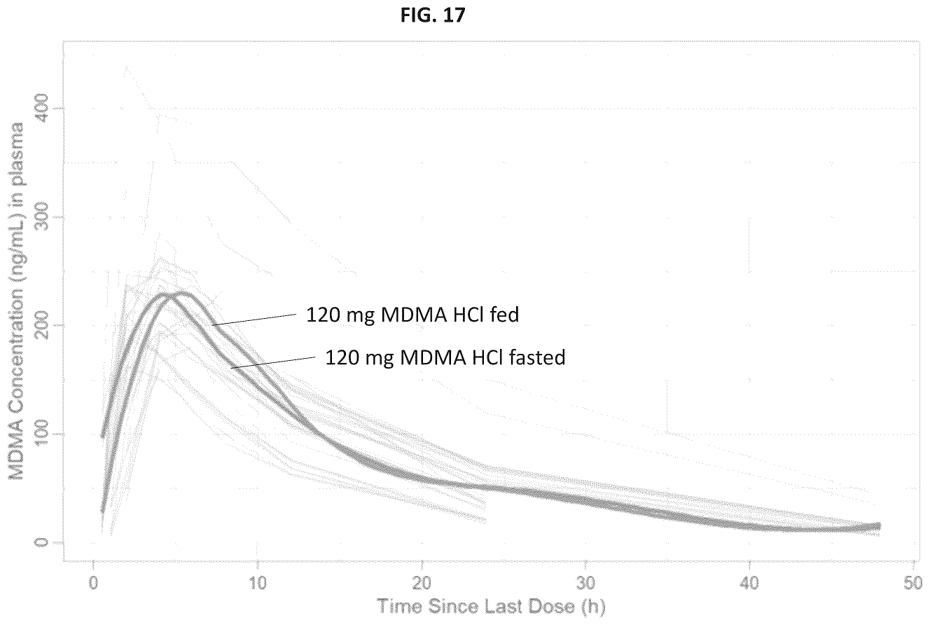

Individual measurable plasma drug concentration of MDMA versus time since last dose, TSLD (semi-log), stratified by study and dose, suggest a single-phase elimination profile (FIG. 15). The data shown in FIG. 16 for individual measurable plasma drug concentration of MDA versus TSLD (semi-log), stratified by study and dose, suggest a multi-phase elimination profile. The effect of fasting status was graphically evaluated using data from the MPKF food effect study and is shown in FIG. 17. A delay was observed in the time to reach maximum concentration (tmax) when patients were fed with a high-fat/high-calorie meal, suggesting a delay in absorption in these subjects.

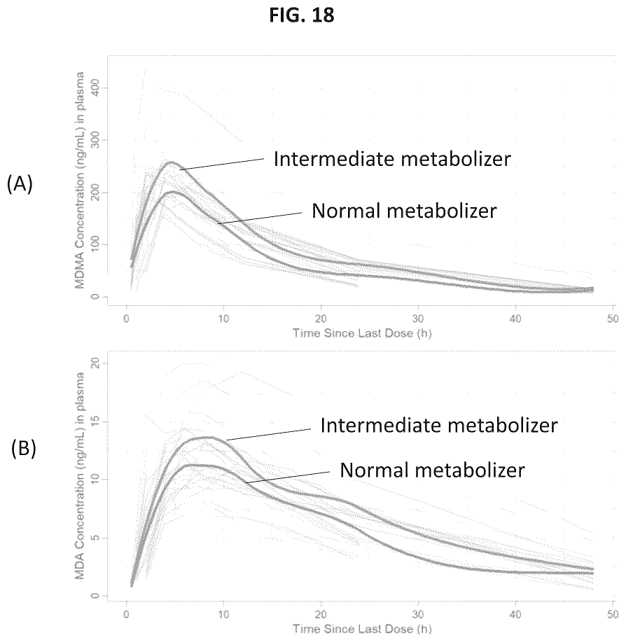

Plasma levels of both MDMA and MDA are influenced by the CYP2D6 enzyme. Therefore, variation in CYP2D6 metabolism was considered as a potential factor influencing the clearance of both analytes. CYP2D6 metabolizer status was available only in the MPKF study, with 8 subjects that were normal metabolizers, and 8 subjects that were intermediate metabolizers. The concentration-time profiles stratified by CYP2D6 status are summarized in FIG. 18.

### Base Structural Model

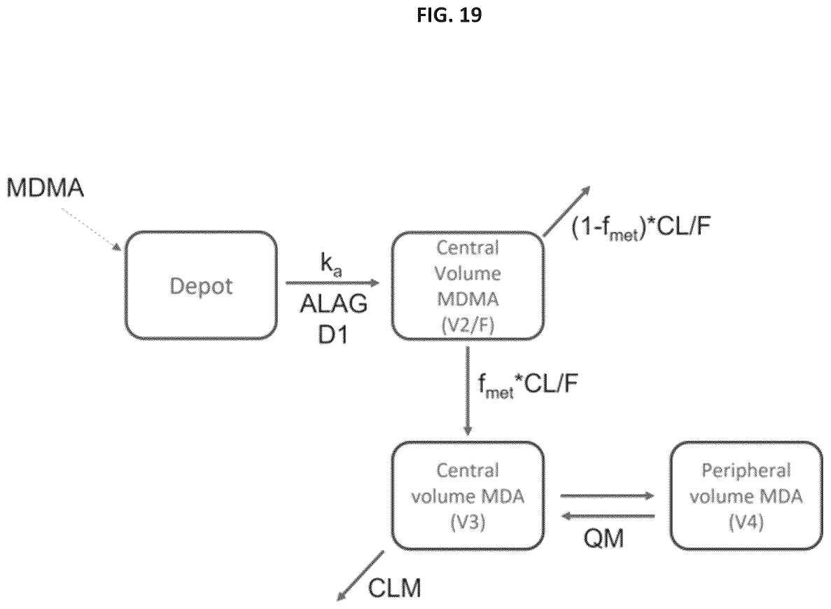

MDMA and MDA were modeled sequentially. FIG. 19 presents the model structure for the two stage PPK model.

### MDMA Base Model

Various base structural models were tested for the MDMA base model. Based on the exploratory graphical analysis, one- and two-compartment structural models with first-order elimination were considered suitable as initial structural models. A one-compartment structure was found to produce a similar fit of the data as compared to the two-compartment model. Therefore, the one compartment model was selected as the most parsimonious model. The addition of an absorption lag reduced the objective function value by more than 600 points. Furthermore, a sequential zero- and first-order absorption process was found to provide a more adequate fit of the absorption profile. Inter-individual variability (IIV) was evaluated on apparent clearance (CL/F), apparent volume (V/F), absorption rate constant (KA), and duration of zero-order absorption (D1). Proportional and combined additive+proportional error models were evaluated. Removal of additive error caused model instability, and therefore the combined error model was selected. The first-order conditional estimation with interaction (FOCE INTER) method as implemented in NONMEM was used for model fitting.

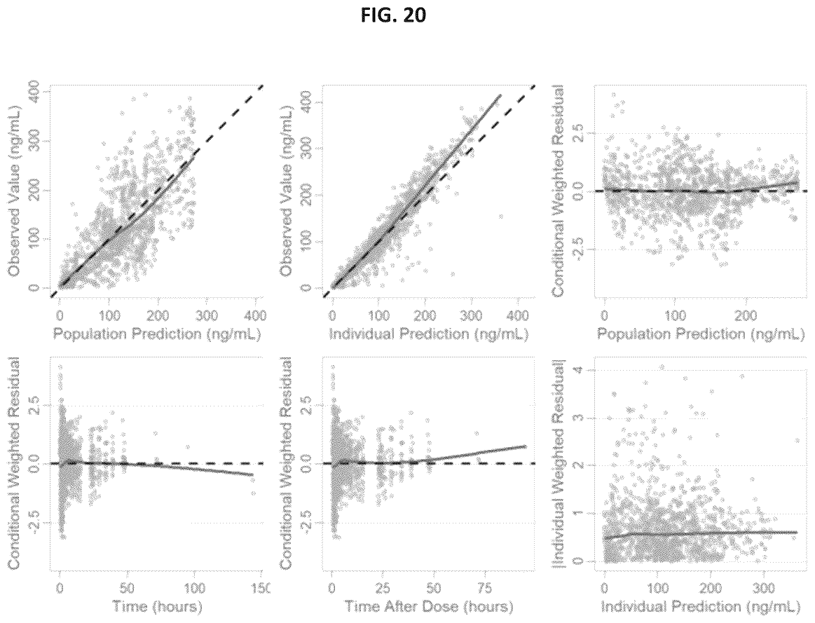

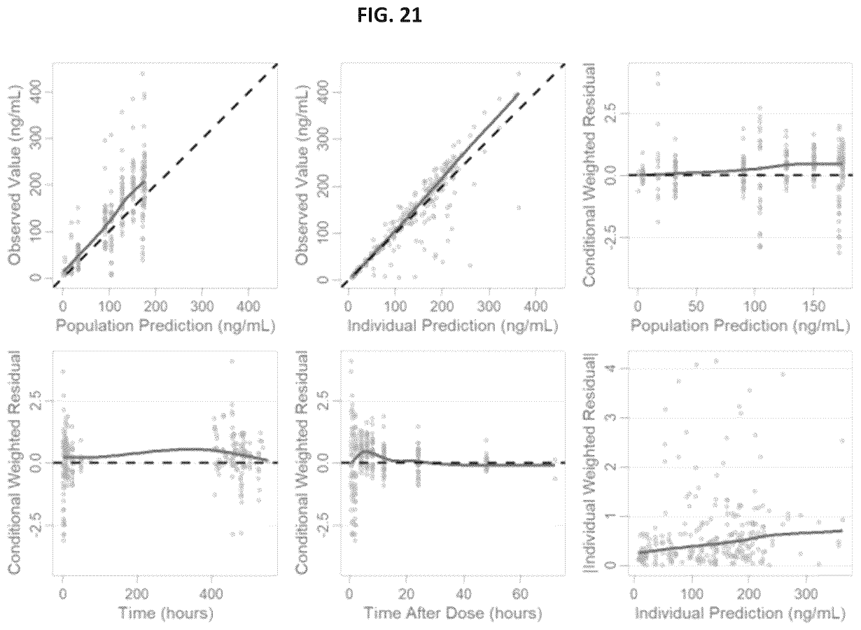

The goodness of fit (GOF) plots for the base MDMA model are shown in FIG. 19. No marked systematic trends were seen in the residual diagnostic plots. GOF plots for the base model for all data are shown in FIG. 20. GOF plots for the base model stratified by study are shown in FIG. 21 (MPKF). Table 19 lists the Base Model Parameter Estimates for MDMA.

TABLE 19
Base Model Parameter Estimates for MDMA.

| | Estimate | % RSE | Shrinkage (%) |
|---|---|---|---|
| Parameter | | | |
| CL/F (L/h) | 38.3 | 5.13 | |
| V2/F (L) | 444 | 3.70 | |
| kₐ (1/h) | 1.14 | 7.58 | |
| ALAG1 (h) | 0.312 | 5.52 | |
| D1 (h) | 0.242 | 2.08 | |
| Prop. Error (ng/mL) | 0.226 | 8.28 | |
| Add. Error (ng) | 6.21 | 24.0 | |
| fₘₑₜ | 0.1 FIXED | — | |
| Random effects | | | |
| IIV on CL/F | 42.5 | 20.5 | 2.2 |
| IIV on V2/F | 28.5 | 18.2 | 4.2 |
| IIV on Ka | 55.9 | 15.6 | 16.6 |
| IIV on D1 | 159 | 6.20 | 18.2 |
| Residual error | | | |
| EPS | 1 FIX | — | 5.1 |

Abbreviations:
Add = additive;
ALAG1 = absorption lag time;
CL/F = apparent central clearance;
CV = coefficient of variation;
D1 = duration of zero order input;
EPS = epsilon;
fmet = fraction of MDMA metabolized to MDA;
IIV = inter-individual variability;
ka = absorption rate constant;
Prop = proportional;
RSE = relative standard error;
V2/F = apparent central volume of distribution

### MDA BASE MODEL

Individual post hoc estimates from the MDMA PPK model were used to simulate MDMA concentrations for the MDA model. One-, two-, and three-compartment structural models with first-order elimination were evaluated. Although the three-compartment model had the lowest OFV, a stable model could not be obtained. A two-compartment model was therefore identified as the best model to describe MDA PK. IIV was evaluated on apparent clearance (CLM), inter-compartmental clearance (QM), and peripheral volume (V4). IIV on V3 was not included as this produced a 0 gradient on V3 during minimization. Proportional and combined additive+proportional error models were evaluated. Removal of additive error caused model instability, and therefore the combined error model was selected. The first-order conditional estimation with interaction (FOCE INTER) method as implemented in NONMEM was used for model fitting.

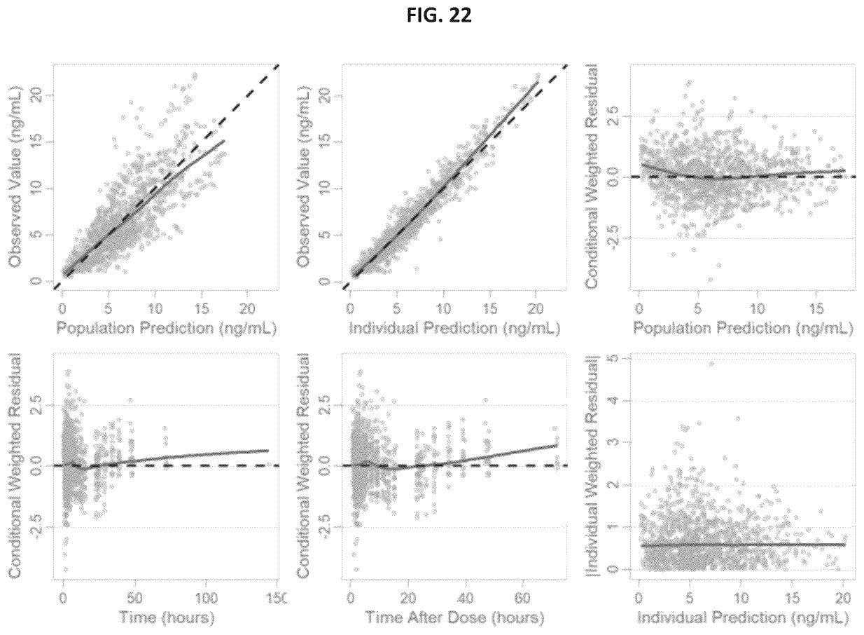

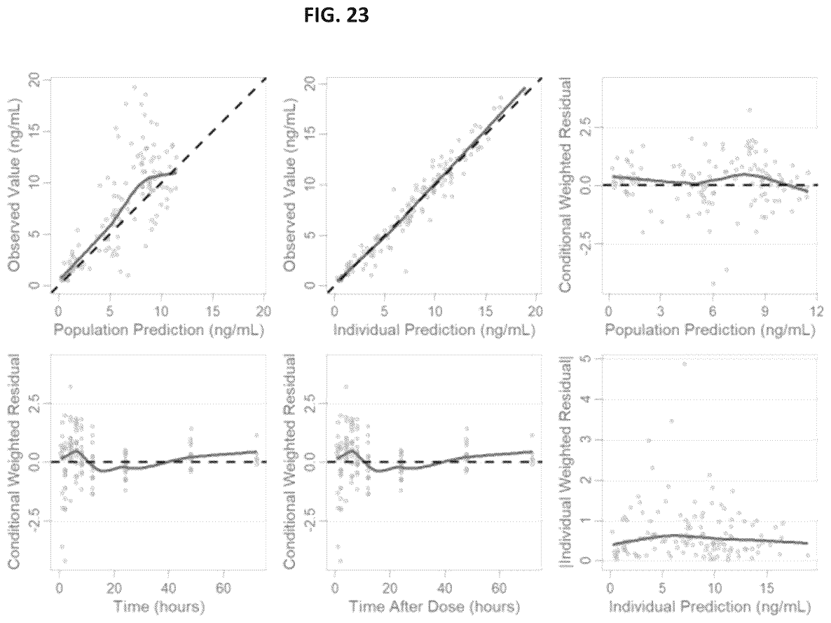

The goodness of fit (GOF) plots for the base MDA model are shown in FIG. 22. No marked systematic trends were seen in the residual diagnostic plots. GOF plots for the base model stratified by study are shown in FIG. 23 (MPFK). One outlier data point was observed at 148 hours after dose and excluded from the plot.

Parameter estimates for the base MDA model are reported in Table 20. All PK parameters were estimated with RSE below 30%. The f-shrinkages for the base model were within an acceptable range for all parameters.

TABLE 20
Base Model Parameter Estimates for MDA

| | Estimate | % RSE | Shrinkage (%) |
|---|---|---|---|
| Parameter | | | |
| CLM (L/h) | 35.6 | 4.31 | |
| V3 (L) | 19.4 | 27.8 | |
| QM (1/h) | 85.7 | 12.0 | |
| V4 (h) | 197 | 6.73 | |
| Prop. Error (ng/mL) | 0.126 | 29.7 | |
| Add. Error (ng) | 0.756 | 18.6 | |
| Random effects | | | |
| IIV on CLM | 37.7 | 19.0 | 3.1 |
| IIV on QM | 55.8 | 29.1 | 7.1 |
| IIV on V4 | 43.5 | 24.1 | 10.7 |
| Residual error | | | |
| EPS | 1 FIX | — | 4.5 |

Abbreviations: Add = additive; CLM = apparent central clearance; CV = coefficient of variation; EPS = epsilon; IIV = inter-individual variability; QM = inter-compartmental clearance; Prop = proportional; RSE = relative standard deviation; V3 = apparent central volume; V4 = peripheral volume

### Model Refinements

Covariates of interest were graphically evaluated for their potential effects on PK parameters prior to the formal covariate analysis. This preliminary step was intended to identify covariates that were likely to have a significant effect on the PK of MDMA and MDA.

Using the base PPK model, a covariate analysis was performed for MDMA. The covariate search was performed as described in the preceeding. The following covariates were included in the SCM: age, body weight, alanine aminotransferase, albumin, creatinine clearance, race, sex, fed status, and study to check for possible differences between the two studies. During the forward covariate selection step, creatinine clearance and study were found to have a significant influence on apparent central clearance. Body weight and study were found to have a significant influence on apparent central volume. Fasting status was found to have a significant influence on the rate of first order absorption. No covariates were removed during the backwards elimination step.

Using the base PPK model, a covariate analysis was performed for MDA. The covariate search was performed as described in the preceeding. The following covariates were included in the SCM: age, body weight, alanine aminotransferase, albumin, creatinine clearance, race, and sex. During the forward covariate selection step, age and body weight were found to have a significant influence on apparent central clearance of MDA. Body was found to have a significant influence on intercompartmental clearance. Sex was found to have a significant influence of peripheral volume. During backward elimination, the age effect on central clearance was removed.

### MDMA Final Model

Model refinements were conducted with the final covariate MDMA model based on the SCM result to obtain the final model. Although body weight and creatinine clearance were correlated, both were included in the covariate search, with body weight as a measure of body size and creatinine clearance as a measure of renal function. Replacing the creatinine clearance effect on central clearance led to an OFV drop of 2.31 points (p=0.13), indicating that the two covariates had a similar overall impact. Therefore, body weight was introduced into the model in place of creatinine clearance, to allow for potentially better interpretation of model effects. IIV between CL/F and V2/F were highly correlated (r=0.6), therefore an Omega block was introduced to capture this correlation, leading to a 22-point drop in OFV.

### Analysis of MDMA Final Model

A bootstrap analysis was performed to assess the robustness of the final PPK model of MDMA. The parameter estimates for the final PPK model together with bootstrap results are presented in Table 21.

TABLE 21
Final Model Parameter Estimates for MDMA PK

| | | | Shrinkage | Bootstrap median | |
|---|---|---|---|---|---|
| | Estimate | % RSE | (%) | (95% CI) | |
| Parameter | | | | | |
| CL/F (L/h) | 41.5 | | 4.70 | 41.8 | (37.7, 45.8) |
| V2/F (L) | 462 | | 3.46 | 461 | (429, 496) |
| kₐ (1/h) | 1.17 | | 8.26 | 1.17 | (0.969, 1.43) |
| ALAG1 (h) | 0.322 | | 5.15 | 0.323 | (0.273, 0.358) |
| D1 (h) | 0.304 | | 18.2 | 0.271 | (0.141, 0.485) |
| fₘₑₜ | 0.1 FIXED | — | | — | |
| Prop. Error (ng/mL) | 0.207 | 9.24 | | 0.208 | (0.164, 0.25) |
| Add. Error MPKF | 2.5 FIX | — | | — | |
| (ng) | | | | | |
| Fed status effect | −0.484 | 18.6 | | −0.496 | (−0.645, −0.239) |
| on KA | | | | | |
| Fasted status | 1.93 | 72.3 | | 1.08 | (0.277, 4.31) |
| effect on KA | | | | | |
| Body weight | 1.03 | 17.2 | | 1.05 | (0.627, 1.4) |
| effect on CL/F | | | | | |
| Study effect on | −0.36 | 16.40 | | −0.359 | (−0.474, −0.23) |
| CL/F | | | | | |
| Body weight | 0.885 | 13.1 | | 0.884 | (0.6, 1.11) |
| effect on V2/F | | | | | |
| Study effect on | −0.263 | 14.40 | | −0.267 | (−0.342, −0.19) |
| V2/F | | | | | |
| Random effects | | | | | |
| IIV on CL/F | 30.5 | 27.2 | 3.2 | | |
| IIV on V2/F | 19.9 | 28.4 | 7.2 | | |
| Corr(CL/F, V2/F) | r = 0.67 | 39.3 | — | | |
| IIV on KA | 60.2 | 18.2 | 14.5 | | |
| IIV on D1 | 153 | 23.1 | 14.1 | | |
| Residual error | | | | | |
| EPS | 1 FIXED | — | 4.8 | | |

Abbreviations: ALAG1 = absorption lag time; CL/F = apparent central clearance; corr = correlation; CV = coefficient of variation; D1 = duration of zero order input; EPS = epsilon; fmet = fraction of MDMA metabolized to MDA; IIV = inter-individual variability; ka = absorption rate constant; r = correlation; RSE = relative standard error; V2/F = apparent central volume of distribution

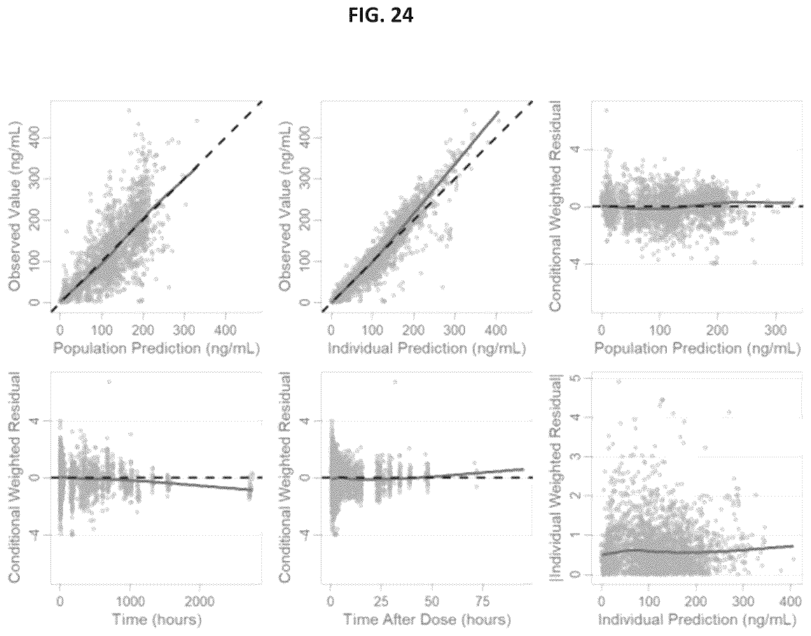

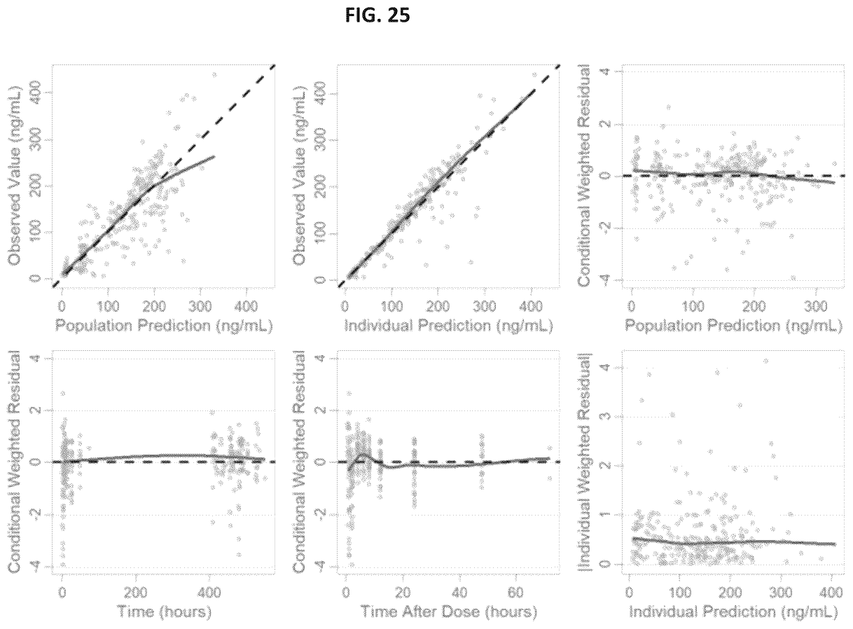

All analysis runs were performed using the FOCEI method in NONMEM. GOF plots for the final MDMA model for all data are shown in FIG. 24. No marked systematic trends were seen in the residual diagnostic plots. GOF plots for the MDMA final model stratified by study are shown in FIG. 25 (MPFK). One outlier data point was observed at 148 hours after dose and excluded from the figures.

### MDA Final Model

The final covariate model based on the SCM was further modified by adding an Omega block to capture the correlation between metabolite clearance, intercompartmental clearance, and peripheral volume, resulting in an 86-point OFV drop. This was selected as the final model.

### Analysis of MDA Final Model

A bootstrap analysis was performed to assess the robustness of the final PPK model of MDA. The parameter estimates for the final PPK model together with bootstrap results are presented in Table 22.

TABLE 22
Final Model Parameter Estimates for MDA PK

| | Estimate | % RSE | Shrinkage (%) | Bootstrap median (95% CI) | |
|---|---|---|---|---|---|
| Parameter | | | | | |
| CLM (L/h) | 79.6 | 10.3 | | 83.9 | (68.8, 103) |
| V4 (L) | 213 | 7.66 | | 213 | (181, 246) |
| Prop. Error (ng/mL) | 0.122 | 32.8 | | 0.118 | (0.0334, 0.17) |
| Add. Error (ng) | 0.776 | 19.6 | | 0.769 | (0.594, 1.08) |
| Body weight | 0.580 | 26.2 | | 0.585 | (0.209, 0.879) |
| effect on CLM | | | | | |
| Body weight | 1.00 | 29.8 | | 1.02 | (0.408, 1.63) |
| effect on QM | | | | | |
| Sex effect on V4 | −0.181 | 38 | | −0.18 | (−0.321, −0.0256) |
| Random effects | | | | | |
| IIV on CLM | 32.0 | 18.7 | 1.3 | | |
| Corr(CLM, QM) | r = 0.82 | 26.3 | — | | |
| IIV on QM | 46.8 | 29.3 | 5.5 | | |
| Corr(CLM, V4) | r = 0.86 | 20.8 | — | | |
| Corr(QM, V4) | r = 0.70 | 32.8 | — | | |
| IIV on V4 | 35.2 | 26.0 | 5.7 | | |
| Residual error | | | | | |
| EPS | 1 FIXED | — | 3.3 | | |

Abbreviations: Add = additive; CI = confidence interval; CLM = apparent central clearance for MDA; corr = correlation; CV = coefficient of variation; EPS = epsilon; IIV = inter-individual variability; PK = pharmacokinetic; Prop = proportional; r = correlation; RSE = relative standard error; QM = intercompartmental clearance for MDA; V3 = apparent central volume of distribution for MDA; V4 = peripheral volume of distribution for MDA

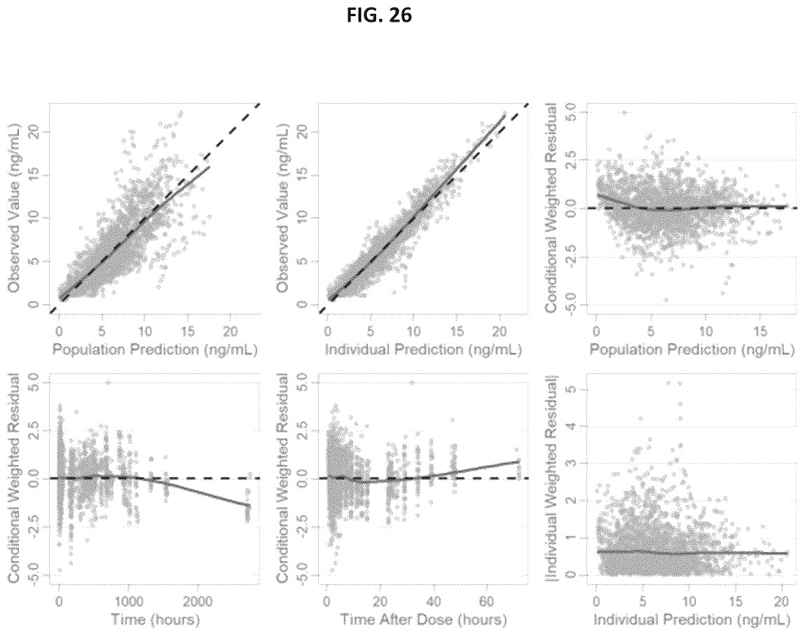

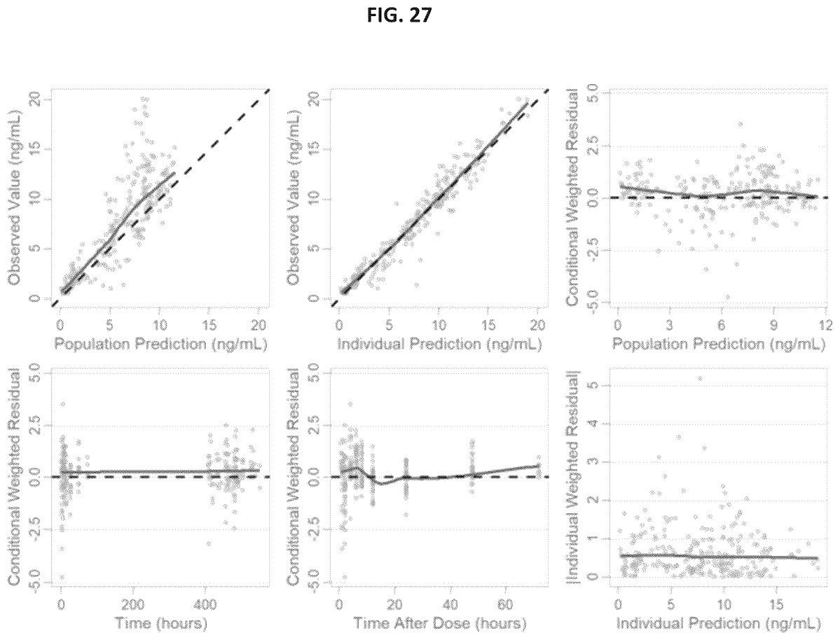

All analysis runs were performed using the FOCEI method in NONMEM. GOF plots for the final MDA model all data are shown in FIG. 26. No marked systematic trends were seen in the residual diagnostic plots. GOF plots for the final model stratified by study are shown in FIG. 27 (MPFK).

### Model Qualification

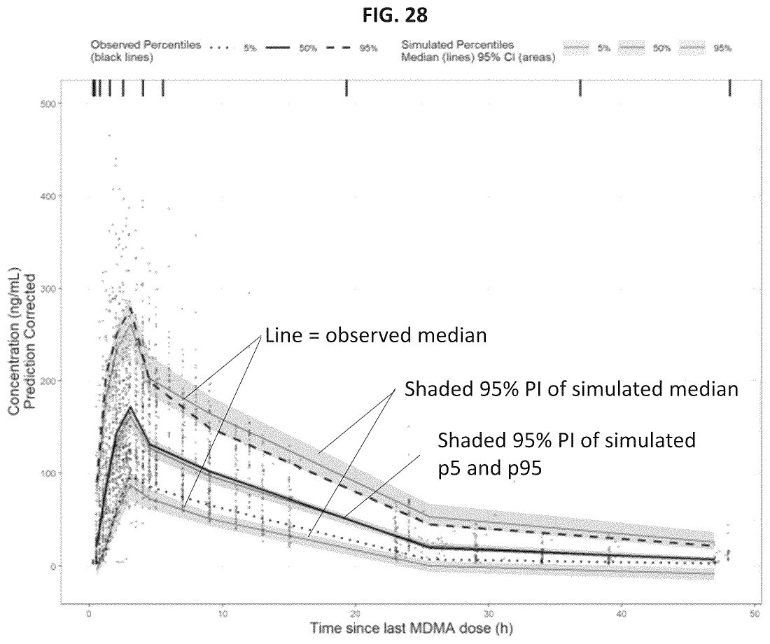

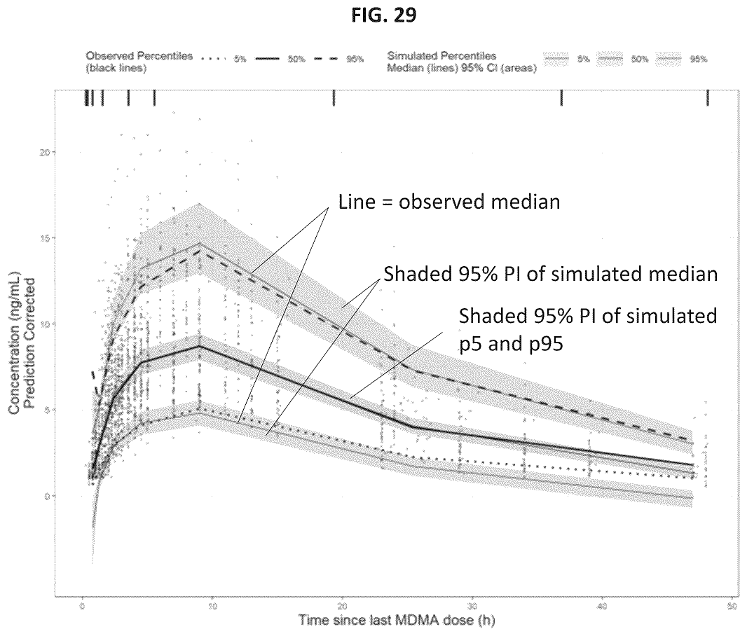

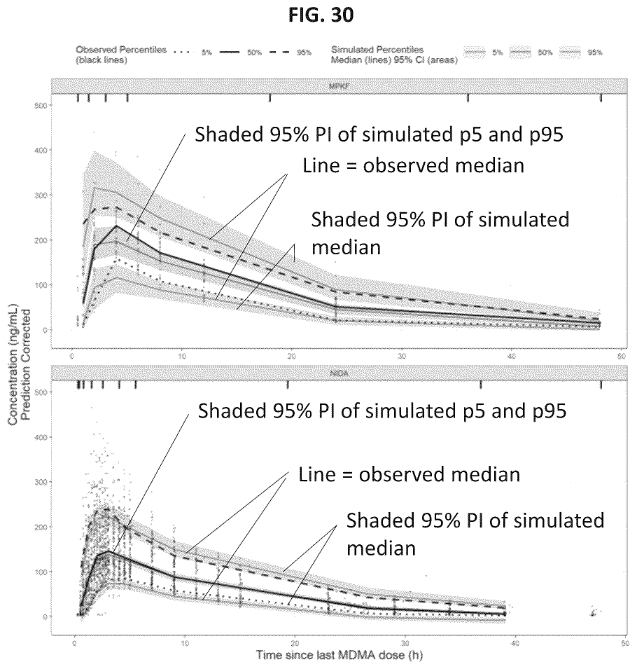

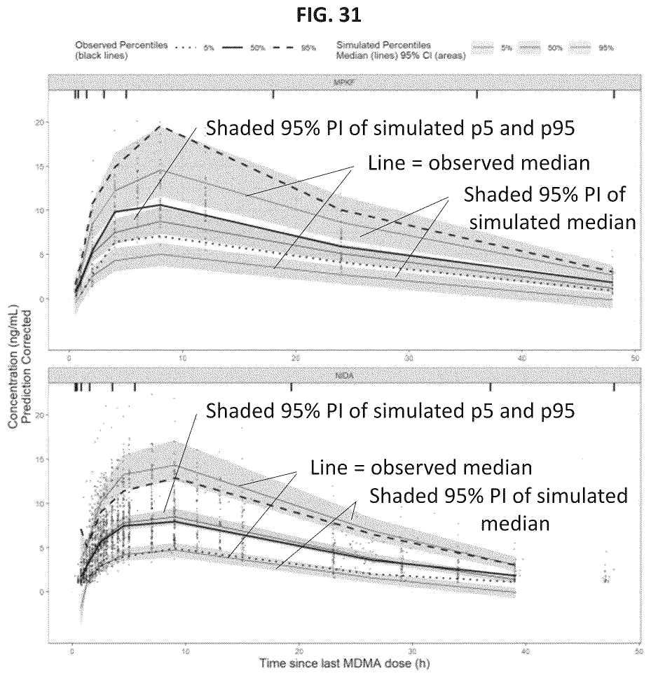

Prediction-corrected VPCs (pcVPCs) for the final MDMA and MDA PPK models were performed and are displayed in FIG. 28 and FIG. 29, respectively. The final PPK models predicted the observed median and 5th and 95th percentile (p5 and p95) of observed concentrations with good accuracy. The observed median profiles are fully captured within the 5th to 95th prediction interval (PI) of the median of simulations for MDMA and MDA across both studies. pcVPC plots stratified by study are presented in FIG. 30 and FIG. 31 for MDMA and MDA, respectively.

### Model Applications

The final models of MDMA and MDA were utilized to obtain individual post hoc estimates of PK parameters. For each patient for whom measurable concentrations were available, PK parameters (AUC₀₋₄₄, Cₘₐₓ, and half-life [t½]) were estimated based on post hoc compartmental PK parameters. Summary statistics for the posthoc parameter estimates are presented in Table 23 and Table 24, and summary statistics for exposures, stratified by study and treatment group, are presented in Table 25. AUC₀₋₄₄ for subjects in the MPKF study were similar between fed and fasted cohorts for both MDMA and MDA. MDMA Cₘₐₓ was higher under fasting conditions, consistent with a delay in absorption (longer Tₘₐₓ) under fed conditions. The half-life of MDMA was between 7.7 h and 8.7 h, with complete clearance of the drug assumed to occur after 5 half-lives, corresponding to 38.5-43.5 hours.

TABLE 23
Summary Statistics of PK Model-Derived
Post Hoc Parameter Estimates

| | |
|---|---|
| Parameter | MPKF (N =16) |

MDMA Apparent Clearance, CL/F (L/h)

| | | |
|---|---|---|
| Geo. mean (Geo. CV %) | 29.3 | (37.3) |

| | |
|---|---|
| Geo. 90% CI | [19.1, 48.6] |

MDMA Apparent Volume of Distribution, V (L)

| | | |
|---|---|---|
| Geo. mean (Geo. CV %) | 368.3 | (19.8) |

| | |
|---|---|
| Geo. 90% CI | [287.3, 466.2] |

^{a}Lag time, ALAG (h)

| | | |
|---|---|---|
| Geo. mean (Geo. CV %) | 0.3 | (0.0) |

Duration of zero order absorption, D1 (h)

| | | |
|---|---|---|
| Geo. mean (Geo. CV %) | 0.4 | (135.7) |

| | |
|---|---|
| Geo. 90% CI | [0.1, 1.6] |

MDA Clearance, CLM (L/h)

| | | |
|---|---|---|
| Geo. mean (Geo. CV %) | 29.7 | (36.4) |

| | |
|---|---|
| Geo. 90% CI | [17.0, 52.0] |

^{a}MDA Central Volume, V3 (L)

| | | |
|---|---|---|
| Geo. mean (Geo. CV %) | 20.8 | (0.0) |

MDA Intercompartmental Clearance, QM (L/h)

| | | |
|---|---|---|
| Geo. mean (Geo. CV %) | 68.4 | (59.5) |

| | |
|---|---|
| Geo. 90% CI | [29.8, 179.8] |

MDA Peripheral Volume, V4 (L/h)

| | | |
|---|---|---|
| Geo. mean (Geo. CV %) | 149.5 | (34.5) |

| | |
|---|---|
| Geo. 90% CI | [81.2, 237.1] |

Half-life MDMA, t½ (h)

| | | |
|---|---|---|
| Geo. mean (Geo. CV %) | 8.7 | (20.4) |

| | |
|---|---|
| Geo. 90% CI | [6.4, 11.3] |

^{a}IIV was not included in the model; population parameters are shown
Abbreviations: CI = confidence interval for the geometric mean; CV = coefficient of variation; D1 = duration of zero-order absorption; Geo = geometric; N = number of subjects; PK = pharmacokinetic

TABLE 24
Summary Statistics of PK Model-Derived
Post Hoc Absorption Rate (1/h)

| | MPKT Fasted | MPKF Fed |
|---|---|---|
| Parameter | (N = 16) | (N = 14) |
| Geo. mean (Geo. CV %) | 3.5 (47.0) | 0.6 (50.2) |
| Geo. 90% CI | [2.0, 8.3] | (0.4, 1.5] |

Absorption rate includes additional number of subjects due to the crossover design of MPKF, with subjects receiving drug in both fed and fasted states. MPKF fed subjects received a high fat meal.
Abbreviations: CI = confidence interval for the geometric mean; CV = coefficient of variation; Geo = geometric; N = number of subjects PK = pharmacokeinetic

TABLE 25
Summary Statistics of PK Model-Derived
Post Hoc Derived Exposures

| | MPKF | |
|---|---|---|
| | Fasted 120 mg | Fed 120 mg |
| Exposure | (N = 16) | (N = 14) |

AUC₀₋₄₄ MDMA (ng*h/mL)

| | | | | |
|---|---|---|---|---|
| Geo. mean | 3410.1 | (37.2) | 3344.2 | (39.5) |

| | | |
|---|---|---|
| (Geo. CV %) | | |
| Geo. 90% CI | [2056.9, 5411.4] | [2043.1, 5760.4] |

Cmax MDMA (ng/mL)

| | | | | |
|---|---|---|---|---|
| Geo. mean | 246.0 | (19.8) | 197.3 | (21.5) |

| | | |
|---|---|---|
| (Geo. CV %) | | |
| Geo. 90% CI | [190.7, 324.0] | [149.3, 271.6] |

AUC₀₋₄₄ MDA (ng*h/mL)

| | | | | |
|---|---|---|---|---|
| Geo. mean | 312.0 | (36.2) | 303.3 | (36.1) |

| | | |
|---|---|---|
| (Geo. CV %) | | |
| Geo. 90% CI | [178.2, 548.5] | [177.9, 506.3] |

Cmax MDA (ng*h/*mL)

| | | | | |
|---|---|---|---|---|
| Geo. mean | 11.8 | (26.1) | 11.3 | (24.6) |

| | | |
|---|---|---|
| (Geo. CV %) | | |
| Geo. 90% CI | [7.8, 17.7] | [7.7, 15.8] |

Abbreviations: CI = confidence interval; CV = coefficient of variation; Geo = geometric; N = number of subjects; SD = standard deviation

### Covariate Effects on MDMA and MDA Exposures

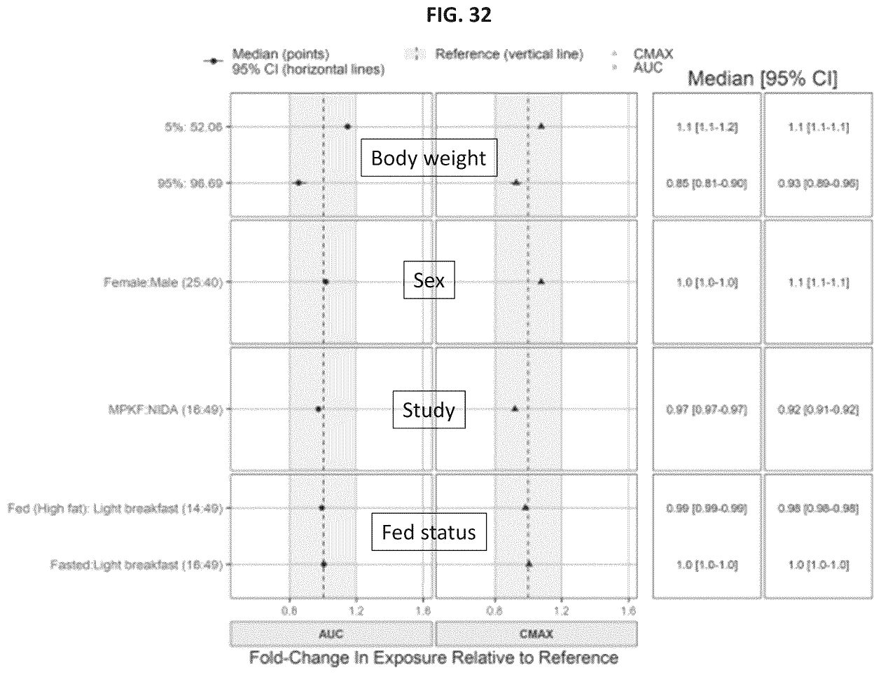

The impact on covariate effects on exposure was evaluated by means of covariate effects plots. Clinical significance was defined as a 20% decrease or a 20% increase in exposure relative to a reference individual. The reference individual was defined as a white male weighing 70 kg, aged 25 years, with baseline creatine clearance of 113 mL/min, and fed a light meal prior to receiving MDMA. Covariate effects plots for MDA are presented in FIG. 32. For MDMA, study and body weight were identified as the only two clinically significant covariates; neither fed status nor sex were clinically significant. Subjects in the 5th percentile of body weight had an overall 30% higher MDMA AUC₀₋₄₄ and Cₘₐₓ, while subjects in the 95th percentile of body weight had an overall 28% lower MDMA AUC₀₋₄₄ and 25% lower Cₘₐₓ.

### CYP2D6 Effect on MDMA and MDA Exposures

Exploratory analysis showed an impact of CYP2D6 status (normal vs intermediate metabolizer) on overall concentration. CYP2D6 status was only available for the MPKF study and could not be included as a covariate in the model.

### PK Simulations

Four dosing scenarios were tested based on expected therapeutic dosing levels. The scenarios listed in Table 26, and comprise:
1) 120 mg MDMA·HCl single dose (equivalent to 102 mg free base MDMA).
2) 120 mg split dose of MDMA·HCl given as 80 mg HCl followed by a 40 mg HCl second dose after 104 minutes (equivalent to 102 mg free base split dose of MDMA given as a 68 mg first dose followed by a 34 mg dose 104 minutes later).
3) 180 mg MDMA·HCl (equivalent to 150 mg free base MDMA).
4) 180 mg split dose of MDMA·HCl given as 120 mg HCl followed by a 60 mg HCl second dose after 104 minutes (equivalent to 150 mg free base split dose of MDMA given as a 100 mg first dose followed by a 50 mg dose 104 minutes later)

TABLE 26
Simulated MDMA Dosing Scenarios

| | Dose at T₀ | | | Dose at T_{104 min} | | | |
|---|---|---|---|---|---|---|---|
| | | MDMA | | | MDMA | | Total |
| | | HCl per | MDMA | | HCl per | MDMA | MDMA |
| | No. of | capsule | HCl Dose | No. of | capsule | HCl Dose | HCl Dose |
| Scenario | Capsules | (mg) | (mg) | Capsules | (mg) | (mg) | ^{Δ}(mg) |
| 1 | 3 | 40 | 120 | — | — | — | 120 |
| 2 | 2 | 40 | 80 | 1 | 40 | 40 | 120 |
| 3 | 3 | 60 | 180 | — | — | — | 180 |
| 4 | 2 | 60 | 120 | 1 | 60 | 60 | 180 |

For each dosing scenario, exposures were calculated after 44 hours (˜5 half-lives of MDMA). Additionally, tmax of MDMA is reached by ˜4 hours for all simulated scenarios; therefore, AUC0-4 (4 hours after dose) was also calculated at this time.

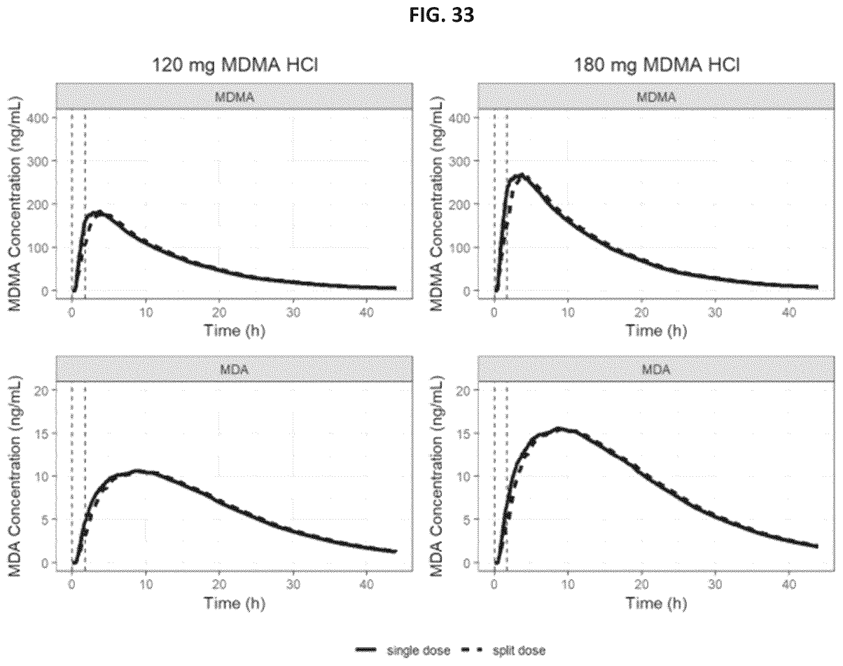

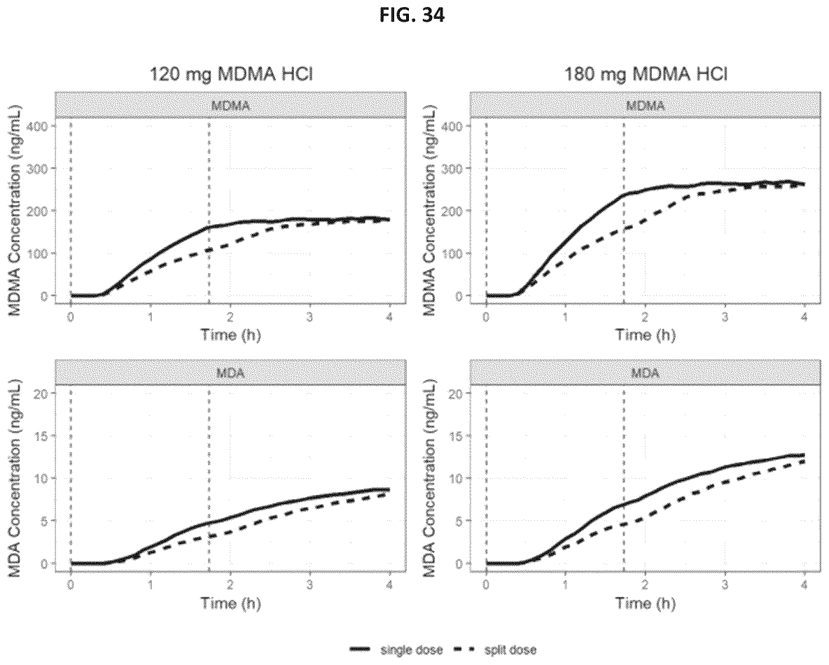

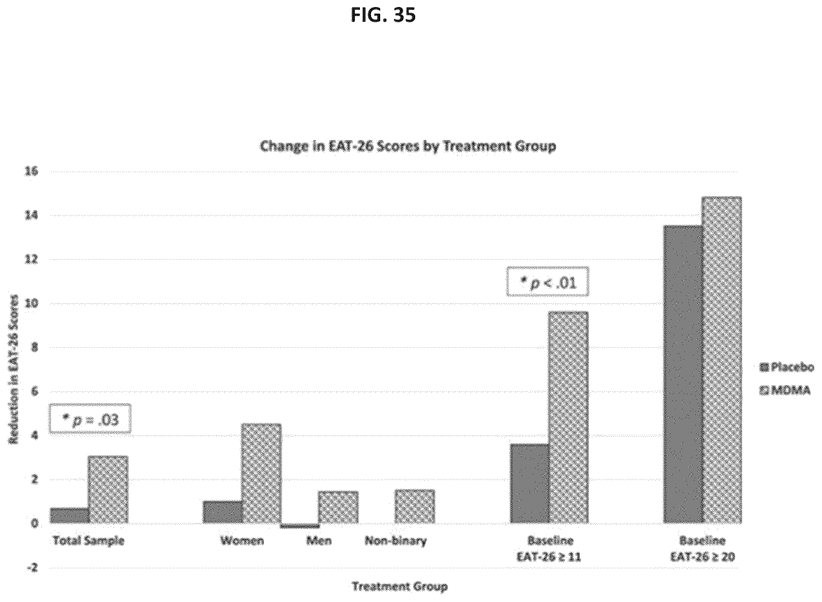

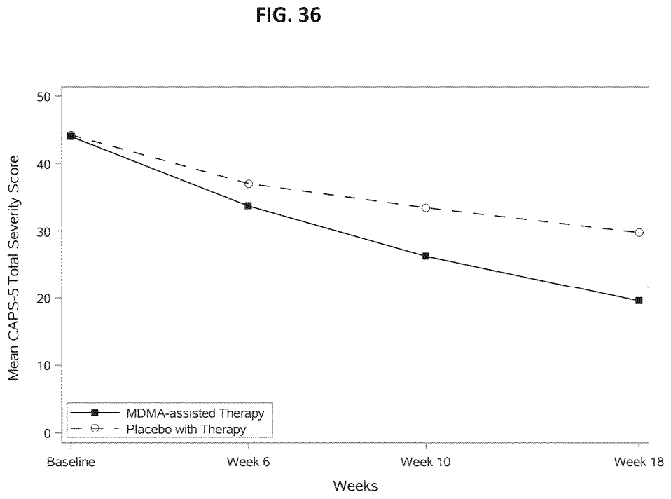

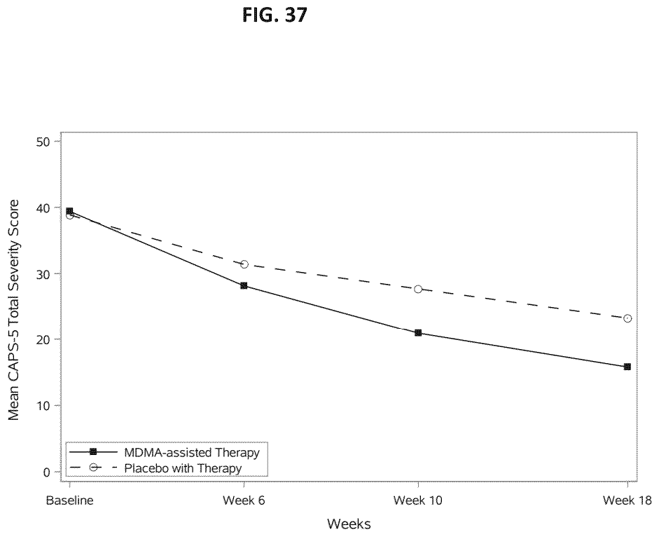

Split dose regimens were compared to single dose regimens to evaluate if a decrease in drug exposure within the first few hours (<4 h) of taking the drug could be achieved while maintaining the overall drug exposure over time. Simulated profiles for MDMA and MDA after 44 hours are presented in FIG. 33 and simulated profiles after the first 4 hours after dose are presented in FIG. 34. The statistical comparisons between single and split dose regimens are presented in Table 27. While exposure was higher with an overall higher dose, neither AUC nor Cₘₐₓ were significantly impacted by split dosing as compared to a single dose for the 44-hour drug profile. However, AUC was substantially lower in the split-dose compared to the single dose administration after the first 4 hours. The lower AUC in the first few hours suggests a possible reduction in adverse events with the split dose.

TABLE 27
Statistical Comparison Between Single Dose and Split Dose Regimens for MDMA and MDA

| | | | | P value | P value |
|---|---|---|---|---|---|
| Group 1 | | Group 2 | | (4 hours) | (44 hours) |
| MDMA AUC, | 120 mg HCl split dose | MDMA AUC, | 120 mg HCl single dose | 0.037 | 1 |
| MDMA AUC, | 180 mg HCl split dose | MDMA AUC, | 180 mg HCl single dose | 0.037 | 1 |
| MDMA Cmax, | 120 mg HCl split dose | MDMA Cmax, | 120 mg HCl single dose | 0.71 | 1 |
| MDMA Cmax, | 180 mg HCl split dose | MDMA Cmax, | 180 mg HCl single dose | 0.71 | 1 |
| MDA AUC, | 120 mg HCl spit dose | MDA AUC, | 120 mg HCl single dose | 0.01 | 1 |
| MDA AUC, | 180 mg HCl spit dose | MDA AUC, | 180 mg HCl single dose | 0.01 | 1 |
| MDA Cmax, | 120 mg HCl split dose | MDA Cmax, | 120 mg HCl single dose | 0.58 | 1 |
| MDA Cmax, | 180 mg HCl split dose | MDA Cmax, | 180 mg HCl single dose | 0.58 | 1 |

P values present a statistical comparison of exposures between single and split dosing regimens. At 4 hours, the AUC refers to AUC₀₋₄. At 44 hours AUC refers to AUC₀₋₄₄

### Discussion

The population PK model was developed using MDMA data from 65 subjects and 2012 PK observations across 2 studies. The selected model structure was a 1-compartment model with sequential zero and first order absorption, absorption lag time, and linear elimination. Between subject variability was applied to CL/F, V/F, KA, and DV. Correlation between CL/F and V/F was captured in the final MDMA model. A combined proportional and additive error model was used. The MDA (metabolite of MDMA) model was developed using 65 subjects and 1871 PK observations across 2 studies. Based on prior literature, 10% of MDMA was assumed to be metabolized to MDA. The selected model was a 2-compartment model with linear elimination. Between subject variability was applied to CLM, QM, and V4. Correlation between these three parameters was captured in the final MDA model. A combined proportional and additive error model was used. Exemplary covariate effects identified in the two models include, but are not limited to:
Increase in MDMA CL/F, V/F, CLM, and QM with increasing body weight
18% lower V4 (MDA peripheral volume of distribution) in female subjects as compared to male subjects.

The population PK model parameters were precisely estimated, except for the effect of fasted subject status on absorption. Based on published literature, the disposition of MDMA and its effects in humans are altered by polymorphic CYP2D6 activity, but the effects are small because of the mechanism-based autoinhibition of CYP2D6. The MDMA AUC and Cₘₐₓ exposure values for subjects at the 5th and 95th percentiles of body weight in the analysis dataset (52 kg and 97 kg, respectively) were predicted to have a significant impact on AUC (30% increase and 28% decrease, respectively), and on Cₘₐₓ (30% increase and 25% decrease, respectively). The effect of fasting status on exposure was not found to be clinically significant ((<20% difference from the values of a 70 kg subject). While absorption rate was slower among subjects fed a high-fat meal (longer Tₘₐₓ), their decrease in Cₘₐₓ was modest compared to subjects receiving MDMA in the fasted state or after a light breakfast, and the net difference in the area under the curve was also not substantially impacted. Neither body weight nor sex were found to substantially impact exposure of MDA. Simulations of 120 mg and 180 mg of MDMA·HCl (102 mg and 150 mg free base equivalent, respectively) indicated that there is little difference in AUC and Cₘₐₓ between a single dose administration and a split dose administration with a 104-minute delay between doses. This is likely because the second dose occurs prior to the Cₘₐₓ of MDMA. However, within the first 4 hours after administration until Cₘₐₓ is reached, the increase in MDMA exposure occurs more gradually with the split dose relative to the single dose administration. Split dosing has been shown to extend the peak of the therapeutic experience and may reduce severity of adverse effects, such as high blood pressure and heart rate, which mostly occur within the first 4 hours and are dose-dependent. Therefore, a split dose administration may be beneficial in reducing side effects that occur early on (i.e. within the first few hours) during treatment, without impacting therapeutic efficacy.

### Conclusions

The PK of MDMA following oral administration was well characterized by a one-compartment model with sequential zero and first-order absorption and linear elimination.
The PK of MDA was well characterized by a two-compartment model with linear elimination.
CL/F, V/F, CLM, and QM increased with increasing body weight.
Ka was 49% lower among subjects fed a high fat diet
Body weight and study had a clinically significant impact on the exposure of MDMA.
Simulations showed that a split dose administration of MDMA reduces AUC within the first 4 hours after dose but does not affect exposure across the complete concentration-time profile.

### Example 9. Evaluation of MDMA Tolerability in Subjects with Moderate Hepatic Impairment Compared to Matched Control Subjects with Normal Hepatic Function

### Introduction

This study aims to evaluate the effect of moderate hepatic impairment on the PK of oral MDMA and its active metabolite, MDA, and to assess the safety and tolerability of oral MDMA in individuals over the age of 18 with moderate hepatic impairment compared to matched control subjects with normal hepatic function. It is a Phase I, open-label study that will enroll a total of 16 eligible participants, with 8 participants who meet the diagnosis of moderate hepatic impairment (class B according to Child-Pugh's criteria), and 8 participants with normal hepatic function.

### Materials and Methods

Participants will be administered a single oral dose of 125 mg MDMA. Blood samples will be collected at pre-dose and at 0.5, 1, 2, 4, 6, 8, 12, and 24 hours post-dose. The plasma concentrations of MDMA and MDA will be analyzed using validated analytical methods. Safety and tolerability will be evaluated by monitoring adverse events, vital signs, and electrocardiograms (ECGs).

### Data Analysis:

The primary objective of the study is to compare the PK parameters of MDMA and MDA in participants with moderate hepatic impairment versus matched control subjects with normal hepatic function. The PK parameters that will be evaluated include Cmax, time to reach Tmax, AUC, and t½. The PK parameters will be compared between the two groups using analysis of variance (ANOVA) or non-parametric tests, as appropriate. Safety and tolerability will be assessed by comparing the incidence and severity of adverse events between the two groups. The data will be summarized using descriptive statistics, and statistical analyses will be performed using appropriate methods. Statistical tests will be two-sided, and a p-value of less than 0.05 will be considered statistically significant. The number of participants reported below is the cumulative number of participants accrued as of the reporting period.

### Example 10. Method of Treating a Subject with MDMA-Assisted Therapy (MDMA-AT)

A flexible dose of MDMA hydrochloride salt (referred to as MDMA throughout) or placebo, followed by a supplemental half-dose unless contraindicated, was administered during the Treatment Period with manualized psychotherapy in three blinded monthly Experimental Sessions. This ˜12-week Treatment Period was preceded by three Preparatory Sessions. During the Treatment Period, each Experimental Session was followed by three Integrative Sessions of non-drug psychotherapy. Experimental Sessions were followed by an overnight stay. The Primary Outcome measure, the change in Clinician Administered PTSD Scale (CAPS-5), was assessed by a blinded centralized Independent Rater (IR) pool multiple times throughout the study. Three blinded manualized Experimental Sessions of psychotherapy assisted by flexible doses of MDMA·HCl or placebo were administered (see Table 28 below). Initial doses per Experimental Session include 80 mg or 120 mg MDMA compounded with mannitol and magnesium stearate or indistinguishable weight placebos comprised entirely of mannitol and magnesium stearate, followed 1.5 to 2 hours later by a supplemental half-dose (40 mg or 60 mg MDMA·HCl or placebo). Total amounts of MDMA·HCl to be administered per Experimental Session range from 80 mg to 180 mg. All drug is encapsulated with HPMC capsules.

TABLE 28
Dose Regimen of MDMA or Placebo.

| Experimental | Initial | | Supplemental | | Min-Max |
|---|---|---|---|---|---|
| Session | Dose | | Dose* | | Cumulative Dose |
| 1 | 80 | mg | 40 | mg | 80 mg to 120 mg |
| 2 | 80 or 120 | mg | 40 or 60 | mg | 80 mg to 180 mg |
| 3 | 80 or 120 | mg | 40 or 60 | mg | 80 mg to 180 mg |

| | |
|---|---|
| Total Cumulative Dose | 240 mg to 480 mg |

In the first Experimental Session, the initial dose was 80 mg MDMA·HCl or placebo. In the second and third Experimental Sessions, the initial dose may be increased to 120 mg MDMA·HCl or placebo unless contraindicated. The choice of whether to keep the dose the same or change it from the first Experimental Session was made by the therapy team the in consultation with the site physician based on observed response, tolerability to the previously administered dose, and discussion with the participant. In each Experimental Session, 1.5 to 2 hours after the initial dose is given, the participant was administered a supplemental half-dose unless contraindicated. If an AE requiring medical attention occurs between the initial and supplemental dose this was evaluated as a potential contraindication by the site physician. If a participant prefers not to take the supplemental dose, the reason was documented. Participants will not know if they have been assigned MDMA or placebo but can indicate if they want the dose to change or remain the same.

The MDMA assisted therapy (MDMA-AT) approach is detailed in the “Manual for MDMA Assisted Therapy in the Treatment of PTSD,” published by MAPS (MDMA Treatment Manual, available at maps.org/treatment-manual). Therapy during MDMA-AT sessions consisted of periods of introspection alternating with periods of communication between the participant and the cotherapist dyad. Participants were encouraged to remain with trauma-related memories, feelings, and/or thoughts as the cotherapist dyad provided support. MDMA-AT sessions lasted 6 to 8 hours and ended after drug effects returned to baseline. Participants remained overnight at the site with a night attendant, except for four participants who did not stay overnight as part of a safety substudy. After each MDMA-AT session, participants received several follow-up visits, including three integrative sessions, where therapists facilitated participants' continued emotional processing, addressed any difficulties following the MDMA-AT session, and helped participants to apply any benefits gained in the MDMA-AT sessions to daily life. Participants worked with the same cotherapist dyad throughout the entire treatment period. The therapeutic approach is detailed in the MDMA Treatment Manual. A detailed treatment protocol is shown in Table 29 below.

TABLE 29
Treatment Protocol.
Treatment Period

| | Visit Duration/ | |
|---|---|---|
| Study Visit | Visit Timing | Brief Description of Events |
| Randomization | .5 hours/ | Complete after enrollment and scheduling |
| | Within 2 week of Baseline | Exp. Session 1. Enter demographics in |
| | CAPS-5/ | Medrio for use in randomization. The |
| | 24 to 48 hours before Exp. | participant does not need to be present for |
| | Session 1 | this. |
| Experimental | 8 hours + overnight/ | 8 hours with overnight stay. Dose is 80 mg |
| Session 1 | Within 2 weeks of Baseline | with supplemental half-dose of 40 mg |
| | CAPS-5 | unless contraindicated. |
| Experimental | 8 hours + overnight/ | The second Experimental Session lasts 8 |
| Session 2 | 21 to 35 days after | hours with an overnight stay. Dose is 80 or |
| | Experimental Session 1 | 120 mg plus supplemental half-dose unless |
| | | contraindicated. |
| Experimental | 8 hours + overnight/ | The third Experimental Session lasts 8 hours |
| Session 3 | 21 to 35 days after | with an overnight stay. Dose is 80 or 120 mg |
| | Experimental Session 2 | plus supplemental half-dose unless |
| | | contraindicated. |

Across 12 U.S. study sites and 2 Canadian sites, a total of 37 unique cotherapist dyads provided MDMA-assisted therapy (MDMA-AT) for treatment under clinical supervision among participants with severe post-traumatic stress disorder (PTSD).

Eligibility criteria included confirmation of severe PTSD, which was defined as having a CAPS-5 Total severity score of 35 or greater. Participants were asked to agree to the study protocol including lifestyle modifications. Exclusionary criteria included past or present psychotic disorder, bipolar I disorder, pregnancy or lactation, current diagnosis of a substance use disorder (except for caffeine or nicotine), uncontrolled hypertension, weighing less than 48 kg, and other medical conditions contraindicated for MDMA such as cardiac conditions or cerebrovascular disease. Participants who were at serious risk of suicide or posed a risk to others were also ineligible. Participants with controlled hypertension underwent additional screening to confirm the absence of clinically significant underlying cardiovascular disease. Participants who were enrolled into the study were asked, under the supervision of a physician, to taper off psychiatric medications and any other medications that might have interfered with the effects or metabolism of MDMA.

### Treatment

The MDMA-AT therapeutic approach is detailed in the “Manual for MDMA-Assisted Therapy in the Treatment of PTSD,” published by MAPS (MDMA Treatment Manual, available at maps.org/treatment-manual). MDMA-AT was conducted over a duration of 9 to 15 weeks. Treatment periods consisted of three preparatory sessions before the first administration of MDMA and three MDMA experimental sessions, in which each session was followed by three integrative sessions. In preparatory sessions, participants met with their cotherapist dyad to develop therapeutic rapport, discuss their PTSD symptoms, and the upcoming MDMA-AT session. Therapists provided information on what to expect during the MDMA-AT sessions, including drug effects and strategies to manage any challenging experiences that may emerge.

Participants were offered a total of three MDMA-AT sessions that were scheduled 3 to 5 weeks apart. In the first experimental session, participants were administered a divided dose of 80 mg MDMA·HCl initial+40 mg MDMA·HCl supplemental. Supplemental doses were administered 1.5 to 2 hours after the initial dose. The purpose of the supplemental dose was to enable a longer period to process trauma during MDMA-AT sessions without significantly impacting the intensity or total duration of pharmacodynamic effects. The second and third experimental sessions offered a dose escalation to divided doses of 80 mg MDMA·HCl+40 mg MDMA·HCl or 120 mg MDMA·HCl+60 mg MDMA·HCl. The nominal difference in MDMA doses between countries was due to drug availability and challenges in import/export of a controlled substance, where U.S. participants received racemic MDMA synthesized by David Nichols, Ph.D. (Purdue University) and Canadian participants received racemic MDMA from Lipomed AG Switzerland.

Therapy during MDMA-AT sessions consisted of periods of introspection alternating with periods of communication between the participant and the cotherapist dyad. Participants were encouraged to remain with trauma-related memories, feelings, and/or thoughts as the cotherapist dyad provided support. MDMA-AT sessions lasted 6 to 8 hours and ended after drug effects returned to baseline. Participants remained overnight at the site with a night attendant, except for four participants who did not stay overnight as part of a safety substudy. After each MDMA-AT session, participants received several follow-up visits, including three integrative sessions, where therapists facilitated participants' continued emotional processing, addressed any difficulties following the MDMA-AT session, and helped participants to apply any benefits gained in the MDMA-AT sessions to daily life. Participants worked with the same cotherapist dyad throughout the entire treatment period. The therapeutic approach is detailed in the MDMA Treatment Manual.

### Example 11. MAPP1 Clinical Trial Summary

MAPP1 was a Phase 3 randomized, placebo-controlled, 2-arm, double-blind, multi-site study conducted to investigate the efficacy and safety of MDMA-AT in participants with severe PTSD. This study is also discussed in Mitchell et. al., Nature Medicine 2021, 27, 1025-1033. This study included 3 experimental sessions of therapy combined with either MDMA·HCl or placebo. During Experimental Session 1, participants received a split dose of 120 mg (80 mg+40 mg).

Participants received an escalated split dose of 180 mg during Experimental Sessions 2 and 3 unless there were tolerability issues or the participant declined. The primary outcome measure, the CAPS-5, evaluated changes in PTSD symptom severity and the secondary outcome measure, the SDS, evaluated changes in functional impairment. Both measures were assessed by a blinded centralized language-specific IR pool.

### Study Population

Overall, 91 participants were randomized in the study (MDMA: 46 participants; placebo: 45 participants; Table 38)Table 38)Table 38) The majority of participants completed Experimental Session 3 (MDMA: 91.3%; placebo: 84.1%). A total of 8 participants (MDMA: 2 participants; placebo: 6 participants) in the MDMA-AT and 6 participants in the placebo with therapy group) terminated treatment early after randomization:
Two placebo participants terminated the study early due to adverse events (AEs) as the primary reason (1 participant with a moderate AE of anxiety and 1 participant with a severe treatment emergent adverse event (TEAE) of insomnia). An additional placebo participant terminated early due to participant choice as the primary reason; however, an AE was included as the secondary reason (severe TEAE of suicide attempt).
Two participants (1 in each treatment group) terminated early due to administrative reasons as the primary reason; secondary reasons for both participants included COVID-19.
One MDMA participant terminated early due to investigator choice; secondary reasons included subject choice, investigator choice, and COVID-19.
One placebo participant terminated early due to risk of COVID-19 contraction as the primary reason.
One participant was randomized to receive placebo but terminated early due to participant declined to participate. This participant did not receive any IP and was not included in the Safety Set.

A total of 4 participants dropped out (2 participants in each group). One placebo participant dropped out due to AEs as the primary reason (severe TEAE of suicidal ideation), 1 MDMA participant dropped out due to participant choice; however, an AE was included as the secondary reason for dropout (severe TEAE of depression), and 2 participants (1 MDMA and 1 placebo) withdrew consent.

TABLE 30
Participant Disposition (All Screened).

| | MDMA | | Placebo | |
|---|---|---|---|---|
| n (%) | N = 46 | | N = 45 | |
| Randomized | 46 | | 45 | |
| Safety Set^{a} | 46 | | 44 | |
| mITT Set^{b} | 46 | | 44 | |
| Visit Completion | | | | |
| Experimental Session 1 | 46 | (100.0) | 44 | (100.0) |
| Experimental Session 2 | 43 | (93.5) | 41 | (93.2) |
| Experimental Session 3 | 42 | (91.3) | 37 | (84.1) |
| Study Termination (Visit 20) | 46 | (100.0) | 44 | (100.0) |

Reason for Study Termination and Primary Reason for Early Termination

| Post-randomization Early Termination | 2 | (4.3) | 6 | (13.3) |
|---|---|---|---|---|
| Adverse Event or Death | 0 | | 2 | (4.4) |
| Subject Chose to Discontinue Treatment | 0 | | 1 | (2.2) |
| Investigator Chose to Discontinue Treatment | 1 | (2.2) | 0 | |
| Administrative Reason | 1 | (2.2) | 1 | (2.2) |
| Subject Declined to Participate | 0 | | 1 | (2.2) |
| Other | 0 | | 1 | (2.2) |
| Dropout | 2 | (4.3) | 2 | (4.4) |
| Subject Chose to Discontinue Treatment | 1 | (2.2) | 0 | |
| AE or Death | 0 | | 1 | (2.2) |
| Withdrawal of Consent | 1 | (2.2) | 1 | (2.2) |

| | | |
|---|---|---|
| Lost to follow up | 0 | 0 |

MDMA = 3,4-methylenedioxymethamphetamine; mITT = modified Intent-To-Treat; N = total number of participants in each group; n = number of participants.

A Post-Randomization Early termination refers to a participant that was enrolled and randomized and then stopped all therapy visits and experimental sessions, however CAPS-5 Assessment visits (V8, V13, V19) may have been completed before termination.

A drop out refers to a participant who was enrolled, randomized, and treated but then withdrew consent for all other protocol activities.

Only primary reason for early termination and dropout are included in the table. Participants may have had secondary reasons for study treatment discontinuation.
a. Received any IMP.
b. Had at Least 1 CAPS-5 Assessment

The majority of participants in the Safety Set were white (76.7%), not Hispanic or Latino (90.0%), and female (65.6%) (Table 39Table 39Tabe 39). The mean age atbaseline was 40.93 years (range of 20.9 to 71.2 years). In general, the demographic characteristics of the treatment groups were similar with the exception of sex; there was a higher percentage of females in the placebo group (72.7%) than in the MDMA-AT group (58.7%).

TABLE 31
Demographics and Baseline Characteristics (Safety Set).

| | MDMA | | Placebo | | Total | |
|---|---|---|---|---|---|---|
| | N = 46 | | N = 44 | | N = 90 | |
| Gender, n (%) | | | | | | |
| Male | 19 | (41.3) | 12 | (27.3) | 31 | (34.4) |
| Female | 27 | (58.7) | 32 | (72.7) | 59 | (65.6) |
| Age, years | | | | | | |
| Mean (SD) | 43.55 | (12.863) | 38.19 | (10.361) | 40.93 | (11.950) |
| Median (Min, Max) | 39.10 | (24.9, 71.2) | 36.59 | (20.9, 62.9) | 38.58 | (20.9, 71.2) |
| Ethnicity, n (%) | | | | | | |
| Hispanic or Latino | 5 | (10.9) | 3 | (6.8) | 8 | (8.9) |
| Not Hispanic or Latino | 41 | (89.1) | 40 | (90.9) | 81 | (90.0) |
| Unknown | 0 | | 1 | (2.3) | 1 | (1.1) |
| Race, n (%) | | | | | | |
| American Indian or Alaska Native | 3 | (6.5) | 0 | | 3 | (3.3) |
| Asian | 2 | (4.3) | 5 | (11.4) | 7 | (7.8) |
| Black or African American | 0 | | 2 | (4.5) | 2 | (2.2) |
| Native Hawaiian or Other Pacific Islander | 0 | | 0 | | 0 | |
| White | 39 | (84.8) | 30 | (68.2) | 69 | (76.7) |
| Multiple | 2 | (4.3) | 6 | (13.6) | 8 | (8.9) |

Max = maximum; MDMA = 3,4-methylenedioxymethamphetamine; Min = minimum; N = total number of participants; n = number of participants; SD = standard deviation.

At baseline, the mean (SD) duration of PTSD was 14.04 (11.473), with a maximum duration of 48.8 years (Table 40Table 40Table 40). Based on enrollment criteria, all participants had a duration of at least 0.5 years. The majority of patients had trauma histories that included developmental trauma events (84.4%) and/or multiple trauma events (87.8%). Both duration of PTSD and trauma histories were similar across treatment groups. History of major depression (MDMA: 91.3%; placebo: 90.9%) was additionally similar across treatment groups.

Per enrollment criteria all participants had severe PTSD (mean CAPS-5 Total Severity score at baseline: 44.1) and 21.1% of participants had the dissociative subtype of PTSD. In addition, the high mean SDS score (6.8 in MDMA-AT group and 7.4 in placebo with therapy group, (_{TableTableTable} 41) indicates that most also had severe functional impairment.

Most participants (97.8%) had previously tried therapy for PTSD, the most common of which were other, EMDR, CBT, and group psychotherapy (_{Table 40Table 40Table 40}). Other types of therapy included the general term of talk therapy and psychotherapy as well as other specific types of therapy. Histories of pharmacologic and non-pharmacologic interventions were similar across treatment groups.

TABLE 32
Baseline Disease Characteristics (Safety Set).

| | MDMA | | Placebo | | Total | |
|---|---|---|---|---|---|---|
| | N = 46 | | N = 44 | | N = 90 | |
| Trauma History, n (%) | | | | | | |
| Veteran Status | 10 | (21.7) | 6 | (13.6) | 16 | (17.8) |
| Served in a combat area | 6 | (13.0) | 5 | (11.4) | 11 | (12.2) |
| Multiple trauma events | 41 | (89.1) | 38 | (86.4) | 79 | (87.8) |
| Developmental trauma events | 40 | (87.0) | 36 | (81.8) | 76 | (84.4) |
| Baseline BDI-II Total Score | | | | | | |
| Mean (SD) | 30.5 | (13.11) | 34.9 | (12.57) | 32.7 | (12.97) |
| Median (Min, Max) | 30.0 | (3, 53) | 36.5 | (10, 56) | 33.5 | (3, 56) |
| Baseline PTSD Duration (years) | | | | | | |
| Mean (SD) | 14.80 | (11.615) | 13.25 | (11.401) | 14.04 | (11.473) |
| Median (Min, Max) | 12.61 | (0.6, 48.8) | 9.58 | (0.7, 46.1) | 10.86 | (0.6, 48.8) |
| Pre-Study PTSD Medication, n (%) | | | | | | |
| Paroxetine | 4 | (8.7) | 4 | (9.1) | 8 | (8.9) |
| Sertraline | 12 | (26.1) | 11 | (25.0) | 23 | (25.6) |
| Baseline CAPS-5 Total Severity Score | | | | | | |
| Mean (SD) | 44.0 | (6.01) | 44.2 | (6.15) | 44.1 | (6.04) |
| Median (Min, Max) | 43.5 | (35, 57) | 44.0 | (35, 62) | 44.0 | (35, 62) |
| Baseline Disease Severity (based on CAPS-5), n (%) | | | | | | |
| Severe (≥35) | 46 | (100.0) | 44 | (100.0) | 90 | (100.0) |
| Baseline CAPS-5 Dissociative Subtype, n (%) | | | | | | |
| No | 40 | (87.0) | 31 | (70.5) | 71 | (78.9) |
| Yes | 6 | (13.0) | 13 | (29.5) | 19 | (21.1) |
| Prior Psychotherapy, n (%) | | | | | | |
| Participants with any previous psychotherapy | 45 | (97.8) | 43 | (97.7) | 88 | (97.8) |
| Cognitive Processing Therapy | 0 | | 1 | (2.3) | 1 | (1.1) |
| Dialectical Behavioral Therapy | 2 | (4.3) | 2 | (4.5) | 4 | (4.4) |
| Eye Movement Desensitization Reprocessing | 24 | (52.2) | 14 | (31.8) | 38 | (42.2) |
| Group Psychotherapy | 21 | (45.7) | 14 | (31.8) | 35 | (38.9) |
| Holotropic Breathwork | 2 | (4.3) | 0 | | 2 | (2.2) |
| Other Cognitive Behavioral Therapy | 14 | (30.4) | 23 | (52.3) | 37 | (41.1) |
| Prolonged Exposure | 2 | (4.3) | 0 | | 2 | (2.2) |
| Psychodynamic | 12 | (26.1) | 11 | (25.0) | 23 | (25.6) |
| Other | 38 | (82.6) | 36 | (81.8) | 74 | (82.2) |

BDI-II = Beck Depression Inventory II; CAPS-5 = Clinician-administered PTSD Scale for DSM-5; DSM-5 = Diagnostic and Statistical Manual of Mental Disorders version 5; Max = maximum; MDMA = 3,4-methylenedioxymethamphetamine; Min = minimum; PTSD = Posttraumatic Stress Disorder; SD = standard deviation. Source Table 14.1.3.1 and 14.1.7

### Efficacy

### Primary Endpoint Analyses

A de jure estimand of treatment efficacy was used to estimate the causal effect of MDMA-AT on PTSD symptom severity as measured by the change from Baseline to Visit 19 in CAPS-S total severity scores in the mITT analysis set. An MMRM analysis of the deljure estimand showed a statistically significant difference (p<0.0001) between treatment arms, with a greater reduction in CAPS-S total severity scores in participants receiving MDMA (−24.50) compared to placebo (−12.64) (Table 33Table 33Table 33). Differences in treatment effect among demographic, dissociative sub-type, and overnight stay subgroups were not observed.

TABLE 33
Change in CAPS-5 Total Severity Scores by Visit-De Jure Estimand (mITT Set).

| | MDMA-AT | | Placebo with Therapy | |
|---|---|---|---|---|
| Statistic by Visit | N = 46 | | N = 44 | |
| Baseline CAPS-5 T1 (Visit 3), n | 46 | | 44 | |
| Mean (SD) | 44.0 | (6.01) | 44.2 | (6.15) |
| Median (min, max) | 43.5 | (35, 57) | 44.0 | (35, 62) |
| CAPS-5 T2 (Visit 8), n | 46 | | 43 | |
| Mean (SD) | 33.7 | (12.50) | 37.0 | (10.70) |
| Median (min, max) | 34.5 | (2, 53) | 38.0 | (8, 62) |
| CFB to CAPS-5 T2 (Visit 8), n | 46 | | 43 | |
| Mean (SD) | −10.3 | (11.10) | 7.1 | (8.69) |
| Median (min, max) | −9.0 | (−39, 8) | 6.0 | (−29, 14) |
| LS mean (95% CI)^{a} | −10.40 | (−13.29, −7.52) | −6.71 | (−9.72, −3.70) |
| LS mean for treatment difference (95% CI)^{a} | −3.69 (−7.93, 0.54) | | | |
| CAPS-5 T3 (Visit 13), n | 42 | | 39 | |
| Mean (SD) | 26.2 | (12.30) | 33.4 | (12.79) |
| Median (min, max) | 28.0 | (0, 53) | 33.0 | (3, 60) |
| CFB to CAPS-5 T3 (Visit 13), n | 42 | | 39 | |
| Mean (SD) | −17.7 | (10.74) | 10.2 | (11.94) |
| Median (min, max) | −17.5 | (−41, 4) | 9.0 | (−34, 10) |
| LS mean (95% CI)^{a} | −17.83 | (−21.33, −14.32) | −9.42 | (−13.07, −5.77) |
| LS mean for treatment difference (95% CI)^{a} | −8.41 (−13.53, −3.29) | | | |
| CAPS-5 T4 (Visit 19), n | 42 | | 37 | |
| Mean (SD) | 19.5 | (13.50) | 29.8 | (12.37) |
| Median (min, max) | 18.5 | (0, 51) | 30.0 | (2, 52) |
| CFB to CAPS-5 T4 (Visit 19), n | 42 | | 37 | |
| Mean (SD) | −24.4 | (11.57) | 13.9 | (11.53) |
| Median (min, max) | −25.0 | (−47, 3) | −16.0 | (−35, 7) |
| LS mean (95% CI)^{a} | −24.50 | (−28.28, −20.71) | 12.64 | (−16.61, −8.66) |

| | |
|---|---|
| LS mean for treatment difference (95% CI)^{a} | −11.86 (−17.41, −6.32) |
| p-value^{a} | <0.0001 |

De jure estimand does not include data after subjects discontinue treatment.
CAPS-5 = Clinician-Administered Posttraumatic Stress Disorder Scale for Diagnostic and Statistical Manual of Mental Disorders, 5^{th} edition; LS = Least Squares; Max = Maximum; MDMA = 3,4-methylenedioxymethamphetamine; min = minimum; mITT = modified Intent-to-treat; SD = Standard Deviation
^{a}LS Mean, LS Mean difference, 95% CI, and p-value of treatment effect at visit 19 are from an MMRM model with treatment group, visit, treatment group by visit interaction, site, and dissociative subtype as fixed effect, and baseline CAPS-5 as a covariate.

A clinically meaningful reduction in CAPS-5 total severity scores and PTSD diagnostic criteria by visit for mITT Set is presented in _{Table 42Table 42Table 42}. A total of 37 (88.1%) participants receiving MDMA-AT met the definition of responder, demonstrating a clinically meaningful≥10-point reduction in CAPS-5 total severity score, compared to 23 (62.2%) of participants receiving placebo at Visit 19. Total Severity Scores no longer met PTSD diagnostic criteria in 28 (66.7%) participants receiving MDMA compared to 12 (32.4%) of participants receiving the placebo at Visit 19.

A total of 14 (33.3%) of participants receiving the MDMA met the definition of remission, having both a CAPS-5 total severity score≤11 and no longer meeting PTSD diagnostic criteria compared to 2 (5.4%) of participants receiving placebo at Visit 19.

TABLE 34
Clinically Meaningful Reduction in CAPS-5 Total Severity
Scores and PTSD Diagnostic Criteria by Visit (mITT Set).

| | MDMA-AT | | Placebo with Therapy | |
|---|---|---|---|---|
| Visit | N = 46 n (%) | | N = 44 n (%) | |
| T2 endpoint (Visit 8) | 46 | | 43 | |
| Responder | 21 | (45.7) | 14 | (32.6) |
| Loss of Diagnosis | 11 | (23.9) | 5 | (11.6) |
| Remission | 4 | (8.7) | 1 | (2.3) |
| Non-Responder | 25 | (54.3) | 29 | (67.4) |
| T3 endpoint (Visit 13) | 42 | | 39 | |
| Responder | 31 | (73.8) | 18 | (46.2) |
| Loss of Diagnosis | 24 | (57.1) | 7 | (17.9) |
| Remission | 5 | (11.9) | 2 | (5.1) |
| Non-Responder | 11 | (26.2) | 21 | (53.8) |
| T4 (primary) endpoint (Visit 19) | 42 | | 37 | |
| Responder | 37 | (88.1) | 23 | (62.2) |
| Loss of Diagnosis | 28 | (66.7) | 12 | (32.4) |
| Remission | 14 | (33.3) | 2 | (5.4) |
| Non-Responder | 5 | (11.9) | 14 | (37.8) |

CAPS-5 = Clinician-Administered PTSD for Diagnostic and Statistical Manual of Mental Disorders, 5^{th} edition; mITT = modified Intent-to-treat; PTSD = Posttraumatic Stress Disorder; SD = Standard Deviation.
Responder: ≥10-point reduction in CAPS-5 Total Severity Score.
Non-Responder: <10-point reduction in CAPS-5 Total Severity Score. Loss of Diagnosis: Does not meet Diagnostic Criteria and is a Responder.
Remission: Does not meet Diagnostic Criteria and CAPS-5 Total Score <=11.

### Key Secondary Endpoint Analyses

The de jure estimand of treatment efficacy was used to determine the effect of MDMA-AT on SDS total score. An MMRM analysis of the de jure estimand showed a statistically significant difference (p=0.0167) between treatment arms, with a greater reduction in SDS total scores in participants receiving MDMA (−3.15) compared to placebo (−1.79) (_{Table 43Table 43Table 43}).

TABLE 35A
SDS Total Scores by Visit (mITT Set).

| | MDMA-AT | | Placebo with | |
|---|---|---|---|---|
| Statistics | N = 46 | | Therapy N = 44 | |
| Baseline (Visit 3) (n) | 46 | | 44 | |
| Mean (SD) | 6.8 | (2.07) | 7.4 | (1.63) |
| Median (min, max) | 6.8 | (1, 10) | 8.0 | (4, 10) |
| Visit 8 (n) | 46 | | 43 | |
| Mean (SD) | 5.0 | (2.78) | 6.1 | (2.25) |
| Median (min, max) | 5.0 | (0, 10) | 6.7 | (0, 9) |
| Visit 13 (n) | 42 | | 39 | |
| Mean (SD) | 4.1 | (2.71) | 5.5 | (2.37) |
| Median (min, max) | 4.7 | (0, 10) | 5.3 | (0, 9) |
| Visit 19 (Primary Endpoint) (n) | 42 | | 37 | |
| Mean (SD) | 3.8 | (2.98) | 5.3 | (2.31) |
| Median (min, max) | 3.4 | (0, 9) | 5.7 | (1, 9) |
| CFB (n) | 42 | | 37 | |
| Mean (SD) | −3.1 | (2.63) | −2.0 | (2.41) |
| Median (min, max) | −2.5 | (−8, 2) | −1.3 | (−7, 3) |
| LSMean (95% CI)^{a} | −3.15 | (−3.90, −2.40) | 1.79 | (−2.58, −1.00) |

| | | | |
|---|---|---|---|
| LSMean for Treatment Difference (95% CI)^{a} | −1.36 | (−2.46, −0.25) | — |

| | | |
|---|---|---|
| p-value^{a} | 0.0167 | — |

CFB = Change from Baseline; LSM = Least Square Means; Max = Maximum; MDMA = 3,4-methylenedioxymethamphetamine; Min = Minimum; mITT = modified Intent-to-treat; SD = Standard Deviation; SDS = Sheehan Disability Scale; ^{a}LS Mean, LS Mean difference, 95% CI, and p-value of treatment effect at Visit 19 were from an MMRM model with treatment group, visit, treatment group by visit interaction, site, and dissociative subtype as fixed effect, and baseline SDS as a covariate.

TABLE 35B
SDS Response Rates at End of Treatment.

| | Number of | Responder | | Remission | |
|---|---|---|---|---|---|
| Study Drug | Patients | n(%) | | n(%) | |
| MDMA + psychological | 42 | 16 | (38.1) | 18 | (42.9) |
| intervention | | | | | |
| Placebo + psychological | 37 | 7 | (18.9) | 5 | (13.5) |
| intervention | | | | | |
| MDMA + psychological | 52 | 28 | (53.8) | 29 | (55.8) |
| intervention | | | | | |
| Placebo + psychological | 42 | 8 | (19.0) | 17 | (40.5) |
| intervention | | | | | |

* Responders were defined as patients with a 4 point or greater reduction from baseline SDS mean item score. Remission was defined as a SDS mean item score <3 indicating mild impairment.

### Safety

All participants (100%) reported at least 1 TEAE over the course of the study (_{Table 44Table 44Table 44}). There were 2 serious TEAEs in the placebo with therapy group (1 participant with 2 events of suicide attempt [1 moderate and 1 severe] and 1 participant with 1 event of suicidal ideation [severe], Listing 14.3.2.2.3). Adverse events of special interest included a subset of AEs involving cardiac function, suicidality, and MDMA abuse. There were 3 (6.5%) participants in the MDMA-AT group and 6 (13.6%) participants in the placebo with therapy group with a TEAE of special interest. The majority of AESIs were associated with suicidality (reported by 9 of 10 participants) and 1 participant in the placebo group reported 2 AESIs that were associated with cardiac function (palpitations and irregular heart rate). There were no AESIs of MDMA abuse. A total of 4 participants discontinued study treatment due to a TEAE (MDMA: 1 participant [severe TEAE of depression]; placebo: 3 participants [1 participant with 2 serious adverse events (SAEs) of suicide attempt, 1 participant with a SAE of suicidal ideation, and 1 participant with 1 severe TEAE of insomnia]. The majority of participants in the MDMA-AT group (97.8%) and the placebo with therapy group (90.9%) had at least 1 temporally related TEAE (TEAEs that occurred during an experiment session or up to 2 days following); none of these events were SAEs. A listing of all TEAEs from MDMA-AT group with ≥5% incidence and twice prevalence of placebo group is provided below in _{Table 37. Table 37. Table 37}.

TABLE 36
Overview of AEs (Safety Set).

| | MDMA | | Placebo | | Total | |
|---|---|---|---|---|---|---|
| | (N = 46) | | (N = 44) | | (N = 90) | |
| | n (%) | | n (%) | | n (%) | |
| Number of subjects with at least 1 TEAE | 46 | (100.0) | 44 | (100.0) | 90 | (100.0) |
| Number of subjects with at least 1 Severe TEAE | 4 | (8.7) | 7 | (15.9) | 11 | (12.2) |
| Subjects with serious or other significant TEAEs | | | | | | |
| At least one Serious TEAE | 0 | (0.0) | 2 | (4.5) | 2 | (2.2) |
| At least one Serious TEAE Leading to Death | 0 | (0.0) | 0 | (0.0) | 0 | (0.0) |
| At least one TEAE of Special Interest | 3 | (6.5) | 6 | (13.6) | 9 | (10.0) |
| Discontinued study treatment due to any TEAE(s) | 1 | (2.2) | 3 | (6.8) | 4 | (4.4) |
| Temporally-Related TEAEs | | | | | | |
| Participants with at least 1 TEAE during | 45 | (97.8) | 40 | (90.9) | 85 | (94.4) |
| Experimental Sessions and 2 Days Following | | | | | | |

| | | | |
|---|---|---|---|
| Participants with at least 1 SAE during | 0 | 0 | 0 |
| Experimental Sessions and 2 Days Following | | | |

TEAEs: AEs starting on or after first day of study intervention through to follow-up visit.
MDMA = 3,4-methylenedioxymethamphetamine;
N = total number of participants in each group;
n = number of participants;
SAE = serious adverse event;
TEAE = treatment emergent adverse event;
TEAE of special interest include a subset of AEs involving cardiac function, suicidality, and MDMA abuse. Discontinued study treatment due to any TEAEs includes all participants that discontinued study treatment due to TEAEs regardless of if AEs were the primary or secondary reason for discontinuing study treatment.

TABLE 37
Treatment-emergent Adverse Events with MDMA
Incidence ≥5% and twice the prevalence of Placebo.

| | MDMA | | Placebo | | Total |
|---|---|---|---|---|---|
| | (N = 46) | | (N = 44) | | (N = 90) |
| TEAE | n (%) | | n (%) | | n (%) |
| Muscle tightness | 28 | (60.9) | 6 | (13.6) | 34 (37.8) |
| Decreased appetite | 24 | (52.2) | 5 | (11.4) | 29 (32.2) |
| Nausea | 14 | (30.4) | 5 | (11.4) | 19 (21.1) |
| Hyperhidrosis | 10 | (21.7) | 1 | (2.3) | 11 (12.2) |
| Feeling cold | 9 | (19.6) | 3 | (6.8) | 12 (13.3) |
| Mydriasis | 7 | (15.2) | 0 | | 7 (7.8) |
| Restlessness | 7 | (15.2) | 0 | | 7 (7.8) |
| Bruxism | 6 | (13.0) | 1 | (2.3) | 7 (7.8) |
| Dizziness postural | 6 | (13.0) | 2 | (4.5) | 8 (8.9) |
| Nystagmus | 6 | (13.0) | 0 | | 6 (6.7) |
| Blood pressure increased | 5 | (10.9) | 0 | | 5 (5.6) |
| Dry mouth | 5 | (10.9) | 2 | (4.5) | 7 (7.8) |
| Feeling jittery | 5 | (10.9) | 0 | | 5 (5.6) |
| Intrusive thoughts | 4 | (8.7) | 0 | | 4 (4.4) |
| Musculoskeletal pain | 4 | (8.7) | 1 | (2.3) | 5 (5.6) |
| Non-cardiac chest pain | 4 | (8.7) | 1 | (2.3) | 5 (5.6) |
| Pain | 4 | (8.7) | 1 | (2.3) | 5 (5.6) |
| Pollakiuria | 4 | (8.7) | 1 | (2.3) | 5 (5.6) |
| Stress | 4 | (8.7) | 0 | | 4 (4.4) |
| Vision blurred | 4 | (8.7) | 1 | (2.3) | 5 (5.6) |
| Vomiting | 4 | (8.7) | 0 | | 4 (4.4) |
| Chills | 3 | (6.5) | 0 | | 3 (3.3) |
| Micturition urgency | 3 | (6.5) | 0 | | 3 (3.3) |
| Muscle twitching | 3 | (6.5) | 0 | | 3 (3.3) |
| Nervousness | 3 | (6.5) | 0 | | 3 (3.3) |
| Pyrexia | 3 | (6.5) | 1 | (2.3) | 4 (4.4) |

| | | | | |
|---|---|---|---|---|
| Somnolence | 3 | (6.5) | 0 | 3 (3.3) |
| Substance use | 3 | (6.5) | 0 | 3 (3.3) |

There were no new serious safety concerns found in MAPP1, including no increase in reported adverse events of special interest in the categories of suicidal ideation or behavior, cardiovascular, or MDMA abuse in the MDMA-AT group as compared to the psychotherapy with placebo group. As expected, based on the known sympathomimetic effects of the MDMA, transient increases in heart rate and blood pressure were observed during experimental sessions in a dose-dependent manner. These transient elevations in vitals did not require clinical intervention, including among the subset of participants with well-controlled hypertension.

### Example 12. MAPP2 Clinical Trial Summary

MAPP2 was a Phase 3 randomized, placebo-controlled, 2-arm, double-blind, multi-site study conducted to investigate the efficacy and safety of MDMA-AT in participants with PTSD of moderate or greater severity. This study included 3 experimental sessions of therapy combined with either MDMA or placebo. During Experimental Session 1, participants received a split dose of 120 mg (80 mg+40 mg). Participants received an escalated split dose of 180 mg during Experimental Sessions 2 and 3 unless there were tolerability issues or the participant declined.

The primary outcome measure, the CAPS-5, evaluated changes in PTSD symptom severity and the secondary outcome measure, the SDS, evaluated changes in functional impairment. Both measures were assessed by a blinded centralized language-specific IR pool.

### Study Population

Overall, 104 participants were randomized in the study (MDMA: 53 participants; placebo: 51 participants). All participants were included in the Safety Set. The majority of participants completed ES3 (MDMA: 100%; placebo: 84.3%) (Table 3Table 38Table 38). A total of 4 participants (all from the placebo group) terminated treatment after randomization:
One placebo participant terminated the study early due to AL as the primary reason (1 participant with a moderate TEAL of abdominal pain).
Three placebo participants terminated the study early due to choosing to discontinue treatment as the primary reason.

A total of 5 participants dropped out (MDMA: 1 participant; placebo: 4 participants) as they chose to discontinue treatment, with 1 placebo participant having an AL included as the secondary reason (mild TEAE of suicidal ideation).

TABLE 38
Participant Disposition (All Screened).

| n (%) | MDMA N = 53 | | Placebo N = 51 | |
|---|---|---|---|---|
| Randomized | 53 | | 51 | |
| Safety Set^{a} | 53 | | 51 | |
| mITT Set^{b} | 53 | | 50 | |
| Visit Completion | | | | |
| Experimental Session 1 | 53 | (100.0) | 51 | (100.0) |
| Experimental Session 2 | 53 | (100.0) | 46 | (90.2) |
| Experimental Session 3 | 53 | (100.0) | 43 | (84.3) |
| Study Termination (Visit 20) | 53 | (100.0) | 51 | (100.0) |

Reason for Study Termination and Primary Reason for Early Termination

| Post-randomization Early | 0 | | 4 | (7.8) |
|---|---|---|---|---|
| Termination | | | | |
| Adverse Event or Death | 0 | | 1 | (2.0) |
| Subject Chose to Discontinue | 0 | | 3 | (5.9) |
| Treatment | | | | |
| Dropout | 1 | (1.9) | 4 | (7.8) |
| Subject Chose to Discontinue | 1 | (1.9) | 4 | (7.8) |

| | | |
|---|---|---|
| Treatment | | |
| Lost to follow up | 0 | 0 |

MDMA = 3,4-methylenedioxymethamphetamine;
mITT = modified Intent-To-Treat;
N = total number of participants in each group;
n = number of participants.
Only primary reason for early termination and dropout are included in the table. Participants may have had secondary reasons for study treatment discontinuation (Listing 16.2.1).
^{a}Received any IMP;
^{b}Had at Least 1 CAPS-5 Assessment Post-treatment.

TABLE 39
Demographics and Baseline Characteristics (Safety Set).

| | MDMA | | Placebo | | Total | |
|---|---|---|---|---|---|---|
| | N = 53 | | N = 51 | | N = 104 | |
| Gender, n (%) | | | | | | |
| Male | 21 | (39.6) | 9 | (17.6) | 30 | (28.8) |
| Female | 32 | (60.4) | 42 | (82.4) | 74 | (71.2) |
| Age, years | | | | | | |
| Mean (SD) | 38.20 | (11.015) | 39.99 | (9.595) | 39.08 | (10.332) |
| Median (Min, | 36.18 (21.3, | | 38.22 (20.9, | | 37.16 (20.9, | |
| Max) | 70.0) | | 66.0) | | 70.0) | |
| Ethnicity, n (%) | | | | | | |
| Hispanic or | 17 | (32.1) | 11 | (21.6) | 28 | (26.9) |
| Latino | | | | | | |
| Not Hispanic or | 36 | (67.9) | 39 | (76.5) | 75 | (72.1) |
| Latino | | | | | | |
| Unknown | 0 | | 1 | (2.0) | 1 | (1.0) |
| Race, n (%) | | | | | | |
| American Indian | 0 | | 2 | (3.9) | 2 | (1.9) |
| or Alaska Native | | | | | | |
| Asian | 5 | (9.4) | 6 | (11.8) | 11 | (10.6) |
| Black or African | 5 | (9.4) | 3 | (5.9) | 8 | (7.7) |
| American | | | | | | |
| Native Hawaiian | 0 | | 1 | (2.0) | 1 | (1.0) |
| or Other Pacific | | | | | | |
| Islander | | | | | | |
| White | 37 | (69.8) | 32 | (62.7) | 69 | (66.3) |
| Multiple | 6 | (11.3) | 7 | (13.7) | 13 | (12.5) |

Max = maximum;
MDMA = 3,4-methylenedioxymethamphetamine;
Min = minimum;
N = total number of participants;
n = number of participants;
SD = standard deviation.

At Baseline, the mean (SD) duration of PTSD was 16.19 (13.3) years, with a maximum duration of 51.5 years (Table 48Table 48Table 48). Based on enrollment criteria, all participants had a duration of at least 0.5 years. Most patients had trauma histories that included developmental trauma exposure (88.5%) and/or multiple trauma (81.7%). Both duration of PTSD and trauma histories were similar across treatment groups. History of major depression (MDMA: 92.5%; placebo: 100%) was additionally similar across treatment groups.

Per enrollment criteria all participants had moderate to severe PTSD (mean [ISD] CAPS-5 Total Severity score at baseline: 39.0 [6.64]). Majority of the participants did not have CAPS-5 dissociative subtype at Baseline (76.9%). The 2 currently FDA-approved drugs for PTSD treatment, sertraline and paroxetine, were used pre-study by 24.0% and 1.9% of participants, respectively (Table 40Table 40Table 40). A total of 100 (96.2%) participants received psychotherapy prior to enrollment (Table 40Table 40Table 40). Prior to study treatment start, a similar percentage of participants in both treatment groups reported to receive CPT, EMDR, other CBT, and other psychotherapies. Majority of the participants (79.8%) received other psychotherapies prior to IMP.

TABLE 40
Baseline Disease Characteristics (Safety Set).

| | MDMA | | Placebo | | Total | |
|---|---|---|---|---|---|---|
| | N = 53 | | N = 51 | | N = 104 | |
| Trauma History, n (%) | | | | | | |
| Veteran Status | 9 | (17.0) | 7 | (13.7) | 16 | (15.4) |
| Served in a combat area | 9 | (17.0) | 6 | (11.8) | 15 | (14.4) |
| Multiple trauma events | 40 | (75.5) | 45 | (88.2) | 85 | (81.7) |
| Developmental trauma events | 49 | (92.5) | 43 | (84.3) | 92 | (88.5) |
| Baseline BDI-II Total Score | | | | | | |
| Mean (SD) | 25.4 | (11.89) | 25.5 | (11.26) | 25.5 | (11.53) |
| Median (Min, Max) | 26.0 | (5, 50) | 26.0 | (2, 50) | 26.0 | (2, 50) |
| Baseline PTSD Duration (years) | | | | | | |
| Mean (SD) | 16.25 | (14.274) | 16.14 | (12.427) | 16.19 | (13.335) |
| Median (Min, Max) | 10.14 | (2.2, 51.5) | 12.93 | (1.2, 49.1) | 11.15 | (1.2, 51.5) |
| Pre-Study PTSD Medication, n (%) | | | | | | |
| Paroxetine | 1 | (1.9) | 1 | (2.0) | 2 | (1.9) |
| Sertraline | 15 | (28.3) | 10 | (19.6) | 25 | (24.0) |
| Baseline CAPS-5 Total Severity Score | | | | | | |
| Mean (SD) | 39.4 | (6.64) | 38.7 | (6.67) | 39.0 | (6.64) |
| Median (Min, Max) | 39.0 | (28, 55) | 38.0 | (28, 56) | 39.0 | (28, 56) |
| Baseline Disease Severity (based | | | | | | |
| on CAPS-5), n (%) | | | | | | |
| Moderate (28-34) | 13 | (24.5) | 15 | (29.4) | 28 | (26.9) |
| Severe (≥35) | 40 | (75.5) | 36 | (70.6) | 76 | (73.1) |
| Baseline CAPS-5 Dissociative Subtype, n (%) | | | | | | |
| No | 40 | (75.5) | 40 | (78.4) | 80 | (76.9) |
| Yes | 13 | (24.5) | 11 | (21.6) | 24 | (23.1) |
| Prior Psychotherapy, n (%) | | | | | | |
| Participants with any previous psychotherapy | 51 | (96.2) | 49 | (96.1) | 100 | (96.2) |
| Cognitive Processing Therapy | 1 | (1.9) | 1 | (2.0) | 2 | (1.9) |
| Dialectical Behavioral Therapy | 4 | (7.5) | 2 | (3.9) | 6 | (5.8) |
| Eye Movement Desensitization Reprocessing | 17 | (32.1) | 18 | (35.3) | 35 | (33.7) |
| Group Psychotherapy | 9 | (17.0) | 15 | (29.4) | 24 | (23.1) |
| Holotropic Breathwork | 0 | (0.0) | 3 | (5.9) | 3 | (2.9) |
| Other Cognitive Behavioral Therapy | 15 | (28.3) | 14 | (27.5) | 29 | (27.9) |
| Prolonged Exposure | 2 | (3.8) | 0 | (0.0) | 2 | (1.9) |
| Psychodynamic | 15 | (28.3) | 11 | (21.6) | 26 | (25.0) |
| Other | 41 | (77.4) | 42 | (82.4) | 83 | (79.8) |

BDI-II = Beck Depression Inventory II;
CAPS-5 = Clinician-administered PTSD Scale for DSM-5;
DSM-5 = Diagnostic and Statistical Manual of Mental Disorders version 5;
Max = maximum;
Min = minimum;
PTSD = Posttraumatic Stress Disorder;
SD = standard deviation

### Efficacy

### Primary Endpoint Analyses

A de jure estimand of treatment efficacy was used to estimate the causal effect of MDMA-AT on PTSD symptom severity as measured by the change from Baseline to Visit 19 in CAPS-S total severity scores in the mITT analysis set. An MMRM analysis of the deljure estimand showed a statistically significant difference (p=0.0004) between treatment groups, with a greater reduction in CAPS-5 total severity scores in participants receiving MDMA (−23.69) compared to placebo (−14.78) (Table 41Table 41Table 41). Differences in treatment effect among demographic, dissociative sub-type, and overnight stay subgroups were not observed.

TABLE 41
Change in CAPS-5 Total Severity Scores by Visit-De Jure (mITT Set).

| Statistic by Visit | MDMA N = 53 | | Placebo N = 50 | |
|---|---|---|---|---|
| Baseline CAPS-5 T1 (Visit 3), n | 53 | | 50 | |
| Mean (SD) | 39.4 | (6.64) | 38.8 | (6.63) |
| Median (Min, Max) | 39.0 | (28, 55) | 39.0 | (28, 56) |
| CAPS-5 T2 (Visit 8), n | 53 | | 50 | |
| Mean (SD) | 28.1 | (12.86) | 31.4 | (10.29) |
| Median (Min, Max) | 30.0 | (2, 51) | 33.0 | (11, 55) |
| Change from Baseline to CAPS-5 T2 (Visit 8), n | 53 | | 50 | |
| Mean (SD) | −11.3 | (11.69) | −7.5 | (8.53) |
| Median (Min, Max) | −12.0 | (−40, 10) | −7.0 | (−30, 7) |
| LS Mean (95% CI)^{a} | −11.36 | (−14.17, −8.55) | −7.22 | (−10.11, −4.32) |
| LS Mean for Treatment Difference (95% CI)^{a} | −4.14 | (−8.19, −0.09) | | |
| CAPS-5 T3 (Visit 13), n | 53 | | 44 | |
| Mean (SD) | 20.9 | (13.42) | 27.7 | (11.79) |
| Median (Min, Max) | 19.0 | (0, 45) | 29.0 | (5, 50) |
| Change from Baseline to CAPS-5 T3 (Visit 13), n | 53 | | 44 | |
| Mean (SD) | −18.5 | (11.82) | −10.8 | (10.76) |
| Median (Min, Max) | −20.0 | (−41, 2) | −13.0 | (−33, 11) |
| LS Mean (95% CI)^{a} | −18.58 | (−21.68, −15.49) | −10.60 | (−13.91, −7.29) |
| LS Mean for Treatment Difference (95% CI)^{a} | −7.98 | (−12.53, −3.44) | | |
| Primary Outcome CAPS-5 T4 (Visit 19), n | 52 | | 42 | |
| Mean (SD) | 15.8 | (12.40) | 23.3 | (12.79) |
| Median (Min, Max) | 15.5 | (0, 44) | 21.5 | (2, 48) |
| Change from Baseline to Primary Outcome | 52 | | 42 | |
| CAPS-5 T4 (Visit 19), n | | | | |
| Mean (SD) | −23.5 | (12.08) | −15.4 | (12.30) |
| Median (Min, Max) | −25.0 | (−44, 9) | −18.0 | (−40, 10) |
| LS Mean (95% CI)^{a} | −23.69 | (−26.94, −20.44) | −14.78 | (−18.28, −11.28) |
| LS Mean for Treatment Difference (95% CI)^{a} | −8.91 | (−13.70, −4.12) | | |

| | |
|---|---|
| p-value^{a} | 0.0004 |

The de jure estimand does not include data after participants discontinued treatment.
CAPS-5 = Clinician Administered PTSD Scale for DSM-5;
CI = confidence interval;
DSM-5 = Diagnostic and Statistical Manual of Mental Disorders version 5;
LS = least squares;
Max = maximum;
MDMA = 3,4-methylenedioxymethamphetamine;
Min = minimum;
mITT = modified intent-to-treat;
MMRM = mixed models repeated measures;
PTSD = posttraumatic stress disorder;
SD = standard deviation.
^{a}LS Mean, LS Mean difference, 95% CI and p-value of treatment effect at Visit 19 are from an MMRM model with treatment group, visit, treatment group by visit interaction, site, and dissociative subtype as fixed effect, and baseline CAPS-5 as a covariat.

A clinically meaningful reduction in CAPS-5 total severity scores and PTSD diagnostic criteria by visit for mITT Set is presented in Table 42Table 42Table 42. A total of 45 (86.5%) participants receiving MDMA-AT met the definition of responder, demonstrating a clinically meaningful 10-point reduction in CAPS-5 total severity score, compared to 29 (69.0%) of participants receiving placebo at Visit 19. Total Severity Scores no longer met PTSD diagnostic criteria in 37 (71.2%) participants receiving MDMA compared to 20 (47.6%) of participants receiving the placebo at Visit 19.

A total of 24 (46.2%) of participants receiving the MDMA-AT met the definition of in remission, having both a CAPS-5 total severity score≤11 and no longer meeting PTSD diagnostic criteria compared to 9 (21.4%) of participants receiving placebo at Visit 19.

TABLE 42
Clinically Significant Reduction in CAPS-5 Total Severity
Scores and PTSD Diagnostic Criteria by Visit (mITT Set).

| | | |
|---|---|---|
| Visit | MDMA | Placebo |
| Responder Criteria | N = 53 | N = 50 |

10-point reduction in CAPS-5 Total Severity Score

| CAPS-5 T2 (Visit 8) | 53 | | 50 | |
|---|---|---|---|---|
| Responder | 28 | (52.8) | 21 | (42.0) |
| Non-Responder | 25 | (47.2) | 29 | (58.0) |
| Loss of Diagnosis | 21 | (39.6) | 11 | (22.0) |
| Remission | 5 | (9.4) | 1 | (2.0) |
| CAPS-5 T3 (Visit 13) | 53 | | 44 | |
| Responder | 41 | (77.4) | 25 | (56.8) |
| Non-Responder | 12 | (22.6) | 19 | (43.2) |
| Loss of Diagnosis | 32 | (60.4) | 15 | (34.1) |
| Remission | 14 | (26.4) | 5 | (11.4) |
| Primary Outcome CAPS-5 T4 (Visit 19) | 52 | | 42 | |
| Responder | 45 | (86.5) | 29 | (69.0) |
| Non-Responder | 7 | (13.5) | 13 | (31.0) |
| Loss of Diagnosis | 37 | (71.2) | 20 | (47.6) |
| Remission | 24 | (46.2) | 9 | (21.4) |

CAPS-5 = Clinician Administered PTSD Scale for DSM-5;
DSM-5 = Diagnostic and Statistical Manual of Mental Disorders version 5;
MDMA = 3,4-methylenedioxymethamphetamine;
mITT = modified intent-to-treat;
PTSD = posttraumatic stress disorder.

### Key Secondary Endpoint Analyses

The de jure estimand of treatment efficacy was used to determine the effect of MDMA-AT on SDS total score in the mITT analysis set. An MMRM analysis of the de jure estimand showed a statistically significant difference (p=0.0271), with a greater reduction in SDS total scores in participants receiving MDMA (−3.31) compared to placebo (−2.11) (Table 43Table 43Table 43).

TABLE 43
SDS Total Scores by Visit (mITT Set).

| | MDMA-AT | | Placebo with | |
|---|---|---|---|---|
| Statistics | N = 53 | | Therapy N = 50 | |
| Baseline (Visit 3) (n) | 53 | | 50 | |
| Mean (SD) | 6.0 | (1.80) | 6.1 | (1.79) |
| Median (min, max) | 6.3 | (1, 10) | 6.2 | (2, 10) |
| Visit 8 (n) | 53 | | 50 | |
| Mean (SD) | 4.1 | (2.60) | 4.6 | (2.36) |
| Median (min, max) | 4.3 | (0, 10) | 4.3 | (0, 9) |
| Visit 13 (n) | 53 | | 44 | |
| Mean (SD) | 2.9 | (2.78) | 4.5 | (2.77) |
| Median (min, max) | 2.7 | (0, 9) | 4.8 | (0, 10) |
| Visit 19 (Primary | 52 | | 42 | |
| Endpoint) (n) | | | | |
| Mean (SD) | 2.7 | (2.67) | 4.0 | (2.82) |
| Median (min, max) | 2.3 | (0, 9) | 3.8 | (0, 9) |
| CFB (n) | 52 | | 42 | |
| Mean (SD) | −3.3 | (2.59) | −2.2 | (2.91) |
| Median (min, max) | −4.0 | (−7, 7) | −1.8 | (−10, 3) |
| LSMean (95% CI)^{a} | −3.31 | (−4.03, −2.60) | −2.11 | (−2.89, −1.33) |

| | | | |
|---|---|---|---|
| LSMean for Treatment | −1.20 | (−2.26, −0.14) | — |
| Difference (95% CI)^{a} | | | |

| | | |
|---|---|---|
| p-value^{a} | 0.0271 | — |

CFB = Change from Baseline
LSM = Least Square Means;
Max = Maximum;
MDMA = 3,4-methylenedioxymethamphetamine;
Min = Minimum;
mITT = modified Intent-to-treat;
SD = Standard Deviation;
SDS = Sheehan Disability Scale.
^{a}LS Mean, LS Mean difference, 95% CI, and p-value of treatment effect at Visit 19 were from an MMRM model with treatment group, visit, treatment group by visit interaction, site, and dissociative subtype as fixed effect, and baseline SDS as a covariate.

### Safety

Overall, 102 (98.1%) participants reported at least 1 TEAE during the study period (Table 52riod (Table 52riod (Table 52). Of these, 2 (3.9%) participants from the placebo group were discontinued due to TEAE (abdominal pain and suicidal ideation). There were no serious TEAEs or deaths reported during the study. Adverse events of special interest included a subset of AEs involving cardiac function, suicidality, and MDMA abuse. There were 6 (11.3%) participants in the MDMA group and 3 (5.9%) participants in the placebo group with an TEAE of special interest. AESIs related to suicidality were reported in 2 MDMA participants and 2 placebo participants. AESIs related to cardiac function were reported in 4 MDMA participants and 1 placebo participant (all palpitations). There were no AESIs of MDMA abuse. All participants in the MDMA-AT group and the majority of participants in the placebo with therapy group (86.3%) had at least 1 temporally related TEAE (TEAEs that occurred during an experiment session or up to 2 days following). A listing of all TEAEs from MDMA-AT group with ≥5% incidence and twice the prevalence of placebo group is provided below in Table 44Table 44Table 44.

TABLE 44
Overall Summary of Treatment Emergent Adverse Events, Serious
Adverse Events, Discontinuations, and Deaths (Safety Set).

| | MDMA | | Placebo | Total | |
|---|---|---|---|---|---|
| | N = 53 n | | N = 51 n | N = 104 n | |
| | (%) | | (%) | (%) | |
| Number of subjects with at least 1 TEAE | 53 | (100.0) | 49 (96.1) | 102 | (98.1) |
| Number of subjects with at least 1 Severe TEAE | 5 | (9.4) | 2 (3.9) | 7 | (6.7) |
| Subjects with serious or other significant TEAEs | | | | | |
| At least one Treatment-emergent SAE | 0 | (0.0) | 0 (0.0) | 0 | (0.0) |
| At least one Treatment-emergent SAE Leading to Death | 0 | (0.0) | 0 (0.0) | 0 | (0.0) |
| At least one TEAE of Special Interest | 6 | (11.3) | 3 (5.9) | 9 | (8.7) |
| Discontinued study treatment due to any TEAE(s) | 0 | (0.0) | 2 (3.9) | 2 | (1.9) |
| Temporally-Related TEAEs | | | | | |
| Participants with at least 1 TEAE during | 53 | (100.0) | 44 (86.3) | 97 | (93.3) |
| Experimental Sessions and 2 Days Following | | | | | |

| | | | |
|---|---|---|---|
| Participants with at least 1 SAE during Experimental | 0 | 0 | 0 |
| Sessions and 2 Days Following | | | |

Treatment emergent adverse events: AEs starting on or after first day of study intervention through to follow-up visit.
MDMA = 3,4-methylenedioxymethamphetamine;
N = total number of participants in each group;
n = number of participants;
SAE = serious adverse event;
TEAE = treatment emergent adverse event.
TEAE of special interest include a subset of AEs involving cardiac function, suicidality, and MDMA abuse. Discontinued study treatment due to any TEAEs includes all participants that discontinued study treatment due to TEAEs regardless of if AEs were the primary or secondary reason for discontinuing study treatment.

TABLE 45
Treatment-emergent Adverse Events with MDMA
Incidence ≥5% and twice the prevalence of Placebo.

| | MDMA | | Placebo | | Total | |
|---|---|---|---|---|---|---|
| | (N = 53) | | (N = 51) | | (N = 104) | |
| TEAE | n (%) | | n (%) | | n (%) | |
| Muscle tightness | 31 | (58.8) | 13 | (25.5) | 44 | (42.3) |
| Nausea | 24 | (45.3) | 11 | (21.6) | 35 | (33.7) |
| Decreased appetite | 19 | (35.8) | 5 | (9.8) | 24 | (23.1) |
| Hyperhidrosis | 18 | (34.0) | 3 | (5.9) | 21 | (20.2) |
| Feeling hot | 14 | (26.4) | 6 | (11.8) | 20 | (19.2) |
| Feeling cold | 11 | (20.8) | 3 | (5.9) | 14 | (13.5) |
| Paraesthesia | 10 | (18.9) | 1 | (2.0) | 11 | (10.6) |
| Chest discomfort | 9 | (17.0) | 2 | (3.9) | 11 | (10.6) |
| Dry mouth | 9 | (17.0) | 4 | (7.8) | 13 | (12.5) |
| Chills | 8 | (15.1) | 1 | (2.0) | 9 | (8.7) |
| Feeling jittery | 8 | (15.1) | 0 | | 8 | (7.7) |
| Restlessness | 8 | (15.1) | 2 | (3.9) | 10 | (9.6) |
| Vision blurred | 8 | (15.1) | 0 | | 8 | (7.7) |
| Bruxism | 7 | (13.2) | 1 | (2.0) | 8 | (7.7) |
| Nystagmus | 7 | (13.2) | 1 | (2.0) | 8 | (7.7) |
| Mydriasis | 6 | (11.3) | 0 | | 6 | (5.8) |
| Tremor | 6 | (11.3) | 0 | | 6 | (5.8) |
| Abdominal pain upper | 5 | (9.4) | 1 | (2.0) | 6 | (5.8) |
| Feeling abnormal | 5 | (9.4) | 2 | (3.9) | 7 | (6.7) |
| Feeling of body | 5 | (9.4) | 0 | | 5 | (4.8) |
| temperature change | | | | | | |
| Hypoaesthesia | 5 | (9.4) | 1 | (2.0) | 6 | (5.8) |
| Palpitations | 5 | (9.4) | 1 | (2.0) | 6 | (5.8) |
| Muscle spasms | 4 | (7.5) | 0 | | 4 | (3.8) |
| Thirst | 4 | (7.5) | 1 | (2.0) | 5 | (4.8) |
| Dissociation | 3 | (5.7) | 0 | | 3 | (2.9) |
| Flushing | 3 | (5.7) | 1 | (2.0) | 4 | (3.8) |
| Gait disturbance | 3 | (5.7) | 0 | | 3 | (2.9) |
| Heart rate increased | 3 | (5.7) | 0 | | 3 | (2.9) |
| Panic attack | 3 | (5.7) | 1 | (2.0) | 4 | (3.8) |

| | | | | | |
|---|---|---|---|---|---|
| Visual impairment | 3 | (5.7) | 0 | 3 | (2.9) |

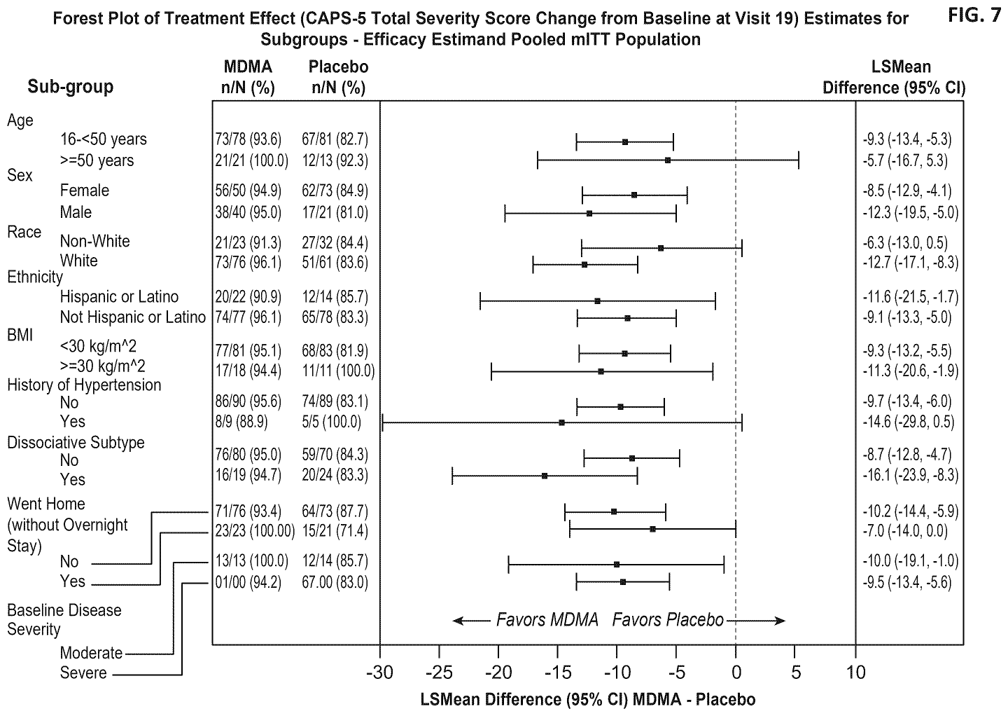

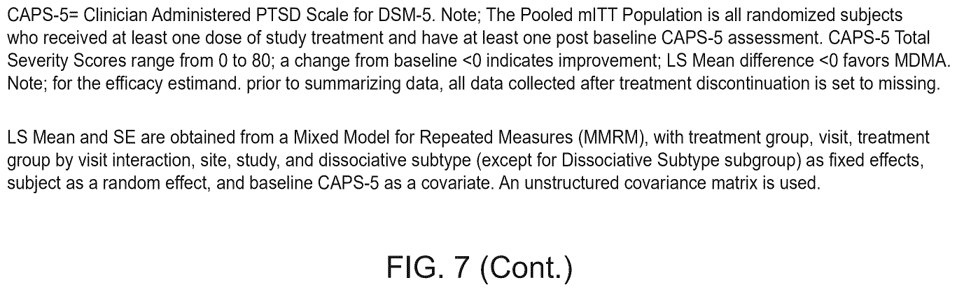

There were no new serious safety concerns found in MAPP2, including no increase in reported adverse events of special interest in the categories of suicidal ideation or behavior, cardiovascular, or MDMA abuse in the MDMA-AT group as compared to the psychotherapy with placebo group. As expected, based on the known sympathomimetic effects of the MDMA, transient increases in heart rate and blood pressure were observed during experimental sessions in a dose-dependent manner. These transient elevations in vitals did not require clinical intervention, including among the subset of participants with well-controlled hypertension. FIG. 7 shows an integrated forest plot of treatment effect for the MAPP1 and MAPP2 clinical trials.

### Example 13. MPLong Clinical Trial Summary of Results

MPLONG is an observational long-term follow-up study for MDMA-AT trial participants intended to provide data on treatment durability as measured by the CAPS-5 and Sheehan Disability Scale (SDS). Participants who elect to enroll in MPLONG completed a LTFU visit at least 6 months following their completion of the parent study. MPLONG opened for enrollment 8 months following the last subject visit of the MAPP1 trial and MAPP1 participants entered MPLONG unblinded to their treatment assignment. Enrollees from the second Phase 3 trial MAPP2, however, complete LTFU with blinding maintained. In all cases, the primary CAPS-5 and secondary SDS assessments are conducted by a centralized blinded independent rater (IR) pool and these data have been maintained in a separate database with restricted access preventing any unplanned analyses. The statistical analysis plan for MPLONG was finalized prior to the sponsor obtaining access to these data.

Overall, 65 participants in the MDMA groups and 57 participants in the placebo groups from the MAPP1 and MAPP2 parent studies contributed to the data for this data cut (27 Feb. 2023).

### Durable Improvement in CAPS-5

The mean change from baseline (CFB) in CAPS-5 scores at LTFU was −28.7 in participants receiving MDMA and −16.4 in participants receiving placebo during the MAPP2 parent study (Table 46Table 46Table 46). These data suggest that improvements in CAPS-5 total severity scores were durable from the end of the study to the LTFU in both the MDMA and placebo treatment groups. Similar results were observed when analyzing the CFB in combined participants from the MAPP1 and MAPP2 parent studies.

TABLE 46
Change from Parent Study (MAPP1 and MAPP2)
Baseline in CAPS-5 Total Severity Score.

| MDMA | | Placebo | |
|---|---|---|---|
| Actual | CFB | Actual | CFB |

Parent Study Visits (MAPP2 Participants Only)

| Baseline, n | 38 | | | | 28 | | | |
|---|---|---|---|---|---|---|---|---|
| Mean (SD) | 39.4 | (6.29) | | | 38.8 | (6.65) | | |
| Median (Min, Max) | 38.5 | (29, 55) | | | 40.0 | (28, 56) | | |
| Visit 8, n | 38 | | 38 | | 28 | | 28 | |
| Mean (SD) | 28.1 | (13.11) | −11.3 | (11.86) | 30.9 | (10.46) | −7.9 | (8.62) |
| Median (Min, Max) | 30.5 | (2, 51) | −12.0 | (−40, 10) | 32.5 | (11, 46) | −8.5 | (−26, 7) |
| Visit 13, n | 38 | | 38 | | 27 | | 27 | |
| Mean (SD) | 19.7 | (13.59) | −19.8 | (12.57) | 26.2 | (10.50) | −12.6 | (10.55) |
| Median (Min, Max) | 18.0 | (0, 45) | −22.0 | (−41, 2) | 29.0 | (5, 41) | −13.0 | (−33, 5) |
| Visit 19, n | 37 | | 37 | | 27 | | 27 | |
| Mean (SD) | 14.8 | (12.28) | −24.5 | (12.29) | 22.7 | (12.63) | −16.0 | (13.14) |
| Median (Min, Max) | 12.0 | (0, 44) | −27.0 | (−44, 9) | 22.0 | (2, 44) | −18.0 | (−40, 10) |

Long Term Follow Up Study (MAPP2 Participants Only)

| Visit 1, n | 38 | | 38 | | 28 | | 28 | |
|---|---|---|---|---|---|---|---|---|
| Mean (SD) | 10.7 | (9.85) | −28.7 | (10.80) | 22.4 | (14.04) | −16.4 | (14.76) |
| Median (Min, Max) | 8.5 | (0, 37) | −30.5 | (−43, 1) | 22.5 | (1, 47) | −16.5 | (−45, 9) |

Parent Study Visits (MAPP1 and MAPP2 Participants)

| Baseline, n | 65 | | | | 57 | | | |
|---|---|---|---|---|---|---|---|---|
| Mean (SD) | 40.8 | (6.09) | | | 41.2 | (6.64) | | |
| Median (Min, Max) | 40.0 | (29, 57) | | | 41.0 | (28, 62) | | |
| Visit 8, n | 65 | | 65 | | 56 | | 56 | |
| Mean (SD) | 29.5 | (11.81) | −11.3 | (10.53) | 32.1 | (10.22) | −8.9 | (8.38) |
| Median (Min, Max) | 31.0 | (2, 51) | −12.0 | (−40, 10) | 34.0 | (8, 50) | −8.5 | (−29, 7) |
| Visit 13, n | 64 | | 64 | | 55 | | 55 | |
| Mean (SD) | 22.0 | (12.91) | −18.8 | (11.40) | 28.2 | (11.63) | −12.9 | (11.32) |
| Median (Min, Max) | 23.0 | (0, 45) | −19.0 | (−41, 2) | 30.0 | (3, 53) | −14.0 | (−34, 10) |
| Visit 19, n | 63 | | 63 | | 56 | | 56 | |
| Mean (SD) | 15.9 | (12.28) | −24.8 | (11.57) | 24.9 | (12.16) | −16.3 | (11.92) |
| Median (Min, Max) | 14.0 | (0, 44) | −27.0 | (−45, 9) | 26.5 | (2, 45) | −18.0 | (−40, 10) |
| Visit 1, n | 65 | | 65 | | 57 | | 57 | |
| Mean (SD) | 11.5 | (10.39) | −29.2 | (11.28) | 24.9 | (13.63) | −16.2 | (12.99) |
| Median (Min, Max) | 8.0 | (0, 40) | −31.0 | (−47, 1) | 27.0 | (1, 58) | −16.0 | (−45, 15) |

A sensitivity analysis was conducted to determine whether there was a detectable difference in the observed trend dependent on the time elapsed since the parent study (Table 47 (Table 47 (Table 47). There was no observable impact of the time window, as durability was maintained in both those with LFTU within a year and those with LTFU greater than 12 months after study termination.

TABLE 47
Sensitivity Analysis of Change from Study Termination in CAPS-5
Total Severity Score by Time Window (MAPP1 and MAPP2).

| | MDMA | | | | Placebo | | | |
|---|---|---|---|---|---|---|---|---|
| | | | Change from | | | | Change from | |
| | Actual | | Study Termination | | Actual | | Study Termination | |
| Study Termination^{a}, n | 65 | | | | 57 | | | |
| Mean (SD) | 16.4 | (12.57) | | | 25.1 | (12.11) | | |
| Median (min, max) | 15.0 | (0, 44) | | | 27.0 | (2, 45) | | |
| LTFU 6-12 Months^{b}, n | 30 | | 30 | | 24 | | 24 | |
| Mean (SD) | 11.9 | (10.29) | −3.7 | (6.76) | 23.7 | (14.07) | 0.0 | (9.12) |
| Median (min, max) | 9.0 | (0, 37) | −2.5 | (−19, 8) | 24.5 | (1, 47) | −0.5 | (−16, 26) |
| LTFU >12 Months^{b}, n | 35 | | 35 | | 33 | | 33 | |
| Mean (SD) | 11.2 | (10.61) | −5.9 | (11.64) | 25.8 | (13.45) | −0.3 | (10.25) |
| Median (min, max) | 6.0 | (0, 40) | −5.0 | (−30, 17) | 28.0 | (2, 58) | 2.0 | (−26, 20) |

^{a}Last available assessment in the parent study (MAPP1 or MAPP2)
^{b}Each subject was included in 1 of the 2 visit windows only

### Assessment of Relapse

Durability of response and frequency of relapse following a treatment response, loss of diagnosis, or remission was also assessed (Table 48(Table 48(Table 48). A higher proportion of patients in the MDMA group compared to the placebo group still met the definition of treatment response at LTFU (MDMA: 58 [89.2%]; placebo: 34 [59.6%]), loss of diagnosis (MDMA: 54 [83.1%]; placebo: 23 [40.4%]), or remission (MDMA: 39 [60%]; placebo: 14[24.6%]).

In addition, there was a low incidence of relapse following treatment response or loss of diagnosis (MDMA: 8 [12.3%]; placebo: 14 [24.6%]), and an even lower incidence of relapse following remission (MDMA: 3 [4.6%]; placebo: 3 [5.3%]).

TABLE 48
Responder Analysis at the LTFU Visit (MAPP1 and MAPP2).

| | | | MDMA | | Placebo | |
|---|---|---|---|---|---|---|
| | | | (n = 65) | | (n = 57) | |
| Category | Response Criteria | Statistic | n (%) | | n (%) | |
| Durable | ≥10 pt reduction in CAPS-5 TSS from | Meets criteria | 58 | (89.2) | 34 | (59.6) |
| Treatment | BL at study termination and LTFU^{a} | Does not meet | 7 | (10.8) | 23 | (40.4) |
| Response | | criteria | | | | |
| Durable Loss | ≥10 pt reduction in CAPS-5 TSS from | Meets criteria | 54 | (83.1) | 23 | (40.4) |
| of Diagnosis | BL and not meeting PTSD diagnostic | Does not meet | 11 | (16.9) | 34 | (59.6) |
| | criteria at LTFU | criteria | | | | |
| Durable | ≤11 CAPS-5 TSS from BL not meeting | Meets criteria | 39 | (60.0) | 14 | (24.6) |
| Remission | PTSD diagnostic criteria at LTFU | Does not meet | 26 | (40.0) | 43 | (75.4) |
| | | criteria | | | | |
| Relapse after | ≥10 point reduction in CAPS-5 TSS from | Meets criteria | 4 | (6.2) | 7 | (12.3) |
| Treatment | BL and ≥10 point increase from study | Does not meet | 61 | (93.8) | 50 | (87.7) |
| Response | termination at LTFU^{a} | criteria | | | | |
| Relapse after | ≥10 point reduction in CAPS-5 TSS from BL | Meets criteria | 4 | (6.2) | 7 | (12.3) |
| Loss of | and not meeting PTSD diagnostic criteria | Does not meet | 61 | (93.8) | 50 | (87.7) |
| Diagnosis | at study termination and ≥10 point | criteria | | | | |
| | increase and meeting PTSD diagnostic | | | | | |
| | criteria at LTFU^{a} | | | | | |
| Relapse of | ≤11 CAPS-5 TSS from BL and not meeting | Meets criteria | 3 | (4.6) | 3 | (5.3) |
| Remission | PTSD diagnostic criteria at study | Does not meet | 62 | (95.4) | 54 | (94.7) |
| | termination and >11 CAPS-5 TSS at LTFU^{a} | criteria | | | | |

^{a}Study termination is the visit for the last available assessment in the parent study (MAPP1 or MAPP2);
subjects can be included in more than one category

### Example 14. MDMA Product Characteristics

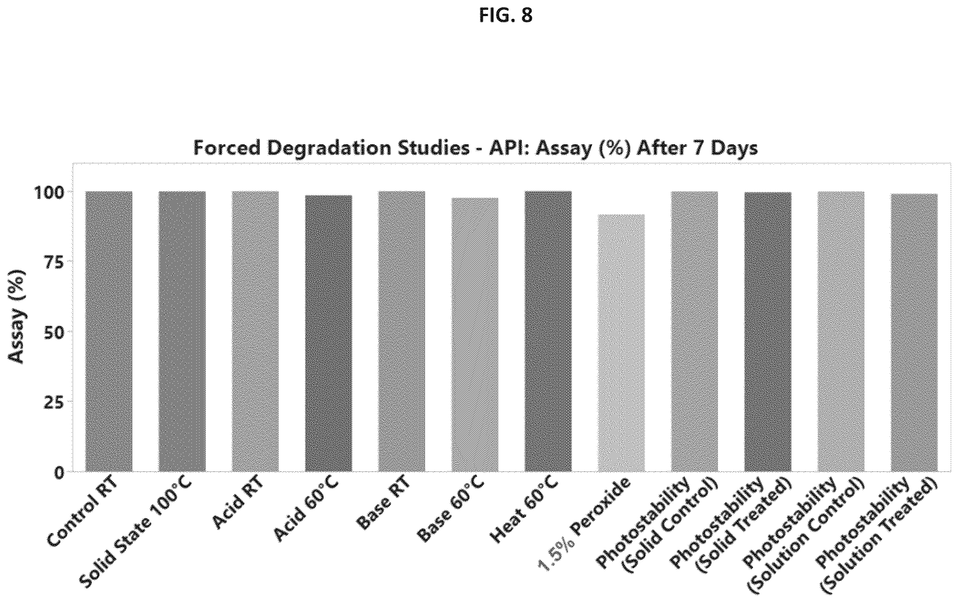

The crystalline, anhydrous form (Form 1) of MDMA·HCl is used for clinical development and commercialization purpose. The API shows minimal degradation after 7 days forced degradation (FIG. 8).

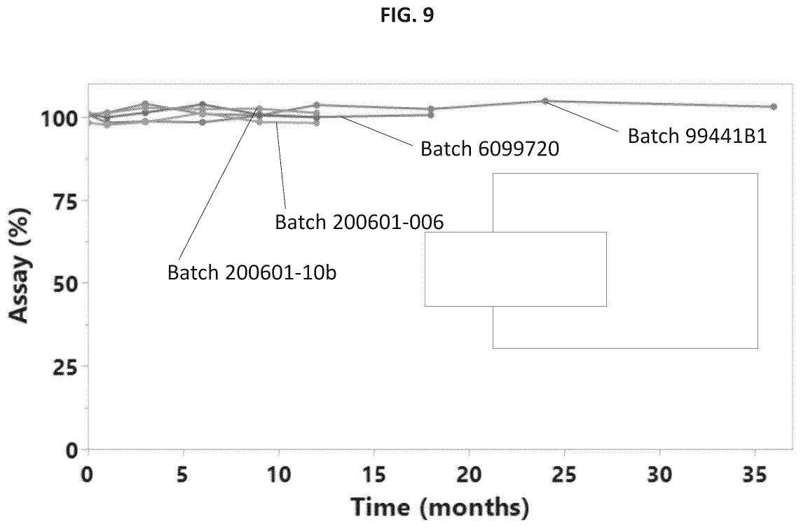

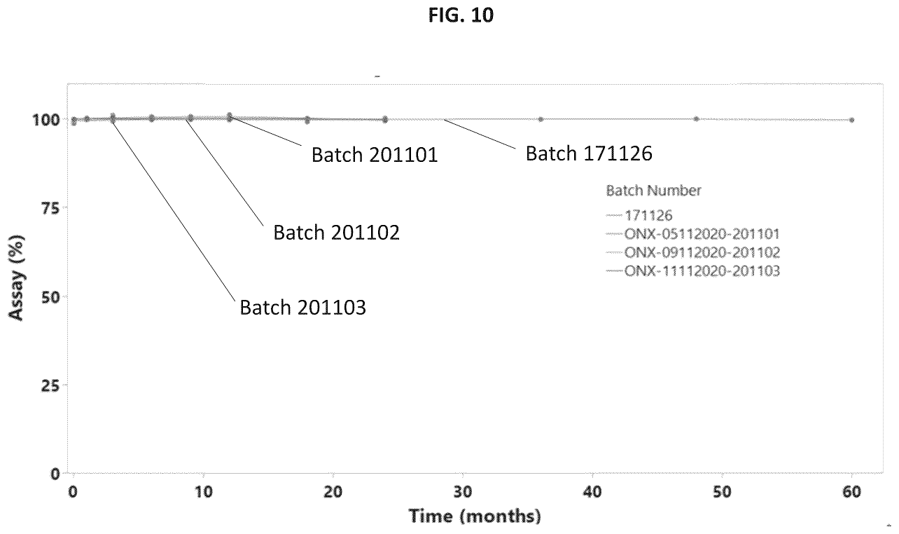

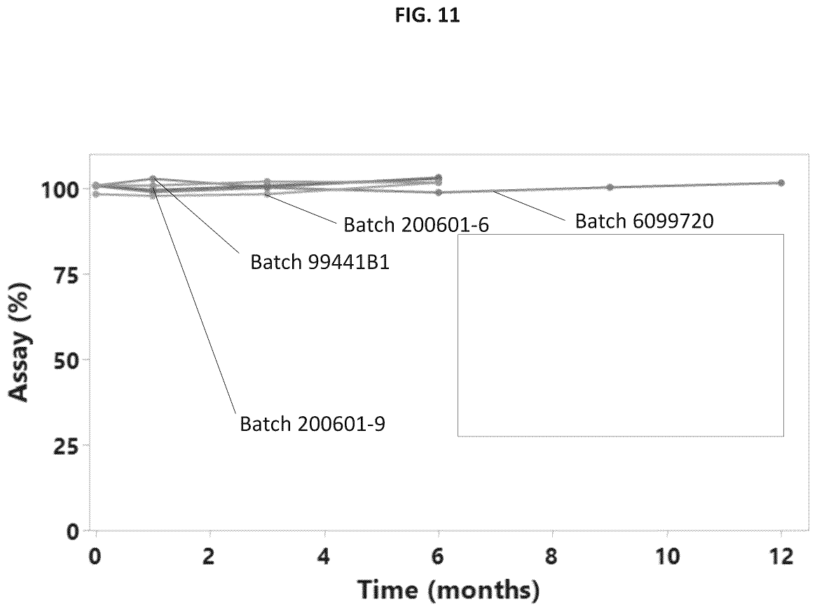

The long-term chemical stability of MDMA·HCl, Form I appears in FIG. 9 and FIG. 10. As can be seen in the results, the long term chemical stability of MDMA·HCl at 25 degrees celsius at 60% relative humidity (RH) remains constant for up to 60 months, with no change in appearance or increase in degradation products. Even the effects of higher humidity and temperature do not affect the chemical stability of MDMA·HCl, Form I API for up to 12 months (FIG. 11).

### Example 15. Assessment of Eating Disorder Psychopathology Before and After MDMA-Assisted Therapy (MDMA-AT)

Exploratory data on ED psychopathology were analyzed that were collected as part of a Phase 3 trial (trial ID: NCT03537014, which is herein incorporated by reference in its entirety), which was a double-blind randomized controlled study that compared efficacy and safety of MDMA-AT between placebo and MDMA groups for treatment of PTSD. EAT-26 was included as a pre-specified exploratory measure to assess participants' attitudes about eating and food in addition to the presence of previously undetected EDs. EAT-26 assessments were collected at baseline (visit 4) and study termination (visit 20). This self-reported questionnaire consists of 25 components, each rated on a six-point scale of 0 (Never) to 3 (Always), plus an additional component rated from 0 (Always) to 3 (Never). The 27th item addresses occurrence and frequency of specific eating behaviors, such as binge eating. Scores were combined to produce a total EAT-26 score, ranging from 0 to 78, with lower values indicating fewer symptoms. Participants meeting a total cut-off score of 20 or greater are considered likely candidates for having an ED diagnosis and warrant referral to a qualified professional. Lowering the cut-off score to 11 has been shown to improve sensitivity rates in a wider range of EDs such as binge eating disorder (BED) and eating disorder not otherwise specified (EDNOS) (Orbitello et al., 2006).

Among a total of 90 participants who were randomized and received treatment, 89 participants completed the EAT-26 assessment at baseline, and seven participants withdrew from the study and were missing follow-up data (3 MDMA, 4 placebo). A total of 82 of 90 participants (91.1%) completed both baseline and follow-up EAT-26 assessments and were included in the final analysis. This preliminary analysis of an exploratory measure included only completers of both EAT-26 scores at baseline and follow-up assessments to avoid imputation of data that would attenuate the accuracy of results. Of the 89 initial participants, 15 (15.7%) met criteria for a current ED (binge eating disorder (BED): n=5; other specified feeding and eating disorder (OSFED): n=9), and 13 others (14.6%) had a previous history of an ED (anorexia nervosa−binge-purge type (AN-BP); bulimia nervosa (BN): n=6; OSFED: n=6). The baseline sample consisted of participants who were majority female (65.2%), identified as women (62.9%), non-Hispanic White or Latino (89.9%), college graduates (70.8%), and the mean (SD) age was 41.0 (12.00) years. In total, 17 participants had been prescribed sertraline, of which 8 and 9 were assigned to the MDMA and placebo treatment groups, respectively. Furthermore, 6 participants had been prescribed paroxetine, which were equally distributed between MDMA and placebo treatment groups (3 and 3, respectively). At baseline, BMI (kg/m2) scores were in the ‘normal’ range (BMI 18.5-24.9) in 56.2%, ‘overweight’ (BMI 25.0-29.9) in 28.1%, and ‘obese’ (BMI≥30) in 15.7% of participants. There were no treatment group differences in demographic variables or baseline ACE, BDI-II, CAPS-5, or lifetime C-SSRS assessments. Sample demographics and baseline characteristics are summarized in Table 1. Mean BMI (SD) was 26.0 (4.8) kg/m2 in the MDMA-AT group and 24.8 (4.2) kg/m2 in the PLAC-AT group (t=1.3, p=0.2).

At baseline, 13 (15%) of the 89 participants starting the study had EAT-26 scores≥20, which is defined as within the “clinical” range (Garfinkel and Newman, 2001; Garner et al., 1982), and 28 (31.5%) had total EAT-26 scores≥11, which has been defined as an “at risk” range (Orbitello et al., 2006). Among the study completers (n=82), 11 (13.4%) had baseline EAT-26 scores in the “clinical” range (≥20) and 24 (29.3%) had EAT-26 scores in the “at risk” range (≥11). Otherwise, there were no significant differences in baseline EAT-26 scores between the placebo and MDMA groups in study completers.

In the total sample, there was a significant treatment group difference in EAT-26 change scores between placebo and MDMA groups after adjusting for baseline EAT-26 scores [F (2,79)=4.68, p=0.0335; Hedge's g=0.33]. The MDMA group had a statistically significant within-subject mean (SD) reduction (improvement) in EAT-26 scores of −3.04 (6.24) from baseline to follow-up (p=0.02), and this reduction in EAT-26 scores was significantly greater compared to a reduction of −0.68 (8.04) in the placebo group.

Additional subset analyses indicated participants with greater baseline EAT-26 scores generally had significantly greater improvement at follow-up. Approximately 12 (30.0%) placebo and 12 (28.6%) MDMA participants indicated having a baseline EAT-26 score≥11; and 6 (15.0%) placebo and 5 (11.9%) MDMA participants had baseline EAT-26 score≥20. In the baseline EAT-26≥11 subset sample, the MDMA group (women=7, men=4, non-binary=1) had a statistically significant within-subject mean (SD) reduction in EAT-26 scores of −9.58 (7.59) (p=0.0007), and this was significantly greater compared to a reduction of −3.58 (14.29) in the placebo group (women=9, men=3) [F (2,21)=9.45; p=0.0058; Cohen's d=0.52]. Analysis of reliable and clinically meaningful change in the EAT-26≥11 subset sample showed that only the MDMA group yielded an RCI score indicative of reliable change (RCI=−2.16), compared to an RCI score of −0.43 for the placebo group. In women, the difference in change scores among those with baseline EAT-26≥11 was statistically significant between MDMA vs. placebo [F (2, 14)=17.68; p=0.0009; Hedge's g=0.63). Analysis of reliable and clinically meaningful change in women with EAT-26≥11 showed that the MDMA group produced an RCI score indicative of reliable change (RCI=−2.90), which was not seen in the placebo group (−0.50).

As shown in FIG. 41, in the baseline EAT-26≥20 subset sample, there was a statistically significant within-subject mean (SD) reduction in EAT-26 scores in both the placebo group (women=6) (−13.50, SD=12.35; p=0.01) and MDMA group (women=5, men=1) (−14.08, SD=8.96; p=0.0047); however, the treatment group difference was not statistically significant. Analysis of reliable and clinically meaningful change in the EAT-26≥20 subset sample showed that only the MDMA group yielded an RCI score indicative of reliable change (RCI=−2.83), compared to an RCI score of −1.85 for the placebo group. The reduction in EAT-26 score was determined to be clinically meaningful for both MDMA and placebo groups. In women, the difference in change scores among those with baseline EAT-26≥20 was statistically significant between MDMA vs. placebo [F (1, 7)=5.75; p=0.0478; Hedge's g=0.60)]. The change in EAT-26 score for women with baseline EAT-26≥20 from baseline to follow-up was indicative of reliable change (RCI=−18.80) and was determined to be clinically meaningful. Most significantly, in the placebo group, the proportion of those with EAT-26≥20 did not change from baseline to follow-up (n=6, 15.0%); whereas in the MDMA group, the proportion of participants was reduced from 5 (11.90%) to 1 (2.38%).

### Example 16. Evaluation of an Open-Label, Multi-Site Phase 2 Study of the Safety and Feasibility of MDMA-Assisted Therapy for Eating Disorders

This study will aim to evaluate the safety and feasibility of open-label MDMA-assisted therapy with a flexible dose of MDMA and adjunctive caregiver support in reducing eating disorder symptoms for 16 participants over the age of 18 with Anorexia Nervosa, Restricting-Type (AN-R), or Binge Eating Disorder (BED). The protocol was amended to update the IMP packaging in alignment with new standards and to allow a lower supplemental dose regimen of 34 mg MDMA for participants who receive an initial dose of 100 mg MDMA. Supplemental doses for any eating disorder participant (ED-P) did exceed half of the initial dose.

### Materials and Methods

The study enrolled 16 participants over the age of 18 with AN-R or BED, of which 12 participants who met DSM-5 criteria for AN-R, and 6 participants who met DSM-5 criteria for BED are eligible to enroll. Participants received a flexible dose of MDMA (75-125 mg) in conjunction with psychotherapy for three sessions. If required, supplemental doses of up to half of the initial dose were administered, with a maximum of 50 mg MDMA (equivalent to 60 mg MDMA·HCl) per supplemental dose. Participants were assessed at baseline, post-treatment, and 6-month follow-up using various measures, including the eating disorder questionnaire and depression inventory questionnaires. Adverse events were monitored and recorded throughout the study.

### Data Analysis:

Descriptive statistics were used to summarize demographic and clinical characteristics of the sample. Mixed-effects regression models were used to explore the effects of MDMA-assisted therapy on primary and secondary outcomes, accounting for site as a random effect. Adverse events were tabulated.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

1-32. (canceled)

33. A pharmaceutical composition comprising about 34 mg or about 50 mg of 1-(1,3-benzodioxol-5-yl)-N-methylpropan-2-amine hydrochloride (MDMA) on a free base basis and one or more pharmaceutically acceptable excipients, wherein the 1-(1,3-benzodioxol-5-yl)-N-methylpropan-2-amine hydrochloride is present as particles with a Dv50 from about 100 μm to about 200 μm.

34. The pharmaceutical composition of claim 33, wherein the Dv50 is from about 120 μm to about 180 μm.

35. The pharmaceutical composition of claim 33, characterized in that greater than or equal to 80% of the mass of the MDMA particles dissolves in water at 30 minutes.

36. The pharmaceutical composition of claim 33, characterized in that greater than or equal to 95% of the mass of the MDMA particles dissolves in water at 30 minutes.

37. The pharmaceutical composition of claim 33, characterized in that greater than or equal to 99% of the mass of the MDMA particles dissolves in water at 30 minutes.

38. The pharmaceutical composition of claim 33, characterized in that at least 80% of the mass of the MDMA particles dissolves in water at 15 minutes.

39. The pharmaceutical composition of claim 35, wherein the water has a pH of about 1.2, about 4.5, or about 6.8.

40. The pharmaceutical composition of claim 33, wherein the composition comprises:
about 15% to about 74.9% by weight of 1-(1,3-benzodioxol-5-yl)-N-methylpropan-2-amine hydrochloride (MDMA);
about 25% to about 75% by weight of mannitol; and
about 0.1% to about 10% by weight of magnesium stearate.

41. The pharmaceutical composition of claim 40, wherein the composition comprises about 50% by weight of the 1-(1,3-benzodioxol-5-yl)-N-methylpropan-2-amine hydrochloride, about 49% by weight of mannitol, and about 1% by weight of magnesium stearate.

42. The pharmaceutical composition of claim 40, wherein the composition comprises 50% by weight of the 1-(1,3-benzodioxol-5-yl)-N-methylpropan-2-amine hydrochloride, 49% by weight of mannitol, and 1% by weight of magnesium stearate.

43. The pharmaceutical composition of claim 40, wherein the composition comprises about 48% to about 49.9% by weight of mannitol.

44. The pharmaceutical composition of claim 40, wherein the composition comprises about 49% by weight of mannitol.

45. The pharmaceutical composition of claim 40, wherein the composition comprises about 0.1% to about 2% by weight of magnesium stearate.

46. The pharmaceutical composition of claim 40, wherein the composition comprises about 1% by weight of magnesium stearate.

47. The pharmaceutical composition of claim 40, wherein the composition consists of:
about 15% to about 74.9% by weight of 1-(1,3-benzodioxol-5-yl)-N-methylpropan-2-amine hydrochloride (MDMA) having an average particle size of 50 μm to 400 μm, a Dv90 of less than 420 μm, and a particle size range of less than 400 μm;
about 25% to about 75% by weight of mannitol; and
about 0.1% to about 10% by weight of magnesium stearate.

48. The pharmaceutical composition of claim 40, wherein the composition is in the form of a tablet.

49. The pharmaceutical composition of claim 40, wherein the composition is in the form of a capsule.

50. The pharmaceutical composition of claim 49, wherein the capsule comprises hydroxypropylmethylcellulose (HPMC).

51. The pharmaceutical composition of claim 33, wherein the composition comprises about 34 mg or about 50 mg of 1-(1,3-benzodioxol-5-yl)-N-methylpropan-2-amine hydrochloride on a free base basis.

52. The pharmaceutical composition of claim 33, wherein the composition comprises about 34 mg of 1-(1,3-benzodioxol-5-yl)-N-methylpropan-2-amine hydrochloride on a free base basis.

53. The pharmaceutical composition of claim 33, wherein the composition comprises about 50 mg of 1-(1,3-benzodioxol-5-yl)-N-methylpropan-2-amine hydrochloride on a free base basis.

54. The pharmaceutical composition of claim 33, wherein the particles of 1-(1,3-benzodioxol-5-yl)-N-methylpropan-2-amine hydrochloride comprise less than 10% 1-(1,3-benzodioxol-5-yl)-N-methylpropan-2-amine hydrochloride monohydrate.
